(19) United States
(12) Patent Application Publication
(10) Pub. No.: US 2022/0218834 A1
(43) Pub. Date: Jul. 14, 2022

(54) APOHEMOGLOBIN-HAPTOGLOBIN COMPLEXES AND METHODS OF USING THEREOF

(71) Applicants: OHIO STATE INNOVATION FOUNDATION, Columbus, OH (US); THE REGENTS OF THE UNIVERSITY OF CALIFORNIA, Oakland, CA (US)

(72) Inventors: Ivan SUSIN PIRES, Cambridge, MA (US); Andre PALMER, Bexley, OH (US); Pedro J. CABRALES AREVALO, La Mesa, CA (US)

(21) Appl. No.: 17/613,351

(22) PCT Filed: May 20, 2020
(86) PCT No.: PCT/US2020/033836

§ 371 Date: Nov. 22, 2021

Related U.S. Application Data
(60) Provisional application No. 62/850,315, filed on May 20, 2019.
(60) Provisional application No. 62/850,329, filed on May 20, 2019.
(60) Provisional application No. 62/994,736, filed on Mar. 25, 2020.

(51) Int. Cl. A61K 47/64; A61K 49/00; A61K 41/00; A61K 47/52; A61K 47/55; A61K 49/14; A61P 7/06

(52) U.S. Cl. CPC A61K 47/6445; A61K 47/64; A61K 49/0056; A61K 49/0036; A61P 7/06; A61K 41/0071; A61K 47/52; A61K 47/552; A61K 49/14; A61K 49/0032

(57) ABSTRACT

Provided herein are apohemoglobin-haptoglobin complexes as well as apohemoglobin-haptoglobin complexes comprising an active agent coordinated thereto. Methods of using these compositions are also described.

### CROSS-REFERENCE TO RELATED APPLICATIONS

This application claims benefit of priority of U.S. Provisional Application No. 62/850,315, filed May 20, 2019, U.S. Provisional Application No. 62/850,329, filed May 20, 2019, and U.S. Provisional Application No. 62/994,736, filed Mar. 25, 2020, each of which is hereby incorporated herein by reference.

### STATEMENT REGARDING FEDERALLY SPONSORED RESEARCH OR DEVELOPMENT

This invention was made with Government Support under Grant Nos. R56HL123015, R01HL126945, R01EB021926, and R01HL138116 awarded by the National Institutes of Health. The Government has certain rights in the invention.

### BACKGROUND

CD163 is a membrane receptor molecule expressed on macrophages and monocytes. In some cases, CD163 may be amongst the most highly expressed receptor on macrophages and monocytes and functions as an endocytic receptor for hemoglobin-haptoglobin complexes. In this role, CD163 is believed to take up more than 1 g of hemoglobin each day.

Various pathophysiological conditions can lead to states of hemolysis. Hemolysis is characterized by the rupture of red blood cells (RBCs), which releases toxic cell-free hemoglobin (Hb) into the circulation. In addition to cell-free Hb toxicity, the breakdown of cell-free Hb into heme and apoglobin leads to the toxic overload of free heme in vivo. Finally, breakdown of heme into iron, biliverdin and carbon monoxide can also lead to iron buildup in vivo. Hemolytic conditions include, but are not limited to, malaria, red blood cell transfusion, beta-thalassemia, sickle-cell anemia, severe burns, radiation poisoning, surgery, extracorporeal circulation and others. Currently, there lacks an effective treatment for states of hemolysis, with patients relying on blood-transfusions to replaced lost red blood cells without treatment of cell-free Hb, free heme and/or iron toxicity.

Macrophages and monocytes are part of the innate immune defence and play a central role in many infectious, autoimmune, and malignant diseases such as cancer. In autoimmune/inflammatory disease such as rheumatoid arthritis, macrophages and monocytes are the main source of inflammatory molecules such as TNF-alpha, known to be of crucial importance in disease progression. In many infectious diseases such as tuberculosis (TB) and HIV, macrophages and monocytes can also harbor infectious agents. A few malignant diseases have their origin in cells of the monocytic/macrophage lineage such as histiocytic sarcoma. Further macrophages have a central role in immune evasion of cancers and may be a key target for new and improved immunotherapies for cancer treatment.

Direct targeting of drugs to macrophages and monocytes (for example, to down-regulate production of inflammatory cytokines, to kill intracellular organisms, or to kill malignant cells) may therefore have significant impact on certain diseases without influencing other cells in the body. The targeting may therefore increase the therapeutic index of the drug.

### SUMMARY

Patients suffering from states of hemolysis may benefit from scavengers that can detoxify Hb, heme and/or free iron so that these toxic species can be neutralized and safely cleared from the body. Thus, development of new scavenger proteins for Hb, heme and iron is a promising treatment modality for states of hemolysis.

During pathophysiological conditions characterized by extensive hemolysis (e.g., sickle cell anemia, malaria, red blood cell transfusion, etc.), free heme and cell-free hemoglobin (Hb) are released into the blood stream. Once released, free heme and Hb cause a variety of side-effects such as vasoconstriction, hypertension, oxidative tissue injury and kidney damage. Thus, treatment of hemolytic conditions would benefit from scavengers of free heme and cell-free Hb such as hemopexin (Hx) and haptoglobin (Hp), respectively.

Apohemoglobin (apoHb) is a protein that is produced by removing heme from Hb. Therefore, the vacant heme-binding pockets of apoHb possess a high affinity for heme. While apoHb has shown heme-binding activity in vitro, its use for hemolysis treatment has not been explored.

A major issue with potential in vivo administration of apoHb as a heme scavenger would be its low thermal stability at physiological temperature and short circulatory half-life (would be equivalent to Hb dimers, on the order of 30 min). Similar to Hb, apoHb can react with Hp to form a stable protein complex. The apoHb-Hp complex is more stable at physiological temperature compared to free apoHb, and maintains its ability to bind heme. Further, the apoHb-Hp complex can not only scavenge heme via the bound apoHb, but could also scavenge free Hb by exchanging bound apoHb for Hb due to the irreversibility of Hb-Hp binding. These two potential mechanisms for treating the side products of hemolysis with this novel therapeutic are summarized in FIG. 24. Upon heme-binding, the resulting Hb-Hp complex is much less toxic than free heme or cell-free Hb, and is ready for in vivo clearance via CD163 mediated macrophage uptake. During states of hemolysis, Hp is quickly saturated with cell-free Hb causing a toxic buildup of cell-free Hb in the plasma. Advantageously, administration of the apoHb-Hp complex to treat states of hemolysis would not rely on the body's already depleted stores of Hp in the plasma to scavenge free heme and Hb. The general concept for the production of the apoHb-Hp complex is depicted in FIG. 25.

ApoHb can also be used for drug delivery applications. Yet, these applications would rely on the presence of Hp in the plasma for targeted drug delivery of the apoHb-drug conjugate to macrophages and monocytes and could be deterred by the instability of free apoHb at physiological temperature. Binding the apoHb-drug conjugate to Hp could prevent these issues and improve drug delivery to CD163+ macrophages and monocytes. Targeting CD163+ macrophages and monocytes would be beneficial under conditions of inflammation which induce high expression of CD163 receptors on the surface of macrophages and monocytes. Additionally, certain types of cancers (such as breast cancer) have tumor associated macrophages and monocytes with high CD163 expression which could facilitate targeted drug delivery.

A benefit for therapeutics/diagnostics delivered via apoHb-Hp is the potential for long circulatory half-life. Although Hp-Hb complexes can be quickly removed from the circulation due to the high specificity of CD163+ macrophage capture, saturation of these recepting macrophages and monocytes can prolong the long half-life of the complex. Furthermore, given one the natural functions of Hp is to prevent cell-free Hb extravasation into tissue space (due to the large size of the Hp-Hb complex), apoHb-Hp-drug complexes should have very low rates of “leakage” into the tissue space.

### DESCRIPTION OF DRAWINGS

FIG. 1 is a schematic illustration of processes used to produce active apoHb.

FIG. 2 is a schematic illustration of the apoHb TFF production process.

FIG. 3A shows the absorbance spectra of apoHb, DCNh and rHbCN

FIG. 3B shows DCNh-titration assay plots for apoHb produced via EtOH-TFF. The top graph corresponds to the processed data of the equilibrium 420 nm absorbance of a fixed apoHb concentration with increasing DCNh concentration (major and minor lines fits are shown to demonstrate the presence of an inflection point, which corresponds to the concentration of active apoHb on a per heme basis). The middle graph shows the absorbance values subtracted by the pure DCNh absorbance to highlight the inflection point determined by the apoHb assay. The bottom graph presents the residuals of the major and minor line fits.

FIG. 3C shows DCNh-titration assay plots for apoHb produced by acetone extraction. The top graph corresponds to the processed data of the equilibrium 420 nm absorbance of a fixed apoHb concentration with increasing DCNh concentration (major and minor lines fits are shown to demonstrate the presence of an inflection point, which corresponds to the concentration of active apoHb on a per heme basis). The middle graph shows the absorbance values subtracted by the pure DCNh absorbance to highlight the inflection point determined by the apoHb assay. The bottom graph presents the residuals of the major and minor line fits.

FIG. 4A shows the electrospray mass spectra of Hb under native conditions. Dotted lines indicate deconvoluted spectra. The superscripts (α/β)^{a} and (α/β)^{h} indicate the apo- or holo- protein, respectively.

FIG. 4B shows the electrospray mass spectra of Hb under acidic/denaturing conditions. Dotted lines indicate deconvoluted spectra. The superscripts (α/β)^{a} and (α/β)^{h} indicate the apo- or holo- protein, respectively.

FIG. 4C shows the electrospray mass spectra of apoHb under native conditions. Dotted lines indicate deconvoluted spectra. The superscripts (α/β)^{a} and (α/β)^{h} indicate the apo- or holo- protein, respectively.

FIG. 4D shows the electrospray mass spectra of apoHb under acidic/denaturing conditions. Dotted lines indicate deconvoluted spectra. The superscripts (α/β)^{a} and (α/β)^{h} indicate the apo- or holo- protein, respectively.

FIG. 5A shows the SEC profile of TFF-apoHb and Hb (the elution peak of Hb with UV-visible detection at 280 nm was normalized to a value of 1, and all other values were normalized so that the same mass of apoHb and Hb was shown on the chromatograms).

FIG. 5B compares the SEC elution volume of apoHb-TFF with molecular weight (MW) standards (conalbumin 76 kDa, human Hb 64 kDa, carbonic anhydrase 29 kDa, ribonuclease A 14 kDa, and aprotinin 6.5 kDa).

FIG. 5C shows apoHb samples used for residual heme analysis.

FIG. 5D shows a data table with results from the residual heme analysis of apoHb samples.

FIG. 5E shows a plot of residual heme content with cutoff curves representing 1% and 0.5% residual heme in solution.

FIG. 5F shows the SDS-PAGE of apoHb and Hb.

FIG. 5G shows the SEC profiles of apoHb, Hp and apoHb-Hp mixtures with excess Hp and excess apoHb.

FIG. 5H shows the SEC profiles within the elution region of interest of apoHb, Hp and apoHb-Hp mixtures with excess Hp and excess apoHb.

FIG. 6A shows the SEC-HPLC of concentrated (con) and unconcentrated (uncon) TFF-apoHb samples.

FIG. 6B shows the SEC-HPLC of concentrated (con) and unconcentrated (uncon) TFF-apoHb samples within the elution region of interest and magnified 10× for the tetrameric apoHb elution region.

FIG. 6C shows the RP-HPLC of concentrated (con) and unconcentrated (uncon) TFF-apoHb samples.

FIG. 6D shows the far UV CD of concentrated (con) and unconcentrated (uncon) TFF-apoHb samples.

FIG. 7A show the absorbance spectra of native Hb, pure heme, and hemichrome (the molar concentration on a per heme basis for each species was approximately 60 μM).

FIG. 7B show the absorbance spectra of rHb from TFF-apoHb, pure heme, and hemichrome (the molar concentration on a per heme basis for each species was approximately 60 μM).

FIG. 7C shows a schematic of the hemichrome removal process.

FIG. 7D shows representative absorbance spectra of rHb at each stage of the hemichrome removal process with curve fits from the spectral deconvolution software standardized to a total heme concentration of 25 μM.

FIG. 7E shows the final results from spectral deconvolution at each stage of the hemichrome removal process. The lines trace the fate of unwanted species (i.e. heme and hemichromes).

FIG. 8A show 02 equilibrium curves for native Hb, and rHb from TFF-apoHb and acetone extraction methods. Native hHb, TFF rHb and acetone rHb had P₅₀s of 11.33±0.02, 11.52±0.02 and 10.50±0.02 mm Hg and n of 2.60±0.01, 2.37±0.01 and 2.14±0.01, respectively.

FIG. 8B is a plot showing the representative O₂ dissociation kinetic time courses for native Hb and rHb produced via TFF. Data was fit to a single exponential equation to yield k_{off,O}_{₂} of 35.8±0.2 s⁻¹ and 36.8±0.3 s⁻¹ for TFF rHb and native Hb, respectively.

FIG. 8C shows representative CO association kinetic time courses for native Hb and TFF rHb.

FIG. 8D is a plot of kₐₚₚ for CO association at varying CO concentrations. Data was fit to a linear function to regress k_{on,CO} of 180±17 and 175±4 nM/s for native Hb and rHb, respectively.

FIG. 9A shows the storage of apoHb in liquid form at 37° C. Samples were stored at initial apoHb concentrations of either 33.80±0.36 mg/mL active apoHb with 41.4±2.77 mg/mL total protein (con) or 1.47±0.01 mg/mL active apoHb with 1.99±0.17 mg/mL total protein (uncon).

FIG. 9B shows the storage of apoHb in liquid form at 22° C. Samples were stored at initial apoHb concentrations of either 33.80±0.36 mg/mL active apoHb with 41.4±2.77 mg/mL total protein (con) or 1.47±0.01 mg/mL active apoHb with 1.99±0.17 mg/mL total protein (uncon).

FIG. 9C shows the storage of apoHb in liquid form at 4° C. Samples were stored at initial apoHb concentrations of either 33.80±0.36 mg/mL active apoHb with 41.4±2.77 mg/mL total protein (con) or 1.47±0.01 mg/mL active apoHb with 1.99±0.17 mg/mL total protein (uncon).

FIG. 9D shows the storage of apoHb at −80° C. Samples were stored at initial apoHb concentrations of either 33.80±0.36 mg/mL active apoHb with 41.4±2.77 mg/mL total protein (con) or 1.47±0.01 mg/mL active apoHb with 1.99±0.17 mg/mL total protein (uncon).

FIG. 9E shows the storage of apoHb in lyophilized form at −80° C.

FIG. 10A shows the RP-HPLC of stored TFF-apoHb.

FIG. 10B shows the far UV CD of stored TFF-apoHb.

FIG. 10C shows the change in UV-visible spectra of TFF-apoHb at 22° C. as a function of storage time.

FIG. 10D shows the change in UV-visible spectra of stored and fresh TFF-apoHb at 22° C.

FIG. 10E shows the SEC-HPLC of TFF-apoHb stored for more than one year at 4° C. and after transferring the year-old samples to storage at 22° C.

FIG. 10F shows the SEC-HPLC within the elution region interest of TFF-apoHb stored for more than one year at 4° C. and after transferring the year-old samples to storage at 22° C. (with 10× magnification of the elution region of tetrameric apoHb).

FIG. 11A shows the full SEC-HPLC chromatogram of a single TFF-apoHb sample at different concentrations and injection volumes.

FIG. 11B shows FIG. 11A in the region of interest for TFF-apoHb tetramers and dimers.

FIG. 11C shows the decrease in tetrameric TFF-apoHb content upon addition of Hp to the sample.

FIG. 11D shows the elution volume shift to higher elution volumes for Hb and TFF-apoHb at low protein concentrations.

FIG. 12 schematically illustrates the general process for the purification of haptoglobin via tangential flow filtration of Cohn Fraction IV without the use of fumed silica (grey arrows indicate the fluid flow direction).

FIG. 13 schematically illustrates the general process for the purification of haptoglobin via tangential flow filtration of Cohn Fraction IV using fumed silica (grey arrows indicate the fluid flow direction).

FIG. 14 schematically illustrates the general process used to purify both Hp (Stages 2 and 3), and the Hb, heme and iron scavenging protein cocktail (Stage 4). HMW=high MW fraction. LMW=low MW fraction. Arrows indicate the direction of flow.

FIG. 15A shows a representative graph for the quantification of hemoglobin binding capacity (HbBC) of Hp samples based on fluorescence titration.

FIG. 15B shows a representative graph for the quantification of hemoglobin binding capacity (HbBC) of Hp samples based on the SEC-HPLC AUC method.

FIG. 16A is a plot showing the analysis of HbBC and total protein recovery at each stage of processing based on the TFF method without the use of fumed silica.

FIG. 16B shows the HPLC-SEC analysis at each stage of the Hp purification process via tangential flow filtration of Cohn Fraction IV. Dotted lines show the Hp-Hb complexes formed with the samples exposed to excess Hb. The chromatogram of samples exposed to excess Hb (dotted lines) ends prior to the elution time of free Hb (9.6 min) given that the signal from excess free Hb peak would overwhelm the signal from the Hp-Hb peak.

FIG. 17A shows SDS-PAGE under non-reducing conditions at each of the processing stages of Hp purification via TFF of Cohn Fraction IV without the use of fumed silica. Lanes were loaded with 30 μg of protein. Densitometric analysis indicated Hp eluting bands composed of >70% of Stage 2 proteins and >75% of Stage 3 proteins.

FIG. 17B shows SDS-PAGE under reducing conditions at each of the processing stages of Hp purification via TFF of Cohn Fraction IV without the use of fumed silica. Lanes were loaded with 27 μg of protein. Densitometric analysis indicated Hp eluting bands composed of >70% of Stage 2 proteins and >75% of Stage 3 proteins.

FIG. 17C shows the normalized total ion intensity from trypsin digestion mass spectrometry at each of the processing stages of Hp purification via TFF of Cohn Fraction IV without the use of fumed silica. Abbreviations: AT: α-1 antitrypsin, ACT: α-1 antichymotrypsin, Hb: hemoglobin, Tf: transferrin, ApoA1: apolipoprotein A1, Hpr: haptoglobin-related protein, ApoA2: apolipoprotein A2, ApoJ: apolipoprotein J, HSA: human serum albumin, Hp: haptoglobin, A2M: α-2 macroglobulin, Cp: ceruloplasmin, ITH4: Inter-alpha-trypsin inhibitor H4, IgHA1: immunoglobulin heavy constant alpha 1, IgKC: immunoglobulin kappa constant, IgKLC: immunoglobulin kappa light chain, PON1: paraoxonase 1, IgHG2: immunoglobulin heavy constant gamma 2, CFB: complement factor B, AGT: angiotensinogen, Hpx: hemopexin, VDB: vitamin-D binding protein.

FIG. 17D shows the normalized total ion intensity of selected protein components. Abbreviations: AT: α-1 antitrypsin, ACT: α-1 antichymotrypsin, Hb: hemoglobin, Tf: transferrin, ApoA1: apolipoprotein A1, Hpr: haptoglobin-related protein, ApoA2: apolipoprotein A2, ApoJ: apolipoprotein J, HSA: human serum albumin, Hp: haptoglobin, A2M: α-2 macroglobulin, Cp: ceruloplasmin, ITH4: Inter-alpha-trypsin inhibitor H4, IgHA1: immunoglobulin heavy constant alpha 1, IgKC: immunoglobulin kappa constant, IgKLC: immunoglobulin kappa light chain, PON1: paraoxonase 1, IgHG2: immunoglobulin heavy constant gamma 2, CFB: complement factor B, AGT: angiotensinogen, Hpx: hemopexin, VDB: vitamin-D binding protein.

FIG. 18A shows the results from the analysis of total protein and hemoglobin binding capacity recovery after addition of fumed silica and the two washing steps.

FIG. 18B shows the HPLC-SEC chromatograms of FIV suspension and of the supernatants after addition of fumed silica and two washes with PBS. Dotted lines show the sample mixed with excess Hb.

FIG. 19A shows an analysis of total protein and HbBC recovery at each stage of processing for the TFF purification method with the use of fumed silica.

FIG. 19B shows the SEC-HPLC chromatograms of the end-product of each stage of TFF purification with the use of fumed silica. The chromatogram of samples with excess Hb (dotted lines) ends prior to the elution time of free Hb (9.6 min) given that the signal from the excess free Hb peak overwhelmed the signal from the Hp-Hb peak.

FIG. 20A shows the SDS-PAGE analysis of Hp fractions in Stages 0-3 from the TFF process used to purify Hp from FIV using fumed silica under non-reducing conditions. Stage 0 and 1 were run on 4-20% polyacrylamide gels while Stage 2 and Stage 3 were run on 10-20% polyacrylamide gels, leading to the difference in elution pattern of the polymeric species. Purity via densitometric analysis of the SDS-PAGE gels was shown to be >80% pure for Stage 2 and >95% pure for Stage 3.

FIG. 20B shows the SDS-PAGE analysis of Hp fractions in Stages 0-3 from the TFF process used to purify Hp from FIV using fumed silica under reducing conditions. Stage 0 and 1 were run on 4-20% polyacrylamide gels while Stage 2 and Stage 3 were run on 10-20% polyacrylamide gels, leading to the difference in elution pattern of the polymeric species. Purity via densitometric analysis of the SDS-PAGE gels was shown to be >80% pure for Stage 2 and >95% pure for Stage 3.

FIG. 20C shows the normalized total ion intensity from trypsin digestion mass spectrometry for Stages 0-3 of the Hp purification process via TFF of Cohn Fraction IV with the use of fumed silica. Abbreviations: AT: α-1 antitrypsin, ACT: α-1 antichymotrypsin, Hb: hemoglobin, Tf: transferrin, ApoA1: apolipoprotein A1, Hpr: haptoglobin-related protein, ApoA2: apolipoprotein A2, ApoJ: apolipoprotein J; HSA: human serum albumin, Hp: haptoglobin, A2M: α-2 macroglobulin, ITH4: inter-alpha-trypsin inhibitor H4, IgHA1: immunoglobulin heavy constant alpha 1, IgKC: immunoglobulin kappa constant, Hpx: hemopexin, VDB: vitamin-D binding protein. PZP: pregnancy zone protein, HCII: heparin cofactor II, CFB: complement factor B.

FIG. 20D shows the normalized total ion intensity from trypsin digestion mass spectrometry of selected proteins for Stages 0-3 of the Hp purification process via TFF of Cohn Fraction IV with the use of fumed silica. Abbreviations: AT: α-1 antitrypsin, ACT: α-1 antichymotrypsin, Hb: hemoglobin, Tf: transferrin, ApoA1: apolipoprotein A1, Hpr: haptoglobin-related protein, ApoA2: apolipoprotein A2, ApoJ: apolipoprotein J; HSA: human serum albumin, Hp: haptoglobin, A2M: α-2 macroglobulin, ITH4: Inter-alpha-trypsin inhibitor H4, IgHA1: immunoglobulin heavy constant alpha 1, IgKC: immunoglobulin kappa constant, Hpx: hemopexin, VDB: vitamin-D binding protein. PZP: pregnancy zone protein, HCII: heparin cofactor II, CFB: complement factor B.

FIG. 21 schematically illustrates a general procedure to remove hydrophobic ligands from proteins.

FIG. 22 schematically illustrates a general manufacturing strategy for the purification of a target protein via tangential flow filtration which employs protein complex formation using a target protein binding protein. Starting with a mixture of proteins/particulates (1) (example: cell lysate, human plasma, etc.), the mixture is filtered through a membrane with an appropriate MWCO that permeates the TP along with low MW impurities (2). Then a TPBM (i.e. antibody or equivalent, etc.) specific to the TP is introduced into the filtrate, forming a TP-TPBM protein complex that is larger than the MWCO of the original membrane (3). The solution with the newly formed TP-TPBM protein complex is then refiltered through the same MWCO membrane leading to retention of the isolated TP-TPBM protein complex of interest and removal of low MW impurities (4). The isolated TP-TPBM protein complex can then be dissociated to yield free TP and TPBM via appropriate buffer exchange under conditions that would facilitate their dissociation (5). With the individual species (TP and TPBM) dissociated in solution, the TP can be separated from the TPBM using a MWCO membrane that is between the MW of the TP and TPBM (6). Note: both the TPBM in the retentate and TP in the filtrate can be buffer exchanged via TFF into appropriate buffers to remove the dissociating agent and concentrate the separated TP and TPBM.

FIG. 23 schematically illustrates a general manufacturing strategy for the purification of low molecular weight (MW) haptoglobin (Hp) via tangential flow filtration which employs protein complex formation (in the example provided the complex mixture consisted of Cohn Fraction IV and the dissociating agent was urea).

FIG. 24 schematically illustrates the proposed mechanisms for scavenging free heme and cell-free Hb using the apohemoglobin-haptoglobin complex.

FIG. 25 is a schematic illustration of the production of the apohemoglobin-haptoglobin complex.

FIG. 26 shows the general production scheme for the purification of the Hb-Hp protein complex from Cohn fraction IV paste using TFF

FIG. 27 provides a diagram showing the dissociation of Hb from the Hb-Hp complex to isolate Hp. Numbers in brackets indicate the number of diafiltration volumes.

FIG. 28 shows an SDS-PAGE of the purified Hb-Hp complex and mixture of Hp and Hp-Hb obtained from dissociation and separation of Hb from the purified Hb-Hp complex. Lane 1: Isolated Hb-Hp complex. Lane 2: Mixture of Hp and Hb-Hp. Lane 3: 100 kDa permeate. Lane 4: 100 kDa permeate with added Hb. Abbreviations: transferrin (Tf); haptoglobin (Hp), human serum albumin (HSA), hemoglobin (Hb), beta chain of haptoglobin (β Hp), alpha chain of haptoglobin (a Hp).

FIGS. 29A-J compare the hypothetical and experimentally measured HPLC-SEC elution chromatogram at each stage of the Hb-Hp purification process. Human Cohn Fraction IV was used as source of Hp (the TP) and after filtering the protein mixture through a 100 kDa TFF module, Hb was added as a TPBM to form the Hb-Hp complex (TP-TPBM) and the sample was re-filtered through the original 100 kDa TFF module. FIG. 29A shows hypothetical HPLC-SEC protein elution chromatogram for Stage 1 (protein mixture). FIG. 29B shows experimental HPLC-SEC protein elution chromatogram for Stage 1 (protein mixture). FIG. 29C shows hypothetical (HPLC-SEC protein elution chromatogram for Stage 1*(protein mixture and TPBM). FIG. 29D shows experimental (HPLC-SEC protein elution chromatogram for Stage 1*(protein mixture and TPBM). FIG. 29E shows hypothetical HPLC-SEC protein elution chromatogram for Stage 2 (low MW mixture). FIG. 29F shows experimental HPLC-SEC protein elution chromatogram for Stage 2 (low MW mixture). FIG. 29G shows hypothetical HPLC-SEC protein elution chromatogram for Stage 3 (low MW mixture and TPBM). FIG. 29H shows experimental HPLC-SEC protein elution chromatogram for Stage 3 (low MW mixture and TPBM). FIG. 29I shows hypothetical HPLC-SEC protein elution chromatogram for Stage 4 (isolated TP-TPBM complex). FIG. 29J shows experimental HPLC-SEC protein elution chromatogram for Stage 4 (isolated TP-TPBM complex).

FIG. 30 shows combined HPLC-SEC chromatograms at different stages of the Hb-Hp purification process

FIG. 31 is the HPLC-SEC chromatogram of apoHb, Hp and apoHb-Hp (with excess apoHb) solutions. The full elution chromatogram appears in the top graph, and elution between 6.5 and 11 min appears in the bottom graph. Wavelength detection was set to 280 nm to detect protein, and 405 nm to detect residual Hb or heme.

FIG. 32 is the UV-visible absorbance spectra of apoHb, Hp, and Al-PC mixtures between 200 and 700 nm (top) and 300-700 nm (bottom).

FIG. 33 is the absorbance spectra of apoHb, Hp, and Mn-IX mixtures between 200 and 700 nm (top) and 300-700 nm (bottom).

FIG. 34 shows proposed mechanisms for scavenging heme and cell-free Hb via the apoHb-Hp complex.

FIGS. 35A and 35B show HPLC-SEC of apoHb binding to Hp. FIG. 35A is an HPLC-SEC of apoHb, Hp and mixtures of apoHb and Hp. FIG. 35B shows a close-up of elution curves of apoHb, Hp and apoHb-Hp.

FIGS. 36A and 36B show HPLC-SEC of Hb exchange for apoHb bound to Hp and heme exchange from heme-albumin to apoHb bound to Hp. FIG. 36A is an HPLC-SEC of apoHb, Hp, apoHb-Hp and mixtures of apoHb-Hp with Hb. FIG. 36B is an HPLC-SEC of mixtures of apoHb-Hp with heme-albumin (heme-HSA).

FIGS. 37A-37C show HoloHb exchange for apoHb in the apoHb-Hp complex in the plasma compartment of guinea pigs. FIG. 37A shows the plasma concentration versus time (0-360 minutes) of total holoHb, Hp bound to holoHb, and unbound holoHb and indicates nearly complete holoHb exchange for apoHb within the initial minutes following apoHb-Hp administration. FIG. 37B shows AUC₀₋₃₆₀ₘᵢₙ values for total holoHb (Hp bound+unbound), Hp bound holoHb and unbound holoHb. Significant differences were observed between AUC₀₋₂₆₀ₘᵢₙ for total versus free holoHb (p<0.0001) and Hp bound versus free holoHb (p<0.0001), based on a one-way ANOVA with a multiple comparisons test for parametric data (n=5/comparison). FIG. 37C shows HPLC-SEC traces (0-60 minutes) showing holoHb completely replacing apoHb in the Hb-Hp complex after the administration of holoHb and apoHb-Hp to guinea pigs. Representative plasma samples with holoHb (denoted (Hb)), apoHb-Hp complex (no absorbance at 413 nm) and the rapid appearance of Hp bound holoHb (denoted Hb-Hp) within the initial post dosing plasma samples, indicating rapid binding and detection (at λ=413 nm) of holoHb in exchange for apoHb. Note—representative chromatograms are shown until 60 minutes, due to rapid and complete holoHb exchange for apoHb.

FIGS. 38A-38C show heme transfer from heme-albumin to apoHb in the apoHb-Hp complex in the plasma compartment of guinea pigs. FIG. 38A shows plasma concentration versus time (0-360 minutes) of total heme, transferred heme and non-transferred heme and indicates transfer of heme over the initial six hours post heme-albumin and apoHb-Hp administration. FIG. 38B shows AUC values for total heme (transferred+non-transferred), heme (transferred), and heme (non-transferred). Significant differences were observed between AUC₀₋₃₆₀ₘᵢₘ values for total versus non-transferred heme (p<0.0001) and between AUC₀₋₃₆₀ₘᵢₙ values for transferred versus non-transferred heme (p=0.0003), based on a One-way ANOVA with a multiple comparisons test for parametric data (n=5/comparison). FIG. 38C shows HPLC-SEC traces (0-360 minutes) showing heme-albumin, apoHb-Hp (no absorbance at 413 nm) in the Hb-Hp complex after the administration of heme-albumin followed by apoHb-Hp to guinea pigs. Representative plasma samples show the transfer of heme (at λ=413 nm) from heme-albumin to apoHb bound to Hp to ultimately generate holoHb-Hp (denoted Hb-Hp).

FIGS. 39A-39C show data for Systemic parameters (MAP and HR) and microcirculatory parameter (FCD) for measurements made during baseline, following pretreatment and following Hb challenge for all experimental groups: the control, pretreatment with only apoHb, pretreatment with only Hp, and pretreatment with the apoHb-Hp complex. FIG. 39A shows mean arterial pressure (MAP), FIG. 39B shows heart rate (HR), and FIG. 39C shows functional capillary density (FCD). Significant differences are indicated with symbols: † compared to BL and ‡ compared to treatment (P<0.05).

FIGS. 40A-40D show data related to diameter, velocity and flow of small, medium, large-sized arteriole vessels and venules—in FIG. 40A for small-sized arteriole vessels (20-40 μm), in 40B for mid-sized arteriole vessels (40-60 μm), in 40C for large arterioles (60-100 μm), and in 40D for venules (30-80 μm)—relative to baseline during baseline, following pretreatment and following Hb challenge for all experimental groups: the control, pretreatment with only apoHb, pretreatment with only Hp, and pretreatment with the apoHb-Hp complex. † compared to BL and ‡ compared to dosing groups (P<0.05).

FIGS. 41A-41E show systemic hemodynamic and microcirculatory response to heme-albumin and attenuation by apoHb-Hp. Measurements were made relative to baseline during baseline, following pretreatment and following heme-albumin challenge for experimental groups—control, pretreatment with the apoHb-Hp complex. FIG. 41A shows Mean arterial pressure (MAP) and FIG. 41B heart rate (HR). Microcirculatory changes: FIG. 41C shows arteriolar diameter, FIG. 41D shows arteriolar blood flow and FIG. 41A shows functional capillary density (FCD). Significant differences are indicated within group comparisons (P<0.05).

FIG. 42 is an illustration of the role of Hp in inhibiting extravasation of low MW PolyHb species. (a) Hb (1) binds with Hp (2), which significantly increases the size of the resulting Hb-Hp complex (3). Similarly, PolyHb (4) can bind with Hp (2) to form a PolyHb-Hp complex with increased molecular diameter. (b) In circulation, low MW PolyHb (6) can freely extravasate through the endothelial cell wall (8) via the endothelial gap junction (9). Once past the endothelial wall, the extravasated PolyHb (10) accumulates in the intima (11) where it scavenges the NO produced by the endothelial cells that regulate smooth muscle cell (12) contraction. (c) When PolyHb binds to Hp the resulting PolyHb-Hp complexes (13) are too large to pass through the endothelial gap junction. This effectively limits PolyHb extravasation into the tissue space and NO scavenging.

FIGS. 43A-43F shows the biophysical properties of the PolyHb used in this study. FIG. 43A shows the 02 equilibrium curves for PolyHb and Hb with 3 runs per sample. FIG. 43B shows a comparison of the time course for deoxygenation in the presence of 1.5 mg/mL sodium dithionite for oxygenated Hb and PolyHb. For deoxygenation, the reactions were monitored at 437.5 nm and 20° C. in 0.1 M pH 7.4 PBS. FIG. 43C shows the time courses for the NO dioxygenation reaction with Hb and PolyHb at 12.5 μM NO and 25 μM NO. Dots represent experimental data, and the corresponding solid lines of the same color represent curve fits to the data. NO dioxygenation reactions were monitored at 420.0 nm and 20° C. in 0.1 M pH 7.4 PBS. FIG. 43D shows a comparison of the NO dioxygenation rates for hHb, and PolyHb. For kinetics, the data shows an average of 10 kinetic traces for each sample. The error bars indicate the standard deviation from 10 replicates. FIG. 43E shows a representative intensity distribution of the hydrodynamic diameter of PolyHb. FIG. 43F shows a summary of the biophysical properties of unmodified Hb and PolyHb.

FIGS. 44A-44D show HPLC-SEC and rapid kinetics of Hp binding to Hb and PolyHb. FIG. 44A shows HPLC-SEC chromatograms of Hb and PolyHb with and without Hp. The absorbance was monitored at 413 nm to detect heme. The peak for unmodified Hb elutes at ˜9.6 minutes. Each chromatogram was normalized to the peak area under the curve prior to Hp addition. The molar ratio of Hb to Hp was 1.5:1. The molar ratio of PolyHb to Hp was 1:2. FIG. 44B shows the percent composition based on the approximate size order was determined with a gaussian deconvolution of the resulting chromatograms. FIG. 44C shows time courses of Hp (0.25 Hb tetramer binding basis) and Hb/PolyHb (on a Hb tetramer molar basis) were fit to monoexponential equations (dashed lines). Experimental data shows an average of 10 kinetic traces. The reactions were monitored by the fluorescence emission using a 310 nm high-pass filter at 20° C. PBS (0.1 M, pH 7.4) was used as the reaction buffer. FIG. 44D shows second-order rate constants of Hp binding to Hb/PolyHb derived as a function of Hb concentration on a Hb tetramer molar basis.

FIGS. 45A and 45B show systemic hemodynamics measured throughout the study. (FIG. 45A) HR and (FIG. 45B) MAP measured at baseline, after administration of saline, apoHb, or apoHb-Hp, and after 20% isovolemic exchange transfusion of PolyHb. Top panels show the measured values. Bottom panels show values normalized to the baseline of the same animal. Grey lines connect measurements obtained in the same animal. * : P<0.05 compared between groups. a: P<0.05 compared to the saline administration group at the same timepoint. b: P<0.05 compared to apoHb administration group at the same timepoint. (n=5 animals per group).

FIGS. 46A and 46B show the diameters of blood vessels measured with intravital microscopy. (FIG. 46A) Arteriole and (FIG. 46B) venule diameters measured at baseline, after administration of saline, apoHb, or apoHb-Hp, and after 20% isovolemic exchange transfusion of PolyHb. Top panels show the measured values. Bottom panels show values normalized to the baseline of the same vessel in the same animal. Grey lines connect measurements obtained in the same blood vessel. * : P<0.05 compared between groups. a: P<0.05 compared to the saline administration group at the same timepoint. (n=5 animals per group, 6 vessels per animal).

FIGS. 47A-47D show blood velocity and flow rates in blood vessels measured with intravital microscopy. (FIG. 47A) Arteriole and (FIG. 47B) venule flow velocity measured at baseline, after administration of saline, apoHb, or apoHb-Hp, and after 20% isovolemic exchange transfusion of PolyHb. (FIG. 47C) Arteriole and (FIG. 47D) venule volumetric flow rates are also shown at the same conditions. Top panels show the measured values. Bottom panels show values normalized to the baseline of the same vessel in the same animal. Grey lines connect measurements in the same blood vessel. * : P<0.05 compared between groups. a: P<0.05 compared to the saline administration group at the same timepoint. b: P<0.05 compared to the apoHb administration group at the same timepoint. (n=5 animals per group, 6 vessels per animal).

FIG. 48 shows FCD measured at baseline, after administration of saline, apoHb, or apoHb-Hp, and after 20% isovolemic exchange transfusion of PolyHb. Top panels show the measured values. Bottom panels show values normalized to the baseline FCD of the same animal. Grey lines connect measurements obtained in the same animal. * : P<0.05 compared between groups. a: P<0.05 compared to the saline administration group at the same timepoint. b: P<0.05 compared to the apoHb administration group at the same timepoint. (n=5 animals per group).

FIG. 49 depicts a general schematic for the synthesis of the apoHb-Al—PC-Hp (APH) complex.

FIG. 50 is a schematic describing the production of apoHb-Al—PC-Hp using TFF (top) and flow chart for assessing further Al-PC addition (bottom).

FIGS. 51A-51C depict Al-PC binding to apoHb and synthesis of the APH complex. FIG. 51A is a UV-visible absorbance spectra of Al-PC in EtOH, PBS, and bound to apoHb in PBS (0.77 μM of Al-PC was added to each solution; the apoHb concentration was set to ˜18 μM). Addition of Al-PC to pure EtOH (FIG. 51B) and to apoHb in PBS (FIG. 51C).

FIGS. 52A and 52B depict production of the final APH product. FIG. 52A are absorbance spectra of repeated additions of Al-PC to the APH complex before diafiltration and the absorbance spectra after diafiltration of the last addition (normalization to account for slight volume differences after each Al-PC addition). Absorbance at 680 nm after each addition of Al-PC into APH in PBS (inset of A). FIG. 52B are absorbance and fluorescence spectra of APH. Sample at ˜27 μM (based on the apoHb concentration), reaching an absorbance of ˜2.0 AU/cm at 680 nm (˜1.3 mg/mL of total APH with ˜13 μM of Al-PC).

FIGS. 53A-53E depict the size and stability of APH. Hydrodynamic diameter of pure apoHb (FIG. 53A), pure Hp (FIG. 53B) and APH (FIG. 53C) measured via DLS. HPLC-SEC elution chromatograms for the three species (FIG. 53D). Retention of Al-PC in the APH complex incubated at 37° C. in human plasma and PBS (FIG. 53E).

FIGS. 54A-54C depict the comparison of Al-PC binding to apoHb at 0.35 mg/mL and Hp at 1.0 mg/mL (FIG. 54A); HSA at 0.35 and 3.5 mg/mL (FIG. 54B); and rHb at 0.33 mg/mL and Hb at 0.35 mg/mL (FIG. 54C).

FIGS. 55A-55E depict second order rate constant determination for displacement of Al-PC from the APH complex by heme in heme-albumin. Decrease in 680 nm absorbance over time at (FIG. 55A) 37° C. and (FIG. 55C) 4° C. Pseudo-first order rate constant at different concentrations of heme-albumin at (FIG. 55B) 37° C. and (FIG. 55D) 4° C. (FIG. 55E) Second order rate constant comparison at 37° C. and 4° C.

FIGS. 56A and 56B depict: (FIG. 56A) Generation of singlet oxygen by 1 μM of Al-PC in ethanol or PBS compared to singlet oxygen generation by APH (at an equivalent 1 μM concentration of Al-PC) in PBS under irradiation with a laser (λ=670 nm, energy density of 0.75 J/cm²); all groups were significantly different (p<0.05). (FIG. 56B) In vitro cell uptake of Al-PC from APH by murine and human cancerous cells and murine and human noncancerous cells. Cell-lines: murine cancer—4T1; human cancer—MDA-MB-231; murine normal—NOR-10; human normal—MCF 10A.

FIGS. 57A-57D depict cell viability of murine and human cancerous cells and murine and human noncancerous cells exposed to (FIG. 57A) laser alone (λ=670 nm) at various energy densities, (FIG. 57B) apoHb-Hp without laser irradiation, (FIG. 57C) Al-PC without laser irradiation, and (FIG. 57D) APH without laser irradiation. Cell-lines: murine cancer—4T1; human cancer—MDA-MB-231; murine normal—NOR-10; human normal—MCF 10A.

FIGS. 58A-58E depict dell viability of murine (FIG. 58A) and human (FIG. 58B) cancerous cells exposed to APH (0.165 μM equivalent concentration of Al-PC) and laser irradiation (λ=670 nm) at various energy densities. (FIG. 58C) DNA fragmentation and cell death via apoptosis (FIG. 58D) or necrosis (FIG. 58E) after PDT with exposure of cells to 0.165 μM equivalent concentration of Al-PC using APH for 30 minutes followed by the application of laser irradiation (λ=670 nm) at different energy densities. MC—murine cancer cells (4T1). HC—human cancer cells (MDA-MB-231). MN—murine normal cells (NOR-10). HN—human normal cells (MCF 10A).

FIG. 59 depicts UV-visible absorbance spectra of apoHb in PBS at increasing dilutions with Al-PC in 100% EtOH. ApoHb at −0.35 mg/mL.

FIG. 60 depicts the reaction of Al-PC with apoHb monitored via UV-visible absorbance spectrometry. 200 μL of Al-PC in EtOH was mixed with 2 mL of apoHb in PBS at −0.35 mg/mL.

FIG. 61 depicts UV-visible absorbance spectra of apoHb-Al-PC before and after buffer exchange using dialysis.

FIG. 62 is the HPLC-SEC chromatogram of apoHb, Hp, apoHb-Hp, and Mn-IX mixtures. Wavelength detection was set to 280 nm to detect protein, and 486 nm to detect Mn-IX. The full elution chromatogram appears in the top graph, and elution between 6 and 11 min without 280 nm detection appears in the bottom graph.

FIG. 63 is the HPLC-SEC chromatogram of apoHb, Hp, apoHb-Hp, and Al-PC mixtures. Wavelength detection was set to 280 nm to detect protein, and 680 nm to detect Al-PC. The full elution chromatogram appears in the top graph, and elution between 6 and 11 min without 280 nm detection appears in the bottom graph.

FIGS. 64A-B show the toxicity studies with apoHb-Hp treatment in β-thalassemia mice. FIG. 64A shows the animal body weight at baseline (BL) and after six weeks of treatment with apoHb-Hp or vehicle. FIG. 64B shows the animal body weight tracked continuously over six weeks of treatment with apoHb-Hp or vehicle (Mean is presented; error bars omitted for clarity). N=8/group

FIGS. 65A-B show liver and spleen weight and liver functions tests in β-thalassemia mice treated with apoHb-Hp. FIG. 65A shows liver and spleen weight after six weeks of apoHb-Hp treatment compared to the vehicle control. FIG. 65B shows liver function panel focusing on alanine amino transferase (ALT), aspartate amino transferase (AST), and alkaline phosphatase (ALP) after six weeks of apoHb-Hp treatment compared to the vehicle control. N=8/group

FIGS. 66A-F shows red-blood cell parameters in β-thalassemia mice treated with apoHb-Hp. FIG. 66A shows the RBC count. FIG. 66B show the Hb concentration. FIG. 66C shows the hematocrit. FIG. 66D shows RBC count relative to baseline. FIG. 66E shows Hb concentration relative to baseline. FIG. 66F shows the hematocrit relative to baseline. Values were measured every two weeks starting at baseline (BL) for apoHb-Hp treated animals and the vehicle. Symbols: † compared to BL ‡, compared to week 2 and &, compared to week 4 (P<0.05). N=8/group.

FIGS. 67A-D show reticulocyte percentage and RBC distribution width percentage measured every two weeks starting at baseline (BL) for apoHb-Hp treated animals and the vehicle control treated animals. FIG. 67A shows reticulocyte percentage. FIG. 67B shows the RBC distribution width percentage. FIG. 67C shows the reticulocyte percentage relative to baseline. FIG. 67D shows the RBC distribution width relative to baseline. †, compared to BL (P<0.05). N=8/group

FIGS. 68A-D shows serum iron concentration, transferrin saturation, serum transferrin concentration and saturated transferrin at the third and sixth week of apoHb-Hp treatment compared to the vehicle control. FIG. 68A shows serum iron concentration. FIG. 68B shows transferrin saturation. FIG. 68C shows serum transferrin concentration. FIG. 68D shows saturated transferrin. †, compared to week 3 (P<0.05). N=8/group

FIGS. 69A-F shows representative liver and spleen iron staining and iron quantification after six weeks of apoHb-Hp treatment. FIG. 69A shows representative liver iron staining in apoHb-Hp treated animals. FIG. 69B shows representative liver iron staining in vehicle control. FIG. 69C shows total iron per gram of tissue in the liver after six weeks of apoHb-Hp treatment compared to the vehicle control. FIG. 69D shows representative spleen iron staining after six weeks of apoHb-Hp treatment. FIG. 69E shows representative spleen iron staining after six weeks on the vehicle control. FIG. 69F shows total iron per gram of tissue in the spleen after six weeks of apoHb-Hp treatment compared to the vehicle. Blue stained areas represent sites of iron accumulation. Data are presented as mean±SD. N=4 animals/group.

FIG. 70 shows the proposed mechanism of action in apoHb-Hp treatment of β-thalassemia. ApoHb-Hp scavenges cell-free Hb and heme, which prevents the cascade of effects that lead to more severe spleen and liver damage and associated pathophysiologies. Red lines indicate the effects of cell-free Hb and heme directly reduced upon apoHb-Hp treatment. Orange lines indicate the secondary effects of apoHb-Hp treatment.

### DETAILED DESCRIPTION

### Definitions

As used herein, the term “tangential-flow filtration” refers to a process in which the fluid mixture containing the components to be separated by filtration is recirculated at high velocities tangential to the plane of the filtration membrane to reduce fouling of the filter. In such filtrations a pressure differential is applied along the length of the filtration membrane to cause the fluid and filterable solutes to flow through the membrane (i.e. filter). This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is continually processed downstream.

As used herein, the term “ultrafiltration” is used for processes employing membranes rated for retaining solutes having a molecular weight between about 1 kDa and 1000 kDa.

As used herein, the term “reverse osmosis” refers to processes employing membranes capable of retaining solutes of a molecular weight less than 1 kDa such as salts and other low molecular weight solutes.

As used herein, the term “microfiltration” refers to processes employing membranes in the 0.1 to 10 micron pore size range.

As used herein, the expression “transmembrane pressure” or “TMP” refers to the pressure differential gradient that is applied along the length of a filtration membrane to cause fluid and filterable solutes to flow through the filter.

The term “hydrophobic,” as used herein, refers to a ligand which, as a separate entity, exhibits a higher solubility in a non-aqueous solution (e.g., octanol) than in water.

The term “conjugated protein,” as used herein, refers to a protein complex that includes an apoprotein and one or more associated hydrophobic ligands. The one or more hydrophobic ligands may by covalently or non-covalently associated with the apoprotein. Examples of conjugated proteins include, for example, lipoproteins, glycoproteins, phosphoproteins, hemoproteins, flavoproteins, metalloproteins, phytochromes, cytochromes, opsins, and chromoproteins.

The phrase “mild denaturing,” as used herein refers to a process which reversibly disrupts the secondary, tertiary, and/or quaternary structure of the conjugated protein, thereby facilitating separation of the hydrophobic ligand from the apoprotein. Mild denaturing can be distinguished from harsher conditions, which cleave the peptide backbone, primarily produce insoluble protein upon denaturation/renaturation, and/or disrupt protein structure to a degree such that the protein loses its biological function upon refolding.

The terms “isolating,” “purifying,” and “separating,” as used interchangeably herein, refer to increasing the degree of purity of a polypeptide or protein of interest or a target protein from a composition or sample comprising the polypeptide and one or more impurities (e.g., additional proteins or polypeptides).

Apohemoglobin-Haptoglobin Complexes

Provided herein are apohemoglobin-haptoglobin (apoHb-Hp) complexes.

In some embodiments, the apoHb-Hp complex can comprise apohemoglobin (apoHb) and haptoglobin (Hp) at a weight ratio of at least 1:1 (e.g., at least 1:1.1, at least 1:1.2, at least 1:1.3, at least 1:1.4, at least 1:1.5, at least 1:1.6, at least 1:1.7, at least 1:1.8, at least 1:1.9, at least 1:2, at least 1:2.1, at least 1:2.2, at least 1:2.3, at least 1:2.4, at least 1:2.5, at least 1:2.6, at least 1:2.7, at least 1:2.8, at least 1:2.9 or at least 1:3). In some embodiments, the apoHb-Hp complex can comprise apoHb and Hp at a weight ratio of 1:3 or less (e.g., 1:2.9 or less, 1:2.8 or less, 1:2.7 or less, 1:2.6 or less, 1:2.5 or less, 1:2.4 or less, 1:2.3 or less, 1:2.2 or less, 1:2.1 or less, 1:2 or less, 1:1.9 or less, 1:1.8 or less, 1:1.7 or less, 1:1.6 or less, 1:1.5 or less, 1:1.4 or less, 1:1.3 or less, 1:1.2 or less, or 1:1.1 or less).

The apoHb-Hp complex comprises apoHb and Hp at a weight ratio ranging from any of the minimum values described above to any of the maximum values described above. For example, in some embodiments, the apoHb-Hp complex can comprise apoHb and Hp at a weight ratio of from 1:1 to 1:3 (e.g., from 1:1.5 to 1:2.5, or from 1:1.7 to 1:2.2, or from 1:2.5 to 1:3).

In some embodiments, the Hp can be prepared using the ultrafiltration methods described below. For example, Hp can be prepared from plasma or fraction thereof (e.g., plasma fraction IV, plasma fraction V, a fraction of precipitated plasma (from salting out, or equivalent) or a combination thereof).

In certain embodiments, the Hp can have an average molecular weight of at least 70 kDa (e.g., at least 80 kDa, at least 90 kDa, at least 100 kDa, at least 150 kDa, at least 200 kDa, at least 250 kDa, at least 300 kDa, at least 350 kDa, at least 400 kDa, at least 450 kDa, at least 500 kDa, at least 550 kDa, at least 600 kDa, at least 650 kDa, at least 700 kDa, at least 750 kDa, at least 800 kDa, at least 850 kDa, at least 900 kDa, or at least 950 kDa). In certain embodiments, the Hp can have an average molecular weight of 1,000 kDa or less (e.g., 950 kDa or less, 900 kDa or less, 850 kDa or less, 800 kDa or less, 750 kDa or less, 700 kDa or less, 650 kDa or less, 600 kDa or less, 550 kDa or less, 500 kDa or less, 450 kDa or less, 400 kDa or less, 350 kDa or less, 300 kDa or less, 250 kDa or less, 200 kDa or less, 150 kDa or less, or 100 kDa or less).

The Hp can have an average molecular weight ranging from any of the minimum values described above to any of the maximum values described above. For example, in some embodiments, the Hp can have an average molecular weight of from 70 kDa to 1,000 kDa (e.g., from 80 kDa to 1,000 kDa, from 90 kDa to 800 kDa, from 80 kDa to 1,000 kDa, or from 80 kDa to 800 kDa).

In some embodiments, the apoHb can be prepared using the ultrafiltration methods described below. The apoHb prepared by various methods possess the same chemical identity (primary structure) and primarily the same quaternary conformation compared to apoHb prepared by existing precipitation or liquid-liquid extraction methodologies. The apoHb produced by the ultrafiltration methods described herein can exist in aqueous solution primarily as an αβ dimer without the use of reducing agents (2-mercaptoethanol, dithiothreitol). In contrast, previous methodologies may produce non-native tetramers (β₂β₂) that require reducing agents to form αβ dimers. Furthermore, the apoHb produced in the current methodology is stable for over a week at room temperature and stable at 4° C., −80° C. and in lyophilized form. Previous methodologies produced apoHb that quickly precipitated (approximately 24 hours) when stored at room temperature. In certain embodiments, the apoHb can be characterized by a residual Soret peak having a maximum absorption ranging from 411-417 nm, such as 412 nm (after renaturation/neutralization, but before complexation with Hp). Previous methodologies produced apoHb which had a residual Soret peak at 402-407 nm.

The apoHb-Hp complex can be formed by combining apoHb and Hp at an appropriate weight ratio. By way of example, since apoHb and Hp bind at a 1:1 molar ratio (apoHb αβ dimer binds to an αβ Hp dimer) that equates to 1:1 to 1:3 mass ratio depending on the Hp preparation and/or phenotype. ApoHb-Hp complexes can be formed by mixing apoHb and Hp at a weight ratio of at least 1:1 (e.g., at least 1:1.5, at least 1:2, or at least 1:3). By mixing an excess of apoHb with Hp, saturation of Hp Hb-binding sites can be achieved. Following complexation, the apoHb-Hp complex can be purified using tangential flow filtration (e.g., diafiltration using a 70 kDa TFF module to remove excess apoHb).

The apoHb and Hp can be wild-type proteins, recombinant proteins, or mutants. In certain embodiments, the apoHb can comprise an apoHb mutant which exhibits enhanced stability. Such mutants are known in the art, and described for example in U.S. Pat. No. 7,803,912 to Olson et al. which is incorporated herein by reference. In some examples, the apoHb can include one or more of the following amino acid mutations (the amino acids are specified by their helical location, i.e., A13 represents the thirteenth position along the A helix): α GlyA13 to Ala or Ser; α GlyB3 to Ala, Asp, Glu, or Asn; α CysG11 to Ser, Thr, or Val; β GlyA13 to Ala or Ser; β ProD2 to Ala; β GlyD7 to Lys; β GlyE13 to Ala, Thr, or Asp; βCysG14 to Val, Thr, Ser, or 11e; β ProH3 to Glu, Ala, or Gln; β CysG14 to Thr; β HisG18 to Ile, Leu, or Ala; β ProH3 to Glu; β TyrH8 to Trp or Leu; β ValH11 to Met, Leu, or Phe; or any combination thereof. Other apohemoglobins include, for example, α(H58L/V62F); β(H63L/V67F); αH87G; βH92G; βN108K; αV96W; and combinations thereof.

The apoHb-Hp complex can further include one or more active agents coordinated to the apoHb-Hp complex. In some cases, the active agent can be non-covalently associated with the apoHb-Hp complex. For example, in some cases, the active agent can be a hydrophobic active agent that non-covalently associates with the heme-binding region of apoHb. In other cases, the active agent can be covalently attached to the apoHb, covalently attached to the Hp, or a combination thereof.

In other embodiments, the active agent can be chemically linked (e.g., covalently bound) to an apohemoglobin-binding molecule such as heme. Such a system may present advantages for manufacturing as various drugs are hydrophobic which hinders their use in aqueous chemistry with proteins. Thus, the hydrophobic drugs could be chemically linked to an apohemoglobn-binding molecule, such as heme, in non-aqueous solvents. The linked active agent can then be solubilized via the binding of the apohemoglobin-binding molecules to the heme-binding pocket of apohemoglobin. In such embodiments, the active agent can be covalently tethered using the linking groups described below.

In certain embodiments, active agent can be covalently tethered to the apoHb, Hp, or a combination thereof via a linking group. When present, the linking group can be any suitable group or moiety which is at minimum bivalent, and connects the active agent to the protein. The linking group can be composed of any assembly of atoms, including oligomeric and polymeric chains. In some cases, the total number of atoms in the linking group can be from 3 to 200 atoms (e.g., from 3 to 150 atoms, from 3 to 100 atoms, from 3 and 50 atoms, from 3 to 25 atoms, from 3 to 15 atoms, or from 3 to 10 atoms).

In some embodiments, the linking group can be, for example, an alkyl, alkoxy, alkylaryl, alkylheteroaryl, alkylcycloalkyl, alkylheterocycloalkyl, alkylthio, alkylsulfinyl, alkyl sulfonyl, alkylamino, dialkylamino, alkylcarbonyl, alkoxycarbonyl, alkylaminocarbonyl, dialkylaminocarbonyl, or polyamino group. In some embodiments, the linking group can comprise one of the groups above joined to one or both of the moieties to which it is attached by a functional group. Examples of suitable functional groups include, for example, secondary amides (—CONH—), tertiary amides (—CONR—), secondary carbamates (—OCONH—; —NHCOO—), tertiary carbamates (—OCONR—; —NRCOO—), ureas (—NHCONH—; —NRCONH—; —NHCONR—, or —NRCONR—), carbinols (—CHOH—, —CROH—), ethers (—O—), and esters (—COO—, —CH₂O₂C—, CHRO₂C—), wherein R is an alkyl group, an aryl group, or a heterocyclic group. For example, in some embodiments, the linking group can comprise an alkyl group (e.g., a C₁-C₁₂ alkyl group, a C₁-C₈ alkyl group, or a C₁-C₆ alkyl group) bound to one or both of the moieties to which it is attached via an ester (—COO—, —CH₂O₂C—, CHRO₂C—), a secondary amide (—CONH—), or a tertiary amide (—CONR—), wherein R is an alkyl group, an aryl group, or a heterocyclic group. In certain embodiments, the linking group can be chosen from one of the following:

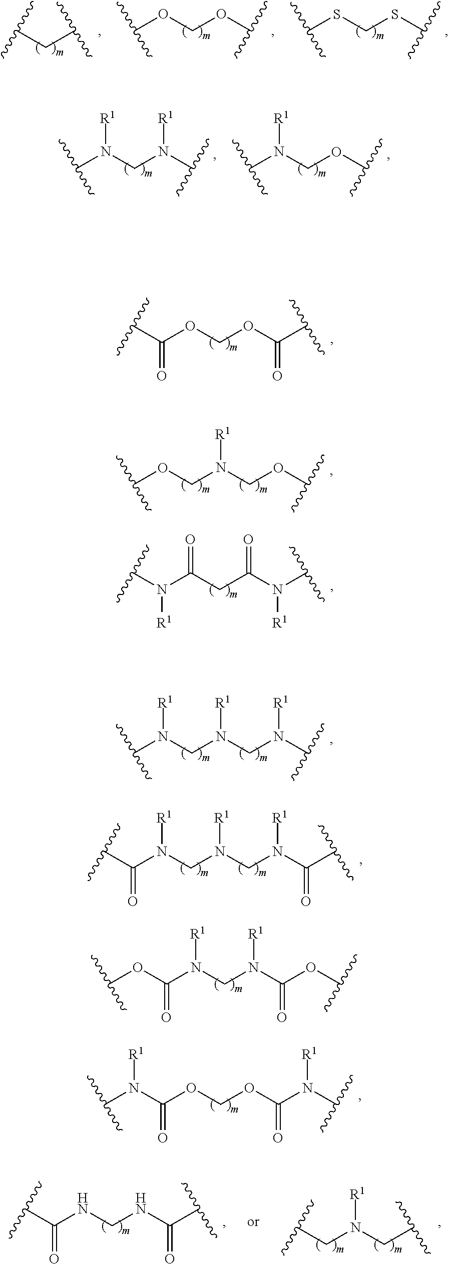

where m is an integer from 1 to 12 and R¹ is, independently for each occurrence, hydrogen, an alkyl group, an aryl group, or a heterocyclic group.

If desired, the linker can serve to modify the solubility of the apoHb, Hp, and/or the apoHb-Hp complex. In some embodiments, the linker can be hydrophilic. In some embodiments, the linker can be an alkyl group, an alkylaryl group, an oligo- or polyalkylene oxide chain (e.g., an oligo- or polyethylene glycol chain), or an oligo- or poly(amino acid) chain.

In certain embodiments, the linker can be cleavable (e.g., cleavable by hydrolysis under physiological conditions, enzymatically cleavable, or a combination thereof). Examples of cleavable linkers include a hydrolysable linker, a pH cleavage linker, an enzyme cleavable linker, or disulfide bonds that are cleaved through reduction by free thiols and other reducing agents; peptide bonds that are cleaved through the action of proteases and peptidase; nucleic acid bonds cleaved through the action of nucleases; esters that are cleaved through hydrolysis either by enzymes or through the action of water in vivo; hydrazones, acetals, ketals, oximes, imine, aminals and similar groups that are cleaved through hydrolysis in the body; photo-cleavable bonds that are cleaved by the exposure to a specific wavelength of light; mechano-sensitive groups that are cleaved through the application of ultrasound or a mechanical strain (e.g., a mechanical strain created by a magnetic field on a magneto-responsive gel).

The active agent can comprise any suitable therapeutic or diagnostic agent.

In some embodiments, the therapeutic agent can comprise a diagnostic agent (e.g., an imaging agent, such as an MRI contrast agent). Suitable diagnostic agents can include molecules that are detectable in the body of a subject by an imaging technique such as X-ray radiography, ultrasound, computed tomography (CT), single-photon emission computed tomography (SPECT), magnetic resonance imaging (MRI), positron emission tomography (PET), optical fluorescent imaging, optical visible light imaging, and nuclear medicine including Cerenkov light imaging. For example, the diagnostic agent can comprise a radionuclide, paramagnetic metal ion, or a fluorophore.

In some cases, the diagnostic agent can comprise a metal chelator. The terms “metal chelator” and “chelating agent” refer to a polydentate ligand that can form a coordination complex with a metal atom. It is generally preferred that the coordination complex is stable under physiological conditions. That is, the metal will remain complexed to the chelator in vivo.

In some cases, the metal chelator is a molecule that complexes to a radionuclide metal or paramagnetic metal ion to form a metal complex that is stable under physiological conditions. The metal chelator may be any of the metal chelators known in the art for complexing a medically useful paramagnetic metal ion, or radionuclide.

In some cases, such as in the case of complexes designed for radiopharmaceutical or radiotherapy applications, it can be convenient to prepare the complexes comprising a radionuclide, at or near the site where they are to be used (e.g., in a hospital pharmacy or clinic). Accordingly, in some embodiments, the complex can comprise a metal chelator uncomplexed with a metal ion. In such embodiments, the complex can be complexed with a suitable metal ion prior to administration. In other embodiments, the complex comprises a metal chelator complexed with a suitable metal ion (e.g., a paramagnetic metal ion or a radionuclide).

Suitable metal chelators include, for example, linear, macrocyclic, terpyridine, and N₃S, N₂S₂, or N₄ chelators (see also, U.S. Pat. Nos. 4,647,447, 4,957,939, 4,963,344, 5,367,080, 5,364,613, 5,021,556, 5,075,099, 5,886,142, the disclosures of which are incorporated by reference herein in their entirety), and other chelators known in the art including, but not limited to, HYNIC, DTPA, EDTA, DOTA, TETA, and bisamino bisthiol (BAT) chelators (see also U.S. Pat. No. 5,720,934). For example, macrocyclic chelators, and in particular N₄ chelators are described in U.S. Pat. Nos. 4,885,363; 5,846,519; 5,474,756; 6,143,274; 6,093,382; 5,608,110; 5,665,329; 5,656,254; and 5,688,487, the disclosures of which are incorporated by reference herein in their entirety. Certain N₃S chelators are described in PCT/CA94/00395, PCT/CA94/00479, PCT/CA95/00249 and in U.S. Pat. Nos. 5,662,885; 5,976,495; and 5,780,006, the disclosures of which are incorporated by reference herein in their entirety. The chelator may also include derivatives of the chelating ligand mercapto-acetyl-glycyl-glycyl-glycine (MAG3), which contains an N₃S, and N₂S₂ systems such as MAMA (monoamidemonoaminedithiols), DADS (N₂S diaminedithiols), CODADS and the like. These ligand systems and a variety of others are described in Liu and Edwards, Chem. Rev. 1999, 99, 2235-2268; Caravan et al., Chem. Rev. 1999, 99, 2293-2352; and references therein, the disclosures of which are incorporated by reference herein in their entirety.

The metal chelator may also include complexes known as boronic acid adducts of technetium and rhenium dioximes, such as those described in U.S. Pat. Nos. 5,183,653; 5,387,409; and 5,118,797, the disclosures of which are incorporated by reference herein, in their entirety.

Examples of suitable chelators include, but are not limited to, derivatives of diethylenetriamine pentaacetic acid (DTPA), 1,4,7,10-tetraazacyclotetradecane-1,4,7,10-tetraacetic acid (DOTA), 1-substituted 1,4,7,-tricarboxymethyl 1,4,7,10 tetraazacyclododecane triacetic acid (DO3A), derivatives of the 1-1-(1-carboxy-3-(p-nitrophenyl)propyl-1,4,7,10 tetraazacyclododecane triacetate (PA-DOTA) and MeO-DOTA, ethylenediaminetetraacetic acid (EDTA), and 1,4,8,11-tetraazacyclotetradecane-1,4,8,11-tetraacetic acid (TETA), derivatives of 3,3,9,9-tetramethyl-4,8-diazaundecane-2,10-dione dioxime (PnAO); and derivatives of 3,3,9,9-tetramethyl-5-oxa-4,8-diazaundecane-2,10-dione dioxime (oxa PnAO). Additional chelating ligands are ethylenebis-(2-hydroxy-phenylglycine) (EHPG), and derivatives thereof, including 5-C1-EHPG, 5-Br-EHPG, 5-Me-EHPG, 5-t-Bu-EHPG, and 5-sec-Bu-EHPG; benzodiethylenetriamine pentaacetic acid (benzo-DTPA) and derivatives thereof, including dibenzo-DTPA, phenyl-DTPA, diphenyl-DTPA, benzyl-DTPA, and dibenzyl-DTPA; bis-2 (hydroxybenzyl)-ethylene-diaminediacetic acid (HBED) and derivatives thereof; the class of macrocyclic compounds which contain at least 3 carbon atoms and at least two heteroatoms (0 and/or N), which macrocyclic compounds can consist of one ring, or two or three rings joined together at the hetero ring elements, e.g., benzo-DOTA, dibenzo-DOTA, and benzo-NOTA, where NOTA is 1,4,7-triazacyclononane N,N′,N″-triacetic acid, benzo-TETA, benzo-DOTMA, where DOTMA is 1,4,7,10-tetraazacyclotetradecane-1,4,7,10-tetra(methyl tetraacetic acid), and benzo-TETMA, where TETMA is 1,4,8,11-tetraazacyclotetradecane-1,4,8,11-(methyl tetraacetic acid); derivatives of 1,3-propylenediaminetetraacetic acid (PDTA) and triethylenetetraaminehexaacetic acid (TTHA); derivatives of 1,5,10-N,N′,N″-tris(2,3-dihydroxybenzoyl)-tricatecholate (LICAM) and 1,3,5-N,N′,N″-tris(2,3-dihydroxybenzoyl)aminomethylbenzene (MECAM). Examples of representative chelators and chelating groups are described in WO 98/18496, WO 86/06605, WO 91/03200, WO 95/28179, WO 96/23526, WO 97/36619, PCT/US98/01473, PCT/US98/20182, and U.S. Pat. Nos. 4,899,755, 5,474,756, 5,846,519 and 6,143,274, each of which is hereby incorporated by reference in its entirety.

In some embodiments, the metal chelator comprises desferrioxamine (also referred to as deferoxamine, desferrioxamine B, desferoxamine B, DFO-B, DFOA, DFB or desferal) or a derivative thereof. See, for example U.S. Pat. Nos. 8,309,583, 4,684,482, and 5,268,165, each of which is hereby incorporated by reference in its entirety for its teaching of desferrioxamine and desferrioxamine derivatives.

As is well known in the art, metal chelators can be specific for particular metal ions. Suitable metal chelators can be selected for incorporation into the self-assembling molecule based on the desired metal ion and intended use of the self-assembling molecule.

Paramagnetic ions form a magnetic moment upon the application of an external magnetic field thereto. Magnetization is not retained in the absence of an externally applied magnetic field because thermal motion causes the spin of unpaired electrons to become randomly oriented in the absence of an external magnetic field. By taking advantage of its property of shortening the magnetic relaxation time of water molecules, a paramagnetic substance is usable as an active component of MRI contrast agents. Suitable paramagnetic transition metal ions include Cr³⁺, Co²⁺, Mn²⁺, Ni²⁺, Fe²⁺, Fe³⁺, Zr⁴⁺, Cu²⁺, and Cu³⁺. In preferred embodiments, the paramagnetic ion is a lanthanide ion (e.g., La³⁺, Gd³⁺, Ce³⁺, Tb³⁺, Pr³⁺, Dy³⁺, Nd³⁺, Ho³⁺, Pm³⁺, Er³⁺, Sm³⁺, Tm³⁺, Eu³⁺, Yb³⁺, or Lu³⁺). In MRI, especially preferred metal ions are Gd³⁺, Mn²⁺, Fe³⁺, and Eu²⁺.

MRI contrast agents can also be made with paramagnetic nitroxides molecules in place of the chelating agent and paramagnetic metal ion.

Suitable radionuclides include ^{99m}Tc, ⁶⁷Ga, ⁶⁸Ga, ⁶⁶Ga, ⁴⁷Sc, ⁵¹Cr, ¹⁶⁷Tm, ¹⁴¹Ce, ¹¹¹In, ¹²³I, ¹²⁵I, ¹³¹I, ¹²⁴I, ¹⁸F, ¹¹C, ¹⁵N, ¹⁷O, ¹⁶⁸Yb, ¹⁷⁵Yb, ¹⁴⁰La, ⁹⁰Y, ⁸⁸Y, ⁸⁶Y, ¹⁵³Sm, ¹⁶⁶Ho, ¹⁶⁵Dy, ¹⁶⁶Dy, ⁶²Cu, ⁶⁴Cu, ⁶⁷Cu, ⁹⁷Ru, ¹⁰³Ru, ¹⁸⁶Re, ¹⁸⁸Re, ₂₀₃Pb, ²¹¹Bi, ²¹²Bi, ²¹³Bi, ²¹⁴Bi, ²²⁵Ac, ²¹¹At, ¹⁰⁵Rh, ¹⁰⁹Pd, ^{117m}Sn, ¹⁴⁹Pm, ¹⁶¹Tb, ¹⁷⁷Lu, ¹⁹⁸Au, ¹⁹⁹Au, 89Zr, and oxides or nitrides thereof. The choice of isotope will be determined based on the desired therapeutic or diagnostic application. For example, for diagnostic purposes (e.g., to diagnose and monitor therapeutic progress in primary tumors and metastases), suitable radionuclides includes ⁶⁴Cu, ⁶⁷Ga, ⁶⁸Ga, ⁶⁶Ga, ^{99m}Tc, and ¹¹¹In, ¹⁸F, ⁸⁹Zr, ¹²³I, ¹²⁴I, ¹⁷⁷Lu, ¹⁵N, ¹⁷O. For therapeutic purposes (e.g., to provide radiotherapy for primary tumors and metastasis related to cancers of the prostate, breast, lung, etc.), suitable radionuclides include ⁶⁴Cu, ⁹⁰Y, ¹⁰⁵Rh, ¹¹¹In, ¹³¹I, ^{117 m}Sn, ¹⁴⁹Pm, ¹⁵³Sm, ¹⁶¹Tb, ¹⁶⁶Dy, ¹⁶⁶Ho, ¹⁷⁵Yb, ¹⁷⁷Lu, ^{186/188}Re, ¹⁹⁹Au, ¹³¹I, and ¹²⁵I ²¹²Bi, ²¹¹At.

In the case of complexes designed to be imaged using PET, radionuclides with short half-lives such as carbon-11 (˜20 min), nitrogen-13 (˜10 min), oxygen-15 (˜2 min), fluorine-18 (˜110 min), or rubidum-82 (˜1.27 min) are often used. In certain embodiments when a non-metal radionuclide is employed, the therapeutic or diagnostic agent comprises a radiotracer covalently attached to the self-assembling molecule. By way of exemplification, suitable ¹⁸F-based radiotracers include ¹⁸F-fluordesoxyglucose (FDG), ¹⁸F-dopamine, ¹⁸F-L-DOPA, ¹⁸F-fluorcholine, ¹⁸F-fluormethylethylcholin, and ¹⁸P-fluordihydrotestosteron.

In the case of self-assembled molecules designed to be imaged using PET, radionuclides with long half-lives such as ¹²⁴I, or ⁸⁹Zr are also often used.

Fluorescent imaging has emerged with unique capabilities for molecular cancer imaging. Fluorophores emit energy throughout the visible spectrum; however, the best spectrum for in vivo imaging is in the near-infrared (NIR) region (650 nm-900 nm). Unlike the visible light spectrum (400-650 nm), in the NIR region, light scattering decreases and photo absorption by hemoglobin and water diminishes, leading to deeper tissue penetration of light. Furthermore, tissue auto-fluorescence is low in the NIR spectra, which allows for a high signal to noise ratio. There is a range of small molecule organic fluorophores with excitation and emission spectra in the NIR region. Some, such as indocyanine green (ICG) and cyanine derivatives Cy5.5 and Cy7, have been used in imaging for a relatively long time. Modern fluorophores are developed by various biotechnology companies and include: Alexa dyes; IRDye dyes; VivoTag dyes and HylitePlus dyes. In general, the molecular weights of these fluorophores are below 1 kDa.

In some embodiments, the diagnostic agent can comprise a radiocontrast agent. In these embodiments, the diagnostic agent can comprise an iodinated moiety. Examples of suitable radiocontrast agents include iohexol, iodixanol and ioversol.

In some embodiments, the active agent can comprise a therapeutic agent. Any suitable therapeutic agent can be incorporated in the complexes described herein. In some examples, the therapeutic agent can comprise an agent to treat or prevent a disease or disorder associated with the overexpression of CD163. For example, in some cases, the therapeutic agent can comprise an anti-cancer agent, an anti-inflammatory agent, an agent that treats or prevents infection, or a combination thereof. In some examples, the active agent can comprise an agent administered to treat hemolytic anemia and other conditions characterized by or associated with hemolysis (e.g., sickle cell anemia, malaria, red blood cell transfusions, thalassemia, autoimmune disorders, bone marrow failure, infections, surgery, severe burns, acute lung injury, the administration of chemotherapeutics, radiation therapy, etc.). In some examples, the active agent can comprise an active agent administered to treat a disease associated with macrophages and monocytes. Such diseases are known in the art and include, for example, heart disease, HIV infection, cancer, fibrotic diseases (e.g., cystic fibrosis), asthma, inflammatory bowel disease, rheumatoid arthritis, and diseases in which macrophages or monocytes function as hosts for intracellular pathogens (e.g., malaria, tuberculosis, leishmaniasis, chikungunya, adenovirus, Legionnaires' disease, and infections caused by bacteria in the genus Brucella such as B. abortus, B. canis, B. melitensis, and B. suis).

In some embodiments, the active agent can comprise an anti-cancer agent. Examples of anti-cancer agents include, but are not limited to, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Aldara (Imiquimod), Aldesleukin, Alemtuzumab, Alimta (Pemetrexed Disodium), Aloxi (Palonosetron Hydrochloride), Ambochlorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Bendamustine Hydrochloride, BEP, Bevacizumab, Bexarotene, Bexxar (Tositumomab and I 131 Iodine Tositumomab), Bicalutamide, Bleomycin, Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPDX, Carboplatin, Carboplatin-Taxol, Carfilzomib, Casodex (Bicalutamide), CeeNU (Lomustine), Cerubidine (Daunorubicin Hydrochloride), Cervarix (Recombinant HPV Bivalent Vaccine), Cetuximab, Chlorambucil, Chlorambucil-Prednisone, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen (Dactinomycin), Crizotinib, CVP, Cyclophosphamide, Cyfos (Ifosfamide), Cytarabine, Cytarabine (Liposomal), Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Envinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), Folfiri, Folfiri-Bevacizumab, Folfiri-Cetuximab, Folfirinox, Folfox, Folotyn (Pralatrexate), FU-LV, Fulvestrant, Gardasil (Recombinant HPV Quadrivalent Vaccine), Gazyva (Obinutuzumab), Gefitinib, Gemcitabine Hydrochloride, Gemcitabine-Cisplatin, Gemcitabine-Oxaliplatin, Gemtuzumab Ozogamicin, Gemzar (Gemcitabine Hydrochloride), Gilotrif (Afatinib Dimaleate), Gleevec (Imatinib Mesylate), Glucarpidase, Goserelin Acetate, Halaven (Eribulin Mesylate), Herceptin (Trastuzumab), HPV Bivalent Vaccine (Recombinant), HPV Quadrivalent Vaccine (Recombinant), Hycamtin (Topotecan Hydrochloride), Hyper-CVAD, Ibritumomab Tiuxetan, Ibrutinib, ICE, Iclusig (Ponatinib Hydrochloride), Ifex (Ifosfamide), Ifosfamide, Ifosfamidum (Ifosfamide), Imatinib Mesylate, Imbruvica (Ibrutinib), Imiquimod, Inlyta (Axitinib), Intron A (Recombinant Interferon Alfa-2b), Iodine 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Kyprolis (Carfilzomib), Lapatinib Ditosylate, Lenalidomide, Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Neupogen (Filgrastim), Nexavar (Sorafenib Tosylate), Nilotinib, Nolvadex (Tamoxifen Citrate), Nplate (Romiplostim), Obinutuzumab, Ofatumumab, Omacetaxine Mepesuccinate, Oncaspar (Pegaspargase), Ontak (Denileukin Diftitox), OEPA, OPPA, Oxaliplatin, Paclitaxel, Paclitaxel Albumin-stabilized Nanoparticle Formulation, Palifermin, Palonosetron Hydrochloride, Pamidronate Disodium, Panitumumab, Paraplat (Carboplatin), Paraplatin (Carboplatin), Pazopanib Hydrochloride, Pegaspargase, Peginterferon Alfa-2b, PEG-Intron (Peginterferon Alfa-2b), Pemetrexed Disodium, Perj eta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Radium 223 Dichloride, Raloxifene Hydrochloride, Rasburicase, R-CHOP, R-CVP, Recombinant HPV Bivalent Vaccine, Recombinant HPV Quadrivalent Vaccine, Recombinant Interferon Alfa-2b, Regorafenib, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Sipuleucel-T, Sorafenib Tosylate, Sprycel (Dasatinib), Stanford V, Sterile Talc Powder (Talc), Steritalc (Talc), Stivarga (Regorafenib), Sunitinib Malate, Sutent (Sunitinib Malate), Sylatron (Peginterferon Alfa-2b), Synovir (Thalidomide), Synribo (Omacetaxine Mepesuccinate), Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Toposar (Etoposide), Topotecan Hydrochloride, Toremifene, Torisel (Temsirolimus), Tositumomab and I¹³¹ Iodine Tositumomab, Totect (Dexrazoxane Hydrochloride), Trametinib, Trastuzumab, Treanda (Bendamustine Hydrochloride), Trisenox (Arsenic Trioxide), Tykerb (Lapatinib Ditosylate), Vandetanib, VAMP, Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELOX, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), and Zytiga (Abiraterone Acetate). These anti-cancer agents are non-limiting, as the skilled artisan would be able to readily identify other anti-cancer agents.

In some embodiments, the active agent can comprise an anti-proliferative agent, e.g., mycophenolate mofetil (MMF), azathioprine, sirolimus, tacrolimus, paclitaxel, biolimus A9, novolimus, myolimus, zotarolimus, everolimus, or tranilast. These anti-proliferative agents are non-limiting, as the skilled artisan would be able to readily identify other anti-proliferative agents.

In some embodiments, the active agent can comprise an anti-inflammatory agent, e.g., corticosteroid anti-inflammatory drugs (e.g., beclomethasone, beclometasone, budesonide, flunisolide, fluticasone propionate, triamcinolone, methylprednisolone, prednisolone, or prednisone); or non-steroidal anti-inflammatory drugs (NSAIDs) (e.g., acetylsalicylic acid, diflunisal, salsalate, choline magnesium trisalicylate, ibuprofen, dexibuprofen, naproxen, fenoprofen, ketoprofen, dexketoprofen, fluribiprofen, oxaprozin, loxoprofen, indomethacin, tolmetin, sulindac, etodolac, ketorolac, diclofenac, aceclofenac, nabumetone, piroxicam, meloxicam, tenoxicam, droxicam, lornoxicam, isoxicam, mefenamic acid, meclofenamic acid, flufenamic acid, tolfenamic acid, celecoxib, rofecoxib, valdecoxib, parecoxib, lumiracoxib, etoricoxib, firocoxib, nimesulide, licofelone, H-harpaide, or lysine clonixinate). These anti-inflammatory agents are non-limiting, as the skilled artisan would be able to readily identify other anti-inflammatory agents.

In some embodiments, the active agent can comprise a drug that prevents or reduces transplant rejection, e.g., an immunosuppressant. Exemplary immunosuppressants include calcineurin inhibitors (e.g., cyclosporine, Tacrolimus (FK506)); mammalian target of rapamycin (mTOR) inhibitors (e.g., rapamycin, also known as Sirolimus); antiproliferative agents (e.g., azathioprine, mycophenolate mofetil, mycophenolate sodium); antibodies (e.g., basiliximab, daclizumab, muromonab); corticosteroids (e.g., prednisone). These drugs that prevent or reduce transplant rejection are non-limiting, as the skilled artisan would be able to readily identify other drugs that prevent or reduce transplant rejection.

In some embodiments, the active agent can comprise a drug that treats or prevents infection, e.g., an antibiotic. Suitable antibiotics include, but are not limited to, beta-lactam antibiotics (e.g., penicillins, cephalosporins, carbapenems), polymyxins, rifamycins, lipiarmycins, quinolones, sulfonamides, macrolides lincosamides, tetracyclines, aminoglycosides, cyclic lipopeptides (e.g., daptomycin), glycylcyclines (e.g., tigecycline), oxazonidinones (e.g., linezolid), and lipiarmycines (e.g., fidazomicin). For example, antibiotics include erythromycin, clindamycin, gentamycin, tetracycline, meclocycline, (sodium) sulfacetamide, benzoyl peroxide, and azelaic acid. Suitable penicillins include amoxicillin, ampicillin, bacampicillin, carbenicillin, cloxacillin, dicloxacillin, flucloxacillin, mezlocillin, nafcillin, oxacillin, penicillin g, penicillin v, piperacillin, pivampicillin, pivmecillinam, and ticarcillin. Exemplary cephalosporins include cefacetrile, cefadroxil, cephalexin, cefaloglycin, cefalonium, cefaloridine, cefalotin, cefapirin, cefatrizine, cefazaflur, cefazedone, cefazolin, cefradine, cefroxadine, ceftezole, cefaclor, cefamandole, cefmetazole, cefonicid, cefotetan, cefoxitin, cefprozil, cefuroxime, cefuzonam, cfcapene, cefdaloxime, cefdinir, cefditoren, cefetamet, cefixime, cefmenoxime, cefodizime, cefotaxime, cefpimizole, cefpodoxime, cefteram, ceftibuten, ceftiofur, ceftiolene, ceftizoxime, ceftriaxone, ceftazidime, cefclidine, cefepime, ceflurprenam, cefoselis, cefozopran, cefpirome, cequinome, ceftobiprole, ceftaroline, cefaclomezine, cefaloram, cefaparole, cefcanel, cefedrlor, cefempidone, cefetrizole, cefivitril, cefmatilen, cefmepidium, cefovecin, cefoxazole, cefrotil, cefsumide, cefuracetime, and ceftioxide.

Monobactams include aztreonam. Suitable carbapenems include imipenem/cilastatin, doripenem, meropenem, and ertapenem. Exemplary macrolides include azithromycin, erythromycin, larithromycin, dirithromycin, roxithromycin, and telithromycin. Lincosamides include clindamycin and lincomycin. Exemplary streptogramins include pristinamycin and quinupristin/dalfopristin. Suitable aminoglycoside antibiotics include amikacin, gentamycin, kanamycin, neomycin, netilmicin, paromomycin, streptomycin, and tobramycin. Exemplary quinolones include flumequine, nalidixic acid, oxolinic acid, piromidic acid, pipemidic acid, rosoxacin, ciprofloxacin, enoxacin, lomefloxacin, nadifloxacin, norfloxacin, ofoxacin, pefloxacin, rufloxacin, balofloxacin, gatifloxacin, repafloxacin, levofloxacin, moxifloxacin, pazufloxacin, sparfloxacin, temafloxacin, tosufloxacin, besifloxacin, clinafoxacin, gemifloxacin, sitafloxacin, trovafloxacin, and prulifloxacin. Suitable sulfonamides include sulfamethizole, sulfamethoxazole, and trimethoprim-sulfamethoxazone. Exemplary tetracyclines include demeclocycline, doxycycline, minocycline, oxytetracycline, tetracycline, and tigecycline. Other antibiotics include chloramphenicol, metronidazole, tinidazole, nitrofurantoin, vancomycin, teicoplanin, telavancin, linezolid, cycloserine, rifampin, rifabutin, rifapentin, bacitracin, polymyxin B, viomycin, and capreomycin. The skilled artisan could readily identify other antibiotics useful in the devices and methods described herein.

In some embodiments, the active agent can comprise an anti-HIV agent. Examples of anti-HIV agents include anti-HIV antibodies, immunostimulants such as interferon, and the like, a reverse transcriptase inhibitor, a protease inhibitor, an inhibitor of bond between a bond receptor (CD4, CXCR4, CCR5, and the like) of a host cell recognized by virus and the virus, and the like.

Specific examples of HIV reverse transcriptase inhibitors include Retrovir® (zidovudine or AZT), Epivir® (lamivudine or 3TC), Zerit® (sanilvudine), Videx® (didanosine), Hivid® (zalcitabine), Ziagen® (abacavir sulfate), Viramune® (nevirapine), Stocrin® (efavirenz), Rescriptor® (delavirdine mesylate), Combivir® (zidovudine+lamivudine), Trizivir® (abacavir sulfate+lamivudine+zidovudine), Coactinon® (emivirine), Phosphonovir®, Coviracil®, alovudine (3′-fluoro-3′-deoxythymidine), Thiovir (thiophosphonoformic acid), Capravirin (5-[(3,5-dichlorophenyl)thio]-4-isopropyl-1-(4-pyridylmethyl)imidazole-2-methanol carbamic acid), Tenofovir (PMPA), Tenofovir disoproxil fumarate ((R)-[[2-(6-amino-9H-purin-9-yl)-1-methylethoxy]methyl]phosphonic acid bis(isopropoxycarbonyloxymethyl)ester fumarate), DPC-083 ((4S)-6-chloro-4-[(1E)-cyclopropylethenyl]-3,4-dihydro-4-trifluoromethyl-2 (1H)-quinazolinone), DPC-961 ((4S)-6-chloro-4-(cyclopropylethynyl)-3,4-dihydro-4-(trifluoromethyl)-2 (1H)-quinazolinone), DAPD ((−)β-D-2,6-diaminopurine dioxolane), Immunocal, MSK-055, MSA-254, MSH-143, NV-01, TMC-120, DPC-817, GS-7340, TMC-125, SPD-754, D-A4FC, capravirine, UC-781, emtricitabine, alovudine, Phosphazid, UC-781, BCH-10618, DPC-083, Etravirine, BCH-13520, MIV-210, abacavir sulfate/lamivudine, GS-7340, GW-5634, GW-695634, and the like.

Specific examples of HIV protease inhibitors include Crixivan® (indinavir sulfate ethanolate), saquinavir, Invirase® (saquinavir mesylate), Norvir® (ritonavir), Viracept® (nelfinavir mesylate), lopinavir, Prozei® (amprenavir), Kaletra® (ritonavir+lopinavir), mozenavir dimesylate ([4R-(4α,5α,6β)]-1-3-bis[(3-aminophenyl)methyl]hexahydro-5,6-dihydroxy-4,7-bis(phenylmethyl)-2H-1,3-diazepin-2-one dimethanesulfonate), tipranavir (3′-[(1R)-1-[(6R)-5,6-dihydro-4-hydroxy-2-oxo-6-phenylethyl-6-propyl-2H-pyran-3-yl]propyl]-5-(trifluoromethyl)-2-pyridinesulfonamide), lasinavir (N-[5(S)-(tert-butoxycarbonylamino)-4(S)-hydroxy-6-phenyl-2(R)-(2,3,4-trimethoxybenzyl)hexanoyl]-L-valine 2-methoxyethylenamide), KNI-272 ((R)—N-tert-butyl-3-[(2S,3S)-2-hydroxy-3-N—[(R)-2-N-(isoquinolin-5-yloxyacetyl)amino-3-methylthiopropanoyl]amino-4-phenylbutanoyl]-5,5-dimethyl-1,3-thiazolidine-4-carboxamide), GW-433908, TMC-126, DPC-681, buckminsterfullerene, MK-944A (MK944 (N-(2(R)-hydroxy-1(S)-indanyl)-2(R)-phenylmethyl-4(S)-hydroxy-5-[4-(2-benzo[b]furanylmethyl)-2(S)-(tert-butylcarbamoyl)piperazin-1-yl]pentanamide)+indinavir sulfate), JE-2147 ([2(S)-oxo-4-phenylmethyl-3(5)-[(2-methyl-3-oxy)phenylcarbonylamino]-1-oxabutyl]-4-[(2-methylphenyl)methylamino]carbonyl-4(R)-5,5-dimethyl-1,3-thiazole), BMS-232632 ((3S,8S,9S,12S)-3,12-bis(1,1-dimethylethyl)-8-hydroxy-4,11-dioxo-9-(phenylmethyl)-6-[[4-(2-pyridinyl)phenyl]methyl]-2,5,6,10,13-pentaazatetradecanedicarboxylic acid dimethyl ester), DMP-850 ((4R,5S,6S,7R)-1-(3-amino-1H-indazol-5-ylmethyl)-4,7-dibenzyl-3-butyl-5,6-dihydroxyperhydro-1,3-diazepin-2-one), DMP-851, RO-0334649, Nar-DG-35, R-944, VX-385, TMC-114, Tipranavir, Fosamprenavir sodium, Fosamprenavir calcium, Darunavir, GW-0385, R-944, RO-033-4649, AG-1859, and the like.

The HIV integrase inhibitor may be S-1360, L-870810, and the like. The DNA polymerase inhibitor or DNA synthesis inhibitor may be Foscavir®, ACH-126443 (L-2′,3′-didehydro-dideoxy-5-fluorocytidine), entecavir ((1S,3S,4S)-9-[4-hydroxy-3-(hydroxymethyl)-2-methylenecyclopentyl]guanine), calanolide A ([10R-(10a,11(3,12a)]-11,12-dihydro-12-hydroxy-6,6,10,11-tetramethyl-4-propyl-2H,6H,10H-benzo[1,2-b:3,4-b′:5,6-b″]tripyran-2-one), calanolide B, NSC-674447 (1,1′-azobisformamide), Iscador (viscum alubm extract), Rubutecan, and the like. The HIV antisense drug may be HGTV-43, GEM-92, and the like. The anti-HIV antibody or other antibody may be NM-01, PRO-367, KD-247, Cytolin®, TNX-355 (CD4 antibody), AGT-1, PRO-140 (CCR5 antibody), Anti-CTLA-4 Mab, and the like. The HIV vaccine or other vaccine may be ALVAC®, AIDSVAX®, Remune®, HIV gp41 vaccine, HIV gp120 vaccine, HIV gp140 vaccine, HIV gp160 vaccine, HIV p17 vaccine, HIV p24 vaccine, HIV p55 vaccine, AlphaVax Vector System, canarypox gp160 vaccine, AntiTat, MVA-F6 Nef vaccine, HIV rev vaccine, C4-V3 peptide, p2249f, VIR-201, HGP-30W, TBC-3B, PARTICLE-3B, and the like, Antiferon (interferon-α vaccine), and the like.

The interferon or interferon agonist may be Sumiferon®, MultiFeron®, interferon-τ, Reticulose, Human leukocyte interferon alpha, and the like. The CCR5 antagonist may be SCH-351125, and the like. The pharmaceutical agent acting on HIV p24 may be GPG-NH2 (glycyl-prolyl-glycinamide), and the like. The HIV fusion inhibitor may be FP-21399 (1,4-bis[3-[(2,4-dichlorophenyl)carbonylamino]-2-oxo-5,8-disodium sulfonyl]naphthyl-2,5-dimethoxyphenyl-1,4-dihydrazone), T-1249, Synthetic Polymeric Construction No 3, pentafuside, FP-21399, PRO-542, Enfuvirtide, and the like. The IL-2 agonist or antagonist may be interleukin-2, Imunace®, Proleukin®, Multikine®, Ontak®, and the like. The TNF-α antagonist may be Thalomid® (thalidomide), Remicade® (infliximab), curdlan sulfate, and the like. The α-glucosidase inhibitor may be Bucast®, and the like.

The purine nucleoside phosphorylase inhibitor may be peldesine (2-amino-4-oxo-3H,5H-7-[(3-pyridyl)methyl]pyrrolo[3,2-d]pyrimidine), and the like. The apoptosis agonist or inhibitor may be Arkin Z®, Panavir®, Coenzyme Q10 (2-deca(3-methyl-2-butenylene)-5,6-dimethoxy-3-methyl-p-benzoquinone), and the like. The cholinesterase inhibitor may be Cognex®, and the like, and the immunomodulator may be Imunox®, Prokine®, Met-enkephalin (6-de-L-arginine-7-de-L-arginine-8-de-L-valinamide-adrenorphin), WF-10 (10-fold dilute tetrachlorodecaoxide solution), Perthon, PRO-542, SCH-D, UK-427857, AMD-070, AK-602, and the like.

In addition, Neurotropin®, Lidakol®, Ancer 20®, Ampligen®, Anticort®, Inactivin®, and the like, PRO-2000, Rev M10 gene, HIV specific cytotoxic T cell (CTL immunotherapy, ACTG protocol 080 therapy, CD4-ζ gene therapy), SCA binding protein, RBC-CD4 complex, Motexafin gadolinium, GEM-92, CNI-1493, (±)—FTC, Ushercell, D2S, BufferGel®, VivaGel®, Glyminox vaginal gel, sodium lauryl sulfate, 2F5, 2F5/2G12, VRX-496, Ad5gag2, BG-777, IGIV-C, BILR-255, and the like may be used in the combination therapy.

Other suitable active agents include porphyrin-based active agents (e.g., porphyrin-based imaging agents, porphyrin-based agents for photodynamic therapy), erythropoietin, hydroxycarbamide (also known as hydroxyurea), corticosteroids, immunosuppressive agents, analgesic agents, agents that induce hemolysis (e.g., rituximab, cephalosporins, dapsone, levodopa, levofloxacin, methyldopa, nitrofurantoin, NSAIDs, penicillin and derivatives thereof, phenazopyridine, quinidine), dexamethasone, conjugates targeting the CD163 receptor (e.g., agents described in U.S. Pat. No. 9,724,426 to Graversen et al. which is incorporated by reference in its entirety), antibiotics; anti-tuberculosis antibiotics (such as isoniazide, ethambutol); anti-retroviral drugs, for example inhibitors of reverse transcription (such as zidovudin) and/or protease inhibitors (such as indinavir); drugs with effect on leishmaniasis (such as meglumine antimoniate); immunosuppressive drugs such as a glucocorticoid (e.g., cortisone and derivatives thereof (such as hydrocortisone); prednisone and derivatives thereof (such as prednisolone, methylprednisolone, methylprednisolone-acetate, methylprednisolone-succinate); dexamethasone and derivatives thereof; triamcinolone and derivatives thereof (such as triamcinolonehexacetonuid, triamcinolonacetonamid); paramethasone; betamethasone; fluhydrocortisone; fluocinolone); methotrexate; cyclophosphamide; 6-mercaptopurin; cyclosporine; tacrolimus; mycophenolate mofetil; sirulimus; everolimus; an siRNA molecule capable of inhibiting synthesis of proinflammatory cytokines (such as TNF); a non-steroidal anti-inflammatory drug (NSAIDs, such as aspirin, ibuprofen); a steroid (such as vitamin D); and a disease-modifying anti-rheumatic drug (DMARDs, such as penicillamin, sulfasalazin, cyclosporine).

In some embodiments, the active agent can comprise a toll-like receptor (TLR) agonist. A “TLR agonist” as used herein, refers to a substance that can combine with a TLR and activate it. By slightly altering the structure of such substances, TLR agonists can be designed to have different stabilities in the body, allowing a certain amount of control over where the substances go, and how long they last. Microbial ligands have been identified for several mammalian TLRs. For example, TLR4 recognizes lipopolysaccharide (LPS), TLR2 interacts with peptidoglycan, bacterial lipopeptides, and certain types of LPS, TLR3 recognizes double-stranded RNA, TLR5 recognizes bacterial flagellin, TLR9 recognizes bacterial DNA.

TLR agonists are well-known in the art and include, for example, but not limited to, lipopolysaccharide (LPS, binds TLR4), Fibrin (binds TLR4), lipoteichoic acid (LTA, binds TLR2), peptidoglycan (PG, binds TLR2), CpG (bacterial DNA, binds TLR9), 7-thia-8-oxoguanosine (TOG or isatoribine, binds TLR7), 7-deazaguanosine (binds TLR7), 7-allyl-8-oxoguanosine (loxoribine, binds TLR7), 7-dezaguanosine (7-deza-G, binds TLR7), imiquimod (R837, binds TLR7), or R848 (binds TLR7). In some embodiments, the TLR agonist can comprise a TLR7 agonist or a TLR9 agonist that is carried by the apoHb-Hp complex for receptor mediated uptake and immune activation within the endosome. In certain embodiments, the TLR agonist can comprise a TLR7 agonist (e.g., an imidazoquinoline such as imiquimod).

The expression of heme oxygenase-1 (H0-1) inhibits vascular inflammation and the induction of apoptosis. Accordingly, in some embodiments, the active agent can comprise an agent which modulates the activity of heme-oxygenase-1 (H0-1) activity. In some embodiments, the modulator of HO-1 is an antagonist, partial agonist, inverse agonist, neutral or competitive antagonist, allosteric antagonist, and/or orthosteric antagonist of HO-1. In some embodiments, the modulator of HO-1 is a HO-1 agonist, partial agonist, and/or positive allosteric modulator. In some embodiments, the agonist, partial agonist, and/or positive allosteric modulator of HO-1 is piperine, hemin, and/or brazilin. In some embodiments, the active agent comprises a protoporphyrin IX complex, such as zinc protoporphyrin IX or tin protoporphyrin IX, that is a HO-1 antagonist.

Pharmaceutical Compositions

The complexes provided herein can be administered in the form of pharmaceutical compositions. These complexes can be prepared as described herein or elsewhere, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal, intramuscular or injection or infusion; or intracranial, (e.g., intrathecal or intraventricular, administration). Parenteral administration can be in the form of a single bolus dose, or may be, for example, by a continuous perfusion pump. In some embodiments, the complexes provided herein are suitable for parenteral administration. In some embodiments, the complexes provided herein are suitable for intravenous administration.

Pharmaceutical compositions and formulations for topical administration may include, but are not limited to, transdermal patches, ointments, lotions, creams, gels, drops, suppositories, sprays, liquids and powders. Conventional pharmaceutical carriers, aqueous, powder or oily bases, thickeners and the like may be necessary or desirable. In some embodiments, the pharmaceutical compositions provided herein are suitable for parenteral administration. In some embodiments, the pharmaceutical compositions provided herein are suitable for intravenous administration. In some embodiments, the pharmaceutical compositions provided herein are suitable for oral administration. In some embodiments, the pharmaceutical compositions provided herein are suitable for topical administration.

Also provided are pharmaceutical compositions which contain, as the active ingredient, a complex provided herein in combination with one or more pharmaceutically acceptable carriers (e.g. excipients). In making the pharmaceutical compositions provided herein, the complex can be mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be, for example, in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, without limitation, lubricating agents such as talc, magnesium stearate, and mineral oil; wetting agents; emulsifying and suspending agents; preserving agents such as methyl- and propylhydroxy-benzoates; sweetening agents; flavoring agents, or combinations thereof.

The complexes can be effective over a wide dosage range and is generally administered in an effective amount. It will be understood, however, that the amount of the compound actually administered will usually be determined by a physician, according to the relevant circumstances, including the condition to be treated, the chosen route of administration, the actual compound administered, the age, weight, and response of the individual subject, the severity of the subject's symptoms, and the like.

The compositions provided herein can be administered one from one or more times per day to one or more times per week; including once every other day. The skilled artisan will appreciate that certain factors can influence the dosage and timing required to effectively treat a subject, including, but not limited to, the severity of the disease or disorder, previous treatments, the general health and/or age of the subject, and other diseases present. Moreover, treatment of a subject with a therapeutically effective amount of a complex described herein can include a single treatment or a series of treatments.

Dosage, toxicity and therapeutic efficacy of the complexes provided herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Complexes exhibiting high therapeutic indices are preferred.

In some embodiments, the composition can further comprise one or more additional peptides or proteins. In certain embodiments, the one or more additional proteins can comprise proteins that detoxify iron, detoxify heme, detoxify Hb or a combination thereof. For example, in some examples, the composition can further comprise transferrin, hemopexin, haptoglobin or a combination thereof. In some embodiments, the composition can further comprise additional (uncomplexed) apohemoglobin, additional (uncomplexed) haptoglobin, or a combination thereof

Methods of Use

The apoHb-Hp complexes described herein can be administered to subjects in need thereof to treat a variety of diseases and disorders.

The apoHb-Hp complexes described herein can be administered to a subject in need thereof, for example, to treat hemolytic anemia and other conditions characterized by or associated with hemolysis. Examples of such conditions include, for example, sickle cell anemia, thalassemia, hemoglobin C disease, hemoglobin SC disease, sickle thalassemia, hereditary spherocytosis, hereditary elliptocytosis, hereditary ovalcytosis, glucose-6-phosphate deficiency and other red blood cell enzyme deficiencies, paroxysmal nocturnal hemoglobinuria (PNH), paroxysmal cold hemoglobinuria (PCH), thrombotic thrombocytopenic purpura/hemolytic uremic syndrome (TTP/HUS), idiopathic autoimmune hemolytic anemia, drug-induced immune hemolytic anemia, secondary immune hemolytic anemia, non-immune hemolytic anemia caused by chemical or physical agents (e.g., chemotherapeutic agents, anti-infective agents), malaria, falciparum malaria, bartonellosis, babesiosis, clostridial infection, severe Haemophilus influenzae type b infection, extensive burns, transfusion reaction, rhabdomyolysis (myoglobinemia), transfusion of stored blood, cardiopulomonary bypass, hemodialysis, red blood cell transfusions, bone marrow failure, hemolytic anemia induced by infection, hemolytic anemia induced by surgery, acute lung injury, radiation-induced hemolytic anemia, and combinations thereof.

In some examples, the complexes described herein can be administered to treat hemolysis associated with sickle cell anemia, malaria, a red blood cell transfusion, thalassemia, an autoimmune disorder, bone marrow failure, an infection, a surgical procedure, a burn, an acute lung injury, sepsis, organ perfusion, the administration of a pharmaceutical agent, the administration of radiation therapy, and a combination thereof. In some examples, the complexes described herein can be administered prophylactically to a subject to prevent damage associated with anticipated hemolysis (e.g., prior to surgery, radiation therapy, acute radiation injury, etc.). In some examples, the complexes described herein can be co-administered with a therapy that induce hemolysis (e.g., a chemotherapeutic agent, an anti-infective agent, a radiation therapy, or a combination thereof).

The complexes described herein can also be added to compositions comprising red blood cells, for example, to stabilize these compositions.

The apoHb-Hp-active agent complexes described herein can also be used to target the delivery of drugs to macrophages or monocytes (e.g., to down-regulate production of inflammatory cytokines, to kill intracellular organisms, or to kill malignant cells). In this way, the complexes can be used to selectively deliver active agents that significant impact certain diseases while minimizing adverse impacts of the active agent on other cells in the body.

In some embodiments, compositions comprising an apoHb-Hp-active agent complex can be administered to a subject in need thereof to treat a disease characterized by the overexpression of CD163. Such diseases are known in the art, and include but not limited to, for example cancer (e.g., breast cancer, Hodgkin Lymphoma), liver cirrhosis, type 2 diabetes, macrophage activation syndrome, Gaucher's disease, sepsis, HIV infection, and rheumatoid arthritis.

In some embodiments, compositions comprising an apoHb-Hp-active agent complex can be administered to a subject in need thereof to treat a disease which involves macrophages or monocytes. Such diseases are known in the art and include, for example, heart disease, HIV infection, cancer, fibrotic diseases (e.g., cystic fibrosis), asthma, inflammatory bowel disease, rheumatoid arthritis, and diseases in which macrophages or monocytes function as hosts for intracellular pathogens (e.g., malaria, tuberculosis, leishmaniasis, chikungunya, adenovirus, Legionnaires' disease, coronavirus (e.g., SARS-CoV-2, SARS, MERS, etc.), and infections caused by bacteria in the genus Brucella such as B. abortus, B. canis, B. melitensis, and B. suis).

The apoHb-Hp complexes described herein (with or without associated active agents) can be administered to a subject in need thereof, for example, to treat hemolytic anemia and other conditions characterized by or associated with hemolysis. Examples of such conditions include, for example, sickle cell anemia, thalassemia, hemoglobin C disease, hemoglobin SC disease, sickle thalassemia, hereditary spherocytosis, hereditary elliptocytosis, hereditary ovalcytosis, glucose-6-phosphate deficiency and other red blood cell enzyme deficiencies, paroxysmal nocturnal hemoglobinuria (PNH), paroxysmal cold hemoglobinuria (PCH), thrombotic thrombocytopenic purpura/hemolytic uremic syndrome (TTP/HUS), idiopathic autoimmune hemolytic anemia, drug-induced immune hemolytic anemia, secondary immune hemolytic anemia, non-immune hemolytic anemia caused by chemical or physical agents (e.g., chemotherapeutic agents, anti-infective agents), malaria, falciparum malaria, bartonellosis, babesiosis, clostridial infection, severe Haemophilus influenzae type b infection, extensive burns, transfusion reaction, rhabdomyolysis (myoglobinemia), transfusion of aged blood, cardiopulomonary bypass, hemodialysis, red blood cell transfusions, bone marrow failure, hemolytic anemia induced by infection, hemolytic anemia induced by surgery, acute lung injury, radiation-induced hemolytic anemia, and combinations thereof.

In some examples, the complexes described herein can be administered to treat hemolysis associated with sickle cell anemia, malaria, a red blood cell transfusion, thalassemia, an autoimmune disorder, bone marrow failure, an infection, a surgical procedure, a burn, an acute lung injury, the administration of a pharmaceutical agent, the administration of radiation therapy, and a combination thereof. In some examples, the complexes described herein can be administered prophylactically to a subject to prevent damage associated with anticipated hemolysis (e.g., prior to surgery, radiation therapy, acute radiation injury, etc.). In some examples, the complexes described herein can be co-administered with a therapy that induce hemolysis (e.g., a chemotherapeutic agent, an anti-infective agent, a radiation therapy, or a combination thereof).

The complexes described herein can also be added to compositions comprising red blood cells, for example, to stabilize these compositions.

The apoHb-Hp complex may also be used to triger CD163+ uptake of drug-conjugated Hp. In addition to administering the full apoHb-Hp-drug complex, only the Hp-drug conjugate or apoHb-drug conjugate could be administered. Then, apoHb or Hp could be administered to induce macrophage and monocyte uptake of the complex. In doing so, the Hp-drug conjugate would have a longer circulatory half-life to perform its desired function. For example, similar to bispecific monoclonal antibodies, Hp could be complexed with a targeting agent and macrophage/monocyte uptake could be trigged by the injection of apoHb. Such a therapeutic approach could be employed to treat cancer. For cancer treatment, Hp conjugated with a cancer cell targeting molecule could be administered to a patient. The Hp conjugated with the cancer cell targeting molecule would circulate until it binds to the surface of the cancer cell. Subsequent administration of apoHb would bind to the Hp attached to the cancer cell. The resulting apoHb-Hp complex attached to the cancer cell would recruit macrophages and monocytes for phagocytosis of the cancerous cell.

In some embodiments, methods can further include administering an agent to a patient to modulate CD163 expression (and by extension circulation and/or delivery of the complexes described herein). For example, methods can comprise administering a gluticosteroid to the patient to increase expression of CD163 or administering an agent (e.g., a gene silencing agent) to decrease expression of CD163.

In some embodiments, the apoHb-Hp complex and an active agent coordinated thereto is administered in combination with an immunotherapy agent, such as an immune checkpoint inhibitor. In some examples, the immune checkpoint inhibitor can comprise an anti-PD1 or anti-PDL1 antibody. In some examples, the immune checkpoint inhibitor can comprise an anti-CTLA4 monoclonal antibody.

In some embodiments, the disease can involve cellular iron accumulation and ferroptosis. In some embodiments, the apoHb-Hp complex and the active agent (e.g., HO-1 enzyme agonist) coordinated thereto are administered in combination with a ferropototic agent, such as Bay117085 or withaferin A.

Methods of Purifying Apohemoglobin

Methods of isolating apoHb protein can comprise (i) contacting Hb with an aqueous solution comprising a water-miscible solvent and a pH modifier, thereby forming a protein solution having a pH of less than 6.5 or greater than 8; and (ii) filtering the protein solution by ultrafiltration against a filtration membrane having a pore size that separates the apohemoglobin from heme, thereby forming a retentate fraction comprising the apoHb and a permeate fraction comprising heme. In some embodiments, methods for isolating an apoHb can further comprise (iii) neutralizing the retentate fraction to isolate the apoHb.

In some examples, the Hb can be present in the protein solution at a concentration from 0.1 mg/mL to 5 mg/mL, such as from 0.5 mg/mL to 3 mg/mL.

The protein solution can have an acidic or basic pH, selected so as to facilitate dissociation of the hydrophobic ligand and the apoprotein.

In some cases, the protein solution can have an acidic pH. In some of these embodiments, the protein solution can have a pH of 6 or less (e.g., 5.5 or less, 5 or less, 4.5 or less, 4 or less, 3.5 or less, 3 or less, or 2.5 or less). In some embodiments, the protein solution can have a pH of 2 or more (e.g., 2.5 or more, 3 or more, 3.5 or more, 4 or more, 4.5 or more, 5 or more, or 5.5 or more).

The protein solution can have a pH ranging from any of the minimum values described above to any of the maximum values described above. For example, in some embodiments, the protein solution can have a pH of from 2 to 6, such as from 3 to 6.

In other cases, the protein solution can have a basic pH. In some of these embodiments, the protein solution can have a pH of greater than 8 (e.g., 8.5 or more, 9 or more, 9.5 or more, 10 or more, or 10.5 or more). In some embodiments, the protein solution can have a pH of 11 or less (e.g., 10.5 or less, 10 or less, 9.5 or less, 9 or less, or 8.5 or less.

The protein solution can have a pH ranging from any of the minimum values described above to any of the maximum values described above. For example, in some embodiments, the protein solution can have a pH of from greater than 8 to 11, such as from greater than 8 to 10.

Generally, the filtration membrane can be rated for retaining solutes having a molecular weight ranging from the molecular weight of the heme to the molecular weight of the apoHb (e.g., such as a membrane rated for retaining solutes having a molecular weight of from about 1 kDa to 100 kDa, such as from about 1 kDa to about 10 kDa).

In connection with the methods described herein, ultrafiltration can comprise direct-flow filtration (DFF), cross-flow or tangential-flow filtration (TFF), or a combination thereof. In certain embodiments, the ultrafiltration can comprise tangential-flow filtration (TFF).

The membranes useful in the filtration steps described herein can be in the form of flat sheets, rolled-up sheets, cylinders, concentric cylinders, ducts of various cross-section and other configurations, assembled singly or in groups, and connected in series or in parallel within the filtration unit. The apparatus can be constructed so that the filtering and filtrate chambers run the length of the membrane.

Suitable membranes include those that separate the desired species from undesirable species in the mixture without substantial clogging problems and at a rate sufficient for continuous operation of the system. Examples are described, for example, in Gabler FR. Tangential flow filtration for processing cells, proteins, and other biological components. ASM News 1984; 50:299-304. They can be synthetic membranes of either the microporous type or the ultrafiltration type. A microporous membrane has pore sizes typically from 0.1 to 10 micrometers, and can be made so that it retains all particles larger than the rated size. Ultrafiltration membranes have smaller pores and are characterized by the size of the protein that will be retained. They are available in increments from 1000 to 1,000,000 Dalton nominal molecular weight limits.

Generally, the filtration membrane can comprise an ultrafiltration membrane. Ultrafiltration membranes are normally asymmetrical with a thin film or skin on the upstream surface that is responsible for their separating power. They are commonly made of regenerated cellulose, polysulfone or polyethersulfone.

In some cases, each filtration step can involve filtration through a single filtration membrane. In other cases, because membrane filters are not perfect and may have pores that allow some intended retentate molecules to slip through, more than one membrane (e.g., two membranes, three membranes, four membranes, or more) having the same pore size can be utilized for a given filtration step. In these embodiments, the membranes can be placed so as to be layered parallel to each other (e.g., one on top of the other) such that filtered fluid sequentially flows through each of the more than one membrane.

Membrane filters for tangential-flow filtration are available as units of different configurations depending on the volumes of liquid to be handled, and in a variety of pore sizes. Particularly suitable for use in the methods described herein, on a relatively large scale, are those known, commercially available tangential-flow filtration units.

The filtration unit useful herein is suitably any unit now known or discovered in the future that serves as an appropriate filtration module, particularly for microfiltration and ultrafiltration. The preferred filtration unit is hollow fibers or a flat sheet device. These sandwiched filtration units can be stacked to form a composite cell. One example type of rectangular filtration plate type cell is available from Filtron Technology Corporation, Northborough, Mass., under the trade name Centrasette. Another example filtration unit is the Millipore Pellicon ultrafiltration system available from Millipore, Bedford, Mass.

The water-miscible solvent can comprise a polar protic solvent. In some embodiments, the water-miscible solvent can comprise an alcohol (e.g., ethanol, methanol, or a combination thereof).

In some embodiments, the aqueous solution can comprise at least 10% by volume (e.g., at least 15% by volume, at least 20% by volume, at least 25% by volume, at least 30% by volume, at least 35% by volume, at least 40% by volume, at least 45% by volume, at least 50% by volume, at least 55% by volume, at least 60% by volume, at least 65% by volume, at least 70% by volume, at least 75% by volume, at least 80% by volume, or at least 85% by volume) alcohol. In some embodiments, the aqueous solution can comprise 90% by volume or less (e.g., 85% by volume or less, 80% by volume or less, 75% by volume or less, 70% by volume or less, 65% by volume or less, 60% by volume or less, 55% by volume or less, 50% by volume or less, 45% by volume or less, 40% by volume or less, 35% by volume or less, 30% by volume or less, 25% by volume or less, 20% by volume or less, or 15% by volume or less) alcohol.

The aqueous solution can comprise a quantity of alcohol ranging from any of the minimum values described above to any of the maximum values described above. For example, in some embodiments, the aqueous solution can comprise from 60% to 90% by volume alcohol (e.g., 60% to 90% by volume ethanol).

In some embodiments, filtering step (ii) can comprise buffer exchange. In certain embodiments filtering step (ii) can comprise continuous diafiltration or dialysis.

Optionally, the retentate fraction can spectroscopically monitored during the continuous diafiltration to monitor separation of the heme from the apoHb. Spectroscopically monitoring the retentate fraction can comprise monitoring a spectroscopic peak (e.g., an absorbance peak) associated with the apoHb and a spectroscopic peak (e.g., an absorbance peak) associated with the Hb. In some embodiments, filtering step (ii) can comprise performing the continuous diafiltration until a relative magnitude of the absorbance peak associated with the apoHb and the absorbance peak associated with the Hb suggest that the apoHb and the Hb are present in the retentate fraction at a molar ratio of at least 9:1 (e.g., at least 10:1, at least 15:1, at least 20:1, at least 25:1, at least 50:1, or at least 100:1).

Methods for isolating an apoHb can further comprise (iii) neutralizing the retentate fraction to isolate the apoHb. In some embodiments, neutralizing step (iii) comprises continuous diafiltration with a buffer solution having a pH of from 6.8 to 7.6.

The purity of isolated apoHb can be assessed using a variety of methods known in the art, including for example, liquid chromatography and/or spectroscopic methods (UV-Vis spectroscopy, fluorescence spectroscopy, etc.). In certain embodiments, the apoHb isolated in step (iii) can comprise less than 1% (e.g., less than 0.75%, less than 0.5%, less than 0.25%, or less than 0.1%) residual heme relative to the concentration of apoHb isolated in step (iii), as measured by a suitable spectroscopic method (e.g., UV Vis spectroscopy).

The apoHb isolated in step (iii) can exhibit excellent stability relative to apoHb isolated using other conventional methodologies. In some embodiments, the apoHb isolated in step (iii) can be stable for a period of at least 7 days (e.g., at least 14 days, at least 30 days, at least 60 days, at least 120 days, or at least 180 days) at 22° C. In certain embodiments, at least 75% (e.g., at least 80%, at least 85%, at least 90%, or at least 95%) of the apoHb remains soluble in solution after storage at 22° C. for 7 days. In certain embodiments, at least 75% (e.g., at least 80%, at least 85%, at least 90%, or at least 95%) of the apoHb remains soluble in solution after storage at 4° C. for 180 days. In certain embodiments, at least 75% (e.g., at least 80%, at least 85%, at least 90%, or at least 95%) of the apoHb remains soluble in solution after storage at −80° C. for 180 days.

In some of these embodiments, at least 65% (e.g., at least 70%, at least 75%, at least 80%, or at least 85%) of the apoHb can retain its activity (i.e., retain its ability to bind heme) after storage at 22° C. for 7 days. In some of these embodiments, at least 65% (e.g., at least 70%, at least 75%, at least 80%, or at least 85%) of the apoHb can retain its activity (i.e., retain its ability to bind heme) after storage at 4° C. for 180 days. In some of these embodiments, at least 65% (e.g., at least 70%, at least 75%, at least 80%, or at least 85%) of the apoHb can retain its activity (i.e., retain its ability to bind heme) after storage at −80° C. for 180 days.

In some embodiments, methods can further comprise lyophilizing the apoHb isolated in step (iii).

Also described are methods of isolating apoHb from a protein solution comprising a Hb. These methods can comprise (i) mildly denaturing the Hb to form a protein solution; and (ii) filtering the protein solution by ultrafiltration against a filtration membrane having a pore size that separates the apoHb from the heme, thereby forming a retentate fraction comprising the apoHb and a permeate fraction comprising the heme. In some embodiments, methods for isolating an apoHb can further comprise (iii) neutralizing the retentate fraction to isolate the apoHb.

In some examples, mildly denaturing the Hb can comprise heating the Hb (e.g., to a temperature of from 40° C. to 60° C.).

In some examples, mildly denaturing the Hb can comprise contacting the Hb with a pH modifier (e.g., with an acid and/or a base). Mildly denaturing the Hb can comprise contacting the Hb with an effective amount of a pH modifier to produce an acidic or basic pH, selected so as to facilitate dissociation of the heme and the apoHb.

In some cases, mildly denaturing the Hb can comprise contacting the Hb with an effective amount of a pH modifier to produce a pH of 6 or less (e.g., 5.5 or less, 5 or less, 4.5 or less, 4 or less, 3.5 or less, 3 or less, or 2.5 or less). In some embodiments, mildly denaturing the Hb can comprise contacting the Hb with an effective amount of a pH modifier to produce a pH of 2 or more (e.g., 2.5 or more, 3 or more, 3.5 or more, 4 or more, 4.5 or more, 5 or more, or 5.5 or more).

Mildly denaturing the Hb can comprise contacting the Hb with an effective amount of a pH modifier to produce a pH ranging from any of the minimum values described above to any of the maximum values described above. For example, in some embodiments, mildly denaturing the Hb can comprise contacting the Hb with an effective amount of a pH modifier to produce a pH of from 2 to 6, such as from 3 to 6.

In other cases, mildly denaturing the Hb can comprise contacting the Hb with an effective amount of a pH modifier to produce a pH of 8 or more (e.g., 8.5 or more, 9 or more, 9.5 or more, 10 or more, or 10.5 or more). In some embodiments, mildly denaturing the Hb can comprise contacting the Hb with an effective amount of a pH modifier to produce a pH of 11 or less (e.g., 10.5 or less, 10 or less, 9.5 or less, 9 or less, or 8.5 or less.

Mildly denaturing the Hb can comprise contacting the Hb with an effective amount of a pH modifier to produce a pH ranging from any of the minimum values described above to any of the maximum values described above. For example, in some embodiments, mildly denaturing the Hb can comprise contacting the Hb with an effective amount of a pH modifier to produce a pH of from 8 to 11, such as from 8 to 10.

In some examples, mildly denaturing the conjugated protein can comprise contacting the Hb with a non-aqueous solvent, such as an alcohol. Examples of such non-aqueous solvents include, for example, ethanol, methanol, isopropanol, butanol, 2-propanol, phenol, or combinations thereof.

In some examples, mildly denaturing the Hb can comprise contacting the Hb with a chaotropic agent (e.g., a salt that can disrupt the structure of a protein by shielding charges and preventing the stabilization of salt bridges). Any salt in principle may be used. Examples of suitable chaotropic agents include, but are not limited to, guanidinium chloride, lithium perchlorate, lithium acetate, magnesium chloride, sodium dodecyl sulfate, thiourea, urea, calcium chloride, and combinations thereof.

ApoHb prepared by the filtration methods described herein (after renaturation/neutralization) can exhibit improved stability and purity as compared to apoHb prepared by existing precipitation and liquid-liquid extraction methodologies.

In some embodiments, the apoHb produced by the filtration methods described herein can be stable for a period of at least 7 days (e.g., at least 14 days, at least 30 days, at least 60 days, at least 120 days, or at least 180 days) at 22° C. In certain embodiments, at least 75% (e.g., at least 80%, at least 85%, at least 90%, or at least 95%) of the apoHb produced by the filtration methods described herein remains soluble in solution after incubation at 22° C. for 7 days. In certain embodiments, at least 75% (e.g., at least 80%, at least 85%, at least 90%, or at least 95%) of the apoHb produced by the filtration methods described herein remains soluble in solution after incubation at 4° C. for 180 days. In certain embodiments, at least 75% (e.g., at least 80%, at least 85%, at least 90%, or at least 95%) of the apoHb produced by the filtration methods described herein remains soluble in solution after incubation at −80° C. for 180 days.

In some of these embodiments, at least 65% of the apoHb can retain its activity (i.e., retain its ability to bind heme) after storage at 22° C. for 7 days. In some of these embodiments, at least 65% of the apoHb can retain its activity (i.e., retain its ability to bind heme) after storage at 4° C. for 180 days. In some of these embodiments, at least 65% of the apoHb can retain its activity (i.e., retain its ability to bind heme) after storage at −80° C. for 180 days.

The apoHb prepared by various methods possess the same chemical identity (primary structure) and primarily the same quaternary conformation compared to apoHb prepared by existing precipitation or liquid-liquid extraction methodologies. The apoHb produced by the ultrafiltration methods described herein can exist in aqueous solution primarily as an αβ dimer without the use of reducing agents (2-mercaptoethanol, dithiothreitol). In contrast, previous methodologies may produce non-native tetramers (a2(32) that require reducing agents to form αβ dimers. Furthermore, the apoHb produced in the current methodology is stable for over a week at room temperature and stable at 4° C., −80° C. and in lyophilized form. Previous methodologies produced apoHb that quickly precipitated (approximately 24 hours) when stored at room temperature. In certain embodiments, the apoHb can be characterized by a residual Soret peak having a maximum absorption ranging from 411-417 nm, such as 412 nm (after renaturation/neutralization, but before complexation with Hp).

Methods for the Purification of Haptoglobin

Haptoglobin (Hp) can be isolated from plasma or a fraction thereof. In some embodiments, methods for isolating Hp from plasma or a fraction thereof can comprise (i) clarifying the plasma or fraction thereof and (ii) filtering the clarified plasma or a fraction thereof by ultrafiltration against a filtration membrane, thereby forming a retentate fraction comprising Hp having a molecular weight of greater than about 100 kDa and a permeate fraction comprising serum proteins and other impurities having a molecular weight of less than about 100 kDa.

The plasma or fraction thereof can comprise plasma fraction IV, plasma fraction V, a fraction of precipitated plasma (from salting out, or equivalent) or a combination thereof.

Clarifying the plasma or a fraction thereof can comprise removing suspended solids from the plasma or fraction thereof. Removing suspended solids from the plasma or fraction thereof can comprise filtering (via ultrafiltration, microfiltration, depth filtration or equivalent) the plasma or a fraction thereof, contacting the plasma or a fraction thereof with a salting out agent (e.g., ammonium sulfate), an adsorbing agent (e.g., ethacridine lactate), or a combination thereof. Further clarification may be implemented through addition of a lipid-binding agent such as fumed silica (such as fumed silica sold under the tradename Aerosil³⁸⁰®, or similar), clay, bentonite, terra alba, active carbon, or a combination thereof.

In some embodiments, the ultrafiltration can comprise tangential-flow filtration.

In some cases, the method can further comprise filtering the permeate fraction comprising serum proteins and other impurities by ultrafiltration against a second filtration membrane, thereby forming a second retentate fraction comprising a blend of proteins having a molecular weight below about 100 kDa and above a cutoff value and a second permeate fraction comprising serum proteins and other impurities having a molecular weight below the cutoff value, wherein the blend of proteins in the second permeate comprises low molecular weight Hp, transferrin, hemopexin, or a combination thereof. The cutoff value can be from about 20 kDa to about 70 kDa, such as from about 25 kDa to about 50 kDa

In these methods, the second retentate fraction can include a blend of proteins (e.g., low molecular weight Hp, transferrin, hemopexin, or a combination thereof) that can bind and detoxify cell-free Hb, free iron, and/or free heme.

In other embodiments, methods for isolating Hp from plasma or a fraction thereof can comprise (i) filtering the plasma or fraction thereof by ultrafiltration against a first filtration membrane, thereby forming a first retentate fraction comprising serum proteins having a molecular weight above a first cutoff value and a first permeate fraction comprising most of the Hp and serum proteins having a molecular weight below the first cutoff value; and (ii) filtering the first permeate fraction by ultrafiltration against a second filtration membrane, thereby forming a second retentate fraction comprising small amounts of Hp2-1, Hp2-2, and serum proteins having a molecular weight below the first cutoff value and above a second cutoff value; and a second permeate fraction comprising Hp2-1, Hp2-2, and serum proteins having a molecular weight below the second cutoff value. In some cases, the method can further comprise (iii) filtering the second permeate fraction by tangential-flow filtration against a third filtration membrane, thereby forming a third retentate fraction comprising Hp2-1 and Hp2-2 having a molecular weight below the second cutoff value and above a third cutoff value; and a third permeate fraction comprising low molecular weight Hp, serum proteins and other impurities having a molecular weight below the third cutoff value. In some cases, the method can further comprise (iv) filtering the third permeate fraction comprising low molecular weight Hp, serum proteins and other impurities by ultrafiltration against a fourth filtration membrane, thereby forming a fourth retentate fraction comprising a blend of proteins having a molecular weight below the third cutoff value and above a fourth cutoff value and a fourth permeate fraction comprising serum proteins and other impurities having a molecular weight below the fourth cutoff value, wherein the blend of proteins in the retentate comprises low molecular weight Hp, transferrin, hemopexin, or a combination thereof.

The first cutoff value can be from about 650 kDa to about 1000 kDa. The second cutoff value can be from about 300 kDa to about 700 kDa. The third cutoff value can be from about 70 kDa to about 200 kDa. The fourth cutoff value can be from about 20 kDa to about 70 kDa. In certain examples, the first cutoff value can be about 750 kDa, the second cutoff value can be about 500 kDa, and the third cutoff value can be about 100 kDa. The fourth cutoff value can be about 30 kDa or about 50 kDa.

The plasma or fraction thereof can comprise plasma fraction IV, plasma fraction V, a fraction of precipitated plasma (from salting out, or equivalent) or a combination thereof.

Clarifying the plasma or a fraction thereof can comprise removing suspended solids from the plasma or fraction thereof. Removing suspended solids from the plasma or fraction thereof can comprise filtering (via ultrafiltration, microfiltration, depth filtration or equivalent) the plasma or a fraction thereof, contacting the plasma or a fraction thereof with a salting out agent (e.g., ammonium sulfate), an adsorbing agent (e.g., ethacridine lactate), or a combination thereof. Further clarification may be implemented through addition of a lipid-binding agent such as fumed silica (such as fumed silica sold under the tradename Aerosil³⁸⁰®, or similar), clay, bentonite, terra alba, active carbon, or a combination thereof.

In some embodiments, the ultrafiltration can comprise tangential-flow filtration.

In these methods, the fourth retentate fraction can include a blend of proteins (e.g., low molecular weight Hp, transferrin, hemopexin, or a combination thereof) that can bind and detoxify free Hb, free iron, and/or free heme.

Hp can also be isolated from a solution (e.g., plasma or a fraction thereof) by exploiting molecular size changes induced by protein complex formation. Such methods can comprise (i) filtering the protein solution by ultrafiltration against a first filtration membrane, thereby forming a first retentate fraction comprising impurities having a molecular weight above a first cutoff value and a first permeate fraction comprising the Hp and impurities having a molecular weight below the first cutoff value; (ii) contacting the first permeate fraction with a binding molecule that selectively associates with the Hp to form a Hp complex having a molecular weight above the first cutoff value; and (iii) filtering the first permeate fraction by ultrafiltration against a second filtration membrane (at the same or above the cut-off of the first membrane), thereby forming a second retentate fraction comprising the Hp complex having a molecular weight above the first cutoff value and a second permeate fraction comprising the impurities having a molecular weight below the first cutoff value.

In some cases, the Hp complex can be isolated (e.g., if the Hp itself is useful, or if the Hp complex is more stable under storage than the Hp or binding molecule). For example, in one example, the binding molecule can comprise apoHb (e.g., prepared as described above). In these embodiments, the resultant Hp complex can be an apoHb-Hp complex described herein.

In other cases, the method can further involve dissociating the Hp complex to re-form the Hp, and isolating the Hp. For example, the method can further comprise (iv) contacting the second retentate fraction with a dissociating agent, thereby inducing dissociation of the Hp complex to the Hp and the binding molecule, and (v) filtering the second retentate fraction to separate the Hp from the binding molecule and the dissociating agent, thereby isolating the Hp.

In some of these embodiments, step (v) can comprise filtering the second retentate fraction by ultrafiltration against a third filtration membrane, thereby forming a third retentate solution comprising the Hp having a molecular weight above a second cutoff value and a second permeate fraction comprising the impurities having a molecular weight below the second cutoff value.

If desired, ultrafiltration may be done with staging to improve separation between retained and filtrated solutes.

By way of non-limiting illustration, examples of certain embodiments of the present disclosure are given below.

### EXAMPLES

### Example 1: Scalable Production of Apohemoglobin Via Tangential Flow Filtration

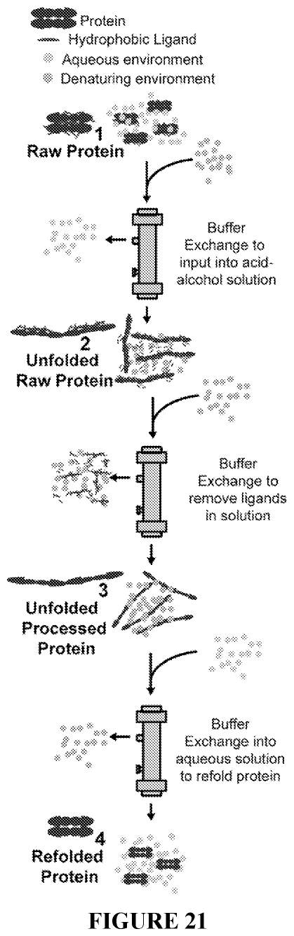

The general schematic for the procedure to remove hydrophobic ligands from proteins employing the invention presented here is shown in FIG. 21.

Apohemoglobin (apoHb) is a dimeric globular protein with two vacant heme-binding pockets that can bind heme or other hydrophobic ligands. Purification of apoHb is based on partial hemoglobin (Hb) unfolding to facilitate heme extraction into an organic solvent. However, current production methods are time consuming, difficult to scale up, and use highly flammable and toxic solvents. In this study, a novel and scalable apoHb production method was developed using an acidified ethanol solution to extract the hydrophobic heme ligand into solution and tangential flow filtration to separate heme from the resultant apoprotein. Total protein and active protein yields were >95% and ˜75%, respectively, with <1% residual heme in apoHb preparations and >99% purity from SDS-PAGE analysis. Virtually no loss of apoHb activity was detected at 4° C., −80° C., and in lyophilized form during long term storage. Structurally, size exclusion chromatography (SEC) and circular dichroism (CD) spectroscopy indicated that apoHb was dimeric with a ˜25% reduction of helical content compared to Hb. Furthermore, mass spectroscopy and reverse-phase chromatography indicated that the mass of the α and β subunits were virtually identical to the theoretical mass of these subunits in Hb and had no detectable oxidative modifications upon heme removal from Hb. SEC confirmed that apoHb bound to haptoglobin at similar ratio to that of native Hb. Finally, reconstituted Hb (rHb) was processed via a hemichrome removal method to isolate functional rHb for biophysical characterization in which the O₂ equilibrium curve, O₂ dissociation and CO association kinetics of rHb were virtually identical to native Hb. Overall, this study describes a novel and improved method to produce apoHb, as well as presents a comprehensive biochemical analysis of apoHb and rHb.

Human hemoglobin (Hb) is the major protein component contained inside human red blood cells (RBCs), and is well known for its role in oxygen (O₂) storage and transport. It is a tetrameric protein (64 kDa), which consists of two pairs of αβ dimers (32 kDa) held together by non-covalent bonds. In each of the four globin chains (2a and 2(3 globins), a single heme prosthetic group is tightly bound inside the hydrophobic heme-binding pocket. Upon removal of heme from Hb, the resulting protein loses some of its helical content compared to native Hb. The resulting apoprotein is referred to as apohemoglobin (apoHb). ApoHb can react with heme to form reconstituted Hb (rHb), which shows virtually no difference in biophysical properties compared to native Hb. The heme-binding ability and heme-induced structural changes of apoHb make it an interesting precursor for studies into in vivo Hb synthesis and recombinant Hb production.

ApoHb is an attractive delivery vehicle for hydrophobic drug molecules, which can bind within the vacant heme-binding pockets. Heme is highly hydrophobic and cytotoxic; however, when bound inside the heme-binding pocket of Hb, its toxicity is reduced, and aqueous solubility increases. In addition to heme, other hydrophobic molecules such as modified hemes or therapeutic drug molecules can bind to the hydrophobic heme-binding pocket of apoHb.

Another exciting property of apoHb is its' clearance through CD163+ macrophages or monocytes. Similar to Hb, apoHb binds to haptoglobin (Hp). Hp is a plasma protein mainly responsible for the clearance of cell-free Hb. The apoHb-Hp/Hb-Hp complex is then recognized and uptaken by CD163+ macrophages and monocytes. This specific mode of clearance allows for targeted drug delivery to macrophages or monocytes. Thus, the ability of apoHb to bind hydrophobic molecules and facilitate targeted delivery towards CD163+ macrophages or monocytes make it a promising hydrophobic drug delivery vehicle.

From these properties, not only can apoHb be used as a drug carrier for hydrophobic molecules (i.e. molecules insoluble in aqueous solution) and targeted drug delivery to macrophages and monocytes, but its high heme affinity could be used to scavenge heme in vivo. States of hemolysis release cell-free Hb, which can lose its heme moiety. Free heme can undergo various redox-reactions, causing oxidation of various tissues. ApoHb could scavenge free heme, and thus forming cell-free Hb that can be cleared through CD163+ macrophages or monocytes. Furthermore, the hydrophobic molecule binding properties of apoHb can be used to bind MRI contrast agent molecules such as Mn-porphyrins (similar structure to normal Hb heme but with switching the Fe metal atom to Mn). The same idea applies to binding of fluorescent molecules to the vacant hydrophobic heme-binding pocket.

Another application of apoHb is its potential use in photodynamic therapy (PDT). PDT has recently been used to effectively treat cancers and other illnesses through the production of reactive oxygen species (ROS). The ROS produced by PDT surpasses the ability of cancer cells to resist cell apoptosis, disrupts the tumor vasculature and promotes shifting the immune system against the tumor. Furthermore, this treatment could be used for cancers like triple-negative breast cancer, in which commonly targeted receptors are not expressed. However, most photosensitizers (PS) lack specificity for tumor cells, have poor solubility, and cause systemic photosensitivity, inducing phototoxic and photoallergic reactions. Many PS targeting mechanisms are expensive, complicated to develop, or leak PS. Fortunately, the high expression of CD163+ tumor associated macrophages (TAM) in cancers could be targeted through their uptake of apohemoglobin (apoHb) via CD163 mediated endocytosis. PS bound to apoHb could improve PDT treatment by not only improving its biocompatibility and effectiveness, but also by specifically targeting a form of TAM that contributes to tumor growth. Furthermore, the metals in PS provide a second pathway for ROS production and anti-tumor immune response. Thus, treatment with PS bound apoHb could enhance the immunological shift against the tumor by lowering macrophage density and stimulating TAM differentiation to an anti-cancer phenotype. This immune change can destroy secondary tumors and prevent cancer metastasis and regression. We have successfully bound aluminum-phthalocyanines (a highly potent PS molecule currently undergoing clinical trials) to apoHb, thus increasing its' solubility in aqueous solution.

The first successful method for producing active apoHb was developed by Faneli et al. in 1958. In Fanelli's acetone extraction method, Hb was added to acidified acetone at −20° C. extracting heme into solution while precipitating the apoprotein (globin). After separation of the solid protein from the liquid phase, the apoprotein was re-dissolved in deionized (DI) water followed by extensive dialysis, yielding apoHb. Another procedure for active apoHb production was later developed by Teale in 1959, and further improved upon by Yonetani. In this process, heme was removed from Hb through exposure to acidified methylethylketone (MEK), forming two immiscible liquid layers. The heme partitions into the organic layer, while the globin partitions into the aqueous layer. After liquid-liquid separation of the layers, the aqueous globin solution underwent extensive dialysis similar to the acetone extraction method to yield apoHb.

However, the aforementioned apoHb production processes have various drawbacks that complicate scale up. First, both processes use highly flammable solvents and require costly separation equipment. Additionally, in the case of MEK extraction (the solvent most likely to be used for scale up), about 40% of the water-rich phase becomes saturated with MEK. Thus, extensive treatment is required to lower the MEK concentration in the aqueous phase before the aqueous phase can be safely discarded. The high MEK concentration in the aqueous phase will also require repeated MEK extractions to significantly lower the heme content of the purified apoHb. In the case of acetone extraction, not only is there an additional safety risk associated with centrifuging a flammable solvent, but the process may also require sequential acetone exposure to sufficiently remove heme, especially due to the possibility of heme entrapment within the protein precipitate. Finally, when acetone and MEK are used as heme extraction solvents at large-scales, buffer-exchange via dialysis will require large volumes of buffer and is a slow process. In this current work, a scalable and simple process for manufacturing apoHb is described.

Tangential flow filtration (TFF) is a size exclusion filtration technique greatly used in industrial biotechnology for purification of biomolecules due to its linear scalability, economic benefits and long membrane lifetime. Additionally, TFF easily facilitates controlled buffer exchange via diafiltration, which is preferable to extensive and lengthy dialysis and can reduce the equipment footprint for production. In this example, TFF was used to produce and purify apoHb using an acidic 80% ethanol solution (v/v) as the heme extraction solvent. It is important to note that ethanol poses a much lower flammability risk compared to previously used heme extraction solvents given that its flashpoint is 20° C. compared to −18° C. and −3° C. for acetone and MEK, respectively.

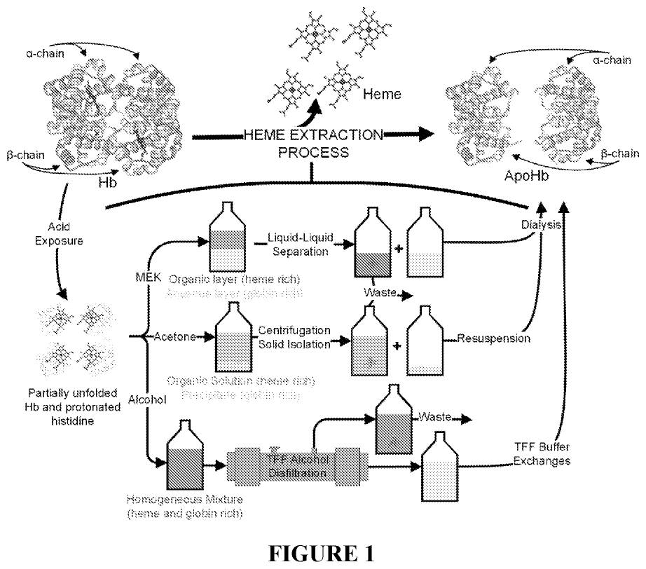

In this TFF process, the Hb precursor in aqueous solution was added to a TFF system filled with an acidic ethanol solution. Then, the mixture of acidic ethanol and Hb underwent continuous diafiltration in the TFF system with acidic ethanol as the diafiltration solution until sufficient heme was extracted from the mixture (i.e., the absorbance ratio of the Soret peak at 412 nm divided by the 280 nm protein peak was lower than 0.1). Next, DI water was used as the diafiltration solution to neutralize the acidic ethanol-Hb solution and to remove ethanol and any free heme from solution. Finally, the desired buffer for apoHb storage and analysis was used as the diafiltration solution for the last diafiltration step (a more detailed procedure for apoHb production via TFF can be found in the Methods Section). FIG. 1 summarizes the prominent methods for producing active apoHb, compared to the method presented in this example.

Analysis of apoHb produced via the TFF purification process (TFF-apoHb) was accomplished by analyzing protein yields and biochemical properties of the resultant apoHb. The stability of apoHb as a function of storage time was also examined at different concentrations and temperatures via quantification of active and total protein of stored apoHb samples. The reconstituted Hb from TFF-apoHb also had its biophysical properties analyzed and compared to native Hb via its absorbance spectrum, O₂ equilibrium curve, O₂ dissociation and carbon monoxide (CO) binding kinetics.

Materials and Methods

Materials. Na₂HPO₄ (sodium phosphate dibasic), NaH₂PO₄ (sodium phosphate monobasic), NaHCO₃(sodium bicarbonate), and hemin chloride were all procured from Sigma Aldrich (St. Louis, Mo.). KCN (potassium cyanide), HCl (hydrochloric acid), acetone, HPLC grade acetonitrile, HPLC grade trifluoroacetic acid (TFA), nylon syringe filters (rated pore size 0.22 μm), and dialysis tubing (rated pore size: 6-8 kDa) were purchased from Fisher Scientific (Pittsburgh, Pa.), while Millex-GP PES syringe filters (rated pore size: 0.2 μm) were purchased from Merck Millipore (Billerica, Mass.). Expired units of human RBCs were generously donated by the Transfusion Service in the Wexner Medical Center at The Ohio State University (Columbus, Ohio).

Hb Preparation. Human Hb for use in this study was prepared via TFF as described by Palmer et al. (Palmer, A. F., Sun, G. & Harris, D. R. Tangential flow filtration of hemoglobin. Biotechnol. Prog. 25, 189-199 (2009)). The concentration of Hb was determined spectrophotometrically based on the Winterbourn equation.

TFF-apoHb Preparation. A KrosFlo Research II TFF system (Spectrum Laboratories, Rancho Dominguez, Ca) with a single 10 kDa polysulfone (PS) hollow fiber (HF) module was used to purify apoHb from Hb. To examine the scalability of the purification process, the process was first performed on TFF filter (P/N: X11S-300-10S) with 20 cm² surface area (MicroKros, Spectrum Laboratories, Rancho Dominguez, Ca) then scaled up to TFF filter (P/N: M11S-360-01S) with 1,050 cm² surface area (MiniKros, Spectrum Laboratories, Rancho Dominguez, Ca). For both size filters, the individual HFs were 0.5 cm in diameter and were 20 cm in length. Purified Hb was added to a 80% (v/v) EtOH/DI water mixture containing 3 mM HCl (acidic ethanol) to achieve a maximum protein concentration of 2 mg/mL. For experiments with microKros filters, 18 mg of Hb was used as the basis. However, experiments utilizing miniKros filters (larger surface area than microKros filters) consisted of three batches with 1 g Hb, three batches with 1.2 g Hb and 8 batches with 2.0 g Hb (ran with two parallel HF modules) as the basis, respectively. The Hb dispersed in the acidic ethanol solution was continuously subjected to diafiltration with 9 times its' initial volume with acidic ethanol to remove heme from solution. After heme removal, the heme-free globin was subjected to diafiltration with DI water with 5 times its volume.

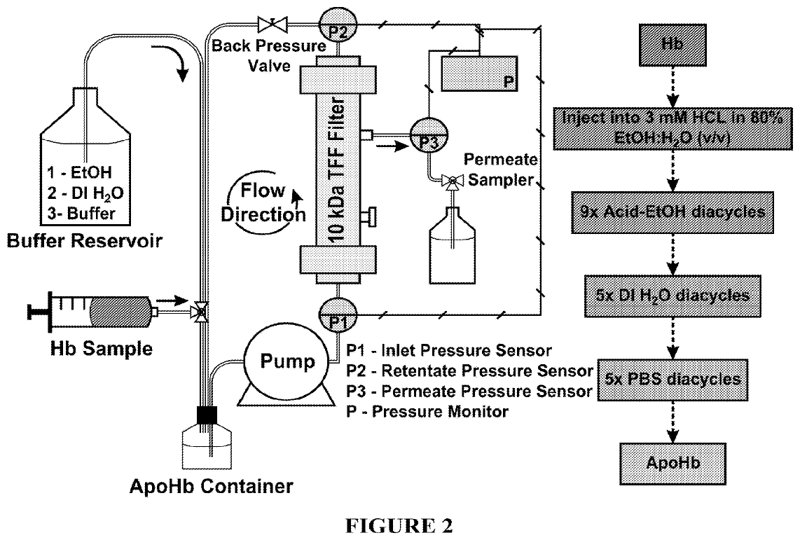

Finally, the apoHb solution was subjected to diafiltration with 5 times its initial volume using a final buffer solution consisting of either phosphate buffered saline (PBS, 10 mM phosphate, 137 mM NaCl, and 2.7 mM KCl, pH 7.4) or 0.1 M phosphate buffer (PB, pH 7.0). During processing, flow rates of 25 mL/min and 1.1 L/min were used for the microKros and miniKros HF modules, respectively. The transmembrane pressure was maintained at 7±1 psi with a back-pressure valve to facilitate optimal permeate flux. For large-scale production, a final concentration step was performed in which the apoHb solution volume was reduced to 50±10 mL then further concentrated on 10 kDa PS microKros filters to a final total protein concentration of 50-115 mg/mL. The entire process was performed in a cold room maintained at 4±1° C. After each run, TFF modules were rinsed with DI water followed by sanitization with 0.5 M NaOH. The modules were stored in 0.1 M NaOH and were extensively washed with DI water prior to use. A schematic of the apoHb TFF production schematic is shown in FIG. 2.

Acetone ApoHb Preparation. The method commonly used to produce apoHb via acetone heme extraction was followed according to the protocol outlined by Fanelli et al.

Total Protein Assays. The total protein concentration of the apoHb solution was measured using a Coomassie Plus Protein assay kit (Pierce Biotechnology, Rockford, Ill.).

ApoHb Activity Assay. The activity of the heme-binding pocket of apoHb was determined via a dicyanohemin (DCNh) incorporation assay. Briefly, analysis of the equilibrium absorbance at 420 nm from apoHb and DCNh mixtures was used to determine the saturation point of apoHb heme-binding pockets with heme. The extinction coefficients of DCNh and rHbCN were 85 mM⁻¹ cm⁻¹ and 114 mM⁻¹ cm⁻¹, respectively.

ApoHb Stability. Three TFF-apoHb batches were prepared. Each batch was divided into three groups to test apoHb stability over time. These groups consisted of unconcentrated, concentrated and lyophilized apoHb. The unconcentrated group was obtained after buffer exchange of apoHb into PBS buffer at a concentration of −2 mg/mL. The remainder of the batch was either sent to be lyophilized or to be concentrated to −40 mg/mL. Immediately after production, apoHb activity and total protein was quantified via the DCNh activity assay and 280 nm absorbance, respectively. Of the concentrated and unconcentrated apoHb groups from each batch, samples were stored at either 37, 22, 4 or −80° C. for subsequent analysis. The lyophilized powder was stored in a closed container at −80° C. Additionally, the stored apoHb groups were reconstituted into rHb and had their absorbance spectra and O₂ dissociation curve measured. After measuring the initial time point (immediately after production), each storage condition was assayed at varying time intervals to measure apoHb activity over time. These time intervals were chosen to capture relevant changes in activity at each storage condition. At 37° C., apoHb was expected to quickly lose activity, so measurements were made every 12 hours. In contrast, apoHb stored at −80° C. was expected to maintain activity for longer time durations. Thus, after initial measurements on a weekly basis, the insignificant changes lead to longer intervals between measurements. Statistical analysis was performed on JMP Pro v 12.2.2 (SAS Institute, Cary, N.C.) and measured concentrations were compared to the initial time point values of each batch. A linear fit with the logarithmic value of the concentration was used to examine the effect of time with the ANOVA test. For time point differences, time was considered as discrete and the TUKEY HSD test was performed.

Mass Spectroscopy. Before analysis, Hb and apoHb samples were buffer exchanged into 100 mM ammonium acetate (Fisher Scientific; San Jose, Calif.) using Micro Bio-Spin™ 6 columns (Bio-Rad; Hercules, Calif.). Samples were tested on a Finnigan LTQ mass spectrometer (Thermo Fisher Scientific, Waltham, Mass.) and analyzed using Xcalibur 2.2 software (Thermo Fisher Scientific, Waltham, Mass.). Samples from the same stock apoHb and Hb were then denatured in 1% acetic acid acetate (Fisher Scientific; San Jose, Calif.) and retested. The mass spectrometer parameters were: spray voltage: 1.5 kV; flow rate: 5 μL/min; capillary temperature: 200° C.; 3 microscans; and 100 ms injection time. The data was deconvoluted using mMass 5.5.0, (Copyright 2018 by Martin Strohalm).

Residual Heme Analysis. The residual heme in apoHb preparations was quantified via size exclusion chromatography (SEC). ApoHb samples prepared via TFF were separated on an analytical BioSep-SEC-53000 (600×7.5 mm) column (Phenomenex, Torrance, Calif.) attached to a Waters 2535 quaternary gradient module, Waters 2998 photodiode array multi-wavelength detector, and controlled using Empower Pro software (Waters Corp., Milford, Mass.). The mobile phase consisted of 50 mM potassium phosphate, pH 7.4. Since pigment-free proteins such as apoHb absorb at 280 nm and heme bound proteins such as Hb have a sharp Soret peak at 400-450 nm, the absorption wavelength was set at λ=280 nm to detect protein (although heme and heme-bound proteins also absorb at 280 nm), and λ=405 and 413 nm to detect protein containing heme. The number of heme molecules retained in apoHb preparations produced via TFF were determined by comparing the Soret spectra of four apoHb samples with the Soret spectra of a Hb sample of known concentration. The number of heme molecules in the apoHb preparation was then compared to the total protein of the sample (on a molar basis) to obtain the percentage of residual heme in each apoHb preparation.

Quaternary Structure. To estimate the quaternary structure of TFF-apoHb, apoHb and protein standards (conalbumin, 76 kDa; hHb, 64 kDa; carbonic anhydrase, 29 kDa; ribonuclease A, 14 kDa; and aprotinin, 6.5 kDa) were analyzed on a SEC column. The known molecular weight (MW) of the standards and their elution volumes were used to determine the coefficients (A, B) of a base 10 exponential function (MW=10^{A*(elution volume)+B}) via non-linear regression. The estimated function parameters were used to estimate the MW of TFF-apoHb based on its elution volume. Samples were separated on an analytical Acclaim SEC-1000 (4.6×300 mm) column (Thermo Fisher Scientific, Waltham, Mass.) attached to a Dionex UltiMate 3000 system (Thermo Fisher Scientific, Waltham, Mass.). The mobile phase consisted of 50 mM potassium phosphate, pH 7.4. The flow rate and UV-visible spectral detection was controlled on Chromeleon 7 software with detection set to A=280 nm to detect protein elution at a flow rate 0.35 mL/min.

Haptoglobin Binding. To analyze haptoglobin (Hp) binding to TFF-apoHb, increasing concentrations of apoHb were mixed with haptoglobin (Hp) and the resultant mixture separated on a SEC column for analysis. Large molecular weight Hp (mixture of Hp2-2 and Hp2-1) was mixed with apoHb with a molecular weight of −32 kDa (dimeric apoHb) and separated on an analytical Acclaim SEC-1000 (4.6×300 mm) column (Thermo Fisher Scientific, Waltham, Mass.) attached to a Dionex UltiMate 3000 system (Thermo Fisher Scientific, Waltham, Mass.). The mobile phase consisted of 50 mM potassium phosphate, pH 7.4. The flow rate and UV-visible spectral detection was controlled on Chromeleon 7 software with detection set to A=280 nm to detect protein elution at a flow rate 0.35 mL/min. The percent change of the area under the curve between pure apoHb and a mixture of excess apoHb and Hp was used to determine the percent of apoHb that was bound to Hp. This percentage was compared to the mass of pure apoHb loaded to determine the Hp binding capacity of apoHb. The same procedure was repeated with Hb for comparison.

Reverse Phase Chromatography. Reverse phase (RP) chromatography was performed with a BioBasic-18 column (Thermo Scientific, Waltham, Mass.) on a Thermo Scientific Dionex Ultimate UHPLC system. The flow rate of the mobile phase was set to 0.75 mL/min. The column was equilibrated with 35% acetonitrile and 65% TFA (0.5 wt %, pH 2.6) for 10 minutes. The gradient was then shifted to 43% acetonitrile over a 1 minute interval. The protein was eluted with an increasing linear gradient of 43 to 47% acetonitrile for 30 minutes. The column was then held at 47% acetonitrile for 20 minutes.

Circular Dichroism. The far UV circular dichroism (CD) spectra of TFF-apoHb was measured on a JASCO J-815 CD spectrometer. Various TFF-apoHb samples and Hb were diluted in DI water to approximately 10 μM. The ellipticity of the samples was measured from 190-260 nm using a 0.1 mm path-length quartz cuvette. The change in alpha helical content of the apoglobin was determined via the ratio of the alpha-helix peak at 222 nm between TFF-apoHb to hHb.

Hb Reconstitution and Preparation. To regenerate the O₂-binding capacity of Hb (i.e. reconstituted Hb, rHb), samples of apoHb were reconstituted with hematin to yield met-rHb and then reduced to yield rHb. First, hematin was added in excess to apoHb to yield met-rHb. The reaction was left overnight at 4±0.5° C. to go to completion. Met-rHb was centrifuged and passed through a 0.22 μm filter before any experiments were conducted. Reduction of met-rHb to yield deoxy-rHb was achieved by adding sodium dithionite at 1.5 mg/mL to met-rHb. The solution was then subjected to diafiltration on a 10 kDa TFF module to remove excess dithionite and any excess heme in solution using a modified HEMOX buffer (135 mM NaCl, 30 mM TES {N-[Tris (hydroxymethyl) methyl]-2-aminoethanesulfonic acid}, 5 mM KCl, pH 7.40±0.02 at 37° C.). During diafiltration, the system was open to the atmosphere to facilitate the conversion of rHb to oxygenated rHb.

Hemichrome Removal and rHb purification. rHb presented an altered absorbance spectra compared to pure Hb corresponding to the presence of hemichromes and/or heme bound to denatured globins. Two methods were developed to remove these unwanted species from solution. For analysis of small dilute samples in a spectrophotometer, passing the solution through a 0.22 μm nylon syringe filter removed the hemichromes by binding them to the hydrophobic filter membrane. However, this method was limited to processing small volumes of material, since the filter membrane would become saturated with these globin-heme species. When larger volumes of rHb were needed for analysis (i.e. more than 1 mg), an oxy-rHb sample was placed under a CO atmosphere to convert rHb into CO-rHb, a highly stable form of Hb. Then, the CO-rHb solution with the unwanted heme-globin complexes was heated to 65±1° C., and left under a CO atmosphere during about 100 minutes to precipitate hemichrome/heme. The hemichrome/heme precipitate was removed and the resulting CO-rHb solution was converted into oxy-rHb by placing it under a pure O₂ stream for 2 hours.

rHb Analysis. Various liganded forms of rHb were analyzed via UV-Vis spectroscopy and compared against native Hb. The oxy-rHb and oxyHb equilibrium binding curves were measured using a Hemox analyzer (TCS Scientific Corp., New Hope, Pa.) at 37° C. The spectra of rHb was measured after one day reaction with excess heme (stage 1), after reduction and diafiltration with the modified HEMOX buffer (stage 2), after placing the rHb mixture under a CO atmosphere (stage 3), after heating the CO-rHb mixture and removing precipitate (stage 4), and after re-oxygenating the rHb sample (stage 5). Spectral deconvolution software was developed in the Python programming language (Python Software Foundation Beaverton, Oreg.) using the non-linear least squares function curve_fit of the SciPy package to determine the fraction of various liganded forms of rHb that contribute to the final spectra of the rHb mixture (i.e. containing metHb, hemichrome, oxyHb, HbCO and heme).

Stopped Flow Kinetics. CO binding to deoxyrHb, and 02 release from oxyrHb were measured using an Applied Photophysics SF-17 microvolume stopped-flow spectrophotometer (Applied Photophysics Ltd., Surrey, United Kingdom). Rapid kinetic measurements were performed using protocols previously described by Rameez and Palmer (Rameez, S. et al. Encapsulation of hemoglobin inside liposomes surface conjugated with poly(ethylene glycol) attenuates their reactions with gaseous ligands and regulates nitric oxide dependent vasodilation. Biotechnol. Prog. 28, 636-645 (2012)). Unmodified human Hb was used as a control. PBS (0.1 M, pH 7.4) was used as the reaction buffer for all kinetic measurements.

Results and Discussion

History of ApoHb Production Methods. The first published report of isolating globin (i.e., mixture of apoHb in its active and inactive forms) from Hb was from 1892 by Bertin-Sans and de Moitessier. In their method, oxygenated blood was coagulated with ether and mixed with a boiling solution of 10% tartaric acid in ethanol and further processed to yield apoHb. In 1898, Schulz made the first analysis of a purified globin solution produced via a mixture of ether and alcohol. Using this method, heme was extracted into the organic ether-ethanol phase leaving the globin in the aqueous phase. Later studies continued to use this protocol with slight modifications to produce globin until 1926. At that time, Hill and Holden noted that globin solutions were not soluble at the isoelectric point of Hb and the few available analyses of rHb were unclear on its biochemical properties. These issues were attributed to extensive globin denaturation (i.e., protein unfolding) from the use of harsh organic solvents at elevated temperatures, which lowered the yield of active apoglobin. Thus, Hill and Holden developed a very rigorous low temperature procedure that avoided the use of alcohol by using kieselguhr in ether to absorb heme. Using this method, Hill and Holden theorized it would not require protein unfolding, since it appeared that kieselguhr would remove heme under non-denaturing conditions. Yet, the theory of producing more active apoglobin due to the reduced protein unfolding step was shown to be highly improbable. Furthermore, Hsien Wu later noted that the major advancement in Hill and Holden's method was the low acidity of the solution and the low temperature of the process, and not because protein unfolding was minimized or abolished. It was also shown that performing Schulz's procedure under Hill and Holden's experimental conditions provided the same results as Hill and Holden. Finally, later studies by Ansos and Mirsky demonstrated the reversibility of protein unfolding, substantiating the idea that protein unfolding was not necessarily harsh for use in heme extraction. This idea of reversible protein unfolding led to the development of the acidic acetone heme extraction method, commonly used to this day. The acid-acetone procedure produces active soluble apoglobin even after unfolding the protein (to the point of precipitation) in acidic acetone.

Upon establishment of the previously mentioned acid-acetone or acid-methyl ethyl ketone (MEK) heme extraction procedures, these methods became the standard for producing apoHb in the literature. However, no modifications or improvements on these methods were made since their conception in the 1950s. These procedures require the use of highly flammable solvents which, when combined with the requirement of centrifugation or liquid-liquid extraction equipment, possess large safety risks for large-scale production. Additionally, since these processes use highly toxic solvents (acetone or MEK), it reduces possible biomedical applications of apoHb due to the presence of residual solvent in the preparation.

More recently developed apoglobin production methods employ acidified alcohols with subsequent separation facilitated by heme agglomeration, precipitation or adsorption on activated charcoal. Yet, these alcohol-derived apoglobins were made for applications in the food industry or for heme production, and did not provide an analysis of the retention of native apoHb activity or extent of heme removal. In this current study, the use of acidic ethanol-water heme extraction combined with TFF allows for the scalable and safe production of apoHb. A key advantage of this process is the absence of strong denaturants such as acetone or other ketones in the process. There is no method in the literature that describes purification of active apoHb from Hb in which both the heme and globin remain in the same phase. Additionally, previous apoHb production methods require extensive dialysis, which can be replaced by the quicker buffer exchange process facilitated by TFF run in diafiltration mode.

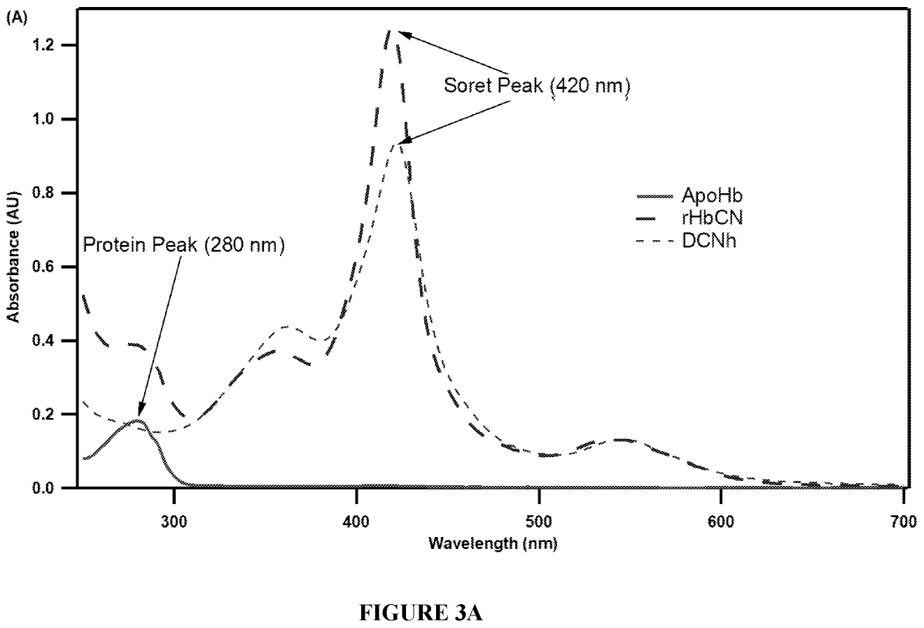

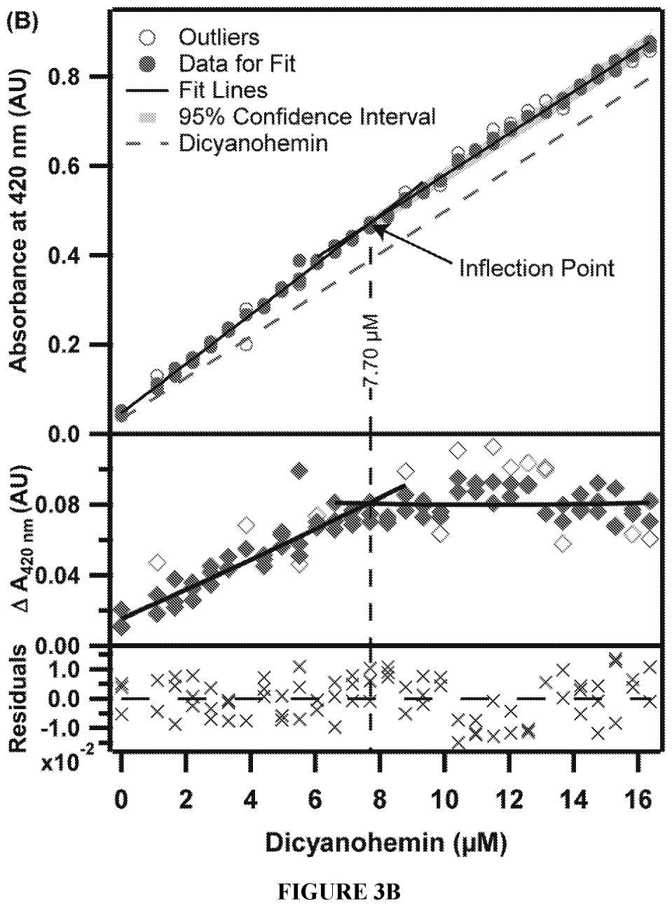

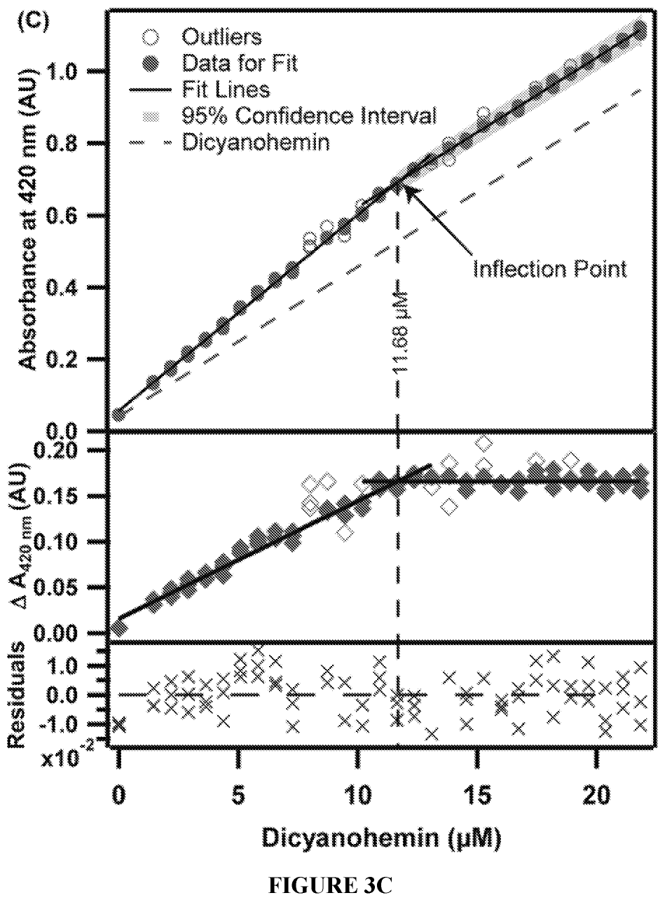

TFF Production of ApoHb. Earlier apoHb studies showed that the majority of purified apoHb product consisted of denatured apoHb with a small fraction of active apoHb in solution. Therefore, for applications where it is important to know the activity of the apoHb (i.e., defined as having a functional heme-binding pocket), apoHb quantification should be performed via an activity assay, and not total protein assays such as UV-absorbance analysis. Previous research quantified apoHb yield via analysis of the soluble apoglobin's absorbance peak at 280 nm (or via total protein assays). However, it has previously been demonstrated that quantification of soluble apoglobin does not accurately indicate the activity of the apoHb preparation, since a mixture of active and inactive apoglobins coexist in solution. As expected for a pigment-free protein, the UV-vis absorbance spectrum of apoHb consists of a single peak at 280 nm, which is shown in FIG. 3A. The presence of residual heme, which originates from porphyrin not removed during the purification process, yielded a slight Soret peak absorbance at ˜412 nm indicating the presence of hemichromes (a type of heme-bound species). A novel process to separate hemichromes from reconstituted Hb (rHb) is described later in this example. However, in previous apoHb purification studies, the residual Soret peak was observed at ˜404 nm, indicating that the bound heme yielded metHb and not hemichrome. The difference in the type of Hb formed from residual heme could have been caused by the dissociation conditions necessary for heme removal from Hb in the acidic ethanol solution of the TFF process compared to the acidic MEK and acetone used in previous apoHb purification schemes. For the latter, the residual heme originated from unextracted porphyrin, which shielded the heme from the extraction solvent. During TFF purification, the porphyrin ligand is likely maintained in dynamic equilibrium between free heme and the heme-protein complex. Since the apoprotein exists unfolded in acidic ethanol, the heme molecules in equilibrium were likely nonspecifically bound to the protein. Thus, when the apoprotein was refolded during the diafiltration process, these nonspecifically bound heme molecules yielded hemichromes. These hemichromes only constitute less than 1% of TFF-apoHb.

Upon heme addition to apoHb, the absorbance of the solution at 280 nm and in the Soret band increases due to the presence of the heme pigment and from the covalent bond of the proximal histidine (His-F8) in apoHb with the heme iron. The final absorbance spectra of reconstituted Hb (rHb) should also be the same as native Hb with the characteristic intense Soret peak and Q-bands (discussed in rHb Analysis section). Since apoHb lacks this intense Soret band, heme extraction from Hb was determined through analysis of the UV-visible spectra of the apoHb solution. Successful heme extraction was determined when less than 1% residual heme could be detected (i.e., when the absorbance ratio between the protein peak at 280 nm to the Soret peak at ˜412 nm is less than 0.1).

The increase in Soret peak absorbance compared to pure heme when the porphyrin is incorporated into apoHb is due to the formation of a covalent bond between His-F8 in apoHb with the iron atom in heme. This difference in Soret peak absorbance is shown in FIG. 3A, and is used in the DCNh-incorporation assay to quantify the number of His-F8-Fe bonds formed (i.e., active heme-binding pockets). In the DCNh-incorporation assay, DCNh is titrated against a constant apoHb concentration until all the heme-binding pockets have been saturated with DCNh and excess DCNh is present in solution. During the initial titration, all the added heme reacts with apoHb to form rHbCN which produces a straight line (major line) in FIGS. 3B and 3C (top graphs). Upon saturation of the available heme-binding pockets, free heme becomes present in solution without the formation of additional covalent bonds between the His-F8 residue of apoHb and the iron atom in DCNh. Therefore, the increase in absorbance at later titration points are due to the absorbance of pure DCNh in solution. Since rHbCN has a higher Soret absorbance than pure DCNh, a minor line with lower slope compared to the major line is observed in FIGS. 3B and 3C (top graphs). The inflection point between the major and minor lines indicates the concentration of heme necessary to saturate the heme-binding pockets of apoHb. This heme-binding assay was used to quantify the concentration of active apoHb on a per heme basis throughout this study.

As shown in FIG. 3B and FIG. 3C, by monitoring the Soret peak at 420 nm, the titration assay can be performed using DCNh (i.e., heme species used in assay) to determine the concentration of active heme-binding pockets. Since the number of active heme-binding sites is dependent on the initial concentration of apoHb in the sample to be tested, this explains the difference in the results between the TFF and acetone produced apoHb samples. FIG. 3B exemplifies the assay when 7.70 μM of active heme-binding sites were present in an apoHb solution produced via TFF. On the other hand, FIG. 3C exemplifies the same assay on an apoHb solution produced from the acetone extraction procedure with 11.86 μM of active heme-binding sites. Subtracting the pure DCNh absorbance from the absorbance of the titration mixture, allows for the detection of a more noticeable slope change as can be seen in the middle graphs in FIGS. 3B and 3C. From these graphs, it is important to note that apoHb produced via TFF (FIG. 3B) and apoHb produced via acetone extraction (FIG. 3C) had the same characteristic slope change indicating the expected heme-binding activity of apoHb. Both the residual plots showed no discernable trend, indicating good fits to the major and minor lines (bottom graphs of FIGS. 3B and 3C).

After each batch, the DCNh-incorporation assay was used to quantify the moles of active apoHb on a per heme basis and the 280 nm absorbance (ε=12.7 mM⁻¹ cm⁻¹) was used to quantify moles of total protein. To determine protein yield, the moles of active apoHb and total protein were compared to the initial moles of heme in the Hb precursor. The yield of the TFF apoHb production method was compared to the acetone method using two size TFF filters (miniKros (surface area of 1,000 cm²) and microKros (surface area of 20 cm²)) to demonstrate the scalability of the TFF process. The results from this analysis are shown in Table 1.

TABLE 1
Summary of results from various apoHb production methods and
the effect of concentration on the activity of apoHb samples.

| | Overall Protein | Overall Active | |
|---|---|---|---|
| Production Method | Yield | ApoHb Yield | N |
| Acetone | 90.5% ± 17.6% | 70.3% ± 10.7%^{a} | 4 |
| MicroKros TFF | 96.6% ± 5.5% | 83.1% ± 5.4% | 6 |
| MiniKros TFF | 98.4% ± 11.0% | 73.4% ± 5.3%^{a} | 14 |

^{a}p < 0.05 compared with microKros TFF

| Active Protein Loss | Total Protein Loss |
|---|---|
| 7.7% ± 1.9%^{b} | 21.3% ± 8.1%^{b} |
| State | % Active Protein |
| Unconcentrated | 62.3% ± 4.8%^{c} |
| Concentrated | 69.5% ± 4.0%^{c} |

^{b,c}p < 0.05 between pairs with same letter

Studies have previously reported total apoHb yields from acetone or MEK extraction to be ˜90%. However, these studies quantified total protein (which includes both active and inactive apoHb) through methods such as protein absorbance at 280 nm. Thus, when comparing total protein yields, apoHb production via TFF (total protein yield of about 95%) had similar values to the commonly used heme extraction methods. Additionally, the total protein yield from acetone extraction agreed with previous reports. As expected, there was more total protein in solution compared to active apoHb in solution indicating that some of the resultant protein in solution lost its activity. It was observed that some protein was adsorbed on the filter membrane, which explains the loss in total protein. Yet, total protein analysis also showed that most of the protein was retained during production and that there was no significant difference between the studied production methods. Thus, since virtually no protein is lost in the TFF process, applications in which only heme-free globin is desired can still benefit from the TFF purification methodology. From the active protein analysis, acetone showed 70.3% yield compared to 83.1% and 73.4% for the microKros and miniKros TFF filters, respectively. Additionally, the yield from the small-scale miniKros filter was significantly different than both other setups. These results demonstrate that TFF production had similar or improved active apoHb yields compared to the acetone extraction method.

TFF-apoHb production with microKros filters had a significantly higher active apoHb yield than both the acetone and miniKros TFF methods (p<0.05). When scaling a TFF system, factors such as shear rate, pressure drop, filter type affect TFF efficiency. Therefore, operational parameters were kept constant when possible between the miniKros and microKros TFF systems in this example. However, the inlet pressure for the miniKros system prevented it from reaching the required flow rate to obtain the same shear rate as the microKros system. Thus, shear rates of 5,900 s⁻¹ and 4,300 s⁻¹, were achieved for the miniKros and microKros systems, respectively. The difference in shear rate could explain the lower permeate flow rate of the miniKros system, since lower shear rates may facilitate protein build up on the membrane. Since the permeate flow rate was not scaled, the diafiltration period was longer on the miniKros system, increasing the time that the protein remained unfolded in the acidified organic solvent. This longer exposure time could explain the lower active protein yield of the miniKros system compared to the microKros system and is a key variable which must be controlled to improve the yield of active apoHb.

During the concentration phase of TFF processing, protein precipitation was observed. Over time, HF membrane fouling decreased permeate flowrate up to 70%, making further concentration non-viable. To explore the effect of this concentration step on active protein yield and activity of the apoHb preparation, apoHb preparations were tested for total protein and active protein before and after concentration. Protein lost during concentration was compared to the initial mass of Hb used for apoHb production. As seen in Table 1, the loss of total protein from the sample was greater than active protein. Since more total protein was lost, the fraction of active protein in solution increased. The higher loss of inactive protein can be explained by the greater instability of inactive apoHb in solution versus active apoHb, facilitating precipitation at higher concentrations of apoHb. Stabilizing agents or alternative buffers may alter or improve these effects and should be considered in future method optimization.

The limit of 2 mg/mL of initial Hb precursor in the acidic ethanol solution was chosen to ensure full heme extraction from the sample. The dissociation of heme from unfolded globin seems to follow the equilibrium between the globin-heme complex and free heme+free globin in solution. Thus, when too high of an initial Hb concentration is loaded into the TFF circuit, the high globin concentration may limit the heme from dissociating from the globin-heme complex. Thus, in trials with a high initial Hb concentration little to no heme would permeate out of the TFF cartridge (undetectable on the absorbance spectrum). This was despite observing a significant amount of heme in the acidic ethanol solution within the TFF flow circuit (analyzed via the ratio of the Soret peak at 412 nm to the 280 nm protein peak). Additionally, the requirement of 9 diacycles for full heme extraction was evidenced by this equilibrium. If no heme retention occurred, the number of diacycles should have been closer to that of a simple buffer exchange (5 to 6 diacycles). Furthermore, when Hb solutions with concentrations of >50 mg/mL were used as the initial basis for the process, the protein rapidly denatured into a red precipitate when it made contact with the 80% acidic ethanol solution. Thus, to minimize this effect, Hb solutions with concentrations of −25 mg/mL were used when adding the holoprotein to the acidic ethanol solution.

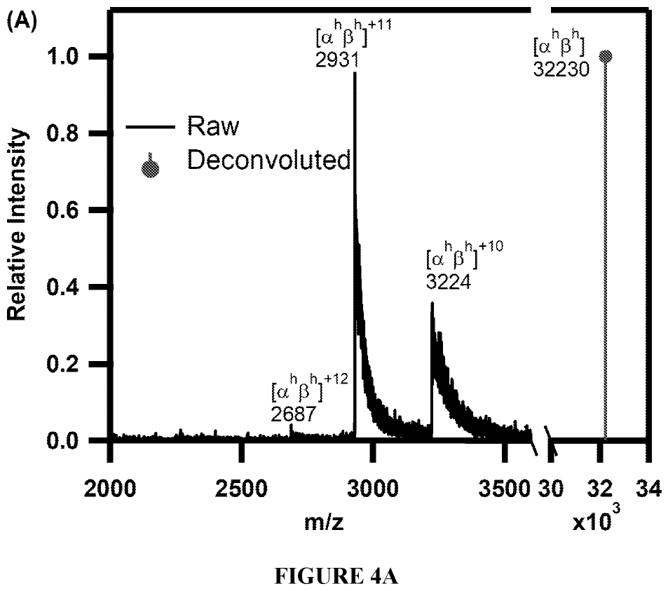

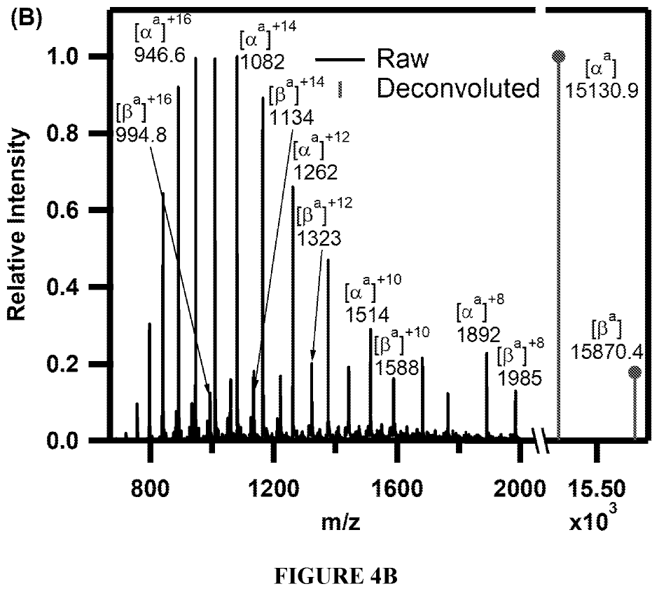

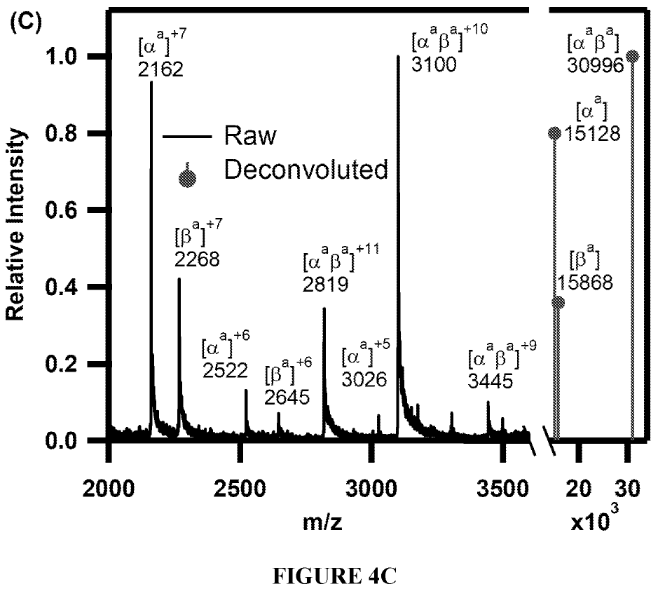

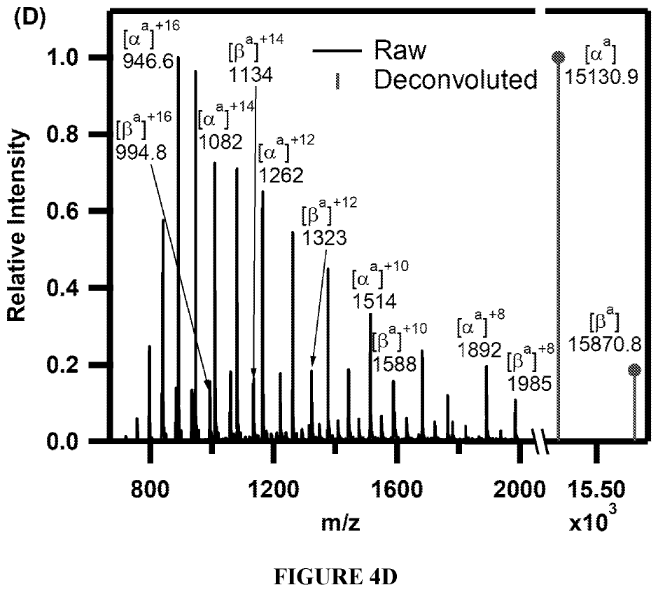

Biophysical Properties of ApoHb produced via TFF. The TFF-apoHb was analyzed via electrospray ionization mass spectroscopy (ESI-MS) to determine if processing caused any amino acid residue modifications or protein damage. FIG. 4 presents the results of these experiments. ESI-MS analysis demonstrated that native Hb was detected only as holodimers (FIG. 4A). Although a predominantly tetrameric holoprotein was expected, the MS equipment did not have the ability to detect the mass of the tetrameric species, thus only αβ dimers were observed. The detection of αβ dimers can be explained by the dimer-tetramer equilibrium of Hb in solution. Additionally, upon protein denaturing under acidic conditions (FIG. 4B), Hb dissociated into its apoglobin monomers (individual α and β chains) and heme was released into solution. Since free heme was not detected (removed during dialysis before MS analysis), the only spectra with heme (indicated by the superscript h) was Hb analyzed under native conditions. All other spectra only detected apoproteins (indicated by the superscript a) When analyzing apoHb with MS under native conditions, the lack of heme in apoHb allowed for detection of apo αβ dimers and apo α/β monomers (FIG. 4C), possibly indicating monomer-dimer equilibrium in solution. Yet, like Hb, under acidic conditions (FIG. 4D), the higher order species dissociated, making the mass spectra of apoHb and Hb nearly identical (same peaks of apo α/β globin chains when comparing FIG. 4B and FIG. 4D). Thus, during Hb MS analysis, apo α/β monomers were only detected under denaturing conditions, while for apoHb MS analysis, apo α/β monomers were detected under both conditions. Although the intensity of α and β globin chains were expected to be similar given their 1:1 molar ratio in Hb and apoHb, it has been shown that conformational differences between the α and β globins allow for more efficient competition of the α chains for charges. This difference causes a higher intensity of α chains in the spectra compared to β chains.

Under native conditions, the observed mass of holo-Hb αβ dimers (FIG. 4A) and apoHb αβ dimers (FIG. 4C) were determined to be 32,230 Da and 30,996 Da, respectively. These results were close to the theoretical mass of 32,226 and 30,994 Da for the holo- and apo- αβ dimer, respectively (difference of two 616 Da heme groups between the holo- and apo- αβ dimers). During Hb analysis under denaturing conditions (FIG. 4B), the apo α globin (theoretical mass of 15126.4 Da) detected had an observed mass of 15130.9 Da while the apo β globin (theoretical mass of 15867.4 Da) had an observed mass of 15870.4 Da. During apoHb analysis, apo α globin was detected under both native (FIG. 4C) and denaturing conditions (FIG. 4D) with observed mass of 15128 Da and 15130.0 Da, respectively. Also during apoHb mass spectral analysis, apo β globin had observed masses of 15868 Da and 15870.8 Da under native (FIG. 4C) and denaturing conditions (FIG. 4D), respectively. The results from ESI-MS analysis demonstrated that apoHb globin chains produced via TFF maintained their structural integrity and were not chemically modified.

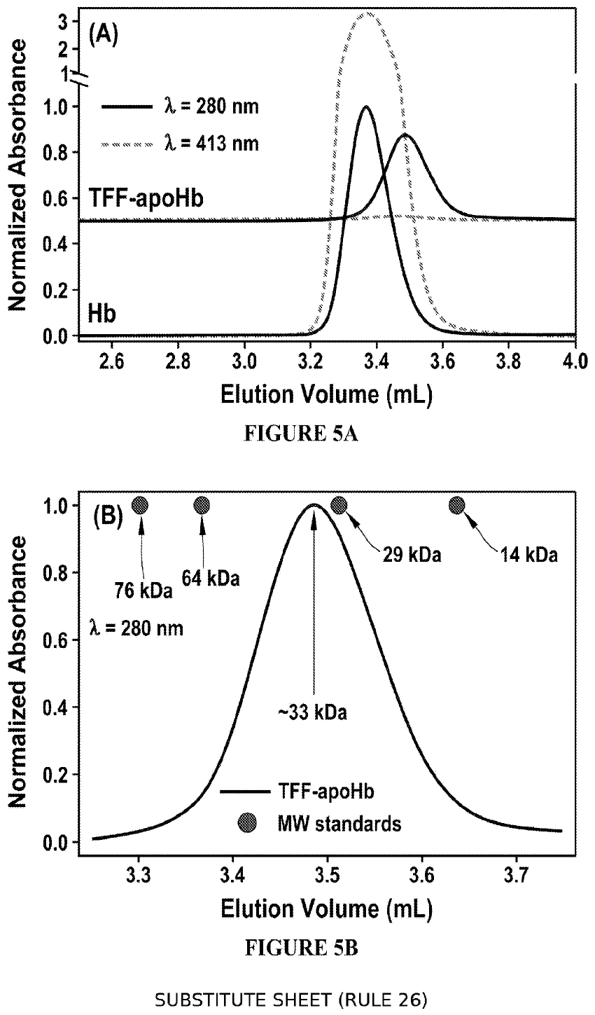

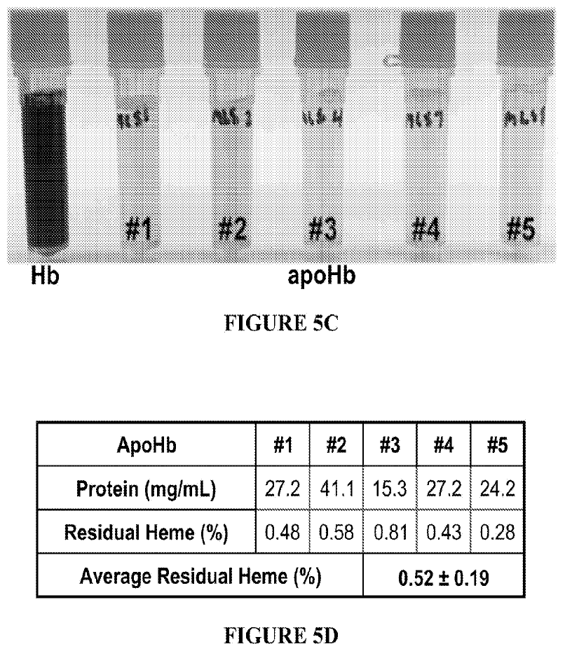

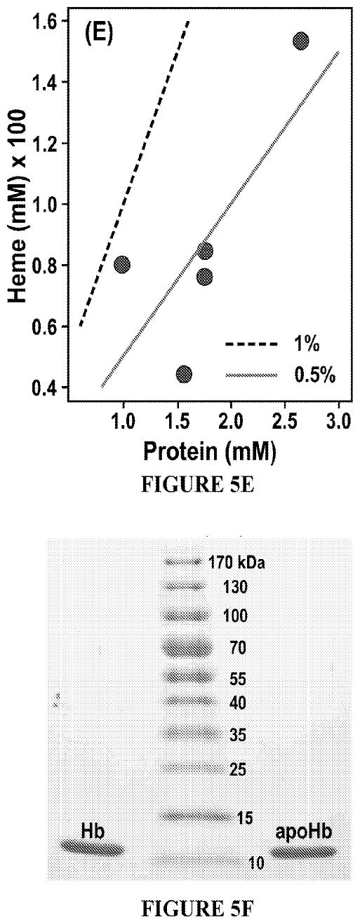

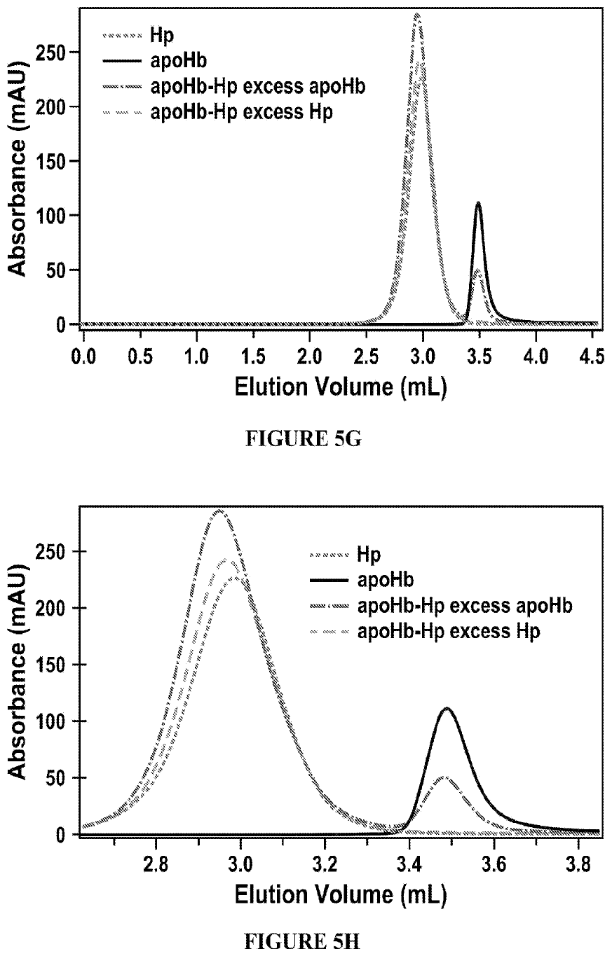

Further analysis of the quaternary structure of apoHb was performed using SEC. The HPLC-SEC profile of TFF-apoHb and human Hb is shown in FIG. 5A. Comparing the chromatogram of TFF-apoHb with that of human Hb, it was observed that there was a major reduction in the absorbance of the Soret peak between the two species. Furthermore, the absorbance at 280 nm was also reduced between the two species. Both of these observations were expected given the high absorbance of heme in the Soret region and at 280 nm. The protein standards with MWs of 76, 64, 29, 14 and 6.5 kDa eluted at 3.30, 3.37, 3.51, 3.64, and 3.82 mL, respectively. Based on these elution times, the MW of apoHb which eluted at 3.5 mL was determined to be about ˜33 kDa. This MW indicated that the apoprotein was primarly an αβ dimer in solution. FIG. 5B shows the elution of TFF-apoHb and the peaks of the MW standards. From these chromatograms, it was also noted that there are no tetrameric species in freshly prepared TFF-apoHb samples. These tetrameric species have been theorized to originate from irreversible disulfide bond formation between apoHb dimers in the apoHb denaturing pathway. Yet, there exists conflicting evidence on whether these disulfide bonds are formed after heme removal, as it has been shown that apoHb precipitates maintain functional thiol groups, while Hb precipitates forms disulfide-bonded tetramers.

SEC was also performed on Hb and apoHb samples to analyze heme content (FIG. 5C). Using the Soret peak of Hb as a reference, the Soret peak of each apoHb batch was used to estimate the amount of residual heme present in the apoprotein preparation after production. FIG. 5D and FIG. 5E shows results from this analysis confirming that less than 1% of residual heme was present in apoHb preparations produced via TFF (compared to the total protein in the sample). As shown in FIG. 5E, the final protein concentration of the sample did not influence the amount of heme remaining in the apoHb preparation, demonstrating that the protein can be concentrated maintaining its desired characteristics. Additionally, the samples normally had ˜0.5% or less residual heme confirming the effectiveness of the TFF heme extraction method in removing heme from the apoHb preparation.

A promising and important characteristic of apoHb is its clearance from the blood stream via CD163+ macrophage and monocyte mediated endocytosis. This in vivo clearance pathway for apoHb is the same for cell-free Hb. Both holo- and apo-Hb first bind serum Hp to form the (Hb/apoHb)-Hp complex, then the (Hb/apoHb)-Hp complex is captured by CD163+ macrophages and monocytes. To analyze if TFF-apoHb was capable of binding Hp, a fixed Hp concentration was mixed with increasing concentrations of apoHb and allowed to react to completion. The components of these mixtures were then separated via SEC-HPLC. As shown in FIGS. 5G and 5H, apoHb eluted at 3.5 mL, while the large MW Hp mixture eluted earlier at 3 mL. The earlier elution volume of Hp was expected, since the Hp used was a mixture of higher MW proteins (>200 kDa). When analyzing the elution profiles, the lack of an elution peak at 3.5 mL for the apoHb-Hp mixtures with excess Hp demonstrated that Hp and apoHb formed a single apoHb-Hp complex at sub-stoichiometric apoHb concentrations. Furthermore, a slightly lower elution volume was observed for the apoHb-Hp complex as indicated by a slight left shift in the elution curve. From the excess apoHb trial, the change of the area under the curve compared to the pure apoHb solution indicated the amount of apoHb that was bound to Hp. From this data, active apoHb had almost the same mass binding ratio to Hp as native Hb (<5% difference), and agreed with previous apoHb:Hp binding studies that demonstrated binding of one αβ Hb dimer per Hp αβ dimer. Therefore, this analysis demonstrates that apoHb produced via TFF retains the ability to bind to Hp.

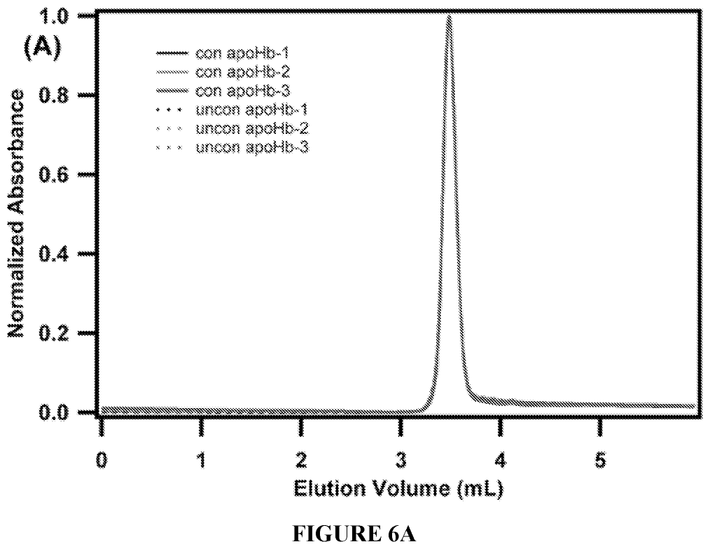

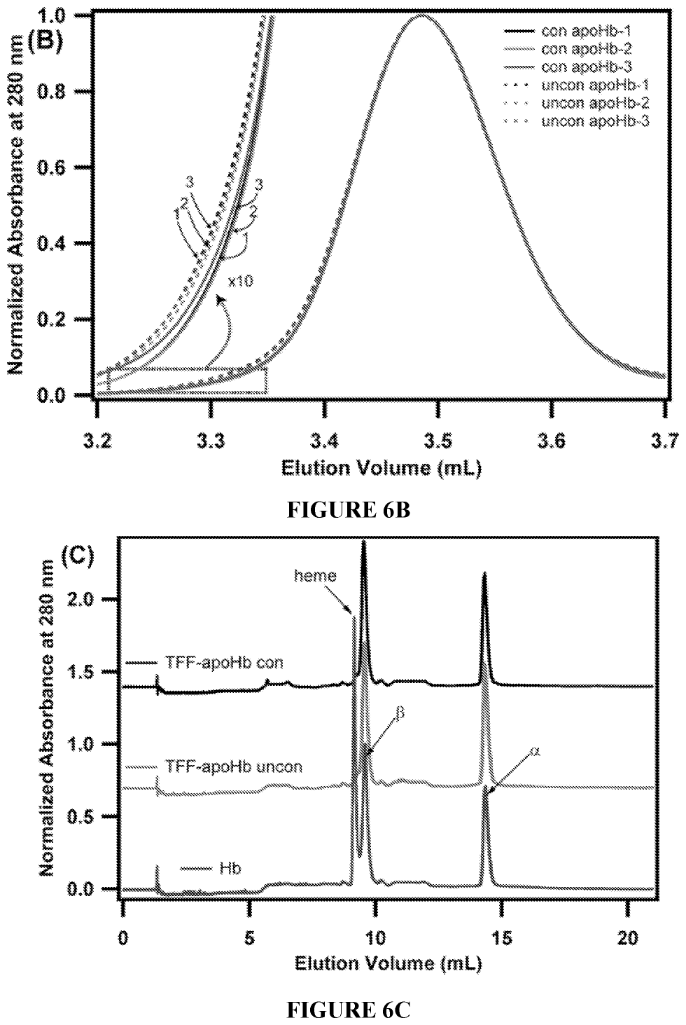

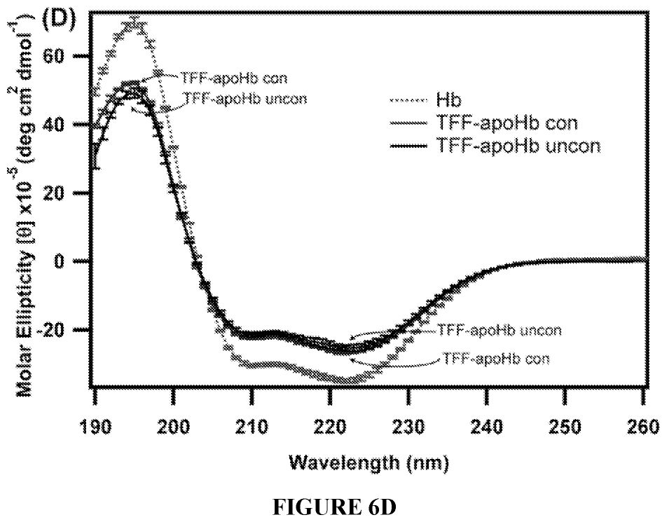

To ensure that no oxidative modifications or disulfide-bonded intermediates were present in our apoHb preparations, three different batches of TFF-apoHb were analyzed via reverse-phase HPLC (RP-HPLC). The samples were first evaluated via SEC-HPLC and, as shown in FIGS. 6A and 6B, the fresh TFF-apoHb preparations had very little variability and did not contain any noticeable amounts of tetrameric or oligomeric species. Interestingly, there may be a slight increase in the presence of apoHb tetramers in the unconcentrated (uncon) samples due to their left shifted elution peak as observed on SEC-HPLC (FIG. 6B). These higher MW species may be linked to the lower relative apoHb activity and lower alpha-helical content of the unconcentrated samples.

RP-HPLC analysis is shown in FIG. 6C. The Hb sample separated into its respective heme, α-chain and β-chain components, while TFF-apoHb samples displayed only α-chain and β-chain components. Furthermore, there was no noticeable difference in the RP-HPLC chromatograms when comparing the unconcentrated or concentrated TFF-apoHb samples with native hHb. Thus, the TFF process did not cause any oxidative modifications to the apoprotein as has been reported by some processes in the literature.

The secondary structure of TFF-apoHb was determined via CD of the far UV region (190-260 nm). This analysis is shown in FIG. 6D. The results indicated that, as previously shown in the literature, apoHb had a ˜25% reduction in alpha-helical content compared to native Hb (given by the ratio of the 222 nm peak, which corresponds to alpha-helices). Furthermore, there seemed to be a slight increase in alpha helical content upon sample concentration. This, similar to the increase in relative activity, may have been due to the removal of inactive apoHb from the sample during concentration.

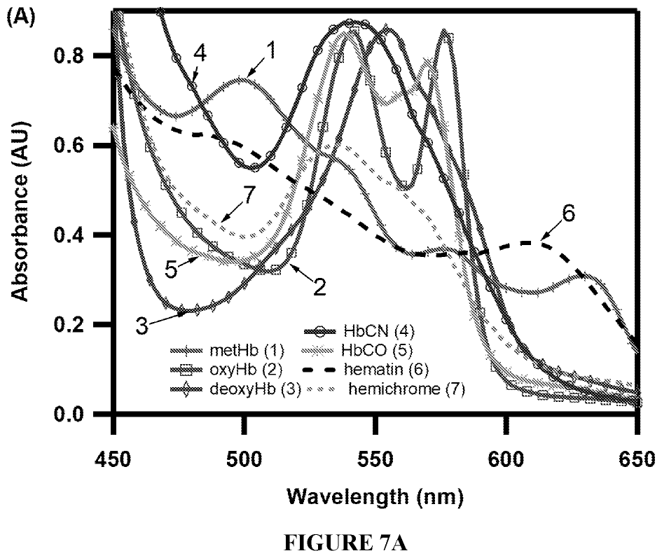

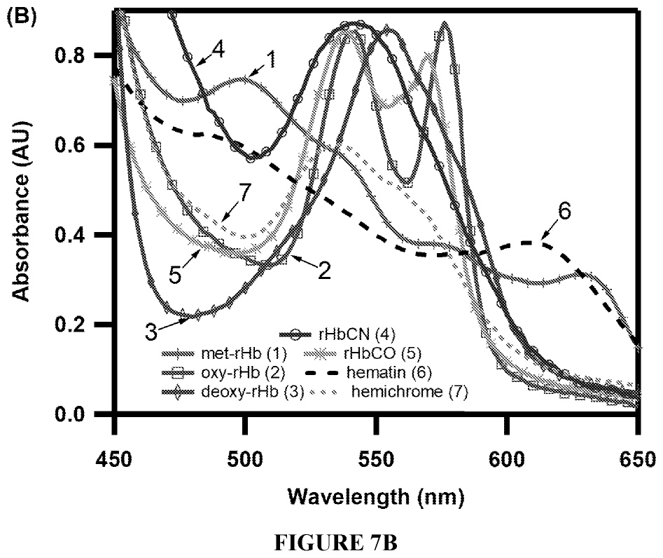

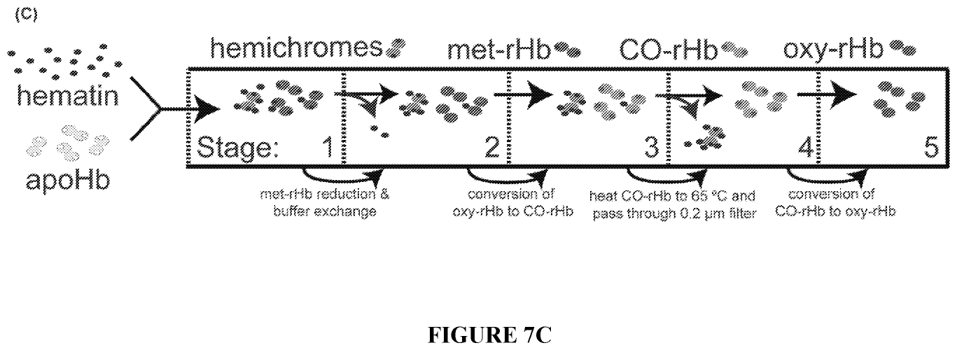

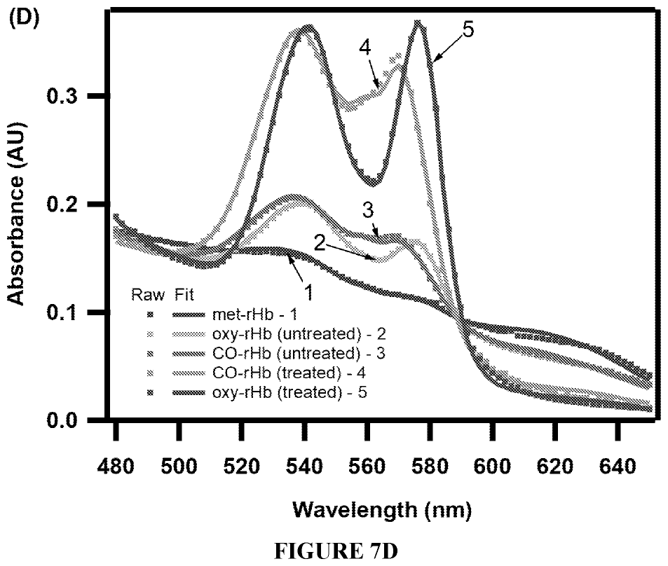

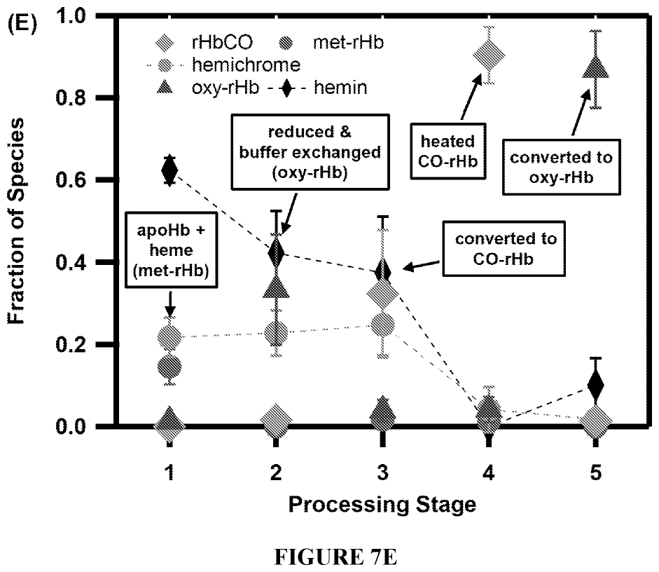

To test the activity of TFF-apoHb, the protein was reconstituted into rHb and the biophysical properties of rHb were analyzed and compared to native Hb. First, TFF-apoHb was reacted with heme and the spectral absorbance of various liganded forms of rHb were compared to native Hb. It was evident that the spectra of rHb in various liganded states shown in FIG. 7B closely resembled that of native Hb shown in FIG. 7A. This indicated that TFF-apoHb can be reconstituted to yield native-like Hb spectra. However, hemichromes or other heme-globin complexes are observed when reconstituting apoHb. Such complexes may convolute spectral analysis of rHb. As shown in FIG. 7D, the spectra of rHb before processing had a distinct offset from pure rHb. When comparing non-processed samples (before heating), it was noted that the offset was due to the presence of hemichromes and unbound heme (pure species spectra shown in FIGS. 7A and 7B), confirming the presence of hemichromes. Yet, the literature contains no method to separate or remove these denatured species. To perform accurate spectral analysis of rHb, we developed a simple method to remove these unstable species, which is shown in FIG. 7C.

Starting from a mixture of excess heme and apoHb, which reacts to form met-rHb, hemichromes, and excess heme (stage 1), the sample is processed to obtain oxy-rHb. Yet, hemichromes and excess non-specifically bound heme could be present in the sample along with oxy-rHb (stage 2). Thus, the mixture from stage 2 was placed under a CO atmosphere to transform oxy-rHb into the more heat stable CO-rHb species (stage 3). HbCO is more resistant to thermal denaturation and precipitation at elevated temperatures (65° C.) compared to other liganded forms of Hb, whereas heme-globin complexes and hemichromes are highly unstable and precipitate even at low (4° C.) temperatures. Thus, when a mixture of CO-rHb and heme-globin complexes is heated, the CO-rHb remains in solution while the unstable species precipitate out of solution (stage 4). After heating and separation of hemichromes, the CO-rHb can be reverted into oxy-rHb under a pure 02 atmosphere and white light illumination (stage 5).

To analyze this unique hemichrome removal method and provide better information on the species present in solution, a spectral deconvolution program was developed and implemented to determine the fraction of rHb species present in solution during the various processing stages in the reconstitution process. As shown in FIG. 7E, hemichromes were present in the sample at significant levels until the sample was heated. It was also observed that, although heme would be expected to be filtered through the HF modules, much of the excess heme added to the apoHb sample was not removed during the modified HEMOX buffer-exchange step after reduction (only a slight decrease in heme content was seen between stage 1 and 2). Since the unbound heme was not removed via filtration, it indicated that some of the heme was nonspecifically bound to the protein in solution. However, it was noted that the nonspecifically bound heme was mostly attached to the hemichromes, since no heme was detected in the CO-rHb spectrum after hemichrome removal upon heat treatment (this event is also represented in FIG. 7C). The higher affinity of heme to nonspecifically bind to hemichromes would be expected since these semi-denatured species should have more exposed hydrophobic regions or altered structural motifs that lead to higher heme-binding affinity. The higher heme binding capacity of hemichromes is also supported by reports in the literature that denatured globin can bind up to thirty heme molecules. This ratio is much higher than the number of available heme-binding pockets present in tetrameric Hb (4 heme binding sites). Although some heme was detected in stage 5, this can be explained by the low signal to noise ratio of the heme species, especially since no heme was detected in the previous CO-rHb spectra of stage 4.

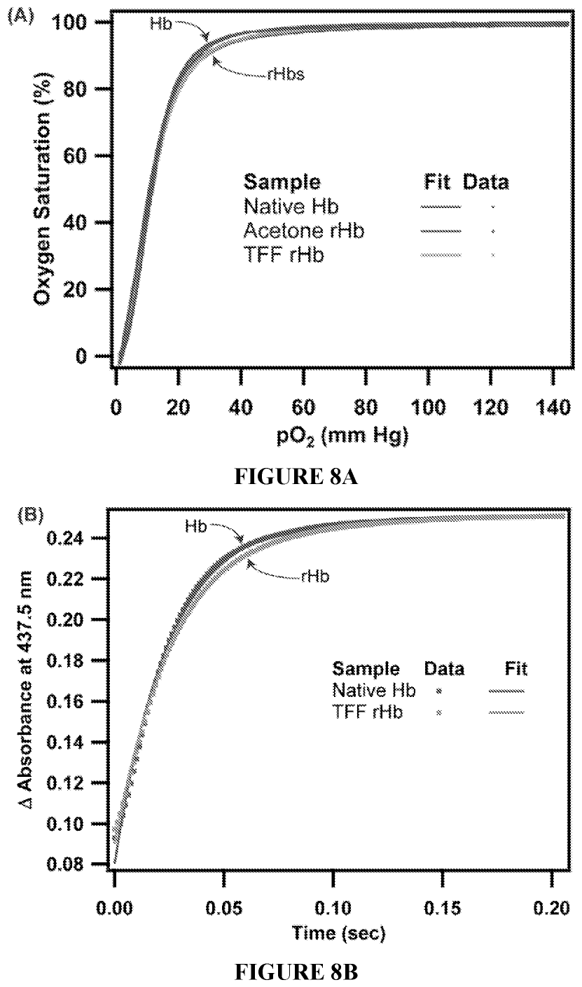

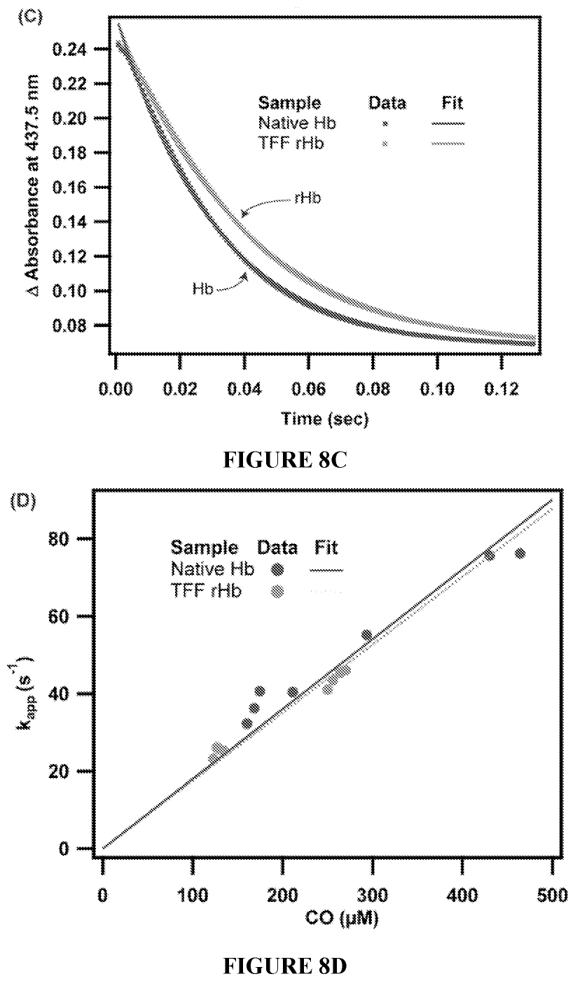

To further analyze the biophysical properties of rHb derived from TFF-apoHb heme extraction, rHb was fully reconstituted back to oxy-rHb and its O₂ equilibrium binding curve measured using a HEMOX Analyzer. From the O₂ equilibrium curve, the P₅₀ (partial pressure of O₂ required to saturate half of the heme binding sites with O₂) and cooperativity coefficient (n) can be regressed. The O₂ dissociation (k_{off,O}_{₂}) and CO binding (k_{on,CO}) kinetics were also measured using stopped-flow UV-visible spectroscopy. FIG. 8 shows representative data sets for native Hb and rHb. FIG. 8A shows representative O₂ equilibrium curves for Hb and rHb. FIGS. 8B and 8C show representative O₂ dissociation and CO association kinetic time courses for Hb and rHb, respectively. Finally, FIG. 8D shows the apparent rate constant for CO association to deoxyHb at varying CO concentrations. This data was fit to a linear equation to regress k_{on,CO}.

Previous studies have shown that rHb produced from acetone extraction has the same biophysical properties compared to native Hb. This was confirmed from the quantitative results listed in Table 2. The P₅₀ of rHb (11.36±0.87 and 11.20±0.43 mmHg for concentrated and unconcentrated rHb, respectively) was similar to native Hb (11.69±0.88 mmHg), with no statistical difference (p<0.05). The cooperativity of rHb from TFF-apoHb (2.14±0.17 and 2.27±0.10 for concentrated and unconcentrated rHb, respectively) was statistically different (p<0.05) compared to native Hb (2.73±0.11). Yet, similar to native Hb, TFF derived rHb had cooperativity greater than 2, which is indicative of cooperative O₂ binding. Additionally, previous studies have shown that Hb which has been oxidized then reduced possess lower cooperativity than native Hb. This effect could have been exacerbated by the formation of reactive free radicals upon use of dithionite for reduction. It has also been shown that, upon reconstitution, the heme can enter the heme pocket in an altered orientation, which also reduces rHb cooperativity. Thus, the lower cooperativity of rHb compared to native Hb can be due to incorrect heme insertion and the necessary reduction step to form oxy-rHb. Also shown in Table 2 are the rate constants for O₂ dissociation from HbO₂ and CO association to deoxyHb. There was no statistical significant difference between the CO association rate constants for native Hb (k_{on,CO}=180±7 nM/s) and TFF rHb (k_{on,CO}=175±4 nM/s). However, the difference in O₂ dissociation between native Hb (k_{off,O}_{₂} s⁻¹=37.68±1.27) and TFF rHb (k_{off,O}_{₂} s⁻¹=33.59±1.10) was significant (p<0.05). Yet, similar to the cooperativity coefficient, the lower rate constants could be due to the processing required to obtain rHb. Overall, compared to previously developed apoHb production methods, rHb derived from the TFF production method maintained native Hb characteristics.

TABLE 2
Biophysical properties of native Hb and TFF produced rHb: O₂
affinity (P₅₀); cooperativity coefficient (n); CO association to
deoxyHb (k_{on, CO}); and O₂ dissociation from oxyHb (k_{off, O2}).

| | O₂ Equilibrium | | | Ligand Binding Kinetics | | |
|---|---|---|---|---|---|---|
| | P₅₀ | | | k_{on, CO} | | |
| Sample | (mm Hg) | N | N | (nM/s) | k_{off, O2} (s⁻¹) | N |
| hHb | 11.69 ± 0.88 | 2.73 ± 0.11 | 10 | 180 ± 7 | 37.68 ± 1.27 | 4 |
| TFF rHb | 11.20 ± 0.43 | 2.27 ± 0.10^{a} | 4 | — | — | — |
| TFF rHb | 11.36 ± 0.87 | 2.14 ± 0.17^{a} | 10 | 175 ± 4 | 33.59 ± 1.10^{a} | 4 |
| Concentrated | | | | | | |

^{a}p < 0.05 compared with hHb

To analyze the stability of TFF-apoHb during storage, the amount of active and total apoHb was assessed under different storage conditions (37, 22, 4, −80° C. or lyophilized) and at two protein concentrations (concentrated [33.80±0.36 mg/mL active apoHb with 41.40±2.77 mg/mL total protein] or unconcentrated [1.47±0.01 mg/mL active apoHb with 1.99±0.17 mg/mL total protein]). The results from this analysis are shown in FIG. 8.

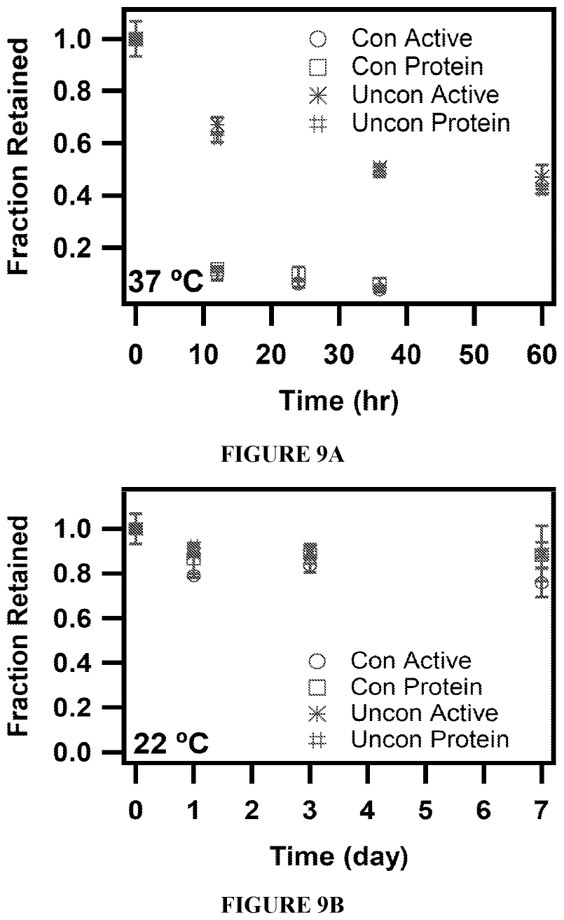

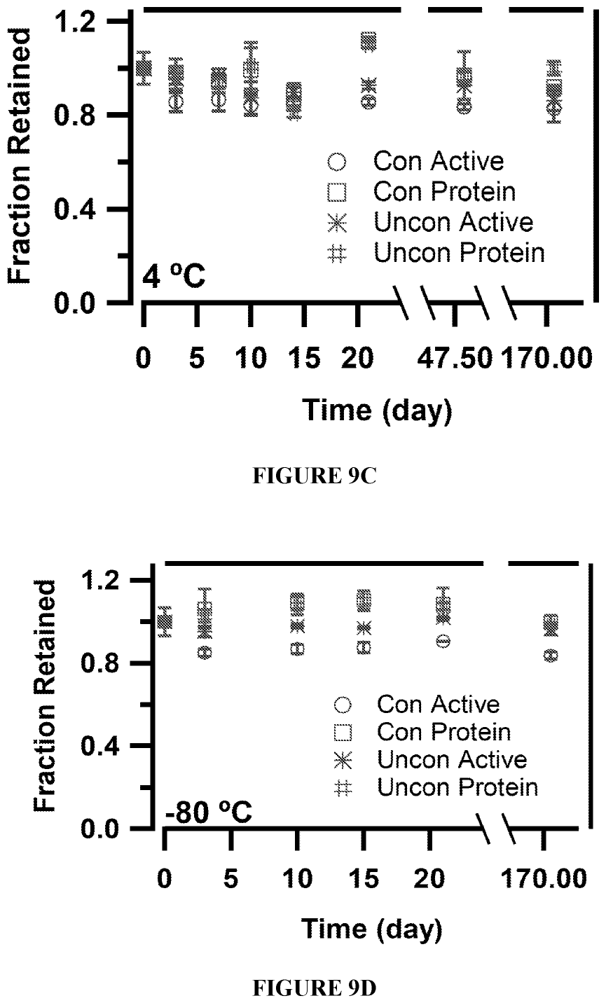

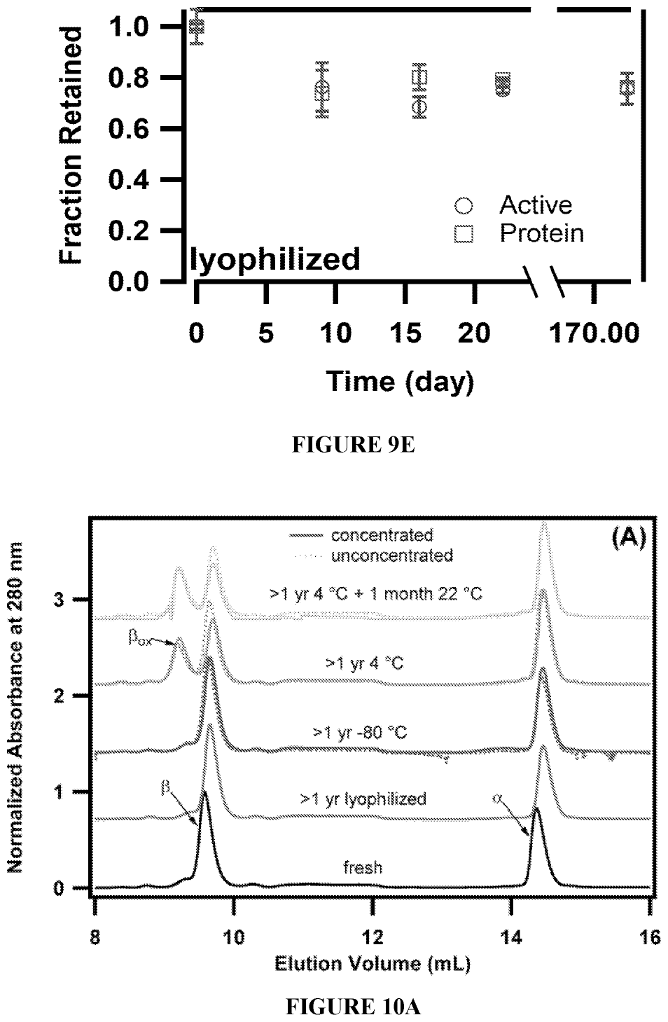

At physiological core body temperature (37° C.) (FIG. 9A), precipitation was visually apparent and the effect of time was statically significant at both apoHb concentrations. Under these conditions, apoHb quickly lost total protein and activity in solution during storage, especially for the concentrated samples. Interestingly, there was no statistically significant difference between the last two time points of the concentrated (24 and 36 h) and unconcentrated (36 and 60 h) samples, indicating some stability after the initial drops. Even though apoHb was not very stable at 37° C., this result does not deter possible biomedical applications of apoHb. When bound to Hp (expected to occur in vivo), the apoHb-Hp complex greatly increases the heat stability of the protein. At room temperature (22° C.) (FIG. 9B), the effect of time was statistically significant on activity, but no significant differences were seen for subsequent time points compared to day three. Additionally, the effect of time on total protein was insignificant. This contradicts the results of a previous study, which observed the rapid loss of apoHb after only a few minutes of storage at room temperature.

While stored at 4° C. (FIG. 9C), the effect of time was significant on the activity of unconcentrated samples, a significant difference was compared to day 0 was only seen on day 10. Additionally, no significant differences were seen after day 10. When stored at −80° C. (FIG. 9D), there was no statistically significant effect of time on apoHb activity or total protein at both concentrations. The higher stability of apoHb at −80° C. was expected, since the frozen sample lowered the likelihood of aggregate formation in solution. Lyophilized apoHb (FIG. 9E) also did not show a significant effect of time on apoHb activity or total protein. There was an initial decrease post-lyophilization, but activity was maintained afterwards (no statistically significant differences were observed). This was an expected result, since the freeze-drying process can damage the protein.

Under all concentration dependent conditions, the concentrated samples had statistically significant lower active apoHb retention compared to unconcentrated samples, indicating that higher storage concentrations led to higher active apoHb loss. This observation can be explained by the higher probability of protein aggregation at higher protein concentrations. On the other hand, total protein retention was not statistically significant between concentrated and unconcentrated samples, except for the 37° C. storage condition. These observations indicated that the concentration of stored samples only significantly influenced apoHb activity. Furthermore, lyophilized apoHb and storage at 37° C. appear to have coinciding total protein and active protein retention, indicating that the protein lost from heating or from the freeze-drying process was not selective for whether the protein was active or not. To further test the biophysical properties of the apoHb samples stored at 4° C., samples were fully reconstituted into rHb after one month of storage and the P₅₀ and cooperativity coefficient did not show any significant difference compared to apoHb samples reconstituted to rHb right after TFF production.

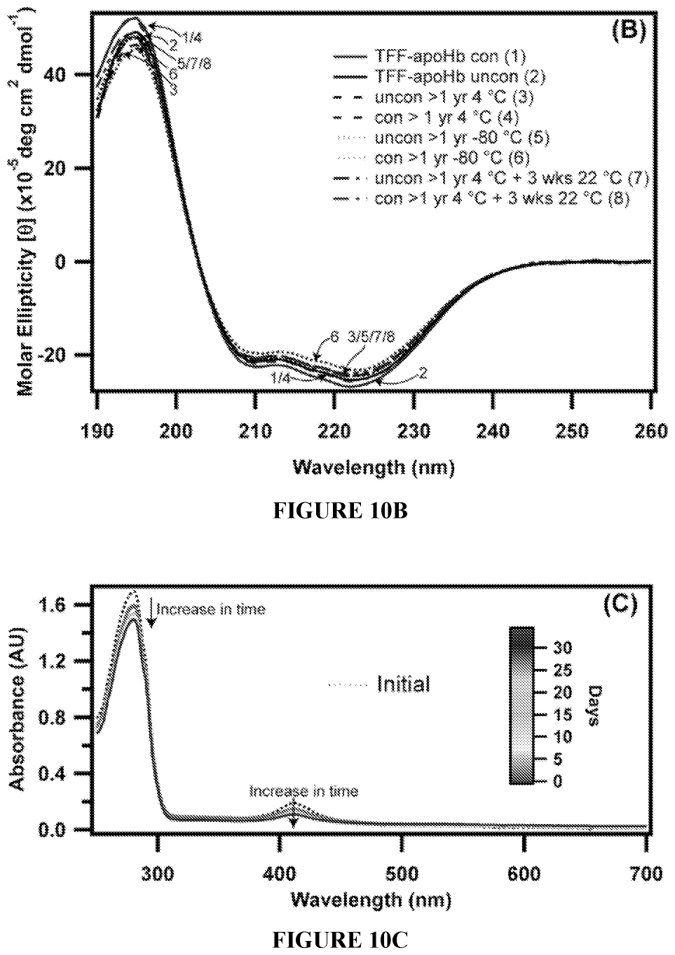

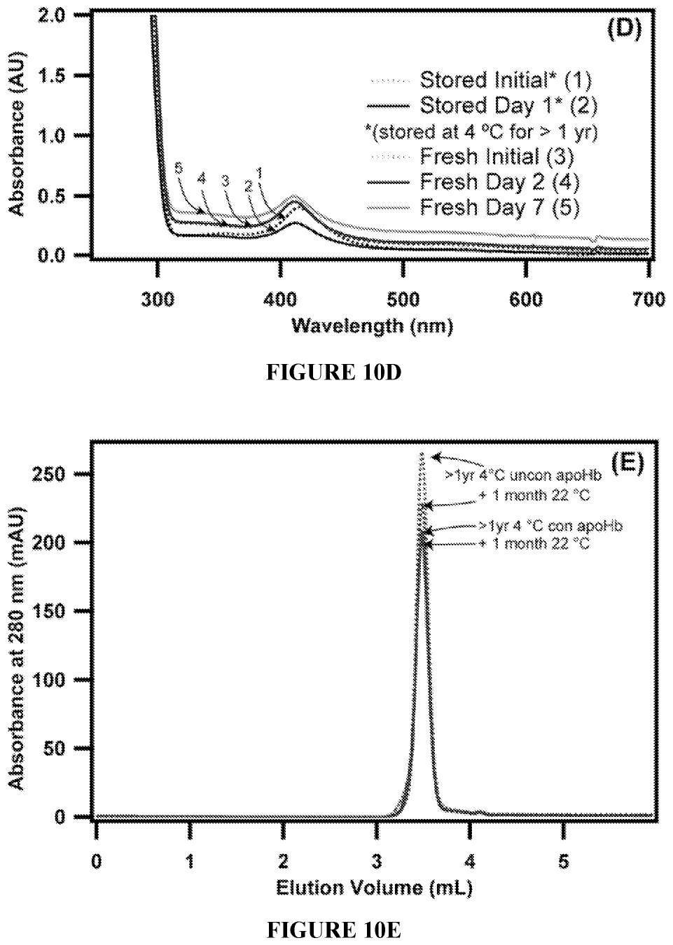

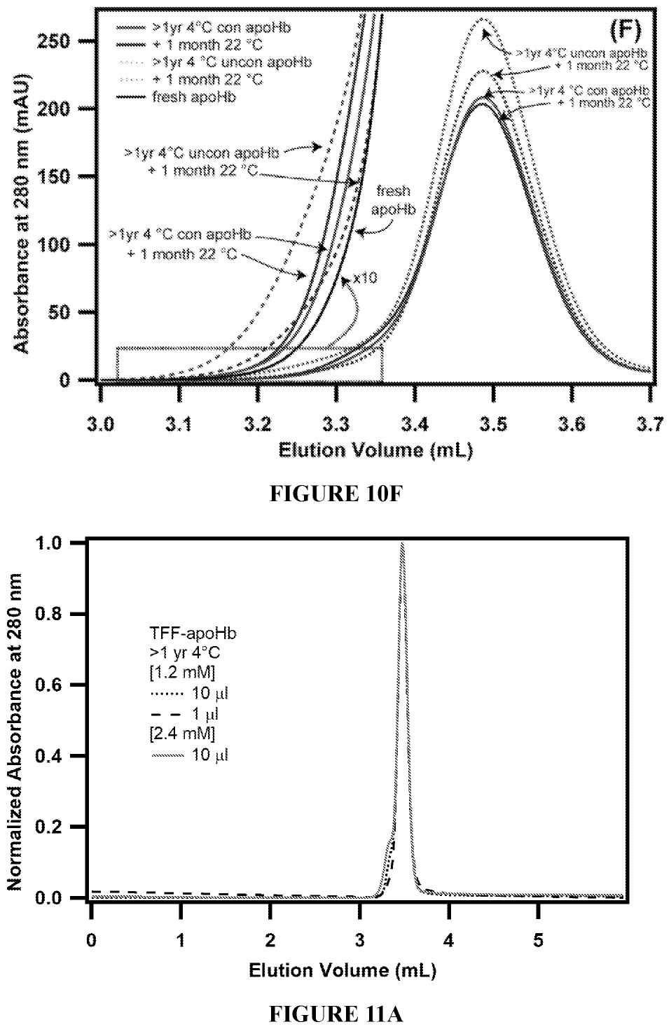

The stability of TFF-apoHb under different storage conditions was also assessed by RP-HPLC, CD and SEC-HPLC. This analysis is shown in FIG. 10. As shown previously, RP-HPLC did not show the presence of disulfide-bonded species nor oxidized forms of apoHb for freshly prepared samples. Furthermore, as shown in FIG. 10A, no oxidative modifications were observed for samples stored at −80° C. or lyophilized for over a year. Yet, samples stored at 4° C. or at 22° C. show an additional β-chain peak (βox), which is attributed to the oxidation of the methionine residue of the β-chain. This oxidative modification was attributed to oxidation of methionine and not to irreversible disulfide bond formation, since oxidation of methionine residues into methionine sulfoxide has been shown to lead to left-shifts on RP-HPLC (due to a decrease in protein hydrophobicity from oxidation of the methionine residue). If irreversible intermolecular disulfide bonds were created, it would be expected that elution of the oxidized species would have been right-shifted due to its higher MW similar to that observed for di-α crosslinked Hb.

To further investigate the effects of prolonged storage on TFF-apoHb, the CD spectra of stored samples was measured to analyze any loss of alpha helical content. All samples were diluted in DI water to approximately 10 μM to remove any interference from salt. The far UV CD spectra was measured for the diluted samples, and these results are shown in FIG. 10B. CD spectral analysis of stored samples showed that there may be slight reduction in the alpha helical content upon prolonged storage. Yet, there was no correlation of the alpha helical content to heme-binding activity or to the amount of β-chain oxidation. Overall, the apoglobin in solution did not show any relevant changes in secondary structure upon prolonged storage of TFF-apoHb.

Given that the literature on apoHb indicates that the apoprotein is unstable at room temperature and that TFF-apoHb was shown to be relatively stable at 22° C., a TFF-apoHb sample that had been stored at 4° C. for over a year was left at 22° C. in a sealed cuvette to monitor protein loss via precipitation. The results from this experiment is shown in FIGS. 10C and 10D for an unconcentrated and concentrated sample, respectively. The protein peak at 280 nm of the concentrated sample was above the upper detection limit of the spectrophotometer, so not much information could be obtained especially given the scattering due to the presence of precipitates in solution. Yet, the protein peak was detectable for the unconcentrated sample. Based on the 280 nm peak, there was an initial quick drop in protein concentration followed by a gradual decrease as a function of storage time. This was the same observation from FIG. 9 with ˜80-90% retention of total protein after one-week storage at 22° C. Since β-chain oxidation did not induce higher rates of apoprotein precipitation, the thermal stability of TFF-apoHb was not altered. Only a gradual increase in the absorbance at 700 nm was observed, which indicated that precipitates formed in solution. The samples stored at 4° C. and 22° C. were also analyzed using SEC-HPLC. Even though there was the presence of βₒₓ chains, their elution did not result in the formation of large quantities of tetrameric species (shown on SEC-HPLC of FIG. 10E). The decrease in the area under the curve (AUC) of the samples was expected given that there was a decrease in the total protein concentration in solution as a function of storage time. Furthermore, there was a slight increase in the relative amount of tetrameric species of the concentrated sample upon storage at 22° C. Interestingly, the amount of tetramers in the unconcentrated sample reduced after it was removed from 4° C. and placed at 22° C. This may be due to the lower thermal stability of the inactive species leading to their precipitation when heated to 22° C. It is noteworthy that TFF-apoHb shows a much higher stability at 22° C. than apoHb produced via other methodologies in the literature, which are highly unstable at 22° C. leading to large precipitate formation at temperatures above 10° C. Furthermore, for both the concentrated and unconcentrated samples, a higher fraction of the heme-bound TFF-apoHb species was lost compared to the loss of heme-free TFF-apoHb. This observation was noticed due to the higher retention of the protein peak compared to the retention of the residual Soret peak as a function of storage time. The residual heme-bound species could have either precipitated or the heme could have bound to the quartz cuvette.

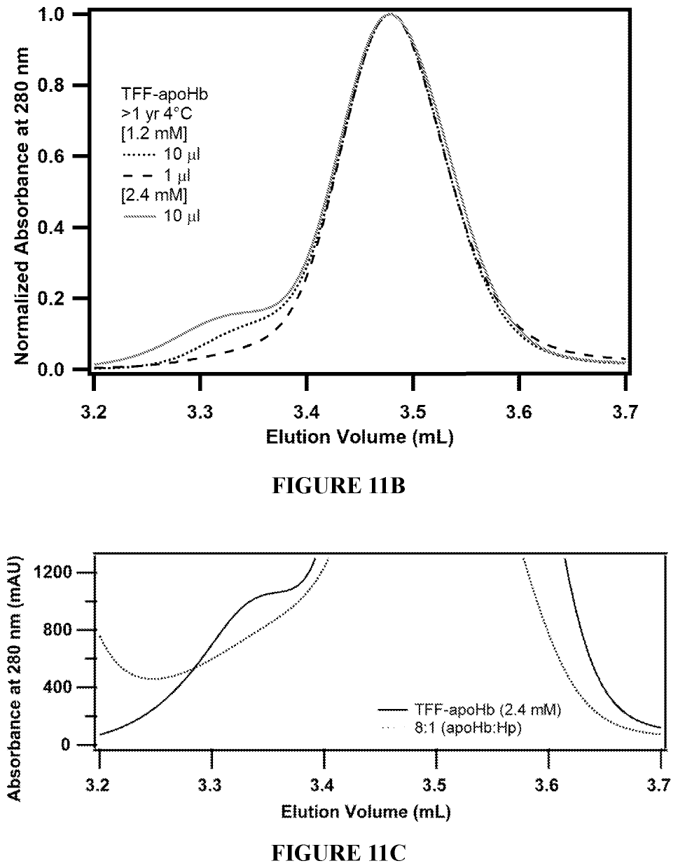

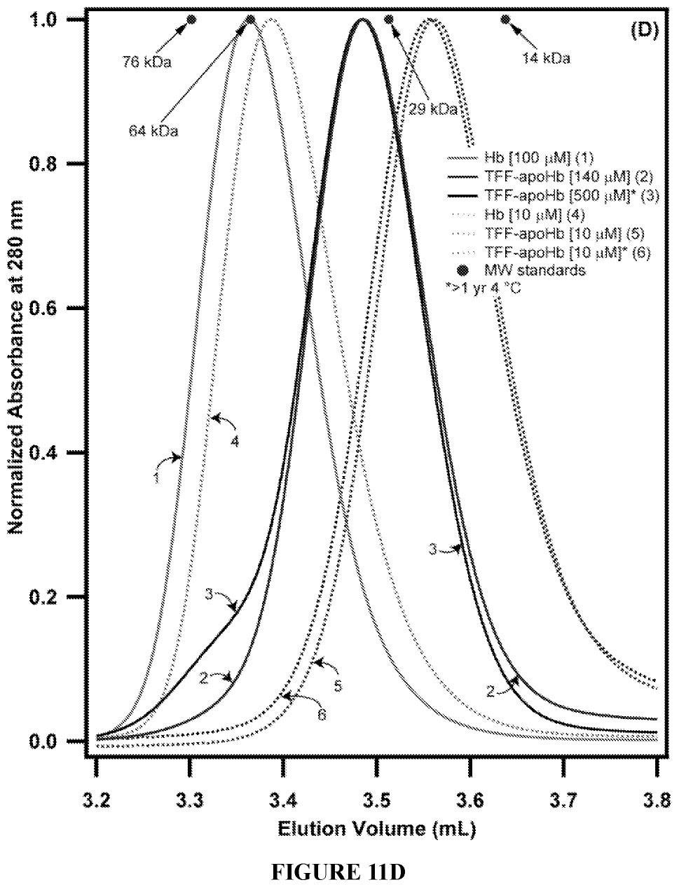

An analysis of the potential tetramer-dimer equilibrium of the apoprotein was performed by testing samples that contained detectable tetramers on the SEC-HPLC. These results are shown in FIG. 11. As shown in FIG. 11A, there was a minimal amount of tetramers even for a sample stored at 4° C. for more than a year. Furthermore, the relative amount of tetramers shown in the elution chromatogram was dependent on sample loading onto the column (FIG. 11B). Although molecules in fast equilibrium in SEC-HPLC elute at an intermediate elution volume of the two species, at slow equilibriums, the formation of two peaks is expected. Furthermore, addition of Hp to a tetramer containing sample led to a decrease in the elution peak of the tetrameric species (FIG. 11C). This decrease may indicate Hp binding to the tetrameric species similar to Hp binding to polymeric Hb. Yet, given the concentration dependence of the tetramers, it would be expected that Hp binding to apoHb dimers shifted the equilibrium towards lower tetramer concentrations. Thus, it was apparent that the relative amount of tetramers in the solution was dependent on protein concentration, which may indicate a tetramer-dimer equilibrium. Interestingly, at sufficiently low protein concentrations (10 μM), both Hb and TFF-apoHb shifted their SEC-HPLC elution to larger volumes, indicating a decrease in apparent MW. This shift is shown in FIG. 11D. For Hb, the shift indicated the tetramer-dimer equilibrium of the hologlobin at low concentrations. In the case of the TFF-apoHb, the elution indicated that at low concentrations, TFF-apoHb existed in a dimer-monomer equilibrium. The same phenomena occurred on a TFF-apoHb sample with some tetrameric apoHb. Although these equilibrium states were observed, the tetramer-dimer equilibrium was not consistent between samples given that the same injection volume at the same concentration of apoHb from two samples showed different amounts of tetrameric species. This may be related to its much lower equilibration rate. Furthermore, from the experiments we performed, there was no detectable correlation between tetramers, βₒₓ or the sample's relative activity. Therefore, there is still a need to investigate these tetrameric species, their equilibrium in solution and potential correlation to apoHb activity and Pox.

Overall, given that there have been various reports in the literature on oligomerization of apoHb prepared via either acid-acetone or MEK methodologies, the formation of these non-native species may be linked to the preparation of apoHb. Not only was there minimal amounts of tetrameric species in our TFF-apoHb preparations, but there were no higher orders species detected under any of the tested storage conditions. Previous SEC-HPLC of MEK-apoHb showed a large percentage of tetrameric species and other higher order aggregates, which required the use of a reducing agent (such as dithiothreitol) during preparation to form dimeric apoHb. Furthermore, these tetrameric species were found to be dissociated at low concentrations, indicating that these species were not formed via irreversible disulfide bonds. Finally, even dimeric apoHb may also dissociate into monomers at low concentrations.

Conclusions

The possible biomedical applications of apoHb are very promising. Yet, its wide-scale use and analysis is restricted by current production methods, which are not easily scalable. New techniques to produce active apoHb have not been presented for decades even though new bioprocessing techniques have been developed. Existing apoHb production protocols require extensive dialysis and the use of highly flammable solvents. The newly proposed acidic alcohol TFF apoHb production method provides an easy and scalable method for producing active apoHb with more than 95% total protein and 75% active protein yields. Through the use of a 80% (v/v) ethanol:water solution for heme extraction, the flammability risks and toxicity issues with residual solvent are drastically lowered compared to previous apoHb production methods. Yet, the most valuable benefit of this new method is the use of an easily and cost-effective scalable process such as TFF for protein purification.

TFF-apoHb has the same characteristics as apoHb produced via previously published methodologies in the literature. These characteristics include: heme-binding activity, Hp binding activity, exists primarily as a dimeric species in aqueous solution, rHb O₂ equilibria and ligand binding kinetics. Yet, unlike apoHb produced previously in the literature, stability studies showed that TFF-apoHb can be stored at 4° C., −80° C. and in lyophilized form without appreciable changes in activity with higher stability at room temperature compared to previous apoHb storage studies. Additionally, ESI-MS analysis of TFF-apoHb demonstrated that it retained its structure without any chemical modifications. RP-HPLC demonstrated that no oxidative modifications were present in freshly prepared TFF-apoHb nor for samples lyophilized or stored at −80° C. Quaternary structure analysis via SEC-HPLC showed that TFF-apoHb αβ dimers did not form appreciable amounts of tetramers over prolonged storage at 4° C. Furthermore, new insight into apoHb oxidation and degradation was provided based on the oxidation of the β-chain of apoHb when stored at 4° C. or 22° C. for prolonged periods of time. Evidence for both tetramer-dimer and dimer-monomer equilibrium of apoHb was also presented. Finally, an improved hemichrome removal procedure was developed that could generate rHb absorbance spectra indistinguishable from native Hb. Also, rHb generated from TFF-apoHb demonstrated that the reconstituted protein maintains native Hb-like O₂ dissociation and CO association kinetics.

Taken together, this example presents an improved method for producing apoHb with a comprehensive analysis of the relevant biophysical properties apoHb and rHb.

### Example 2: Tangential Flow Filtration of Haptoglobin from Plasma or Plasma Fractions

The example describes a scalable process to enable purification of haptoglobin (Hp) from serum proteins in plasma or plasma fractions through the use of tangential flow filtration (TFF). The TFF process brackets Hp in a molecular weight range with low levels of common serum proteins, yielding a product that can include polymeric forms of Hp.

Haptoglobin (Hp) is an α-2 glycoprotein mainly responsible for scavenging cell-free hemoglobin (Hb). Although found in most bodily fluids of mammals, it is present in plasma at concentrations normally ranging from 0.5-3 mg/mL. After binding to cell-free Hb, the Hb-Hp complex is scavenged by CD163+ macrophages and monocytes to clear the organism of toxic cell-free Hb. Cell-free Hb toxicity is attributed to a variety of factors, including Hb extravasation into tissue space which elicits oxidative tissue injury, nitric oxide (NO) and peroxide scavenging, and free heme release. These factors can lead to acute and chronic vascular disease, inflammation, thrombosis, and renal damage. When bound to Hp, the large size of the Hb-Hp complex prevents Hb extravasation into the tissue space, lowering nitric oxide scavenging and vasoconstriction. Furthermore, Hp binding to Hb prevents heme release from Hb, and lowers Hb oxidative damage and inflammation. For these reason, Hp is used as a therapeutic in Japan and is being researched for treatment of various states of hemolysis. Recent studies have also shown various roles Hp plays as a chaperone and in regulation of redox states. In clinical settings, high levels of Hp are indicative of acute inflammation as its expression is upregulated in response to inflammatory cytokines, and low Hp levels are indicative of hemolysis due to receptor-mediated uptake of Hp-Hb complexes.

Hp is a polymorphic protein composed of αβ dimers in which the β polypeptide chain is coded by the same gene, while the α chain can be coded by either the Hp1 or Hp2 codominant alleles. These give rise to three main Hp phenotypes: Hp1-1, Hp2-1 and Hp2-2. The α and β chains are bound through disulfide bonds with the β, α-1 and α-2 chains having a molecular weight (MW) of 36, 9 and 18 kDa, respectively. The α-1 chain has a second cysteine residue after binding to the β chain that allows it to bind to another α chain of an αβ dimer. Thus, Hp1 homozygotes produce tetrameric Hp1-1 (two β and two α-1) species with a MW of about 89 kDa. On the other hand, the α-2 chain has two free cysteine residues when bound to the β chain allowing it to bind to two αβ dimers. This extra cysteine residue leads to the formation of Hp polymers in heterozygote or Hp2 homozygote individuals. Heterozygotes produce Hp2-1, a linear Hp polymer with an average MW of about 200 kDa, which can go up to about 500 kDa. Finally, Hp2 homozygotes produce Hp2-2 which is a cyclic polymer form of Hp with average MW of about 400 kDa ranging from 200 to 900 kDa. All types of Hp bind Hb via a practically irreversible reaction, with a K_{d} ranging from 10¹² to 10¹⁵M. The bond occurs between the β chain of Hp to the β globin of Hb at a stoichiometry of 1:1 for each dimer. Since the MW of Hp differs between phenotypes, the mass stoichiometry is not consistent, with about a 1.3:1 mass binding ratio for Hp1-1:Hb and about a 1.6:1 mass binding ratio for Hp2-2:Hb. Furthermore, Hp 2-2 has been shown to have a higher affinity for the CD163+ receptor, but lower clearance rate through CD163+ uptake. However, the different Hp phenotypes reduce Hb toxicity to the same extent in vivo. Furthermore, Hp2-2 has not been found to have differences in the rate of heme loss, Hb oxidation, and Hb dimer association kinetics in vitro compared to Hpl-1 Although there are no significant differences with its role in Hb scavenging, differences in Hp phenotype have been associated with different rates of cardiovascular disease and cancer as well as different roles with some forms of disease.

In addition to the three main Hp phenotypes, another related Hb scavenging species is haptoglobin related protein (Hpr). Hpr is composed of smaller α and β chains than Hp1-1 and is predominantly found as single αβ dimers, but has been shown to form polymers. With >90% sequence identity to the Hp1 gene, Hpr binds to Hb with high affinity. Unlike Hp, the α chains do not covalently bind to other α chains through disulfide bonds to create αβ polymers, but are thought to connect via non-covalent interactions. The physiological role of Hpr differs from normal Hp as Hpr does not bind to the CD163 receptor and does not have increased expression during states of hemolysis. Instead, Hpr forms a complex with high-density lipoproteins called trypanosome lytic factor 1 (TLF1) and TLF2, which can have a large range of molecular sizes. These complexes have lytic activity against the African cattle parasite Trypanosoma brucei brucei, which use the trypanosoma receptor to obtain iron from the heme of Hb through binding of Hp-Hb complexes. Since Hpr can still bind to Hb in TFL1 and to the trypanosoma receptor, the Hpr-Hb in TFL1 acts as a “trojan horse” against trypanosoma parasites providing humans an innate defense against this disease.

The normal circulatory half-life of Hp is 1.5-2 days in humans, but the half-life of the Hp-Hb complex reduces to −20 min, with a maximum clearance rate of 0.13 mg/mL of plasma per hour. Due to the higher rate of clearance of the Hp-Hb complex, even though Hp production is upregulated during states of hemolysis, the concentration of Hp in plasma is inversely related to the concentration of cell-free Hb in plasma. In addition to its upregulation due to the presence of cell-free Hb in the circulation, Hp synthesis is heavily stimulated during acute phase reactions (inflammation, infection, trauma, and malignancy).

Hp can be used as a therapeutic during conditions that cause states of hemolysis (e.g., chronic anemia, transfusion, etc.). In these states, rupture of red blood cells (RBCs) releases cell-free Hb that can scavenge NO, leading to vasoconstriction as well as formation of free radicals and reactive oxygen species that can lead to oxidative damage of surrounding tissues. Hp upregulation during bacterial infection has been related to iron deprivation of pathogens. For this reason, Hp may be used to treat septic shock. Additionally, Hp or the Hp/serum protein mixtures presented here, may be used in RBC storage solutions to extend the ex vivo shelf-life of these cells by attenuating the side-effects associated with lysed RBCs. These solutions could also be co-administered with RBCs or hemoglobin-based oxygen carriers (HBOCs) to prevent and treat the side-effects associated with cell-free Hb.

Hp has been clinically approved in Japan since 1985. Reports of its use show positive effects against burn injuries, trauma from massive transfusions and as a prophylactic during surgical interventions such as cardiac bypass surgery with extracorporeal circulation. Hp treatment during severe burns has been shown to prevent acute renal failure and reduce kidney damage from surgery.

The biomedical applications of Hp are very promising. Its clinical use in Japan has shown positive effects against burn injuries, trauma from massive transfusions and as a prophylactic during surgical interventions such as cardiac bypass surgery with extracorporeal circulation. Hp treatment during severe burns has been shown to prevent acute renal failure, and reduce kidney damage from surgery. In general, Hp can be used to detoxify cell-free Hb that is present in the systemic circulation via various pathophysiological conditions or RBC transfusions that result in hemolysis. Hp treatment has also been shown to prevent damage from stored RBCs, potentially prolonging its shelf-life.

Yet, its wide-scale use is restricted by current production methods, which are not easily scalable and expensive. Furthermore, treatments with Hp require large quantities of material per dose. Existing Hp production protocols consist of using either Hb-affinity chromatography, hydrophobic interaction chromatography, anion-exchange chromatography, or recombinant Hp expression. Chromatography has low yields and is limited by the protein binding capacity of the column. Furthermore, chromatography requires the use of harsh denaturants to dissociate Hp from the bound chromatography matrix. Finally, recombinant Hp is expensive and cumbersome to manufacture.

This example describes a Hp production method via TFF that provides an easy, scalable and economically efficient method for producing Hp from plasma or plasma fractions. The plasma fraction chosen for this example was human Cohn Fraction IV obtained via the modified Cohn process of Kistler and Nitschmann (See, for example, Kistler, P. & Nitschmann, H. Large Scale Production of Human Plasma Fractions. Vox Sang. 7, 414-424 (1962)). This plasma fraction is known to contain large MW Hp (Hp2-2 and Hp2-1) from pooled plasma. Low MW Hp (small Hp2-1 polymers and Hp1-1) are primarily found in Cohn Fraction V.

Materials and Methods

Materials. Sodium phosphate dibasic, sodium phosphate monobasic, sodium chloride, and fumed silica (S5130) were purchased from Sigma Aldrich (St. Louis, Mo.). 0.2 μm Millex-GP PES syringe filters were purchased from Merck Millipore (Billerica, Mass.). A KrosFlo® Research II tangential flow filtration (TFF) system and hollow fiber (HF) filter modules were obtained from Spectrum Laboratories (Rancho Dominguez, Calif.). Human Fraction IV Paste was purchased from Seraplex, Inc (Pasadena, Calif.).

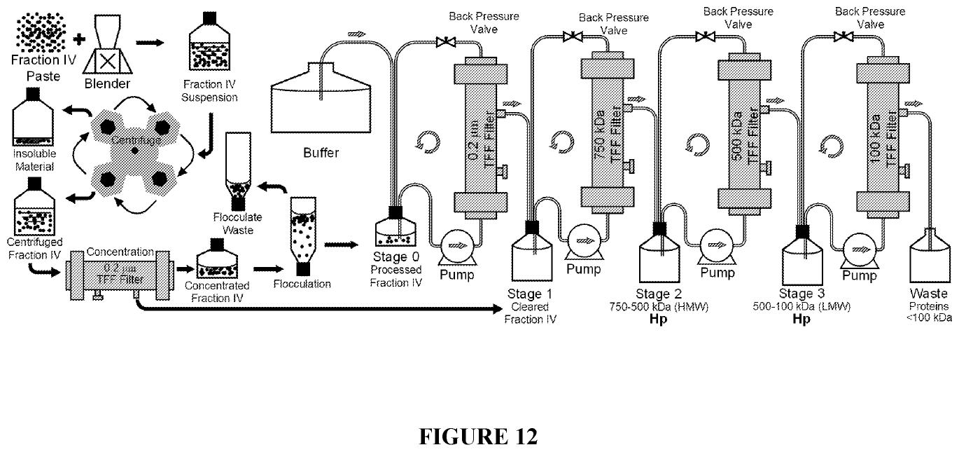

Hp Purification via TFF Without Fumed Silica. 500 g of human Fraction IV (FIV) paste from the modified Cohn process of Kistler and Nitschmann was suspended in 5 L of PBS, and homogenized in a blender. The resulting mixture was stirred overnight at 4° C. The ˜5 L solution was then centrifuged at 3700 g for 45 minutes to remove undissolved lipids. The supernatant was concentrated using a 0.2 μm hollow fiber (HF) filter to 2 L. The 2 L retentate was left to rest for 36 hrs to flocculate low density particles, while the filtrate was kept at 4° C. for further processing. After flocculation of the retentate, low density particles in solution were separated. The higher density fraction (Stage 0) was then concentrated to 800 mL on a 0.2 μm HF filter and subjected to 10 diafiltrations with PBS. The 0.2 μm filtrate (Stage 1) was concentrated to 150 mL and subjected to 100 diafiltrations on a 750 kDa HF filter using a mixture of the 0.2 μm permeate and PBS. The permeate of the 750 kDa (Stage 2) was then concentrated to 150 mL subjected to 40 diafiltrations using PBS on a 500 kDa HF filter. Finally, the permeate of the 500 kDa HF filter (Stage 3) was concentrated to 150 mL and subjected to 100 diafiltrations using PBS on a 100 kDa filter. A diagram of the purification process is shown in FIG. 12 and the characteristics of the filters used are shown in Table 3. The ˜150 mL solutions of both the 750-500 kDa (high molecular weight, BMW) and 500-100 kDa (low molecular weight, LMW) brackets were then concentrated on 100 kDa HF filters to ˜5-10 mL for the BMW and ˜30 mL for the LMW brackets, respectively.

TABLE 3
Hollow fiber filters (Waltham, MA) used for the purification of Hp
from Cohn Fraction IV using tangential flow filtration.

| | Pore | | | Surface Area | |
|---|---|---|---|---|---|
| Type | Size | | Membrane | (cm²) | P/N |
| miniKros | 0.2 | μm | PES | 470 | S02-P20U-10-N |
| miniKros | 750 | kDa | mPES | 790 | S02-E750-05-N |
| miniKros | 500 | kDa | PS | 1000 | S02-S500-05-N |
| miniKros | 100 | kDa | mPES | 1000 | S02-E100-05-N |
| midiKros | 100 | kDa | mPES | 115 | D02-E100-05-N |

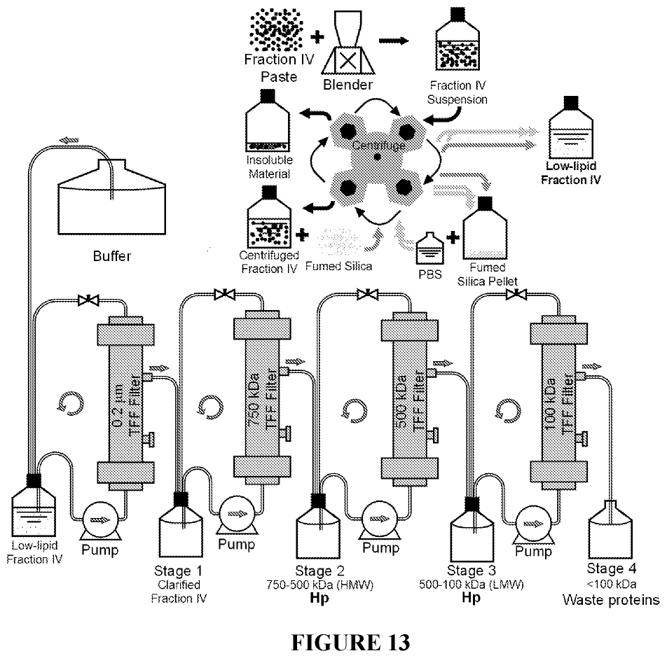

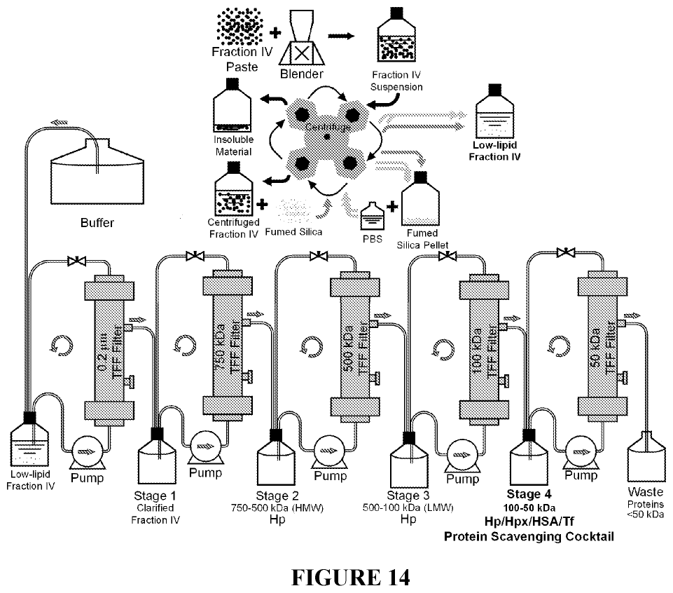

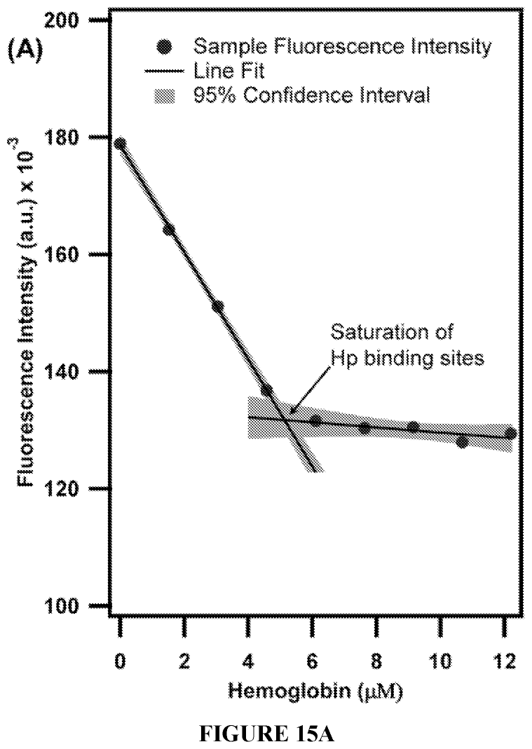

Hp Purification via TFF with Fumed Silica. 500 g of human Fraction IV (FIV) paste from the modified Cohn process of Kistler and Nitschmann was suspended in 5 L of PBS, and homogenized in a blender. The resulting mixture was stirred overnight at 4° C. The ˜5 L solution was then centrifuged at 3700 g for 45 minutes to remove undissolved lipids. Fumed silica (Sigma Aldrich P #55130; St. Louis, Mo.) was then added to the sample at 20 mg/mL concentration and left stirring overnight at 4° C. The solution was then centrifuged to remove the silica agglomerates. Furthermore, the silica pellet was washed twice with PBS to maximize protein recovery. The fumed silica supernatant solution was then concentrated to 800 mL on a 0.2 μm HF filter and filtered for 15 diafiltrations. The 0.2 μm filtrate was concentrated to 150 mL and subjected to 100 diafiltrations using PBS on a 750 kDa HF filter. The permeate was then subjected to 40 diafiltrations using PBS on a 500 kDa HF filter. Finally, the permeate of the 500 kDa HF filter was subjected to 100 diafiltrations using PBS on a 100 kDa filter. A diagram of the purification process is shown in FIG. 13 and the characteristics of the filters used are shown in Table 3. The ˜150 mL solutions of both the 750-500 kDa (high molecular weight, BMW) and 500-100 kDa (low molecular weight, LMW) brackets were then concentrated on 100 kDa HF filters to 5-10 mL for the HMW and −40 mL for the LMW brackets, respectively.

Size Exclusion Chromatography: Samples were separated via size exclusion chromatography (SEC) using an Acclaim SEC-1000 (4.6×300 mm) column (Thermo Fisher Scientific, Waltham, Mass.) attached to a Dionex UltiMate 3000 system (Thermo Fisher Scientific, Waltham, Mass.). The mobile phase consisted of 50 mM potassium phosphate, pH 7.4 at a flow rate of 0.35 mL/min controlled by Chromeleon 7 software. Wavelength absorbance detection was set to λ=280 nm to detect protein, and λ=413 nm to detect Hb. To estimate the average MW of the Hp products, protein standards (conalbumin, 76 kDa; hHb, 64 kDa; carbonic anhydrase, 29 kDa; ribonuclease A, 14 kDa; and aprotinin, 6.5 kDa) were analyzed on the SEC column. The known molecular weight (MW) of the standards and their elution time were used to determine the coefficients (A, B) of a base 10 exponential function (MW=10^{A*(elution time)+B)} via non-linear regression. The estimated function parameters were used to estimate the average MW of Hp products based on their elution time.

Hb Concentration. The concentration of Hb in the samples was measured spectrophotometrically via the Winterbourn equations.

Residual Hb in Hp Preparations. The residual Hb was quantified via the molar extinction coefficient of methemoglobin at its Soret Peak maxima of 404 nm (ε₄₀₄=167 mM⁻¹cm⁻¹).

Residual Apohemoglobin in Hp Preparations. The concentration of residual apohemoglobin (apoHb) was estimated based on a modified version of the abridged dicyanohemin incorporation assay. Briefly, Hp samples were mixed with excess methemealbumin (heme bound to human serum albumin [HSA]) and the mixture left to react for 15 hours at room temperature. The change in absorbance of the reacted mixture compared to the initial sample components was used to estimate the amount of heme exchanged from methemealbumin to apoHb. Given the estimated extinction coefficients for the hemichrome-like apoHb species formed (ε₄₁₂ₙₘ≈120 mM⁻¹cm⁻¹) and methemealbumin (ε₄₁₂nm≈70 mM⁻¹cm⁻¹), the extinction coefficient for the change in absorbance spectra was determined to be 55 mM⁻¹cm⁻¹ at 412 nm.

ELISA. To quantify the concentration of residual protein components in the Hp samples, ELISA kits specific for Hp, transferrin (Tf), human serum albumin (HSA), and hemopexin (Hpx) were used (R&D Systems Catalog #DHAPGO for Hp, and Eagle BioSciences HTF31-K01 for Tf, HUA39-K01 for HSA, and HPX39-K01 for Hpx).

Gel Electrophoresis: The purity of Hp fractions was analyzed via SDS-PAGE using an Invitrogen Mini Gel Tank (Thermo Fisher Scientific, Waltham, Mass.). Wide-range Tris-Glycine gels consisting of pre-cast 4-20% or 10-20% polyacrylamide were used with samples prepared according to the manufacturer's guidelines. Gels were loaded with 20 μL of sample corresponding to approximately 30 μg of protein per lane and tested under reducing (via addition of 0.1 M DTT) and non-reducing conditions. Gels were stained for one and a half hours with Coomassie® Briliant Blue R-250 staining solution, then de-stained overnight. Gels were imaged on a table-top scanner at 300 dpi. To estimate the percent composition of each band, reduced and non-reduced gels were slightly overloaded (˜60 μg of protein) and densitometric analysis was performed on the scanned images using ImageJ. The Hp composition was determined based on the sum of protein bands corresponding to Hp or Hpr α and β chains from the reduced gels subtracted by the composition of the Hb-eluting band on the non-reduced gels (due to the known coelution of Hb α and β chains with the α-1 chain of Hp).

Trypsin Digest Mass Spectrometry. Samples were reduced with 5 mM DTT, incubated at 65° C. for 30 min. Iodoacetamide was then added to a final concentration of 15 mM and the sample was incubated in the dark, at room temperature for 30 minutes. Sequencing grade trypsin was added at a 1:50 ratio and the sample was digested overnight at 37° C. The following day, the samples were acidified with trifluoro acetic acid. The sample was clarified at 13,000 rpm for 5 min in a microcentrifuge, dried in a vacufuge and resuspended in 20 μL of 50 mM acetic acid. Peptide concentration was determined by NanoDrop (i.e. absorbance at 280 nm). Protein identification was performed using nano-liquid chromatography-nanospray tandem mass spectrometry (LC/MS/MS) on a Thermo Scientific Fusion Orbitrap mass spectrometer equipped with an EASY-Spray™ Sources operated in positive ion mode. Samples were separated on an easy spray nano column (Pepmap™ RSLC, C18 2μ 100 A, 75 μm×250 mm Thermo Scientific) using a 2D RSLC HPLC system from Thermo Scientific. Each sample was injected into the μ-Precolumn Cartridge (Thermo Scientific) and desalted with 0.1% formic acid in water for 5 minutes. The injector port was then switched to inject and the peptides were eluted off of the trap onto the column. Mobile phase A was 0.1% formic acid in water and acetonitrile (with 0.1% formic acid) was used as mobile phase B. Flow rate was set at 300 nL/min. Typically, mobile phase B was increased from 2% to 20% in 105 min and then increased from 20-32% in 20 min and again from 32%-95% in 1 min and then kept at 95% for another 4 min before being brought back quickly to 2% in 1 min. The column was equilibrated at 2% of mobile phase B (or 98% A) for 15 min before the next sample injection. MS/MS data was acquired with a spray voltage of 1.7 kV and a capillary temperature of 275° C. The scan sequence of the mass spectrometer was as follows: the analysis was programmed for a full scan recorded between m/z 375-1575 and a MS/MS scan to generate product ion spectra to determine amino acid sequence in consecutive scans starting from the most abundant peaks in the spectrum in the next 3 seconds. To achieve high mass accuracy MS determination, the full scan was performed at FT mode and the resolution was set at 120,000. The AGC Target ion number for FT full scan was set at 4×10⁵ ions, and maximum ion injection time was set at 50 ms. MS/MS was performed using ion trap mode to ensure the highest signal intensity of MS/MS spectra using CID (for 2+ to 7+ charges). The AGC target ion number for ion trap scan was set at 1×10⁴ ions, and maximum ion injection time was set at 30 ms. The CID fragmentation energy was set to 35%. Dynamic exclusion was enabled with a repeat count of 1 within 60 s and a low mass width and high mass width of 10 ppm. Data sets were analyzed as the Total Ion Intensity for each protein (normalized based on the total ion current) using Scaffold 4 (Proteome Software, Inc).

Total Protein Assay. Total protein of the samples was determined via the Bradford Assay.

Hb Binding Capacity of Hp (Fluorescence). The Hb binding capacity (HbBC) of samples was determined based on the fluorescence quenching method described in the literature. Briefly, a Hp sample is mixed with increasing amounts of Hb and the fluorescence emission at 330 nm (with excitation at 285 nm) is measured. Binding of Hb to Hp quenches the fluorescence of Hp, leading to an observable titration curve. This assay was repeated on products of Stage 2 and 3 for three different batches and compared to the SEC method to quantify HbBC.

Hb Binding Capacity of Hp (SEC): The difference in molecular weight (MW) between the Hp-Hb protein complex and pure Hb was used to assess the Hb binding capacity of Hp. Briefly, samples containing Hp were mixed with excess Hb then separated via SEC. The difference in the area under the curve between the pure Hb solution, and the mixture of Hb and Hp was used to assess the HbBC of Hp.

Heme Binding Activity: The activity of the heme-binding pocket of the protein scavenger cocktail was determined via the dicyanohemin (DCNh) incorporation assay. Briefly, increasing amounts of DCNh was added to a constant concentration of sample and the inflection point of the equilibrium absorbance at the Soret maxima was used to determine the molar quantity of heme required to saturate the heme-binding sites of the sample. The mass concentration of the heme-binding proteins was estimated based on an approximate molecular weight of 65 kDa for human serum albumin and hemopexin.

Effect of Fumed Silica: To assess the total loss of protein from the use of fumed silica, three 50 mL samples of suspended Fraction IV at 100 mg/mL were characterized after removal of the unsuspended FIV (mostly lipids), after addition of 20 mg/mL fumed silica with overnight stirring (fumed silica supernatant), and after each of the two washes (washing consisted of replacing the volume of supernatant removed with fresh PBS, and mixing the fumed silica pellet). The volume reduction due to fumed silica addition was approximated to be 15% as described by the manufacturer. Characterization consisted of quantification of HbBC and total protein, and via separation via SEC-HPLC. The percent of HbBC and total protein retained at each stage after fumed silica addition was calculated based on the ratio of the concentration of HbBC and total protein compared to the FIV supernatant prior to silica addition.

Results and Discussion

Hemoglobin Binding Capacity of Hp. Throughout this study, Hp activity was quantified based on the HbBC of the sample. This parameter indicates the mass of Hb that a given volume of the Hp sample can bind. This approach for Hp quantification is also used in Japan where the HbBC is equivalent to the international units (IU) of the therapeutic compositions of Hp. Given that different Hp phenotypes have different mass binding ratios to Hb and that the main role of Hp is as a Hb binding protein, the HbBC provides a more reliable and practical measurement of Hp activity in a Hp sample. This measurement of Hp activity is of critical importance in assessing the efficacy of various Hp production methods, especially those that rely on harsh denaturing conditions to purify Hp in which some of the Hp may be denatured.

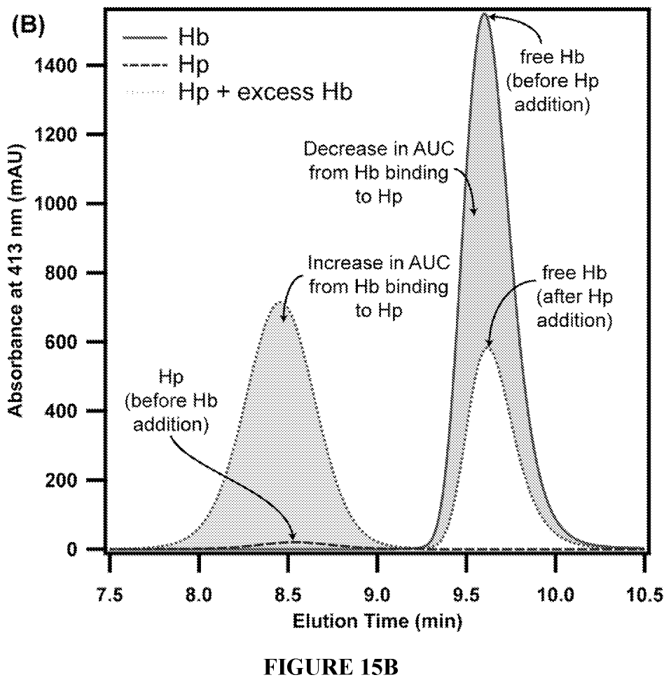

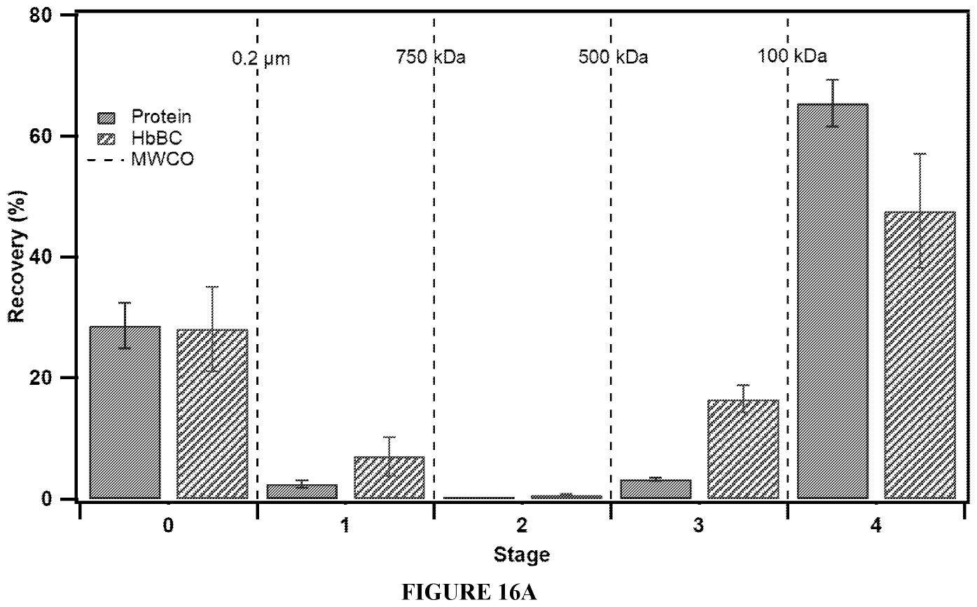

There are various methods to quantify the HbBC of Hp samples. These include spectrophotometric titrations with Hb, immunodiffusion, gel electrophoresis, spectrophotometric differences between deoxygenated Hb and the deoxygenated Hb-Hp complex, differences in peroxidase activity of Hb compared to the Hb-Hp complex, and fluorescence titration of Hp with Hb. Unfortunately, spectrophotometric assays can be dependent on the Hp phenotype and convoluted by other species. Additionally, ELISA can also be used, but differences in polymer sizes can also lead to inaccurate readings. One of the initial methods was based on the fluorescence quenching of Hp tryptophan residues upon Hb binding. In this method, a stock Hp sample is titrated with increasing Hb concentrations and the fluorescence intensity is measured after each addition. The saturation point of Hb binding sites in Hp is determined by the change in slope of the titration curve. FIG. 15A shows an example of a Hp fluorescence titration curve against Hb. Yet, this procedure can be time consuming, requires many data points, and can have large variations from the fitted lines. To streamline and improve HbBC measurements of Hp samples, a new and quick method to determine HbBC was developed. In this method, the difference in size between Hb compared to Hb bound to Hp (i.e. Hb-Hp complex) was used to determine the quantity of Hb bound in Hb-Hp complexes. This was done by monitoring the absorbance at 413 nm during SEC-HPLC of the sample and calculating the area under the curve (AUC) of a known concentration of free Hb, and then repeating this measurement after mixing the Hb with a Hp sample. The decrease in the AUC of the peak corresponding to free Hb after mixing with Hp was directly proportional to the HbBC of the sample, and could be precisely calculated based on the known concentration of Hb. Examples of HPLC-SEC chromatograms of Hp samples using this procedure are shown in FIG. 15B. The HbBC could also be calculated based on the increase in AUC of the peak corresponding to the Hb-Hp complex and is not restricted to only monitoring the 413 nm wavelength (protein peak at 280 nm and others may also be used).

Comparing the fluorescence titration and HPLC-SEC AUC Hp quantification methods for six different Hp containing samples, there was less than 5% variation of the results which, at the concentrations tested, led to less than 1 mg/mL of HbBC variation. Furthermore, the HPLC-SEC method has less than 1% variation in precision. Thus, the 5% variation seen may have been due to the intrinsic variation of the fluorescence titration method. Furthermore, these variations were similar or better than the reported values for previous HbBC quantification methods. The difference in peroxidase activity, had larger variations (7.6% using different standards and 2.6% using same standard curve). Spectrophotometric differences between free Hb and Hb-Hp had more than 10% error. Furthermore, spectrophotometric titration with Hb had about 2% variation within the same sample and ranged from 2-11% when the same sample was tested on a different day. A similar method which employed SEC-HPLC to determine HbBC has been employed previously, in which the AUC of the Hb-Hp complex was divided by the total AUC of the chromatogram. Although relying on the same concept (use of HPLC-SEC to separate Hb from the Hp-Hb complex), the previously used method requires that the Hp does not have any Hb bound to it. Furthermore, slight modifications in the absorbance of Hb-bound species may occur. For example, the change in absorbance from the binding of free cyanomethemoglobin to Hp has been used to quantify HbBC. Using the method presented here in which only the AUC of the Hb peak is used in the analysis, this method removes potential errors from analysis of the AUC from the Hp-Hb complex peak.

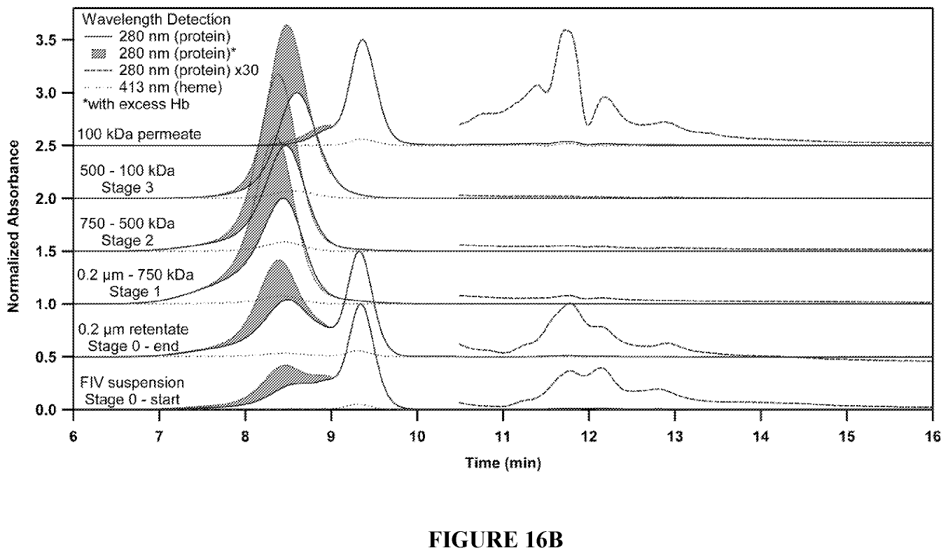

TFF Production of Hp Without Fumed Silica. Production of Hp mixtures via the tangential flow filtration (TFF) procedure described in the Methods Section yielded 5 different stages in which different molecular weight (MW) proteins could be isolated. These 5 stages were the proteins retained on the 0.2 μm HF filter (Stage 0, 0.2 μm retentate), the bracket of proteins between the 0.2 μm and 750 kDa HF filters (Stage 1, 0.2 μm-750 kDa), the bracket between the 750 kDa and the 500 kDa (Stage 2, 750-500 kDa), the bracket between the 500 and 100 kDa HF filters (Stage 3, 500-100 kDa), and lastly, the permeate of the 100 kDa HF filter (Stage 4, <100 kDa). The protein and Hb binding capacity (HbBC) yield at each stage based on the average of four batches without the use of fumed silica is shown in Table 4. The recovery data for total protein and Hb binding capacity (HbBC) at each stage of processing is also shown in FIG. 16A.

TABLE 4
Summary of total protein concentration and HbBC, and analysis of total protein and
HbBC yield of batches without the use of fumed silica.

| | Total | | | | | |
|---|---|---|---|---|---|---|
| | Protein | HbBC | Total | HbBC per | Protein | HbBC |
| STAGE | (mg/mL) | (mg/mL) | Protein (g) | mg (%) | Yield (%) | Yield (%) |
| 0' | 22.0 ± 1.4 | 1.4 ± 0.1 | 115 ± 9 | 6.59 ± 0.97 | 100 | 100 |
| FIV suspension | | | | | | |
| 0 | 31.6 ± 5.2 | 2.0 ± 0.4 | 33.1 ± 5.1 | 6.35 ± 1.1 | 28.69 ± 3.8 | 28.11 ± 6.98 |
| 0.2 μm retentate | | | | | | |
| 1 | 20.7 ± 2.8 | 3.6 ± 1.8 | 2.77 ± 0.68 | 18.4 ± 6.2 | 2.42 ± 0.65 | 7.05 ± 3.18 |
| 0.2 μm-750 kDa | | | | | | |
| 2 | 44.2 ± 7.2 | 12.2 ± 3.9 | 0.191 ± 0.019 | 27.0 ± 5.1 | 0.17 ± 0.03 | 0.71 ± 0.15 |
| 750-500 kDa | | | | | | |
| 3 | 100.1 ± 4.4 | 32.8 ± 1.8 | 3.77 ± 0.20 | 32.8 ± 2.3 | 3.28 ± 0.26 | 16.5 ± 2.3 |
| 500-100 kDa | | | | | | |
| 4 | <1 | — | 75.5 ± 8.1 | 4.9 ± 1.7 | 65.4 ± 3.9 | 47.6 ± 9.4 |
| 100 kDa permeate* | | | | | | |

*values were not measured and include all losses during processing; estimates were based on the initial total protein and HbBC compared to what was recovered in the other stages.

From the results of Table 4 and FIG. 16A, it was apparent that FIV was not primarily composed of soluble proteins. Starting with a ˜100 mg/mL suspension of FIV paste, the solution was only composed of ˜22 mg/mL of soluble total protein. Thus, only approximately 20% of the mass of FIV was composed of soluble proteins. Some of the insoluble material was removed from the centrifugation steps in which approximately 150-200 g of insoluble material was removed. The remaining mass of material was likely retained on the 0.2 μm filter.

Given that most serum proteins have MW smaller than 100 kDa, most of the suspended protein permeated through the 100 kDa filter, leading to 65% of the soluble protein being lost in Stage 4. Furthermore, since Hp can be present over a wide MW range and that FIV contains mostly polymeric Hp, Stage 3 retained a high HbBC fraction of FIV, indicating that Hp was present in this >100 kDa MW bracket. Yet, given that only a small fraction of the total protein of FIV was retained in Stage 3, this stage had the highest HbBC per milligram of total protein. This high HbBC to total protein ratio indicated a high Hp purity in Stage 3. Although at similar HbBC to total protein ratio, only a small quantity (˜4 mL at ˜50 mg/mL) of Stage 2 was purified, with most of the product begin retained in Stage 3 (˜38 mL at 100 mg/mL). Low retention in Stage 2 suggested that the Hp polymers primarily permeated through the 500 kDa filter. Yet, due to the separation on a 500 kDa filter, Stage 2 contained mostly high MW (HMW) Hp polymers, while Stage 3 contained mostly lower MW (LMW) Hp which may allow for testing of the effect of Hp polymer size on the therapeutic index in various disease states. Based on these MW ranges, Hp polymers in Stages 2 and 3 mainly consisted of a mixture of Hp2-2 and Hp2-1, since any Hp1-1 present in FIV (expected to be primarily present in Cohn Fraction V) was too small to be retained in Stage 3.

Unfortunately for the purification of Hp, not only did most proteins permeate through the 100 kDa system, but almost 50% of the initial HbBC also permeated through the system, indicating that these Hp species in FIV were also permeable through the 100 kDa filter (small Hp polymers). Therefore, decreasing the numbers of diafiltrations at the last stage or using a smaller final MW filter could improve retention of these species with a potential drawback of reducing product purity. Furthermore, a large fraction of the initial HbBC (28%) and total protein (29%) was retained on the 0.2 μm filter, indicating that this filter was likely fouled during processing. Thus, starting with a different form of processed human plasma or removing some of the initial fouling particulates may benefit the initial filtration step. Possible solutions could be to introduce a larger filter pore size prior to the 0.2 μm filter or precipitating the protein suspension using ammonium sulfate or other salting-out agents. One promising fraction to use as the starting material could be Cohn Fraction IV-4, which removes many of the lipoproteins from the starting material. Furthermore, as shown later in this study, addition of fumed silica greatly enhanced filtration through the 0.2 μm stage. Stage 1 (0.2 μm-750 kDa) also showed potential as a product, but the sample may have to undergo further PBS diafiltration. Furthermore, via this method, Stage 1 contained large molecular weight proteins that did not seem to bind any Hb (see HPLC-SEC).

HPLC-SEC was performed on each of the processing stages and the results are shown in FIG. 16B (the dotted curve indicates the hemoglobin (Hb) binding capacity at each stage).

In FIG. 16B, it was evident that the LMW species were removed at Stages 2 (750-500 kDa) and 3 (500-100 kDa), but present in the 100 kDa permeate. Furthermore, both the 750-500 and 500-100 kDa brackets showed an almost uniform peak with Stage 2 at ˜8.5 min and Stage 3 at ˜8.6 min. These elution times yielded an estimated MW of 340±20 kDa for Stage 3 and 430±40 kDa for Stage 2. Yet, the small tail-end on the chromatograms of Stage 2 and Stage 3 indicated that the actual average MW was higher than the values obtained from the peaks. Moreover, the HPLC-SEC chromatogram for the raw FIV suspension showed how most of the proteins were smaller than 100 kDa (eluting at times larger than ˜9.3 min). Furthermore, based on the permeate of the 100 kDa, it was noticeable that the filter cut-off allowed passage of some >100 kDa species. Thus, these observations explain the 65% total protein recovery obtained in Stage 4.

From these curves, it was also possible to note that some of the high-MW protein species (elution times of ˜7.5 min or earlier) in Stages 0, 1, 2 and 3 did not show Hb-binding properties. This was noted by the lack of increase in absorbance at 280 nm of these high-MW species when excess Hb was added to the sample. When Hb binds to Hp forming Hb-Hp complexes, the absorbance at the Hp/Hp-Hb elution time increases due to the absorbance of Hb at 280 nm. Thus, if no increase was observed, it indicated that most of these high-MW species did not bind to Hb and were likely impurities that were not removed during the TFF processing. Interestingly, Stage 1 (0.2 μm-750 kDa) may also have therapeutic potential, but the sample had large quantities of large non-Hb binding proteins and may have to undergo further PBS diafiltrations, increasing overall processing time.

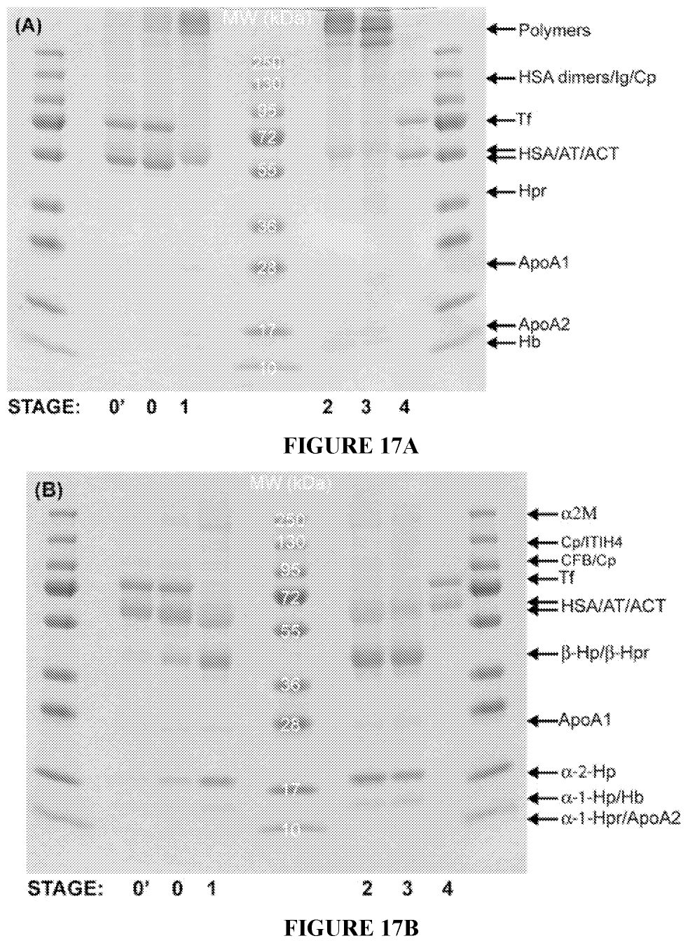

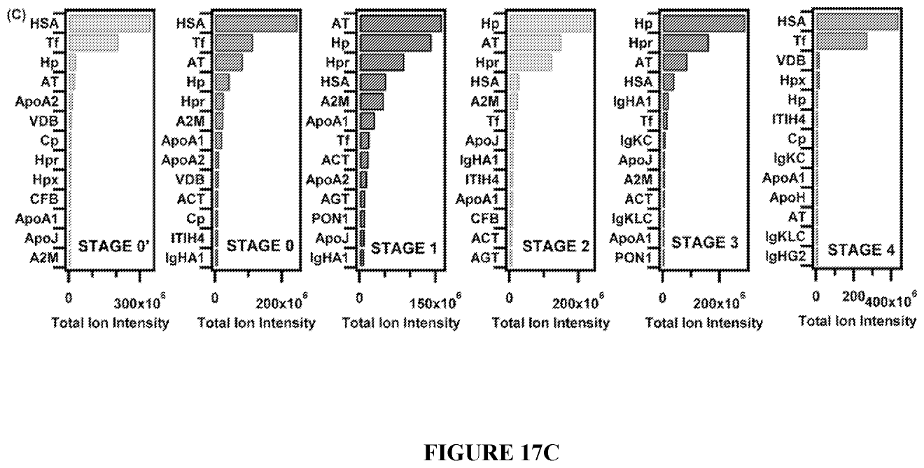

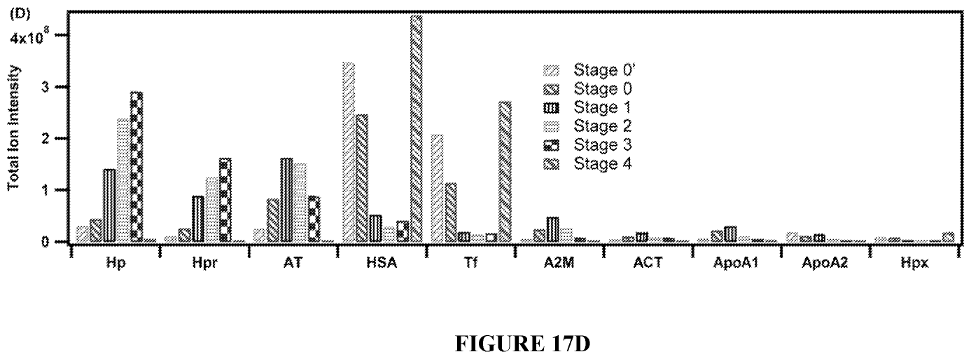

To analyze the protein species in each of the processing stages, SDS-PAGE was performed under both reducing and non-reducing conditions. Furthermore, protein identity was confirmed via trypsin digestion and mass spectroscopy (MS) of the samples. The gels from one representative batch and the percent composition based on the label-free quantitative MS analysis is shown in FIG. 17.

From the results of FIG. 17, it was noted that the most prominent protein bands of the SDS-PAGE could be attributed to the high-abundance proteins identified via MS. Furthermore, the proteins identified via MS were similar to previous reports of FIV-4 MS and the expected components of FIV. Compared to FIV-4, FIV contains proteins of FIV-1, which have large amounts of lipids, explaining the detection of the main components of HDL (apoA1 and apoA2), explaining their detection in MS and SDS-PAGE. In each sample, MS detected over 100 proteins. Yet, most of these impurities were at very low levels and were grouped into a single “Other” category shown in FIG. 17C. To ease analysis of how the proteins were distributed in each stage, the proteins which had more than >1% composition for at least three stages were shown in FIG. 17D.

Based on the increase in relative intensity of the polymeric species in Stages 1-3 of the non-reduced SDS-PAGEs, it was noted that the TFF process was capable of purifying high-MW species present in the FIV suspension (Stage 0). Although these species likely consisted of mostly Hp polymers, other large MW protein species and/or polymerized proteins may also have contributed to the “Polymers” band of FIG. 17A. Furthermore, MS analysis also showed an increase in composition of Hp and Hpr in Stages 1-3. These increases were consistent with the increase in relative HbBC shown in Table 4. Moreover, under non-reducing conditions, a band at ˜60 kDa was present at all stages which was attributed to mainly AT and α-1 antichymotrypsin (ACT) (determined via highest abundance of ˜60 kDa proteins). Transferrin (Tf) was also detected on the SDS-PAGE and in MS of Stages 0-3. Although AT, ACT and Tf are less than 100 kDa and, therefore, would be expected to have permeated through the TFF system; AT and ACT can undergo polymerization when partially denatured and various proteins including AT and Tf can associate with lipoproteins. Given that the fractionation process for production of FIV uses ethanol and acid, this process could also have contributed to partially unfolding and/or oxidizing proteins, leading to their polymerization. Thus, based on size-exclusion purification alone, it would not possible to remove these impurities from the samples. These species were then detected on the non-reducing SDS-PAGE due to dissociation of polymers and/or lipids in the presence of SDS.

Hemoglobin (Hb) and apolipoproteins were also present under the non-reducing SDS-PAGE shown in FIG. 17A. The unfolding of Hp due to the SDS potentially lead to unbinding of Hb to Hp, causing free Hb to appear on the gel. Furthermore, apolipoprotein A1 is a common contaminant in Hp preparations due to Hp binding and could also have been purified with lipoproteins. Apolipoprotein A1 is the major component (70%) of high-density lipoproteins (HDL), which, due to their size, were likely the lipoproteins purified with the high HbBC stages (3 and 4). In addition to apoA1, HDL is composed of ˜15-20% apolipoprotein A2 (apoA2). The presence of HDL corroborated the retention of haptoglobin-related protein (Hpr) which associates with HDL.

On the reduced SDS-PAGE, Hp dissociated into its α and β components. As expected, the majority of the α chains were α-2 which allow for the polymerization of Hp. Furthermore, a higher intensity of the band at ˜60 kDa was detected, likely due to reduction of disulfide bonded albumin polymers and full dissociation of proteins associated with lipoproteins. The presence of these polymeric albumin species would explain the detection of 4-5% HSA via MS on Stages 2 and 3 (FIG. 17C). A high-MW band on the non-reduced SDS-PAGE (˜200 kDa) was found which was attributed to the monomers of α-2 macroglobulin. Other small bands were also detected that were identified via proteins with similar MW of the MS data. Furthermore, various faint bands were found in the 90-130 kDa range, which were attributed to (in order of decreasing MW) inter-alpha-trypsin inhibitor H4 (ITH4), ceruloplasmin (CP), complement factor B (CPB), and plasma protease C1 inhibitor (PC1I). Finally, the other proteins shown under reducing conditions consisted of the same proteins identified on the non-reducing conditions (apoA2 was reduced into its monomeric chains). Interestingly, only a low intensity α-1 Hpr band was seen in Stage 3 which indicated that the MS ion intensity for Hpr may have been incorrectly assigned due to the high (>90%) sequence identity of Hpr to Hpl-1.

Protein purity was also estimated via densitometric analysis of the bands of Stage 2 (HMW) and 3 (LMW). The results are shown in Table 5.

TABLE 5
Purity of Hp products as determined by SDS-PAGE densitometry analysis of
samples purified via TFF without the use of fumed silica.

| | | HMW | | LMW | |
|---|---|---|---|---|---|
| | Species | Stage 2 | +/− | Stage 3 | +/− |
| NON-REDUCED | Polymers | >74.3% | 3.8% | >84.7% | 3.4% |
| | HSA/AT/ACT | <18.3% | 3.2% | <8.4% | 3.3% |
| | Hb/apoHb | <2.9% | 0.3% | <2.0% | 0.8% |
| | Tf | <1.2% | 0.1% | <1.8% | 0.5% |
| | ApoA1 | <1.3% | 0.6% | <1.2% | 0.2% |
| | Hpr | <0.7% | 0.8% | <1.0% | 0.5% |
| | ApoA2 | <1.2% | 0.1% | <0.5% | 0.1% |
| | ApoJ | <0.0% | 0.0% | <0.3% | 0.5% |
| | Other | <0.0% | 0.0% | <0.2% | 0.3% |
| | | HMW | | LMW | |
| | Species | Stage 2 | +/− | Stage 3 | +/− |
| REDUCED | β-Hp/β-Hpr/IgHA1 | >36.8% | 4.7% | >44.9% | 4.4% |
| | α-2Hp/IgKC | >23.9% | 0.2% | >20.7% | 0.5% |
| | AT/HSA/ACT | <19.6% | 5.1% | >11.1% | 5.2% |
| | α-1Hp/Hb/IgKLC/apoHb | <7.1% | 1.4% | <9.9% | 1.6% |
| | ApoA1 | <3.8% | 1.1% | <5.0% | 0.9% |
| | α-2M | <3.3% | 1.4% | <1.5% | 0.6% |
| | α-1Hpr/ApoA2 | <1.0% | 0.2% | <1.2% | 0.1% |
| | ITIH4/Cp | <0.7% | 0.1% | <2.0% | 1.0% |
| | Cp/CFB | <2.5% | 0.5% | <1.6% | 0.9% |
| | Tf | <1.0% | 0.5% | <0.9% | 0.1% |
| | Other (~65-70 kDa) | <0.4% | 0.2% | <1.3% | 0.4% |
| | Other (~15 kDa) | — | — | <0.5% | 0.8% |
| | Hb* | 1.0% | 0.1% | 1.0% | 0.1% |
| | ApoHb** | 2.9% | 1.1% | 3.1% | 0.3% |
| | Total Hp** | 68.0% | 6.6% | 75.3% | 5.0% |

*Amount of Hb was determined via the Soret peak of the sample assuming the presence of methemoglobin (ε_{405nM} = 167 mM⁻¹cm⁻¹)⁷⁸
**ApoHb content estimated via the exchange of heme from heme-albumin to sample (Δε₄₁₂ₙₘ≈55 mM⁻¹cm⁻¹ for generated heme-species).
***Total Hp from the sum of Hp containing bands of the reduced SDS-PAGE subtracted from Hb of the non-reduced SDS-PAGE
Abbreviations: AT: α-1 antitrypsin, ACT: α-1 antichymotrypsin, ATIII: Antithrombin III, Hb: hemoglobin, Tf: transferrin, ApoA1: apolipoprotein A1, Hpr: haptoglobin-related protein, ApoA2: apolipoprotein A2, CLU: clusterin; HSA: human serum albumin, Hp: haptoglobin, α-2M: α-2 macroglobulin, CP: ceruloplasmin, ITH4: Inter-alpha-trypsin inhibitor H4, IgHA1: immunoglobulin heavy constant alpha 1, IgKC: immunoglobulin kappa constant, IgKLC: immunoglobulin kappa light chain, CFB: complement factor B, apoHb: apohemoglobin.

From the densitometry assessment, Stage 2 and 3 were composed of ˜70 and ˜75% Hp, respectively. The major impurity were proteins in the 55-65 kDa range with ˜20 and ˜10% in Stages 2 and 3, respectively. Based on MS, these impurities were primarily composed of human serum albumin (HSA) and α-1 antitrypsin (AT). Due to their similar molecular weights, the proteins were not separately quantified via densitometry. But MS indicated that Stage 2 and 3 had 19% and 11% of AT with 4% and 5% of HSA, respectively. Unfortunately, many impurities were present in the Hp samples, which may have contributed to an inaccurate estimate of Hp purity. Most of these impurities likely originated from proteins associated with lipoproteins and/or proteins that can undergo polymerization. Interestingly, these impurities showed little deviations from batch-to-batch, demonstrating that the purification process was reproducible.

Although convoluted from contaminant proteins, the Hp purity from SDS-PAGE was similar to the expected Hp content based on the average HbBC per mg of total protein of Stage 2 and 3. Using the theoretical the mass ratio of Hp2-2 to Hb, the expected purity of the samples would be approximately 50% and 60% for Stages 2 and 3, respectively. Yet, the Bradford assay may have led to overestimation of total protein due to high concentration of glycoproteins which can also react with the dye used in the assay (Hp can have ˜20% of its total mass attributed to conjugated carbohydrates). Moreover, high MW Hp polymers isolated from serum have been shown to have even higher mass binding ratios than theoretical (>2:1 Hb:Hp), potentially due to tertiary structure steric hindrance. At a mass binding ratio of 2, Stage 3 would have ˜70% Hp, similar to what was obtained from densitometric analysis.

From UV-visible spectrometry, residual Hb contributed ˜1% of the protein mass for the LMW and HMW stages. Yet, based on the SDS-PAGE densitometry, Hb chains consisted of ˜3% of the total mass, indicating that some of the Hp may be bound to apohemoglobin (apoHb). Thus, the residual apoHb content was assessed by adding excess heme-albumin and monitoring the increase in absorbance at the Soret maxima as described in the Methods section. Given that Hp has been shown to not bind appreciably to heme, the change in absorbance was due to heme exchange from HSA to apoHb. Heme binding indicated that some of the residual Hb may have had its heme extracted during the acidic-ethanol fractioning of plasma to obtain Fraction IV. This heme-binding property of the purified Hp sample could be beneficial during hemolytic states in which free heme is also present.

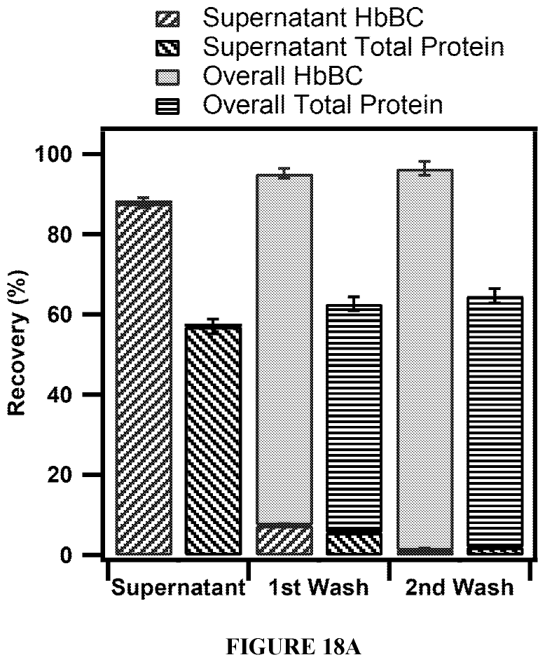

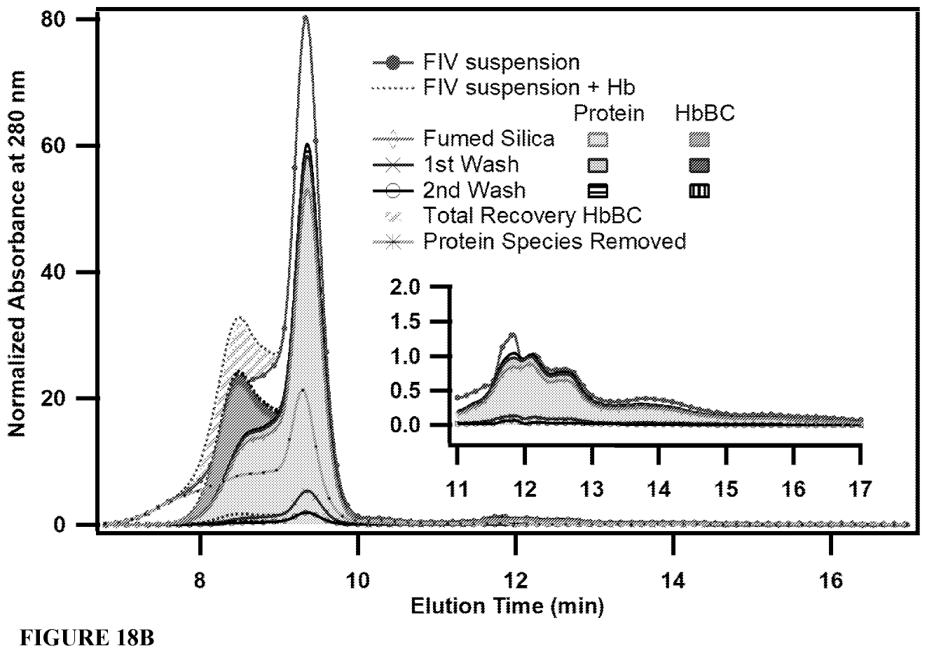

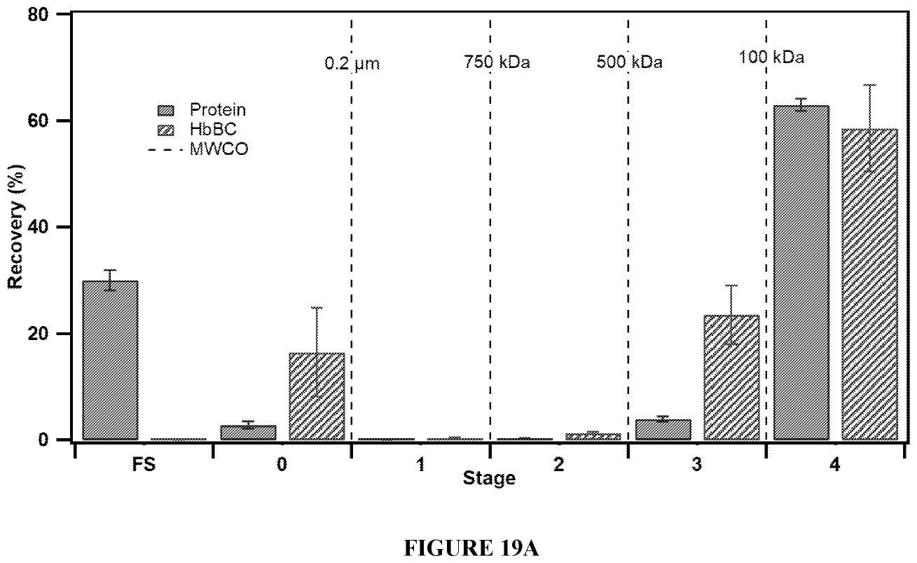

TFF Purification of Hp with Fumed Silica. From the results already shown, it was apparent that lipoproteins were likely the carriers for a large proportion of the impurities in the sample. Fumed silica is commonly used for de-lipidation of serum samples as it is capable of adsorbing lipids. Thus, the effect of using fumed silica on FIV was assessed, and the results are shown in FIG. 18.

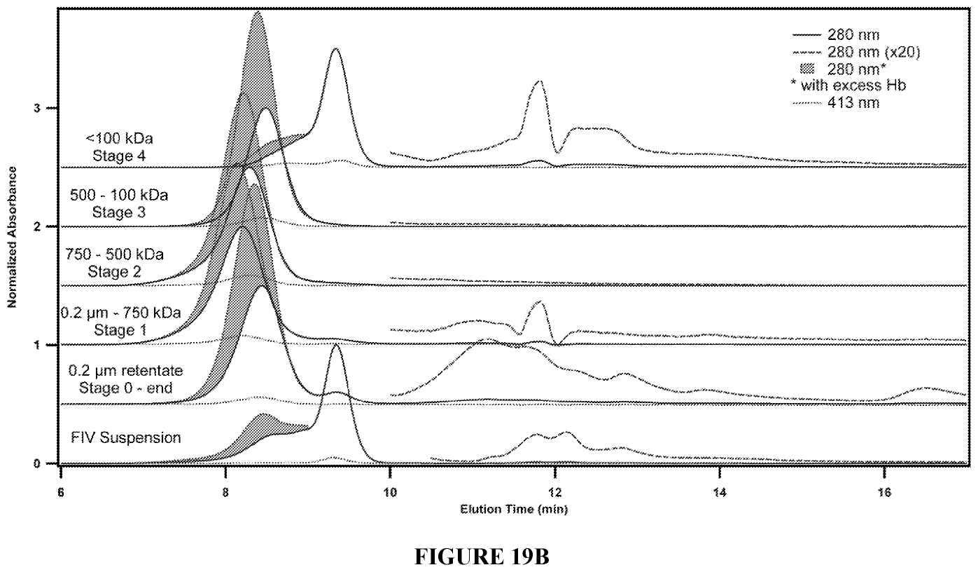

The analysis of percent recovery (FIG. 18A) showed that almost all the HbBC was recovered, but about 30% of the total protein was removed after fumed silica treatment. Thus, prior to TFF purification, the sample was already deprived of impurities. The small loss in HbBC also agreed with the low Hpr content seen on the SDS-PAGE of FIG. 17B given that these Hp species would likely be associated with HDL. The HPLC-SEC chromatogram of these impurities was estimated based on the difference of the FIV suspension and the total sum of chromatograms of the recovered supernatants (FIG. 18B). From the HPLC-SEC chromatograms, it was observed that various MW protein species were removed including the ones eluting at −7.5 min. The species eluting at 7.5 min species were running outside of the exclusion limit of the SEC column and, therefore, were likely >1 MDa species, indicating LDL particles were present in the FIV suspension. Furthermore, as shown in FIG. 16B, and confirmed by the high recovery in FIG. 18, these species were mainly impurities that did not bind to Hp and contributed to the left tail-end of the HPLC-SEC chromatograms of the purified Hp products (Stages 2 and 3). Furthermore, protein was also removed in the MW region of Stages 2 and 3 (˜8.5 min), indicating that these fractions could be isolated with higher Hp purity since no appreciable loss of Hp occurred. Thus, to assess the performance of the TFF system using fumed silica prior to filtration, the process described in the Methods section was tested on three batches. The results of these batches are summarized in Table 6. Total protein and HbBC recovery at each stage are also shown in FIG. 19A.

TABLE 6
Summary of products from each stage of the purification of Hp via TFF using fumed
silica.

| | Total | | | | | |
|---|---|---|---|---|---|---|
| | Protein | HbBC | Total | HbBC per | Protein | HbBC |
| STAGE | (mg/mL) | (mg/mL) | Protein (g) | mg (%) | Yield (%) | Yield (%) |
| 0' | 24.1 ± 3.0 | 1.4 ± 0.2 | 123 ± 12.6 | 5.89 ± 0.84 | 100 | 100 |
| FIV suspension | | | | | | |
| 0 | 5.2 ± 2.8 | 3.0 ± 2.7 | 2.48 ± 0.87 | 51.6 ± 32.6 | 1.96 ± 0.51 | 16.66 ± 8.39 |
| 0.2 μm retentate | | | | | | |
| 1 | 9.3 ± 13.9 | 3.2 ± 4.4 | 0.0701 ± 0.055 | 39.0 ± 22.8 | 0.06 ± 0.05 | 0.27 ± 0.20 |
| 0.2 μm-750 kDa | | | | | | |
| 2 | 53.2 ± 15.1 | 18.6 ± 3.6 | 0.259 ± 0.083 | 35.7 ± 4.5 | 0.2 ± 0.05 | 1.25 ± 0.33 |
| 750-500 kDa | | | | | | |
| 3 | 103.7 ± 6.2 | 51.7 ± 0.6 | 3.48 ± 0.88 | 49.9 ± 2.5 | 2.77 ± 0.44 | 23.87 ± 6.02 |
| 500-100 kDa | | | | | | |
| 4 | — | — | — | 9.65 ± 3.27 | 65.0 ± 0.93 | 57.94 ± 8.97 |
| 100 kDa permeate* | | | | | | |
| Fumed Silica** | — | — | — | — | 30 | 0 |

* *values were not measured and include all losses during processing; estimates were based on the initial total protein and HbBC compared to what was recovered in the other stages.
**loss based on data shown in FIG. 18.

From the results of Table 6 and FIG. 19A, it was evident that fumed silica greatly improved the filtration of proteins through the 0.2 um HF filter, since practically none of the protein was retained at the end of Stage 0 or Stage 1. Moreover, use of fumed silica before TFF lead to at least a one-fold increase in the permeate flow rate of all stages during processing. In doing so, even though more centrifugation was required prior to processing, fumed silica greatly decreased the overall processing time. Finally, not only did fumed silica treatment improve processing, but the two main Hp products (Stage 2 and Stage 3) also had significant improvements. Both Stage 2 and Stage 3 showed significantly higher HbBC per total protein (p<0.05) compared to samples processed without fumed silica indicating that the fumed silica treated samples had a higher proportion of Hp. With fumed silica, although Stage 0 showed a similar Hp composition to that of Stage 3, Stage 0 contained suspended insoluble species that were not easily removable via centrifugation. Thus, this sample was not considered as a main Hp product in this study. Yet, new purification techniques may be developed so that this insoluble matter is removed from the solution. To further characterize each of the retained stages, HPLC-SEC was performed, and the chromatograms are shown in FIG. 19B.

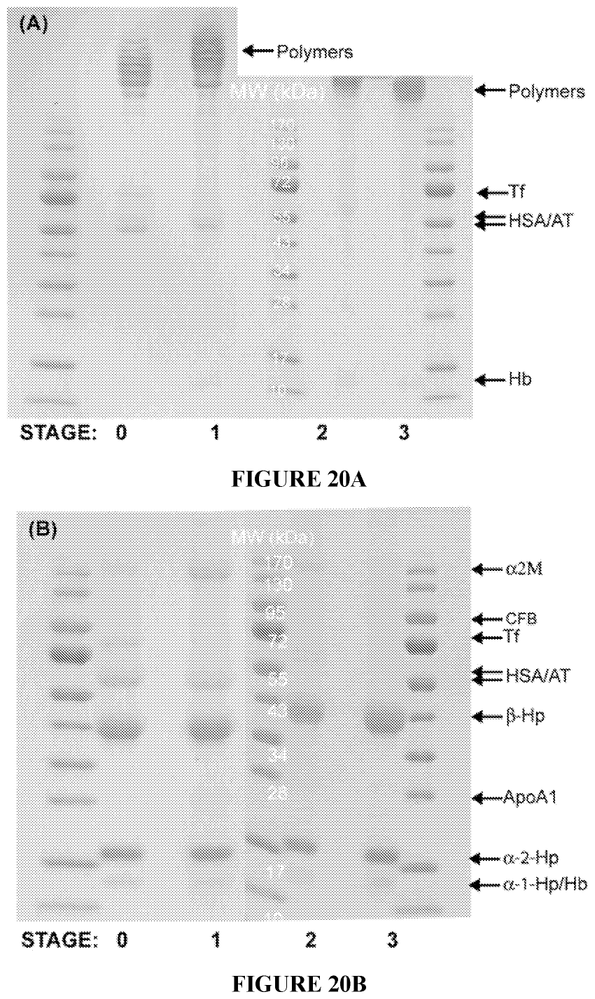

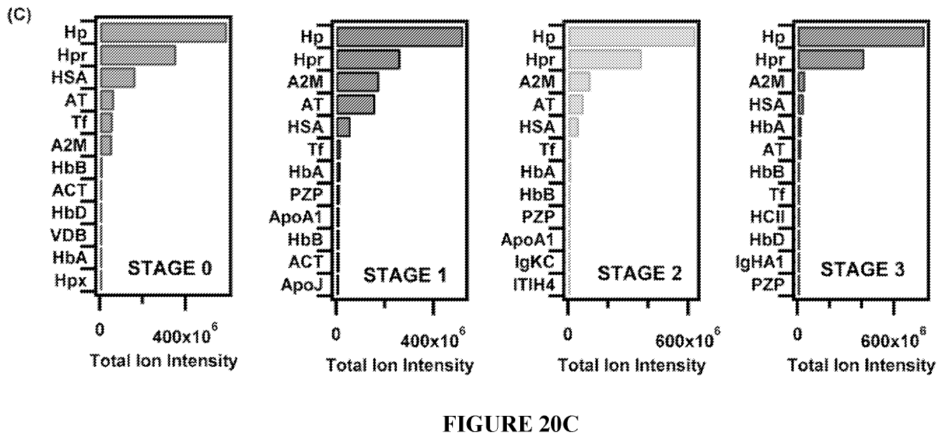

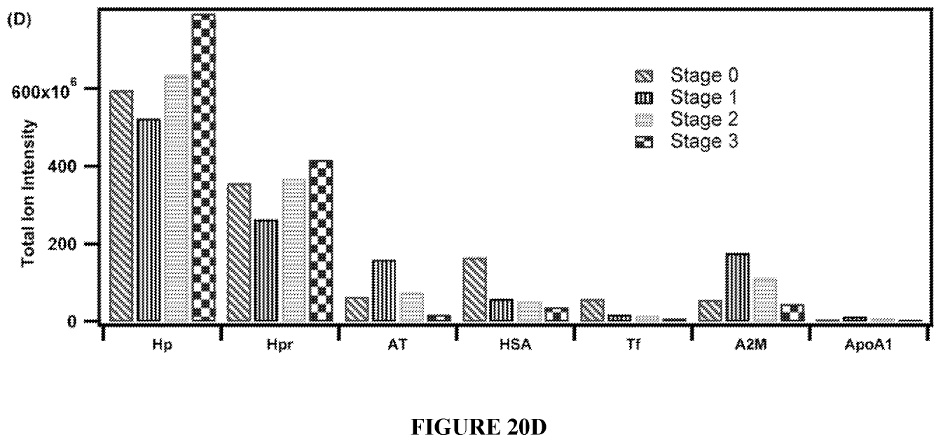

The HPLC-SEC chromatograms followed the same trends for Stages 2 to 4 as the batches that did not use fumed silica. More importantly, Stage 2 and Stage 3 had a reduced left tail-end of the non-Hb-binding proteins but the estimated molecular weight of Stages 2 and 3 increased compared to without the use of fumed silica. Stage 2 and Stage 3 had an average MW of 520±30 kDa and 390±20 kDa, respectively. Thus, even though there was removal of the large ˜7.5 min eluting species, the flux of large MW proteins through the filters improved, yielding larger average sized proteins in each stage. The increase in protein permeation through the filters is also shown by the lack of a peak at ˜9.2 min on Stage 0, indicating high passage of these ˜100 kDa species. Yet, the presence of low MW protein species in Stage 0 indicated that the filter was still fouling. The samples of the main Hp stages (Stage 2 and 3) were also analyzed via SDS-PAGE and trypsin digest MS, the result is shown in FIG. 20.

From the SDS-PAGE, it was apparent that these fractions were primarily composed of Hp. Little of the impurities from the purification method without fumed silica were detected. From a low protein loaded gel (shown in FIG. 20), the purity based on densitometry was >95% for Stage 3. This level of purity is similar or higher than of that previously reported for the commercialized polymeric Hp product produced in Japan. To better identify and estimate protein purity, densitometry analysis of slightly overloaded SDS-PAGE gels was performed and the results are shown in Table 7.

TABLE 7
Purity of Hp products as determined by SDS-PAGE densitometry
analysis of samples purified via TFF with the use of fumed silica.

| | | HMW | | LMW | |
|---|---|---|---|---|---|
| | Species | Stage 2 | +/− | Stage 3 | +/− |
| NON-REDUCED | Polymers | >88.0% | 3.6% | >94.3% | 1.7% |
| | Hb | <3.2% | 1.3% | <3.3% | 1.2% |
| | AT/DBP/ACT | <5.7% | 1.2% | <1.0% | 0.6% |
| | Tf | <0.2% | 0.2% | <0.8% | 0.2% |
| | ApoA1 | <1.5% | 0.5% | <0.2% | 0.2% |
| | ApoA2 | <0.7% | 0.3% | <0.2% | 0.2% |
| | Hpr | <0.3% | 0.3% | <0.1% | 0.1% |
| | apoL1 | <0.2% | 0.2% | <0.0% | 0.0% |
| | Other | <0.1% | 0.2% | <0.0% | 0.0% |
| | | HMW | | LMW | |
| | Species | Stage 2 | +/− | Stage 3 | +/− |
| \REDUCED | β-Hp/β-Hpr | >47.4% | 2.7% | >54.2% | 3.4% |
| | α-2Hp | >30.6% | 1.2% | >31.7% | 1.4% |
| | α-1Hp/Hb | <4.0% | 1.5% | <7.2% | 0.9% |
| | HSA/AT | <7.7% | 2.7% | <3.4% | 1.4% |
| | a2M | <6.5% | 0.3% | <2.0% | 1.1% |
| | ApoA1 | <1.9% | 1.2% | <1.5% | 1.3% |
| | CFB | <1.1% | 0.5% | — | — |
| | Tf | <0.9% | 0.1% | — | — |
| | Hb* | 2.0% | 0.4% | 1.0% | 0.1% |
| | ApoHb** | 2.0% | 1.0% | 3.0% | 0.3% |
| | Total Hp*** | 78.6% | 2.9% | 89.9% | 2% |

*Amount of Hb was determined via the Soret peak of the sample assuming the presence of methemoglobin (ε₄₀₅ₙₘ = 167 mM⁻¹cm⁻¹)⁷⁸
**ApoHb content estimated via the exchange of heme from heme-albumin to sample (ε₄₁₂ₙₘ≈120 mM⁻¹cm⁻¹ for generated heme-species).
***Total Hp from the sum of Hp containing bands of the reduced SDS-PAGE subtracted from Hb of the non-reduced SDS-PAGE
Abbreviations: AT: α-1 antitrypsin, ACT: α-1 antichymotrypsin, Hb: hemoglobin, Tf: transferrin, ApoA1: apolipoprotein A1, Hpr: haptoglobin-related protein, ApoA2: apolipoprotein A2, ApoL1: apolipoprotein L1, HSA: human serum albumin, Hp: haptoglobin, α-2M: α-2 macroglobulin, CP: ceruloplasmin CFB: complement factor B, apoHb: apohemoglobin..

From the purity assessment in Table 7, the BMW fraction was composed of ˜80% pure Hp and the LMW fraction was composed of ˜90% pure Hp. Thus, in agreement with the higher HbBC per mg, it was confirmed that the purity of the two main Hp products had greatly improved with the use of fumed silica. Yet, the purity of Hp is likely higher due to the slight overloading required to detect these impurities on the SDS-PAGE. Furthermore, similar to the process without the use of fumed silica, the method yielded consistent product compositions as demonstrated by the small deviations from densitometric analysis.

Interestingly, based on the average HbBC to total protein ratio and the Hb mass binding ratio of Hp2-2, the expected purity of the LMW Hp fraction (Stage 2) would be ˜85%. Unlike the process without the use of fumed silica, this value was in close agreement with the SDS-PAGE densitometry, indicating that very few protein contaminants were present in the samples. Furthermore, as explained before, the mass binding ratio between Hp2-2 and Hb may be larger than the theoretical value, leading to a closer Hp purity of >95% in agreement with densitometric analysis of SDS-PAGE.

Moreover, the higher purity from the Hp product purified with the use of fumed silica indicated that either the polymerized forms of the non-Hp proteins have a high affinity for the silica (potentially due to higher hydrophobicity from partial protein unfolding) or that most of these proteins were associated with the lipoproteins. Practically, no change in the α-2 macroglobulin fraction was observed indicating that this multimeric (720 kDa) protein species did not have a high affinity for the silica particles.

ELISA was performed on Stages 2 and 3 of the fumed silica treated batches to determine the mass percentage composition of Hp, HSA, and Tf to compare to the values determined by densitometric analysis of the SDS-PAGE. Unfortunately, the Hp ELISA was unable to accurately quantify the large MW Hp polymers in Stages 2 and Stage 3. The ELISA kit resulted in a Hp mass composition of 21±0.2% and 34±0.2% for Stages 2 and 3, respectively. As discussed previously, Hp ELISAs may not provide proper quantification of large MW Hp species. These large MW species may have sterically inhibited binding of Hp to immobilized Hp antibodies. The HSA content was determined to be 0.8±0.01% and 0.8±0.2% for Stages 2 and 3, respectively. This result agreed with MS data that demonstrated relatively similar HSA content for Stages 2 and 3. Thus, the difference in composition for the HSA/AT band on the reduced SDS-PAGE was mainly attributed to the higher AT composition in Stage 2 as seen by the high AT total ion intensity in the MS. The Tf composition was determined to be 0.5±0.09% and 0.3±0.09% for Stages 2 and 3, respectively. These results were similar to the values determined by SDS-PAGE, which showed <1% of Tf for both Stage 2 and Stage 3. Finally, ELISA kits were used to confirm that the hemichrome species formed from heme exchange from heme-HSA to the Hp sample was due to residual apoHb and not residual Hpx. Although Hpx was not detected on MS or apparent on the SDS-PAGE, both denatured apoHb and Hpx form similar hemichrome spectra (bis-histidine bonded heme). Hence ELISA results showed that the Hpx content was 0.1±0.02% and 0.1±0.04% of the total protein for Stages 2 and 3, respectively. Given that the calculated apoHb mass content was ˜3%, practically all heme exchange was due to residual apoHb in Stages 2 and 3 with a minor contribution from Hpx. Furthermore, since Hpx has a larger MW (on a heme basis) than apoHb, if Hpx was the heme-binding species, a mass content of >10% Hpx would have been determined

Although the purified samples were not composed of only Hp, the extra proteins in the HMW and LMW fractions yield a protein cocktail potentially useful for treatments of hemolysis. For example, the α-2 macroglobulin (α2M) protein is a broad specificity protease inhibitor. Furthermore, α2M helps maintain a balanced clotting system by both inhibiting the coagulating protein thrombin and inhibiting the anti-coagulating Protein C system. These characteristics may improve the effectiveness of the Hp product for applications in which the patient has an abnormal balance of the clotting proteins. Furthermore, α2M can help maintain hemodynamic equilibrium after scalding/burning by inhibiting prostaglandin E2 (vasodilator) and restricting loss of plasma volume. Moreover, the anti-inflammatory, anti-fibrosis and anti-oxidant functions of α2M have been linked to its role as a radioprotective agent. These characteristics can improve treatment of hemolytic states due to burn injury or radiation injury. Finally, α2M along with AT have been shown to mediate the binding of Tf to its surface receptor. In doing so, α2M can help with the removal of excess iron potentially released during prolonged states of hemolysis.

For the Hp samples purified without fumed silica, the presence of HSA, Tf and AT may improve the therapeutic effect of the protein mixture compared to pure Hp. HSA is a multifunctional protein, with major roles in the regulation of acid-base balance, oncotic pressure, binding/transport of endogenous and exogenous molecules and drugs (binds to heme upon depletion of hemopexin, potentially improving hemolysis treatment with the Hp protein cocktail), protection against exogenous toxins, maintenance of microvascular integrity and capillary permeability, antioxidant and anticoagulant activity. AT and transferrin have also been shown to bind to heme, which is a highly oxidative by-product of Hb degradation during states of hemolysis. AT, named for its ability to inhibit trypsin, can also inhibit other proteases, which is also known as: α1-antiprotease inhibitor. Due to its anti-proteolytic function, AT has general anti-inflammatory properties. In addition, AT plays a large role in vivo by preventing lung damage via inhibition of neutrophil elastase. Co-treatment of Hp with AT may constitute an improved treatment of pulmonary hypertension as both Hb (and its by-products) and neutrophil elastase have been shown to have deleterious effects. Finally, Tf is an antioxidant protein responsible for iron binding and transport. Thus, iron build-up due to excessive hemolysis could be neutralized by Tf.

As stated previously, apolipoprotein A-1 is the main component of high-density lipoproteins (HDL) and is found in the TLF1 complex (contains Hpr that binds to Hb). HDL therapy has been shown to treat atherosclerosis, improving blood flow and the HDL (expected to be the form in the protein cocktail on the process without fumed silica) has had greater clinical efficacy than pure apolipoprotein A-1 (likely due to its short half-life). In addition to its well-known role in lipid transport, HDL/apolipoprotein A-1 has various pleiotropic effects such as antioxidant, anti-inflammatory, antithrombotic (anticoagulant and increased NO bioavailability) and vasoprotective activities. Furthermore, HDL has been shown to negate the effects of lipopolysaccharides, reducing its pro-inflammatory responses. Finally, apolipoprotein A1 has also been shown to have antimicrobial activity.

To reduce the costs associated with the use of buffers, future studies may aim to optimize the number of diafiltrations at each stage for effective protein transmission and protein purity. Furthermore, by retaining the proteins in Stage 4 using a low MW TFF filter (<50 kDa), the permeated buffer may be used for future processing as it would contain little to no proteins. Bacterial contamination of the buffer is minimized given the filtration through the 0.2 μM and 750 kDa HF filters at the start of the next batch. The fraction retained in Stage 4 may also have therapeutic uses as it provides a promising mixture or proteins. Moreover, the protein mixture in Stage 4 could be used as the starting material for conventional chromatography purification to yield low MW Hp. Finally, the initial protein loading of FIV can also be increased. Dissolving 1 kg of FIV into 5 L of PBS before processing can yield approximately double the Hp yield with no discernable difference in purity compared to that of the 500 g batches.

Although the starting material for FIV consisted of pooled human plasma, which may have safety risks associated with infectious agents, the combination of Cohn acid-ethanol fractionation and TFF processing inherently reduces these risks. TFF clarification with the 0.2 μm and 750 kDa HF filters remove most pathogenic bacteria. In the case of viruses, the Cohn acid-ethanol fractionation process provides an approximate 4 log₁₀ reduction value (LRV) for various viruses. Furthermore, nanofiltration using TFF can add an additional 5 (LRV). Finally, the Hp sample may undergo a final virus reduction step if desired such as solvent/detergent or pasteurization to reach the desired level of pathogen reduction.

One drawback for scalability of the current process is the use of centrifuges for removal of undissolved lipids and/or fumed silica. Cloth or depth filtration may substitute for the centrifugation step used to remove the unsuspended lipids. Furthermore, continuous centrifugation may be employed to separate the fumed silica, as it requires low relative centrifugal force to form a pellet. Finally, given that in both centrifugation steps, the goal was to remove lipids, these steps could be combined into a single centrifugation step to decrease processing time.

Conclusion

Overall, starting from 500 g of Cohn Fraction IV paste and without the use of fumed silica, 1.2 g of HbBC at ˜75% purity was obtained in solutions with ˜100 mg/mL total protein and 33 mg/mL HbBC. With the use of fumed silica, the yield increased to 1.7 g of HbBC at >95% purity in solutions with ˜100 mg/mL total protein and ˜52 mg/mL HbBC.

Future studies may aim at improving processing time by optimizing the number of diafiltrations and buffer usage. Taken together, this study presents a novel and improved method for producing large quantities of large MW Hp (mixture of Hp2-2 and Hp2-1) at >95% purity

### Example 3. Selective Protein Purification Via Tangential Flow Filtration—Exploiting Molecular Size Changes Induced by Protein Complex Formation to Facilitate Separation

This example describes a process to purify a target protein (TP) from a mixture of proteins by exploiting molecular size changes that arise from the formation of a protein complex consisting of the TP and the TP binding molecule (TPBM). Briefly, the method employs tangential flow filtration (TFF) with a defined molecular weight cut off (MWCO) membrane to first permeate the TP+other protein impurities (filtrate) that are below the MWCO of the membrane, as well as set the maximum size/molecular weight of the protein species in the filtrate. A TPBM (could be another protein, antibody, aptamer or some other molecule) is then added to the filtrate to selectively create a protein complex with the TP in the protein mixture that is above the MWCO of the original membrane. With only the complexed TP above the MWCO of the original membrane, it can be selectively separated from the other low MW protein components of the filtrate using the original MWCO membrane. TFF can then be applied to buffer exchange the complexed protein under dissociative conditions and separate the TP from the TPBM using a MWCO membrane that is between the MW of the TP and TPBM.

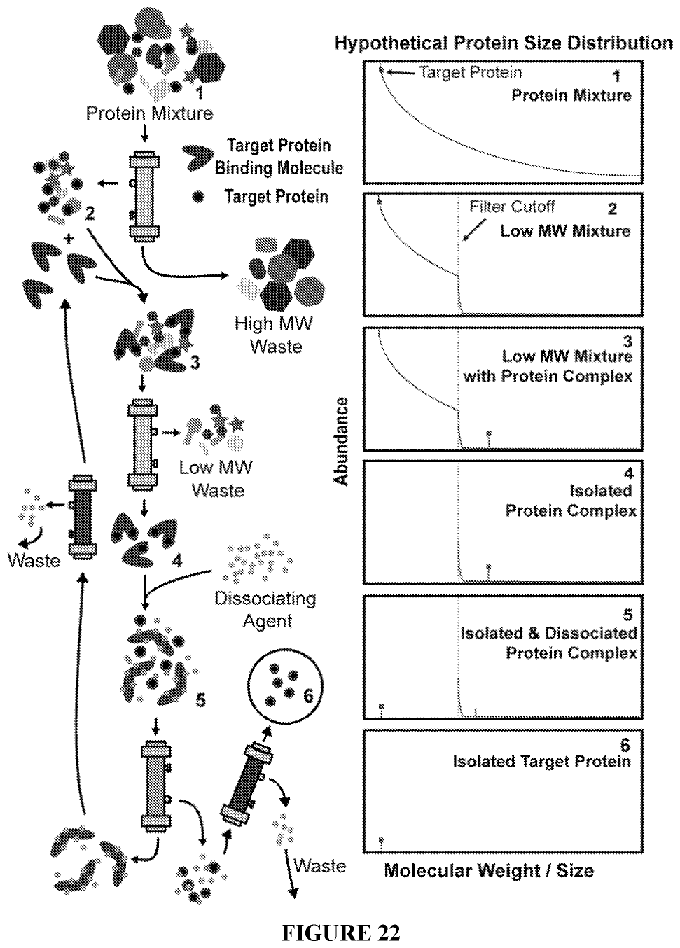

This theoretical strategy is schematically illustrated in FIG. 22. Referring to FIG. 22, starting with a mixture of proteins/particulates (1) (e.g., a cell lysate, human plasma, etc.), the mixture is filtered through a membrane with an appropriate MWCO that permeates the TP along with low MW impurities (2). Then a TPBM (e.g., an antibody or equivalent, etc.) specific to the TP is introduced into the filtrate, forming a TP-TPBM protein complex that is larger than the MWCO of the membrane (3). This solution with the newly formed TP-TPBM protein complex is then refiltered through the same MWCO membrane leading to the retention of the isolated TP-TPBM protein complex of interest (4). The isolated TP-TPBM protein complex can then be dissociated to yield free TP and TPBM via appropriate buffer exchange under conditions that would facilitate their separation (5). With the individual; species (TP and TPBM) dissociated in solution, the TP can be separated from the TP-TPBM protein complex using a MWCO membrane that is between the MW of the TP and TPBM (6). Note: both the TPBM in the retentate and TP in the filtrate can be buffer exchanged via TFF into appropriate buffers to remove the dissociating agent and concentrate the TP and TPBM.

General Strategies

Example Strategy 1—Purification of a 20 kDa TP using IgG antibody specific to TP. In a mixture of cell lysate (may need prior clarification through 0.2 micron filter and/or 50 nm filter), filter all material through a 70 kDa MWCO membrane. Add immunoglobulin G (IgG, commonly used antibody type) antibody specific to the TP into the filtrate. This will create an antibody-TP protein complex with MW >70 kDa (˜190 kDa, assuming two antigen binding-sites per antibody). The ˜190 kDa protein complex with the 20 kDa TG is now in a mixture with other proteins <70 kDa. Thus, this solution can be re-filtered through the 70 kDa MWCO membrane to retain the TP-antibody protein complex. The isolated TP-antibody complex can then be buffer exchanged into appropriate conditions to dissociate the TP-antibody complex (i.e. altered pH, salt concentration, or other appropriate denaturing condition). With the TP dissociated in solution from the antibody, refiltering the solution through the 70 kDa MWCO membrane will lead to the 20 kDa TP in the filtrate, and the 150 kDa antibody will be in the retentate. With the TP and antibody isolated, these species can be buffer exchanged into appropriate buffers on 10 kDa and 70 kDa membranes, respectively to yield purified TP and antibody. It is important to note that polyclonal antibodies may form aggregates at equimolar concentrations, thus excess target protein or excess antibody can be used for purification with these antibody species. On the other hand, monoclonal antibodies would not have the same issue as they only form specific complexes.

Example Strategy 2—Purification of a 200 kDa TP Using IgG (Non-Reduceable Protein, IgM or Equivalent Large TPBM could be Used for its Purification as in Example Strategy 1) Antibody Specific to TP. In a mixture of cell lysate (may need prior clarification through 0.2 micron filter and/or 50 nm filter), filter all material through a 300 kDa MWCO membrane. Add IgG antibody specific to TP of interest into the filtrate. This will create a TP-antibody protein complex with MW>300 kDa (˜350-670 kDa). The 200 kDa TP is now in an >300 kDa TP-antibody protein complex in a mixture with other proteins <300 kDa. Re-filter the protein solution through a 300 kDa MWCO membrane. The TP-antibody complex will be retained on the 300 kDa MWCO membrane. If the TP does not dissociate from the TP-antibody complex under reducing conditions, reduction of IgG will allow for its separation from the TP on a 100 kDa filter. The 200 kDa TP will be retained on the 100 kDa MWCO membrane, while the reduced components of IgG will enter the permeate. Both the TP and IgG components can be diafiltered into appropriate buffers. It is important to note that polyclonal antibodies may form aggregates at equimolar concentrations, thus excess target protein or excess antibody can be used for purification with these antibody species. On the other hand, monoclonal antibodies would not have the same issue as they only form specific complexes.

Example Strategy 3—Tagged recombinant proteins. Recombinant proteins can be synthesized with tags that facilitate their purification outlined in the Example Strategies above, removing the requirement for affinity columns. Instead of binding to affinity beads in columns, tags would bind to molecules that have larger MW than the MWCO of the employed membrane. For example, in a mixture of cell lysate with the strep-tagged 10 kDa recombinant protein (TrP), filter all material through a 30 kDa MWCO membrane. Add streptavidin to create a TrP-streptavidin complex with MW>60 kDa. The ˜60 kDa TrP-streptavidin complex is now in a mixture with other proteins <30 kDa. Thus, this solution can be re-filtered through the 30 kDa MWCO membrane to retain the TrP-streptavidin complex. The isolated protein complex can be buffer exchange into appropriate conditions to dissociate the protein complex. With the TrP dissociated in solution from the streptavidin, refiltering through the 30 kDa MWCO membrane will lead to the 10 kDa TrP in the filtrate, and the 50 kDa streptavidin will be in the retentate. With the TrP and streptavidin isolated, these species can be buffer exchanged into appropriate buffers on 1 kDa and 30 kDa membranes, respectively to yield purified TrP and streptavidin. This strategy employs currently known tagged recombinant protein technology, but improvement of the technologies for the proposed TFF purification could greatly ease its use. For example, polymerizing or developing large maltose/streptavidin/glutathione-bound molecules could improve applicability of this size exclusion method with currently used tagging systems (e.g., maltose-binding protein, strep-tag, glutathione-S-transferase, split-intein, etc.).

Purification of Haptoglobin (Hp) from Human Plasma or a Plasma-Derived Fractions Via Protein Complex Formation.

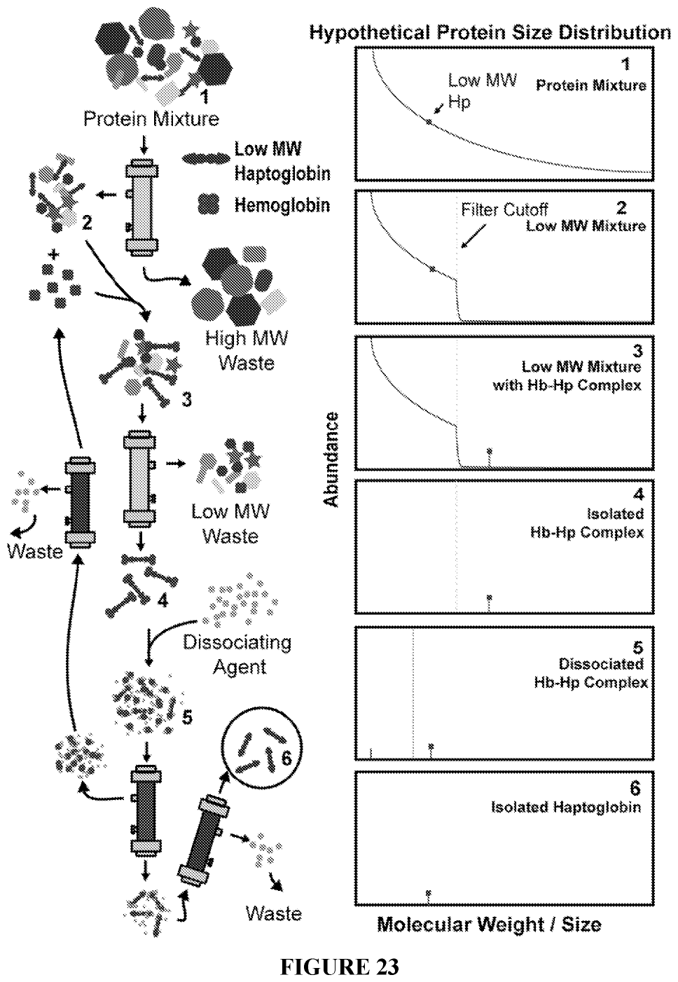

The theoretical manufacturing scheme for the presented technology is shown in FIG. 23.

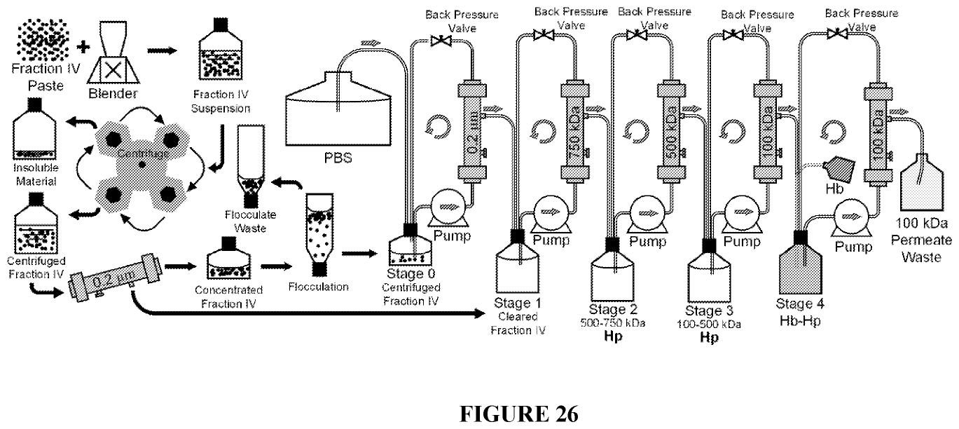

500 g of human Fraction IV (FIV) paste from the modified Cohn process of Kistler and Nitschmann was suspended in 5 L of PBS, and homogenized in a blender. The resulting mixture was stirred overnight at 4° C. The ˜5 L solution was centrifuged for 40 min at 3700 g to remove insoluble particulates (mostly lipoproteins). Then, the supernatant was concentrated using a 0.2 μm hollow fiber filter to 2 L The retentate was left to rest for 36 hrs to flocculate low density particles, while the filtrate was kept at 4° C. for further processing. After flocculation of the retentate, low density particles in solution were separated. The higher density fraction was then concentrated ˜1 L on a 0.2 μm hollow fiber filter and subjected to 10 diafiltrations with PBS. The 0.2 μm filtrate was concentrated to 150 mL and subjected to 100 diafiltrations on a 750 kDa hollow fiber filter using a mixture of the 0.2 μm permeate and PBS. The permeate was then subjected to 40 diafiltrations using PBS on a 500 kDa hollow fiber filter. Finally, the permeate of the 500 kDa hollow fiber filter was subjected to 100 diafiltrations using PBS on a 100 kDa hollow fiber filter (intermediate stages comprising of 750 and 500 kDa hollow fiber filtration were employed prior to the 100 kDa hollow fiber filter to avoid filter fouling). Hemoglobin (Hb) was then continuously added to the permeate from the 100 kDa hollow fiber filter to form the Hb-Hp complex, while maintaining the solution with excess Hb to bind all Hp in the permeate. The filtrate/Hb mixture was then subjected to diafiltration (100 or 200×) on a 100 kDa hollow fiber filter using fresh PBS to remove excess Hb and low molecular weight (MW) proteins. The resulting Hb-Hp complex was then centrifuged for 30 min at 3000 g to remove any insoluble particulates. The 100 diafiltration trial yielded 200 mL of 2 mg/mL Hb-Hp complex, while the 200 diafiltration trial yielded 200 mL of 0.8 mg/mL Hb-Hp complex. The diagram for the purification process is shown in FIG. 26. The solutions were then concentrated on a 100 kDa filter. The final products consisted of 5 mL solutions at 43.4 mg/mL and 17.2 mg/mL of Hb-Hp. The discrepancy in final product yields was attributed to differences in the number of diafiltrations performed with the Hb-Hp complex solution (100 or 200×).

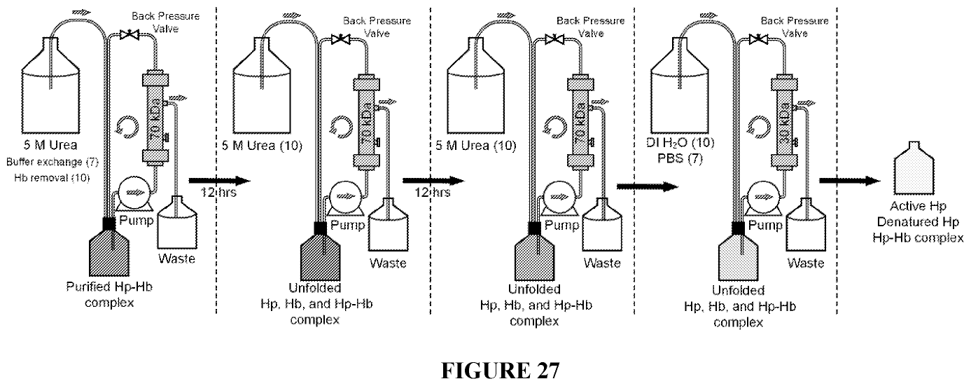

To facilitate dissociation of Hb from the purified Hb-Hp complex, 7 mL of Hb-Hp at 2 mg/mL was buffer exchanged (7 diacycles) into a 5 M Urea solution at a pH 10 using a 70 kDa hollow fiber filter. The resulting unfolded protein mixture was then subjected to 10×diafiltration using the urea solution with a rest period of 12 hr in between processing to yield a total of 30 diacycles. The solution was then diafiltered for 10 diacycles into DI water followed by 7 diacycles into PBS using a 30 kDa hollow fiber filter. The schematic of this process is shown in FIG. 27 and the characteristic of all the filters used is shown in Table 8. The final product was finally concentrated to 3.2 mL, yielding a solution with 9.2 mg/mL of protein (Bradford assay) and 1.8 mg/mL of Hb (spectrophotometric analysis).

TABLE 8
Hollow fiber filters (Spectrum Laboratories, Rancho Dominguez, CA)
used for the dissociation and purification of pure Hp from purified
Hb-Hp using tangential flow filtration.

| Type | Pore Size | | Membrane | Surface Area (cm²) | P/N |
|---|---|---|---|---|---|
| miniKros | 0.2 | μm | PES | 470 | S02-P20U-10-N |
| miniKros | 750 | kDa | mPES | 790 | S02-E750-05-N |
| miniKros | 500 | kDa | PS | 1000 | S02-S500-05-N |
| miniKros | 100 | kDa | mPES | 1000 | S02-E100-05-N |
| microKros | 70 | kDa | mPES | 20 | C02-E070-05-N |
| microKros | 30 | kDa | mPES | 20 | C02-E030-05-N |

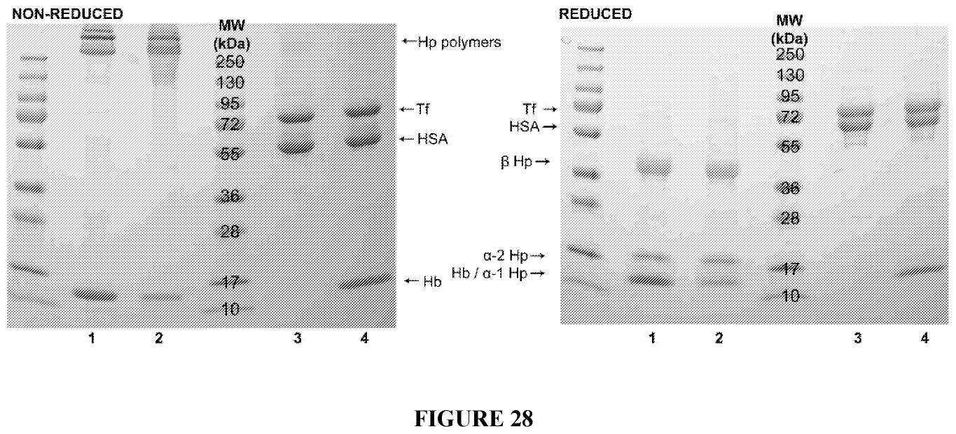

The SDS-PAGE of the purified Hb-Hp complex and recovered pure Hp from one batch is shown in FIG. 28.

From the SDS-PAGE, practically no impurities could be detected (>98% pure) for the purified Hp-Hb. Furthermore, from both SDS-PAGE band intensity analysis and the spectrophotometrically determined amount of Hb bound to the purified Hp species, the Hp to Hb mass binding ratio was calculated to be 1.6:1. This is the same mass binding ratio assuming one Hp2-2 dimer is bound to one Hb dimer. Urea treatment was not successful in removing all of the bound Hb. Using the 1.6:1 mass binding ratio, SDS-PAGE analysis indicated that about 20% of the Hp was still bound to Hb. In comparison, using total protein and spectrophotometric analysis, the product consisted of 25% active Hp, 29% Hb-Hp complex and 52% inactive Hp (denatured). Furthermore, compared to the starting Hb-Hp complex, 52% of Hp was lost during diafiltration, 12% was active, 13% remained bound to Hb and 23% was denatured. These results could be improved through optimization of the protein unfolding conditions in urea to avoid protein denaturing and via selection of a lower MWCO hollow fiber filter for the diafiltrations to avoid loss of protein.

From the Hb binding fluorescence assay, the final solution had a Hb binding capacity of 0.5 mg/mL, which indicated that only 10% of the initial Hb binding capacity was recovered. This large loss of Hp was attributed to loss of protein and protein denaturing during urea diafiltration as well as retained Hb in the product.

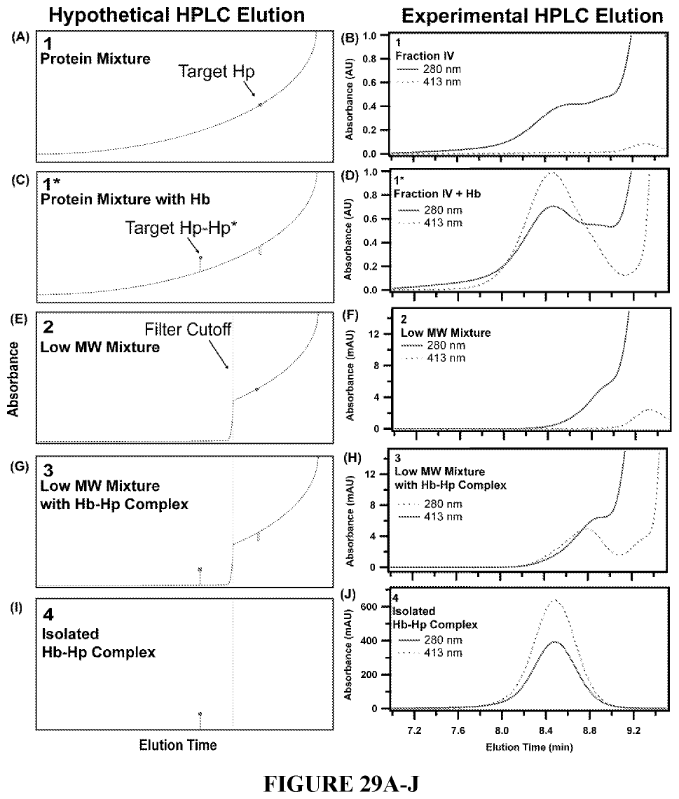

During the purification of the Hp-Hb complex, samples were taken at different stages of the process. These samples were analyzed on an HPLC-SEC column and the results were compared to the theoretically predicted separation based on the schematic in FIG. 22. The comparison of predicted versus experimental results is shown in FIG. 29.

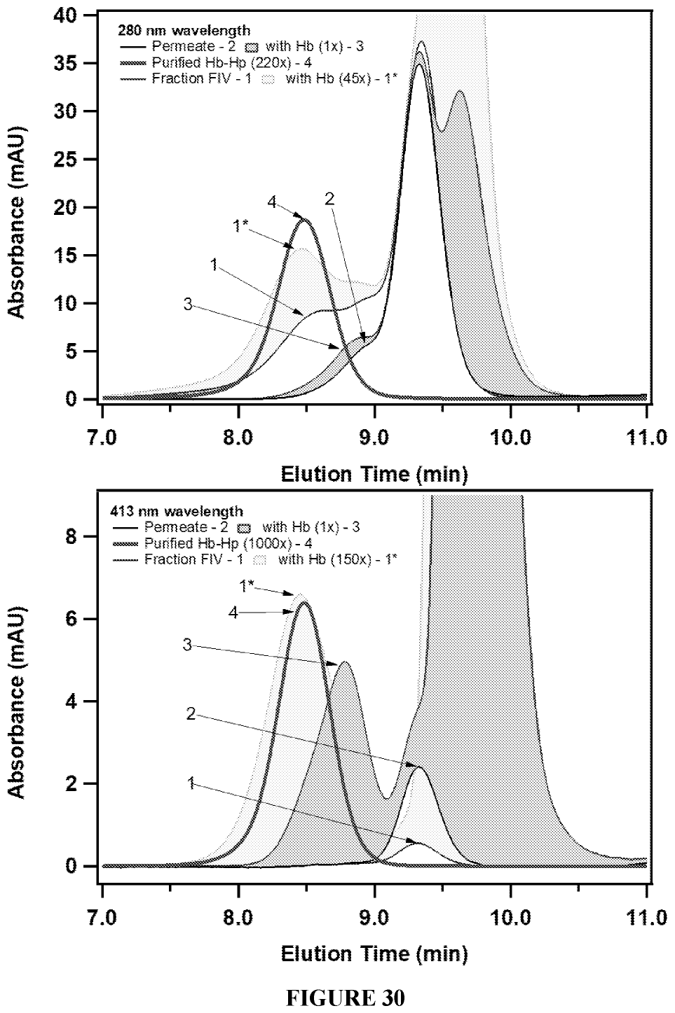

From these results, the addition of Hb to Fraction IV increased the amount of large molecular weight species (compare 1 to 1*). These species matched our purified product (4). Furthermore, the permeate analysis (2) showed that the unbound Hp did not easily permeate the 100 kDa hollow fiber filter. This can be seen due to a lower relative abundance of the Hb-Hp complex when Hb was added comparing Fraction IV to the permeate (1* to 3). Another observation was that, by comparing 2 to 4, it was noticeable that the Hb-Hp complex was capable of permeating through the 100 kDa hollow fiber filter. This observation agreed with the different Hb-Hp yields obtained via 100× or 200 diafiltrations. Combining the chromatograms into one figure better represented the statements regarding retention of unbound target Hp and permeation of the target Hb-Hp complex. The combined elution chromatograms at 280 and 413 nm wavelength detection is shown in FIG. 30.

### Example 4. Preparation of Apohemoglobin-Haptoglobin Complex

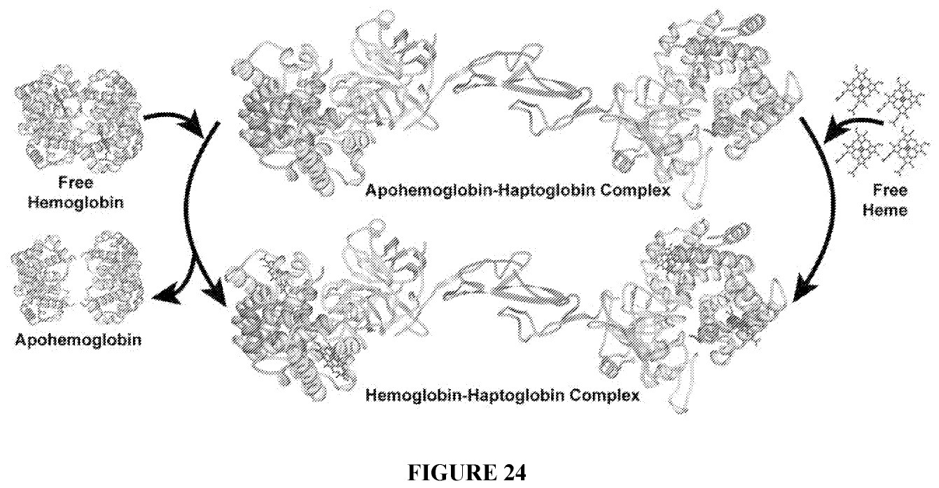

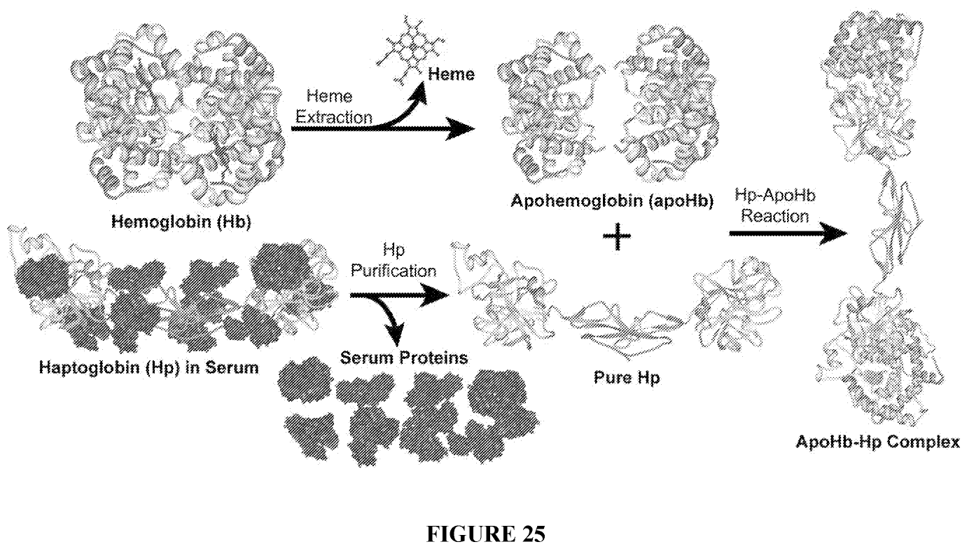

The general mechanism of action for the ability of the apoHb-Hp complex to scavenge cell-free Hb and heme is shown in FIG. 24. The general process for producing the apoHb-Hp complex is shown in FIG. 25.

Apohemoglobin-Haptoglobin Complex Preparation. The apoHb-Hp complex can be made by reacting apoHb with Hp. The high binding affinity drives the reaction for complex formation. A Hp solution with a Hb binding capacity (HbBC) of 24 mg/mL was mixed with an apoHb solution with 37 mg/mL of active apoHb at a 1:1 and 1:4 volume ratio. The resultant mixture was separated on a size exclusion chromatography (SEC) column for analysis. Large molecular weight Hp (Hp2-2 and Hp2-1) was mixed with apoHb with a molecular weight of 31 kDa (dimeric apoHb) and separated on an analytical Acclaim SEC-1000 (4.6×300 mm) column (Thermo Fisher Scientific, Waltham, Mass.) attached to a Dionex UltiMate 3000 system (Thermo Fisher Scientific, Waltham, Mass.). The mobile phase consisted of 50 mM potassium phosphate, pH 7.4. The flow rate and UV-visible spectral detection was controlled using Chromeleon 7 software with detection set to A=280 nm to detect protein elution at a flow rate 0.35 mL/min. The percent change of the area under the curve between a pure apoHb solution and a mixture of apoHb-Hp with excess apoHb was used to determine the percentage of apoHb that was bound to Hp. This percentage was compared to the mass of pure apoHb loaded to determine the Hp binding capacity of apoHb. This value was compared to the HbBC of the Hp sample.

Results

As seen in FIGS. 5G and 5H, apoHb-Hp complex formation can be assessed via SEC-HPLC. Similar to the trials of FIGS. 5A and 5B, the apoHb eluted at an elution volume of 3.5 mL. Based on the data from FIGS. 5G and 5H, the apoHb binding capacity can be determined via the change in the area under the curve of a pure apoHb solution versus a mixture of apoHb-Hp complex with excess apoHb. This analysis indicated similar mass binding ratios with less than 2% difference compared to the mass binding ratio of Hb.

The apoHb-Hp complex can be made to have pure complex or excess of one of the species (apoHb or Hp). Excess of either apoHb or Hp may allow for targeted treatment of different conditions characterized by higher free heme or free Hb.

### Example 5. Apohemoglobin-Haptoglobin Complexes Attenuates the Pathobiology of Circulating Acellular Hemoglobin and Heme

Haptoglobin (Hp) is the plasma protein that binds and clears cell-free hemoglobin (Hb), while apohemoglobin (apoHb, i.e. Hb devoid of heme) can bind heme. Therefore, the apoHb-Hp protein complex should facilitate holoHb-apoHb dimer exchange and apoHb-heme intercalation. Thus, it was hypothesized that apoHb-Hp could facilitate both Hb and heme clearance, which if not alleviated could have severe microcirculatory consequences. In this example, apoHb-Hp and Hb/heme ligand interactions were characterized, and their in vivo consequences were assessed. Hb exchange and heme binding with the apoHb-Hp complex was studied with transfer assays using size exclusion-high performance liquid chromatography coupled with UV-visible spectrophotometry. Exchange/transfer experiments were conducted in guinea pigs dosed with Hb or heme-albumin followed by a challenge with equimolar amounts of apoHb-Hp. Finally, systemic and microcirculatory parameters were studied in hamsters instrumented with a dorsal window chamber via intravital microscopy. In vitro and in vivo Hb exchange and heme transfer experiments demonstrated proof-of-concept Hb/heme ligand transfer to apoHb-Hp. Dosing with the apoHb-Hp complex reversed Hb- and heme-induced systemic hypertension and microvascular vasoconstriction, reduced microvascular blood flow and diminished functional capillary density. Therefore, this example highlights the apoHb-Hp complex as a novel therapeutic strategy to attenuate the adverse systemic and microvascular responses to intravascular Hb and heme exposure.

Introduction

Erythropoiesis and hemolysis occur at similar rates in healthy organisms, thus maintaining the total population of red blood cells (RBCs) in the circulation. RBC homeostasis results in the balanced cycle of erythropoiesis and hemolysis, along with the effective transport and clearance of hemoglobin (Hb) and its degradation products (heme and iron). Genetic hemolytic diseases (e.g. sickle cell anemia and thalassemia), acquired hemolytic infections (e.g. gram positive and malarial hemolysins) as well as hemolytic xenobiotic toxins (e.g. heavy metals, dapsone and phenyl hydrazine) damage the RBC, disrupting this delicate equilibrium by increasing the rate of hemolysis. Increased intravascular hemolysis and the Hb degradation product heme progress hemolytic diseases, such as sickle cell anemia, malaria and sepsis, which affect millions of patients every year.

The presence of Hb outside of the erythrocyte's protective physiologic system exposes Hb to a structurally unstable environment due to the increased dilutional dynamic equilibrium between tetrameric (α₂β₂) and dimeric components (αβ), which are more prone to autoxidation and extravasation relative to tetrameric Hb. Acellular Hb (α₂β₂) dissociation into αβ dimers results in autoxidation, structural destabilization of heme within its binding pocket, and subsequent transfer of heme prosthetic groups to lipids, proteins and bacterial receptor complexes. In addition, acellular Hb and its byproducts can scavenge nitric oxide (NO) and increase oxidative stress, cause hypertension, vasoconstriction, kidney injury, and cardiovascular lesions.

In the circulatory intravascular compartment, acellular Hb and one of its byproducts, heme, are bound and cleared by the plasma proteins haptoglobin (Hp) and hemopexin (Hpx), respectively. The ability of Hp to control acellular Hb exposure is dependent on the primary binding site for Hb αβ dimers. Irreversible binding between the Hp β chain and Hb αβ dimers occurs at a 1:1 stoichiometric ratio. This interaction provides a critical antioxidant function by protecting key amino acids in Hb that are vulnerable to oxidation, preventing heme release and compartmentalizing Hb within a stable protein complex that does not extravasate out of the circulatory intravascular compartment into the parenchymal tissues. During hemolytic diseases, Hp acts as the primary defense, stabilizing acellular Hb. Upon Hp saturation the dimerized excess Hb releases heme, which is captured by Hpx. In addition to Hpx, serum albumin can act as a secondary heme scavenger. However, albumin's affinity for heme is lower than that of Hpx and the heme-albumin complex does not fully protect from heme-mediated toxicity.

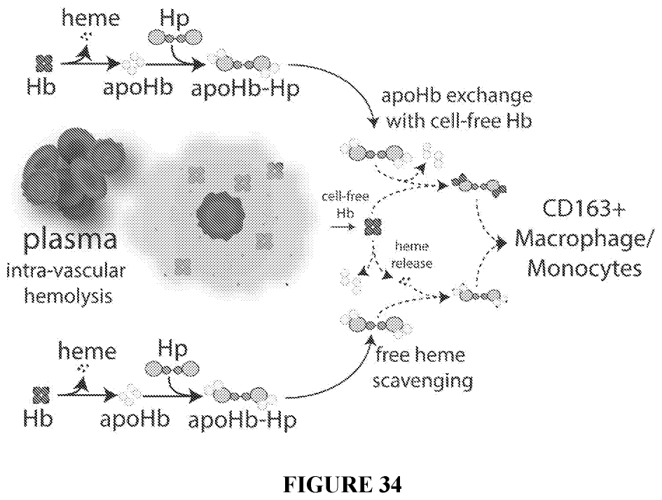

Apohemoglobin (apoHb) is produced by removing heme from Hb. This apoprotein has a high affinity region in the unoccupied hydrophobic heme-binding pocket for a number of ligands, but the highest affinity of apoHb is for heme. Some of the prior examples have illustrated the binding of heme to apoHb and the complexation of apoHb to Hp in vitro, forming an apoHb-Hp complex. In pathophysiological states of heme excess, the use of apoHb or apoHb-Hp may offer an advantage by scavenging and clearing excess heme through the monocyte/macrophage CD163 surface receptor. However, no study has explored the potential in vivo heme scavenging properties of apoHb or apoHb-Hp. Moreover, it has been observed that the apoHb-Hp complex, in addition to heme-binding, can exchange Hp bound apoHb dimers for holoHb dimers, serving a dual role of Hb and heme scavenger in vivo (see FIG. 34).

It was hypothesized that apoHb-Hp could facilitate heme transfer during states of high heme stress or holoHb exchange for apoHb dimers during acellular Hb exposure. Further, we hypothesized that these ligand interactions could attenuate the adverse microcirculatory consequences of heme and acellular Hb. In the present example, a sequential approach was tested for characterizing in vitro and in vivo apo-Hb-Hp and heme/Hb ligand interactions using a guinea pig model to assess time dependent heme transfer and Hb exchange. To test the effect of apoHb for holoHb exchange on vascular response, apoHb, Hp and apoHb-Hp complex pre-treatment followed by Hb administration as a 10% top-load was evaluated. Next, the effect of heme transfer to apoHb-Hp on the vascular response to heme-albumin was tested after apoHb-Hp as a 20% blood volume exchange. Both experimental designs were conducted in conscious Golden Syrian hamsters instrumented with the dorsal window chamber model to quantify systemic and microvascular hemodynamic responses to heme and acellular Hb.

Methods

Hb Preparation

Human Hb for this study was prepared via tangential flow filtration as described in the examples. Expired units of human red blood cells were generously donated. The concentration of Hb was determined spectrophotometrically. All Hb (also referred to as holoHb) used in this study was of human origin and derived via this method with over 97% oxyHb prior to use.

Apohemoglobin Preparation

The apoHb used in this study was prepared via tangential flow filtration based on the acidic-ethanol heme-extraction procedure as described in the examples. The absence of residual heme was verified by measuring the ratio of absorbance between the Soret peak (λmax=412−413 nm) and 280 nm to ensure less than 1% residual heme in the product. The heme-binding capacity of apoHb preparations was 80%.

Haptoglobin Preparation

Human Hp was purified from human Cohn fraction IV (FIV) purchased from Seraplex (Pasadena, Calif.). The final protein solution was composed of a mixture of Hp2-1 and Hp2-2 Hp polymers with an average MW of 400-500 kDa.

Heme-Albumin Preparation

A 4 mM heme-albumin stock solution was prepared by dissolving 65 mg Hemin (Sigma) in 100 mM NaOH at 37° C. and incubating for 1 h at 37° C. with 20% human serum albumin (HSA) and purchased from Grifols (Los Angeles, Calif.). The pH was carefully adjusted to 7.45 with 14 mM orthophosphoric acid/317 mM NaCl followed by sterile filtration.

Total Protein Assays

To estimate the total protein concentration of the apoHb solution, a Bradford assay was performed (Coomassie Plus Protein assay kit, Pierce Biotechnology, Rockford, Ill.). Additionally, spectrophotometric analysis of the 280 nm peak for each sample was used to estimate the total protein concentration using the millimolar extinction coefficients of apoHb found in the literature.

In Vitro Hb Exchange and Heme Binding by the ApoHB-Hp Complex

The apoHb-Hp complex was formed by reacting apoHb with Hp, and complex formation was confirmed by analysis of the mixture via size exclusion HPLC (HPLC-SEC). Heme binding to the apoHb-Hp complex was assessed by mixing the apoHb-Hp complex with heme-albumin prior to HPLC-SEC. Furthermore, apoHb exchange for Hb within the apoHb-Hp complex was determined by reacting the apoHb-Hp complex with Hb. HPLC-SEC was performed using an Acclaim SEC-1000 (4.6×300 mm) column (Thermo Fisher Scientific, Waltham, Mass.) attached to a Dionex UltiMate 3000 system (Thermo Fisher Scientific, Waltham, Mass.). The mobile phase consisted of 50 mM potassium phosphate, pH 7.4 at a flow rate of 0.35 mL/min controlled by Chromeleon 7 software. Wavelength absorbance used for monitoring the protein concentration was set to λ=280 nm, λ=405 nm to detect heme (bound to apoHb or albumin), and λ=413 nm to detect Hb, as it corresponds to the intermediate wavelength between residual heme in apoHb (λ=412 nm) and prevalent holoHb form used in the study, oxyHb (λ=415 nm).

In Vivo Hb Exchange and Heme Binding by the ApoHb-Hp Complex

In vivo heme transfer (heme-albumin) and Hb (acellular Hb) exchange with apoHb-Hp was studied in male Hartley guinea pigs (400-600 grams). Guinea pigs were infused with acellular Hb (0.75 μmol protein) (n=5) or heme-albumin (0.75 μmol protein) (n=5) followed by apoHb (0.75 μmol protein) bound to Hp. All materials were pre-filtered using a 0.22 pm syringe filter prior to injection. Plasma concentrations of all components were evaluated over a 6-hour period by UV-visible spectrophotometry and analytical HPLC-SEC. Plasma concentrations versus time are expressed as heme (μm/ml). On days of surgery, guinea pigs were dosed subcutaneously with ketoprofen (5 mg/kg) for pain management and then anesthetized via intraperitoneal injection with a cocktail of ketamine HCl (100 mg/kg) and xylazine HCl (5 mg/kg) (Phoenix Scientific Inc., St. Joseph, Mo. USA). Sterilized PESO tubing catheters were placed into the left external jugular vein, and left carotid artery and exteriorized at the back of the neck, all surgical sites were treated topically with bupivacaine HCl (2.5 mg/ml) (AuroMedics, Windsor, N.J., USA), and closed with 4-0 surgical silk internal sutures and external surgical staples. After 24-hours of post-surgical recovery, conscious guinea pigs were randomly allocated to dosing groups. Blood samples (150 μL) were collected through the carotid artery at baseline, after acellular Hb or heme-albumin albumin infusion, immediately after apoHb-Hp infusion (time 0) then at 0.25, 0.50, 0.75, 1.00, 1.25, 1.50, 3.00 and 6.00 hours.

Plasma Hemoglobin Analysis

Blood samples were centrifuged at 2,000 rpm for 10 min immediately after collection. Plasmas were diluted in 50 mM phosphate-buffered saline and analyzed on the same days of blood collection using a Carey 60 UV-visible spectrophotometer (Agilent Technologies, Santa Clara, Calif.). Oxy ferrous Hb [HbFe²⁺O₂] and ferric Hb [HbFe³⁺] concentrations were determined based on the extinction coefficients for each species. Molar extinction coefficients used to calculate Hb concentrations in heme equivalents were: 15.2 mM⁻¹ cm⁻¹ at 576 nm for Hb(O₂) and 4.4 mM⁻¹ cm⁻¹ at 631 nm for ferric Hb using 50 mM potassium phosphate buffer, pH 7.0 at ambient temperature, in both cases. Total heme was calculated by adding these values.

HPLC-SEC Anlysis of Hb-Hp Complexes in Plasma

Plasma samples (50 μL) were injected into a BioSep-SEC-S3000 (600 7.5 mm) SEC column (Phenomenex, Torrance, Calif.). The SEC column was attached to a Waters 2535 Quaternary Gradient Module pump and Waters 2998 Photodiode Array Detector controlled by a Waters 600 controller using Empower 2 software (Waters, Milford, Mass.), wavelength monitoring was the same as used for in vitro analysis (280, 405 and 413 nm).

Window Chamber Preparation in Golden Syrian Hamsters

Studies were performed in 55-65 g male Golden Syrian Hamsters (Charles River Laboratories, Boston, Mass.) fitted with a dorsal window chamber. The hamster window chamber model is widely used for microvascular studies without anesthesia. The complete surgical technique has been described previously. Two days after window implantation, arterial and venous catheters filled with heparinized saline solution (30 IU/mL) were implanted into the carotid and jugular vessels. Catheters were tunneled under the skin, exteriorized at the dorsal side of the neck, and securely attached to the window frame.

Inclusion Criteria

Hamsters were considered suitable for experiments if systemic parameters were as follows: heart rate (HR)>340 beats/min, mean arterial blood pressure (MAP)>80 mm Hg, systemic Hct>45%, and arterial 02 partial pressure (pAO₂)>50 mm Hg. Additionally, hamsters with signs of low perfusion, inflammation, edema, or bleeding in their microvasculature were excluded from the study. Guinea pigs were included in the study if they were deemed healthy and met the weight range criteria of 400-600 g.

Experimental Setup

The unanesthetized animal was placed in a restraining tube with a longitudinal slit from which the window chamber protruded, then fixed to the microscopic stage for transillumination with the intravital microscope (BX51WI, Olympus, Japan). Animals were given 20 minutes to adjust to the tube environment and images were obtained using a CCD camera (4815, COHU, San Diego, Calif.). Measurements were carried out using a 40× (LUMPFL-WIR, numerical aperture 0.8, Olympus) water immersion objective.

Systematic Parameters

The MAP and HR were monitored continuously (MP150, Biopac System Inc., Santa Barbara, Calif.). Hct was measured from centrifuged arterial blood samples taken in heparinized capillary tubes. Hb content was determined spectrophotometrically (B-Hemoglobin, Hemocue, Stockholm, Sweden). Arterial blood was collected in heparinized glass capillaries (50 μL) and immediately analyzed for pO₂, pCO₂, and pH (ABL90; Radiometer America, Brea, Calif.). Arterial Hb saturation was measured using an IL482 CO-Oximeter (Instrumentation Laboratory, Lexington, Mass.).

Microhemodynamics

Arteriolar and venular blood flow velocities were measured using the photodiode cross-correlation method (Photo-Diode/Velocity, Vista Electronics, San Diego, Calif.). The measured centerline velocity (V) was corrected according to blood vessel size to obtain the mean RBC velocity. A video image-shearing method was used to measure blood vessel diameter (D). Blood flow (Q) was calculated from the measured values as Q=π/4D² V.

Functional Capillary Density (FCD)

Functional capillaries, defined as capillary segments that have transit of at least one RBC in a 60 second period in 10 successive microscopic fields, were assessed in a region of 0.46 mm². The FCD (cm⁻¹) was calculated as the total length of RBC perfused capillaries divided by the viewing area (0.46 mm²).

Vascular Response to Acellular Hb

Hamsters were pre-treated with apoHb (30 mg/mL, 100 μL, n=6), Hp (15 mg/mL, 50 μL, n=6), apoHb-Hp complex (45 mg/mL, apoHb 15 mg/mL+Hp 30 mg/mL, 150 μL, n=6), or vehicle (saline equal volume as study groups, n=6). Hamsters were then subjected to a 10% hypervolemic infusion of purified acellular human Hb (50 mg/mL, 500 μL) and systemic and microvascular responses were measured after 30 minutes. All materials were pre-filtered using a 0.22 μm syringe filter prior to injection.

Vascular Response to Heme Albumin

Hamsters were either pre-treated with apoHb-Hp complex (45 mg/mL, apoHb 15 mg/mL+Hp 30 mg/mL-0.15 mL and 6.75 mg total dose, n=6), or vehicle (saline equal volume as study groups, n=6). Then hamsters were dosed with heme-albumin (2.0 mg/mL, 2.0-2.8 mg total dose) via a 20% blood volume exchange transfusion. All materials were pre-filtered using a 0.22 μm syringe filter prior to injection. Systemic hemodynamics and microcirculatory function were measured after 30 minutes.

Statistical Analysis

For in vivo Hb exchange and heme transfer studies performed in guinea pigs (n=5/group), all data are represented as mean values±SD. AUC values were derived using the linear trapezoidal rule and data were compared using a One-way ANOVA with Tukey's multiple comparisons test for parametric data in GraphPad Prism 8.3, GraphPad Software Inc., San Diego, Calif. For microcirculatory studies, data within each group were analyzed using a two-way ANOVA for repeated measurements. When appropriate, post hoc analyses were performed with the Dunnett's multiple comparisons test. Microhemodynamic data are presented as ratios relative to baseline values and absolute values are reported in the supplementary tables. A ratio of 1.0 signifies no change from baseline, while lower and higher ratios are indicative of changes proportionally lower and higher than baseline (i.e., 1.5 represents a 50% increase from the baseline level). The same blood vessels and capillary fields were monitored throughout the study, such that direct comparisons to their baseline levels could be performed, allowing for more reliable statistics on small sample populations. All statistics were calculated using GraphPad Prism 6 (GraphPad Software, Inc., San Diego, Calif.). Changes were considered significant if p values were less than 0.05.

Results

In Vitro HoloHb Exchange and Heme Binding by the ApoHb-Hp Complex

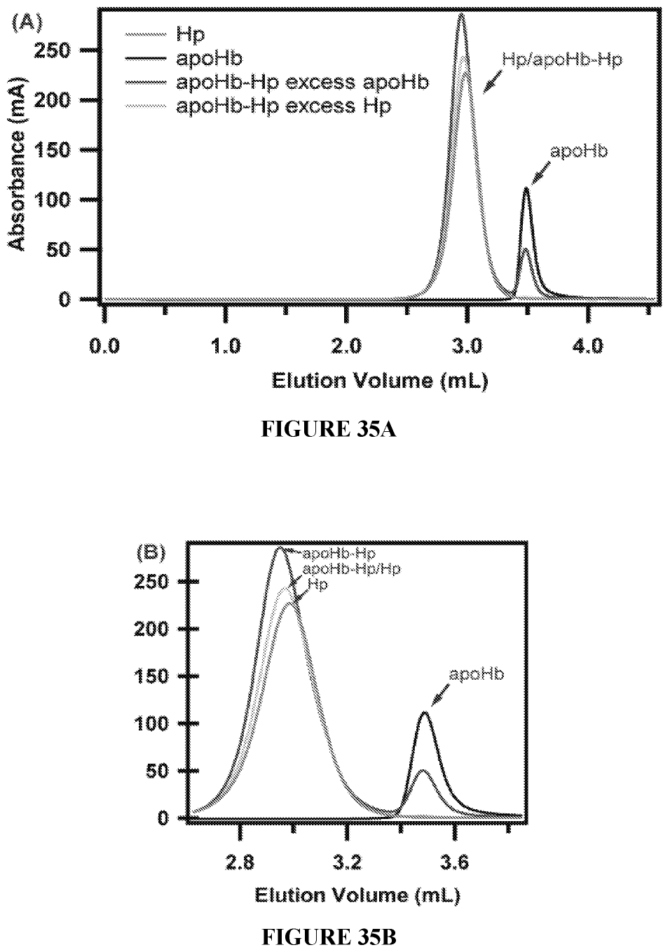

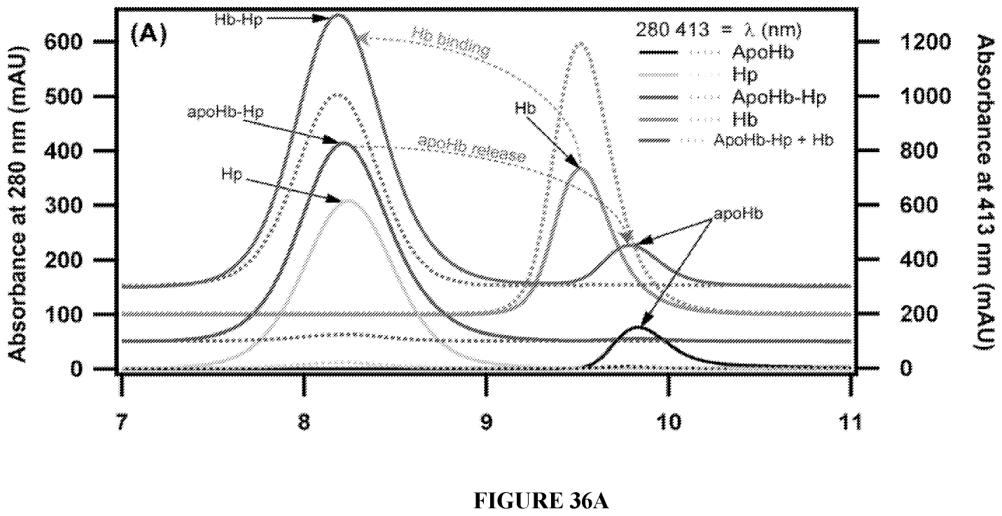

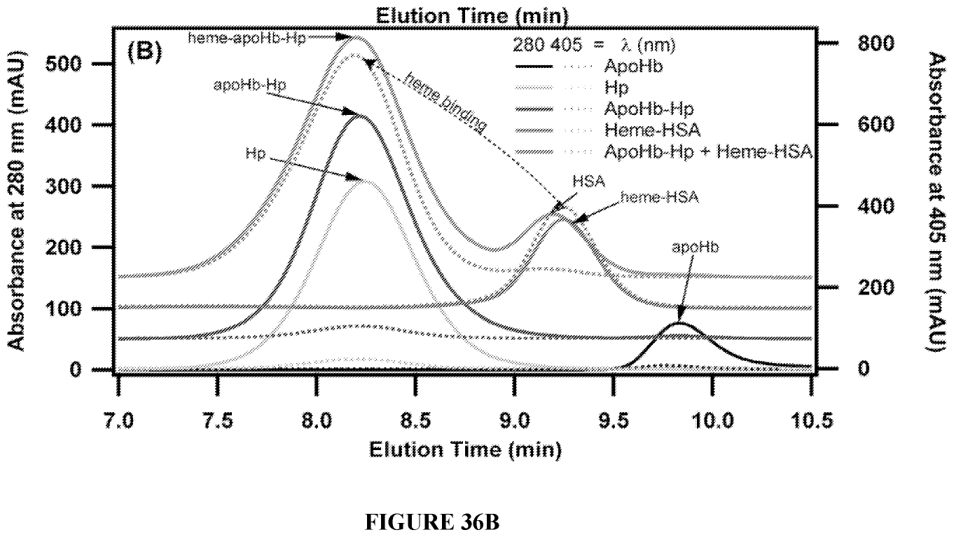

Formation of the apoHb-Hp complex in vitro can be achieved by reacting the two proteins (apoHb+Hp). The difference in molecular weight (MW) of Hp (˜400 kDa in this study) compared to apoHb (˜32 kDa) allowed for assessment of apoHb-Hp complex formation via HPLC-SEC. This is shown in the SEC chromatograms of FIGS. 35A and 35B. Based on results from in vitro mixing, and analysis by HPLC-SEC, apoHb bound to Hp to form the apoHb-Hp complex. Based on the change in the AUC of the apoHb peak, the total mass of apoHb αβ dimers bound to Hp was comparable to the total mass of holoHb αβ dimers bound to Hp, demonstrating stoichiometric binding of apoHb to Hp. Further, in vitro heme-binding and Hb exchange with the apoHb-Hp complex was assessed via HPLC-SEC. The apoHb-Hp complex was mixed with either free holoHb (composed of over 97% oxyHb) or heme-albumin. When holoHb was mixed with apoHb-Hp (see FIG. 36A), Hb-apoHb exchange occurred, as evidenced by an increase in absorbance at 413 nm. Furthermore, free apoHb eluted in the same chromatogram, further indicating detachment of apoHb from the apoHb-Hp complex. This observation agrees with previous reports regarding the lower association equilibrium of apoHb to Hp compared to that of holoHb to Hp. Moreover, when apoHb-Hp was mixed with heme-albumin (see FIG. 36B), heme was transferred from albumin to the apoHb in the apoHb-Hp complex given that the absorbance of free albumin (405 nm) decreased while the absorbance of apoHb-Hp (405 nm) complex increased. Given the high Hp binding affinity for Hb and the high heme-binding affinity of apoHb, the scavenging reactions (for Hb and heme) occurred nearly instantaneously. Even though all Hp binding sites were saturated with apoHb prior to the experiment, there was no noticeable effect on Hb binding to Hp as apoHb was quickly displaced from the apoHb-Hp complex. Furthermore, since both Hb exchange with apoHb-Hp or heme-binding to apoHb-Hp yield a similar complex composed of the holoprotein bound to Hp, in an in vivo hemolytic condition, the complex would not be expected to preferentially scavenge heme versus Hb. In future studies we expect to analyze the kinetics and equilibrium of the apoHb-Hp complex when both Hb and heme are present.

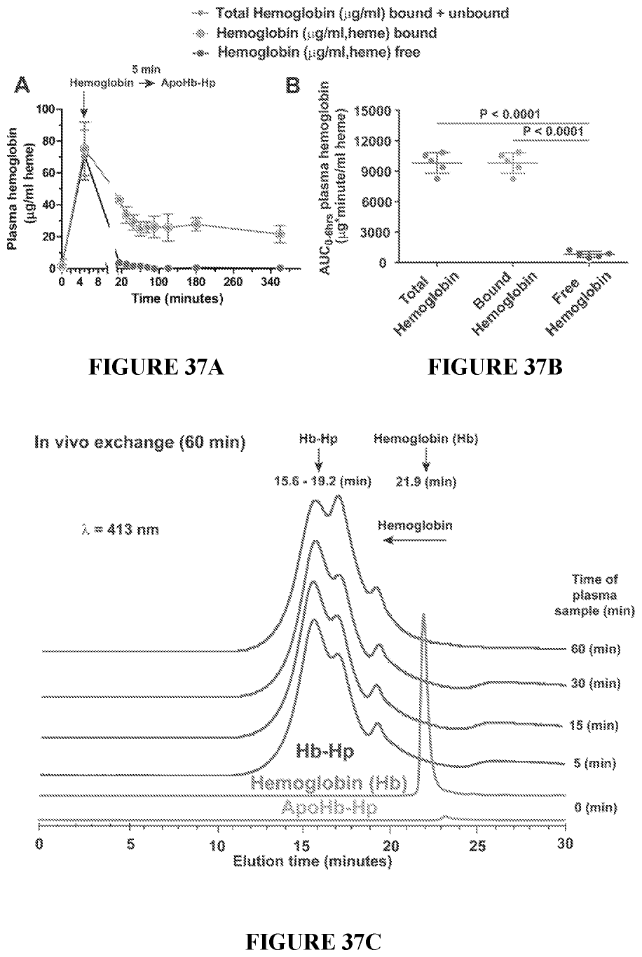

In Vivo HoloHb Exchange for ApoHb within the Hb-Hp Complex Guinea pigs were administered holoHb (denoted as Hb) followed by apoHb-Hp and time dependent blood sampling for analysis of Hp-bound and -unbound Hb plasma concentrations versus time are shown in FIG. 37A. Maximum Hp-bound holoHb concentrations (Cₘₐₓ) occurred rapidly, with a time of maximum concentration (Tₘₐₓ) occurring within the 5 minute timeframe of holoHb administration (Hp-bound holoHb=75.1±16.8 μg/ml, as heme). Unbound holoHb concentrations in plasma remained low-to-undetectable (3.80±2.17 μg/ml, as heme) after the initial exchange of apoHb for holoHb αβ dimers in Hp was observed in the plasma concentration versus time curves (see FIG. 37A). The derived areas under the plasma concentration versus time curves (AUC _{(0-360 min)}) were 9818±1026 μg× minute/ml (total=Hp-bound+unbound), 9001±986.1 μg× minute/ml (Hp-bound) and 805.4±133.6 μg× minute/ml (unbound) for holoHb. These data demonstrate that Hp-bound apoHb αβ dimers were completely exchanged for holoHb αβ dimers, indicated by significantly higher Hp-bound holoHb versus unbound AUC _{(0-360 min)} values (p<0.0001) shown in FIG. 37B. The rapid accumulation and stability holoHb bound to Hp is observed by the representative HPLC-SEC chromatographs with detection at 413 nm shown in FIG. 37C.

In Vivo Heme Transfer from Heme-Albumin to ApoHb within the ApoHb-Hp Complex

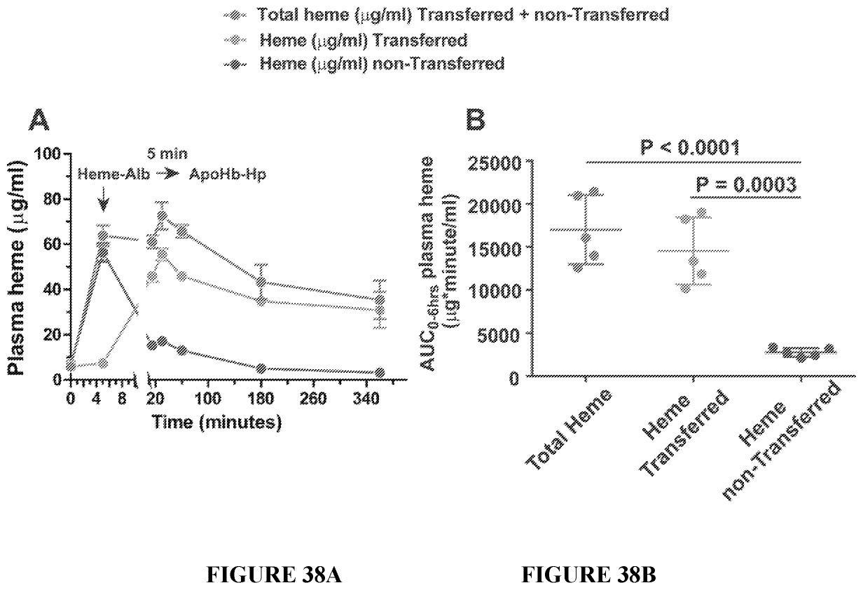

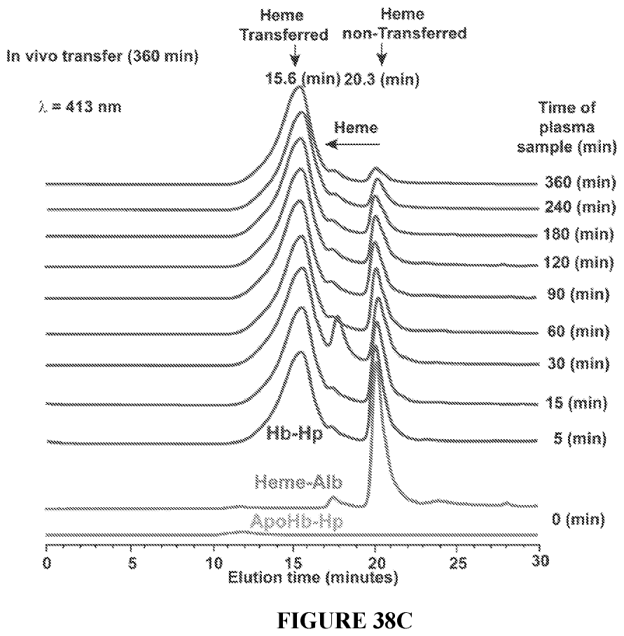

Guinea pigs were administered heme-albumin followed by apoHb-Hp. Time dependent blood sampling was conducted for analysis of heme transfer from heme-albumin to apoHb-Hp resulting in heme intercalated into apoHb αβ dimers, generating bound holoHb αβ dimers (denoted Hb-Hp) and their plasma concentrations over time as shown in FIG. 38A. The transfer of heme from heme-albumin to apoHb-Hp occurred more slowly in vivo. Cₘₐₓ values for heme intercalation into apoHb-Hp were 55.5±2.60 μg/ml (as heme concentration) occurring at a Tₘₐₓ=30 minutes. The areas under the plasma concentration versus time derived curves (AUC _{(0-360 min)}) were 12,597±4015.0 μg× minute/ml (total heme=heme-albumin+heme-apoHb-Hp), 10,163±3922.0 μg× minute/ml (transferred heme=heme-apoHb-Hp, denoted Hb-Hp) and 2125±511.6 μg×minute/ml (non-transferred heme=heme-albumin). Total and heme-albumin transferred heme were significantly greater (p<0.0001 and p=0.0003) compared to non-transferred heme, respectively as shown in FIG. 38B. The time course of heme transfer from heme-albumin to apoHb-Hp to generate Hb-Hp within the guinea pig plasma compartment is shown in the representative HPLC-SEC chromatographs with detection at 413 nm shown in FIG. 38C.

Microvascular Impact of Pretreatment with ApoHb, Hp and ApoHb-Hp Complex Followed by Acellular Hb Infusion

Systemic Parameters

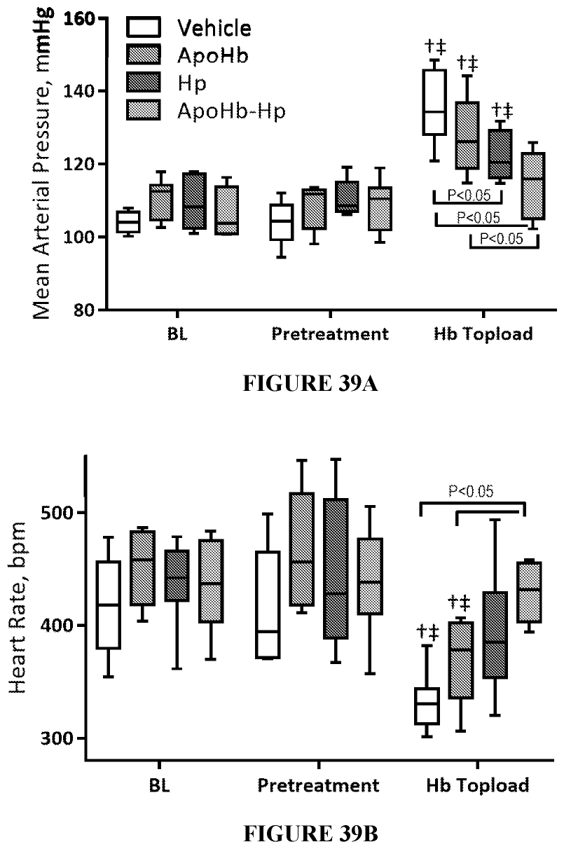

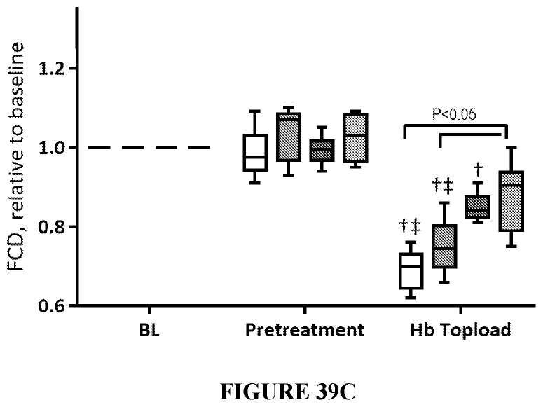

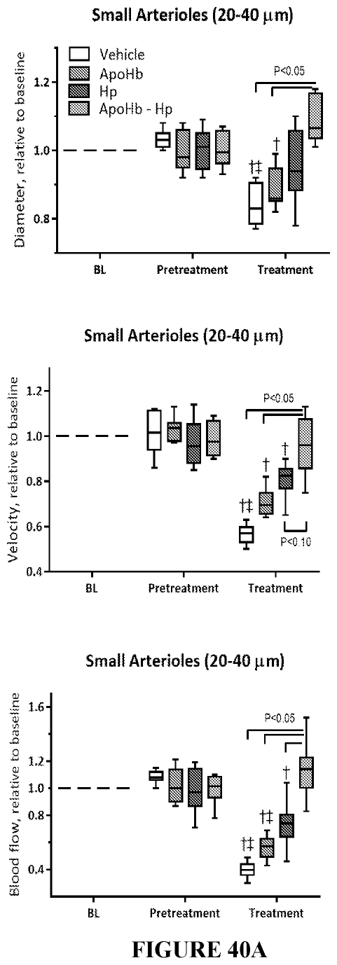

The MAP and HR after pretreatment with vehicle, apoHb, Hp or apoHb-Hp, and subsequent challenge with acellular Hb dosing are presented in FIGS. 39A and 39B. The MAP and HR were not statistically different between groups at baseline or after pre-treatment with the test materials. After administration of acellular Hb, vehicle-dosed animals, and animals treated with apoHb or Hp alone demonstrated elevated MAP compared to baseline and pretreatment (p<0.05). After Hb administration, both Hp and apoHb-Hp showed lower MAP than vehicle alone. However, only treatment with the apoHb-Hp complex maintained the animal's MAP at baseline and pretreatment levels. The HR after Hb infusion was inverse, but similar to the change in MAP. Acellular Hb infusion caused a statistically significant decrease in HR from baseline and pre-treatment for the vehicle and apoHb groups. There were no significant changes in HR in response to acellular Hb challenge for animals pretreated with Hp and apoHb-Hp. Furthermore, vehicle-treated, and apoHb-treated animals showed lower HR compared to the apoHb-Hp treated group post acellular Hb transfusion.

Functional Capillary Density

The FCD after pretreatment with vehicle, apoHb, Hp or apoHb-Hp, and challenge with acellular Hb dosing are presented in FIG. 39C. Pretreatment with test materials did not result in any adverse changes in FCD. FCD decreased for vehicle-treated and apoHb-treated animals following acellular Hb transfusion compared to baseline and pre-treatment. ApoHb-Hp attenuated the Hb-driven loss of FCD, as there were no statistical differences between time points, and this group's FCD was significantly higher than that of vehicle and apoHb alone following Hb transfusion. Hp alone also attenuated the loss of FCD due to Hb challenge, but to a lesser extent than the apoHb-Hp complex. These data suggest that Hp and apoHb-Hp pre-treatment result in a comparable response to Hb exposure and support the concept of holoHb-apoHb exchange within the apoHb-Hp complex.

Microhemodynamics

The changes in arteriolar and venular hemodynamics relative to baseline are shown for all four experimental groups. The arterioles were segmented into various vessel diameters: small arterioles from 20 to 40 μm, mid-size arterioles from 40 to 60 μm and large arterioles from 60 to 100 μm. All venules were very consistent and grouped into a single venule group from 30 to 80 μm in vessel diameter.

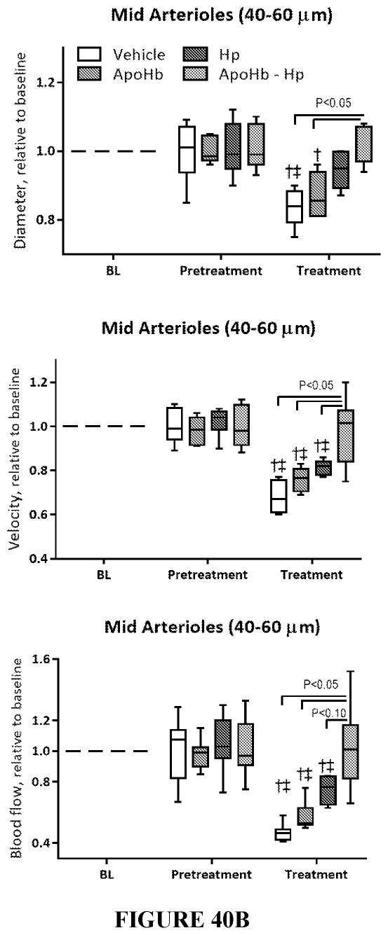

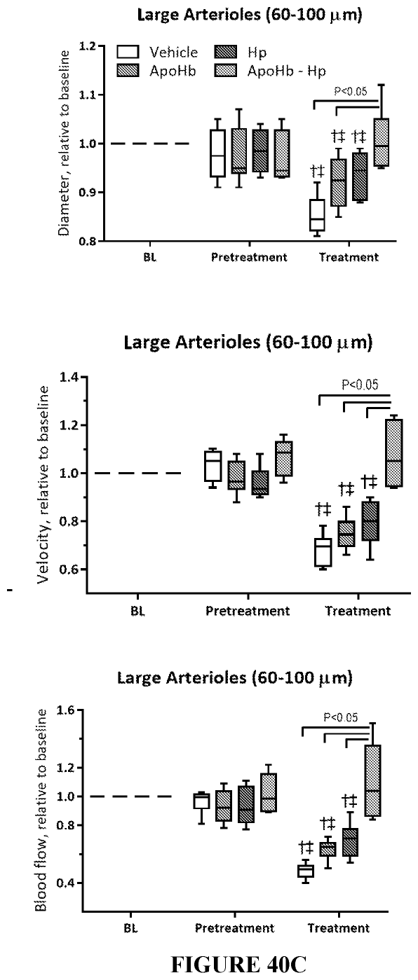

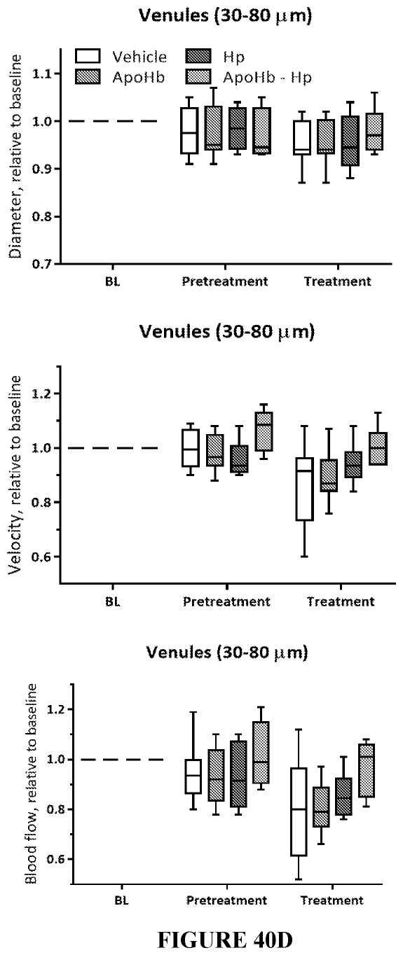

Small Arterioles (20-40 μm): The diameter, velocity and flow, relative to baseline, for small arterioles, ranging from 20-40 μm in diameter are shown in FIG. 40A, with baseline values shown in Tables 7-9 below. There were no differences observed between baseline and pre-treatment groups. Following Hb administration, the vehicle group (untreated animals) (n=18 vessels) demonstrated decreased microvascular responses (i.e. decreased diameter, velocity and flow) compared to baseline and pre-treatment groups. Following Hb administration, animals infused with apoHb alone (n=14 vessels) also demonstrated decreased microvascular responses compared to baseline and pre-treatment groups in small vessel diameter, velocity, and flow. The small arteriolar diameter of animals infused with Hp alone (n=16 vessels) did not changed from baseline, however, their respective velocity and flow decreased compared to the other pre-treatment groups. The small arteriolar diameter of animals dosed with Hp did not changed after Hb administration, thus, the Hp prevented vasoconstriction. The administration of apoHb-Hp improved diameter, velocity and flow compared to basal levels and in response to Hb dosing.

TABLE 9
Baseline diameter for small arterioles in μm.

| | Control | ApoHb | Hp | ApoHb-Hp |
|---|---|---|---|---|
| 25% Percentile | 27.49 | 28.03 | 30.90 | 31.43 |
| Median | 33.00 | 31.53 | 32.55 | 31.45 |
| 75% Percentile | 33.68 | 31.65 | 32.56 | 34.31 |

TABLE 10
Baseline velocity for small arterioles in mm/s.

| | Control | ApoHb | Hp | ApoHb-Hp |
|---|---|---|---|---|
| 25% Percentile | 1.96 | 2.11 | 2.69 | 2.98 |
| Median | 3.44 | 3.00 | 3.26 | 3.10 |
| 75% Percentile | 3.46 | 3.08 | 3.34 | 3.16 |

TABLE 11
Baseline flow for small arterioles in nL/s.

| | Control | ApoHb | Hp | ApoHb-Hp |
|---|---|---|---|---|
| 25% Percentile | 1.25 | 1.40 | 2.14 | 2.21 |
| Median | 2.69 | 2.32 | 2.48 | 2.31 |
| 75% Percentile | 2.86 | 2.56 | 2.90 | 2.51 |

Mid-Sized Arterioles (40-60 μm): The diameter, velocity and flow, relative to baseline, for medium sized arterioles, ranging from 40-60 μm in diameter are shown in FIG. 40B, with baseline values shown in Tables 10-12 below. There were no differences observed between baseline and pre-treatment groups. Following Hb administration, the vehicle group (untreated animals) (n=18 vessels) demonstrated a decrease in vessel diameter, velocity and flow compared to baseline and pre-treatment. ApoHb alone (n=16 vessels) demonstrated a decrease in diameter, velocity, and flow compared to baseline and pre-treatment. Following Hb administration, animals infused with Hp alone (n=19 vessels) demonstrated no differences in diameter compared to baseline or pre-treatment, however, velocity and flow were decreased. Following Hb administration, the apoHb-Hp complex was not different from baseline or pre-treatment. However, after Hb dosing the apoHb-Hp group did demonstrate an increase in vessel diameter, velocity and flow compared to the control and apoHb groups but did increase in arteriole velocity compared to Hp dosing alone.

TABLE 12
Baseline diameter for large arterioles in μm.

| | Control | ApoHb | Hp | ApoHb-Hp |
|---|---|---|---|---|
| 25% Percentile | 66.99 | 75.61 | 68.1 | 67.91 |
| Median | 76.46 | 80.69 | 71.59 | 69.74 |
| 75% Percentile | 78.66 | 82.49 | 72.75 | 70.44 |

TABLE 13
Baseline velocity for large arterioles in mm/s.

| | Control | ApoHb | Hp | ApoHb-Hp |
|---|---|---|---|---|
| 25% Percentile | 13.59 | 22.39 | 18.86 | 17.65 |
| Median | 27.32 | 33.16 | 24.97 | 21.55 |
| 75% Percentile | 27.85 | 33.48 | 27.16 | 25.04 |

TABLE 14
Baseline flow for large arterioles in nL/s.

| | Control | ApoHb | Hp | ApoHb-Hp |
|---|---|---|---|---|
| 25% Percentile | 3.64 | 4.84 | 5.17 | 6.05 |
| Median | 5.363 | 6.43 | 5.93 | 6.54 |
| 75% Percentile | 5.60 | 6.51 | 6.20 | 6.60 |

Large Arterioles (60-100 μm): The diameter, velocity and flow, relative to baseline, for large arterioles, ranging from 60-100 μm in diameter are shown in FIG. 40C, with baseline values shown in Tables 13-15 below. There were no differences observed between baseline and pre-treatment groups. Following Hb dosing, the vehicle group (n=22 vessels) demonstrated decreases compared to baseline and treatment for diameter, velocity and flow. Similarly, apoHb (n=16 vessels) and Hp (n=19 vessels) pre-treatment followed by Hb dosing, demonstrated decreases in diameter, velocity, and flow compared to baseline and pre-treatment. Following Hb dosing, the apoHb-Hp complex showed no change compared to baseline or pre-treatment. The group comparisons demonstrated a difference in large arteriole diameter between the control and apoHb groups compared to apoHb-Hp group. Large arteriole velocity and flow were significantly decreased in control, apoHb and Hp groups following Hb dosing compared to the apoHb-Hp group.

TABLE 15
Baseline diameter for large arterioles in μm.

| | Control | ApoHb | Hp | ApoHb-Hp |
|---|---|---|---|---|
| 25% Percentile | 66.99 | 75.61 | 68.1 | 67.91 |
| Median | 76.46 | 80.69 | 71.59 | 69.74 |
| 75% Percentile | 78.66 | 82.49 | 72.75 | 70.44 |

TABLE 16
Baseline velocity for large arterioles in mm/s.

| | Control | ApoHb | Hp | ApoHb-Hp |
|---|---|---|---|---|
| 25% Percentile | 13.59 | 22.39 | 18.86 | 17.65 |
| Median | 27.32 | 33.16 | 24.97 | 21.55 |
| 75% Percentile | 27.85 | 33.48 | 27.16 | 25.04 |

TABLE 17
Baseline flow for large arterioles in nL/s.

| | Control | ApoHb | Hp | ApoHb-Hp |
|---|---|---|---|---|
| 25% Percentile | 3.64 | 4.84 | 5.17 | 6.05 |
| Median | 5.363 | 6.43 | 5.93 | 6.54 |
| 75% Percentile | 5.60 | 6.51 | 6.20 | 6.60 |

Venules (30-80 μm): The diameter, velocity and flow, relative to baseline, for venules, ranging from 30-80 μm in diameter are shown in FIG. 40D, with baseline values are shown in Tables 16-18 below. There were no differences observed between baseline and pre-treatment groups. After Hb dosing, the vehicle, apoHb, Hp and apoHb-Hp groups demonstrated similar diameter, velocity and flow-based responses.

TABLE 18
Baseline diameter for venules in μm.

| | Control | ApoHb | Hp | ApoHb-Hp |
|---|---|---|---|---|
| 25% Percentile | 37.87 | 36.81 | 34.00 | 31.72 |
| Median | 48.74 | 53.39 | 48.28 | 46.14 |
| 75% Percentile | 78.21 | 78.50 | 74.08 | 74.13 |

TABLE 19
Baseline velocity for venules in mm/s.

| | Control | ApoHb | Hp | ApoHb-Hp |
|---|---|---|---|---|
| 25% Percentile | 0.98 | 1.19 | 1.02 | 0.89 |
| Median | 1.40 | 1.62 | 1.34 | 1.35 |
| 75% Percentile | 2.26 | 2.58 | 2.46 | 2.31 |

TABLE 20
Baseline flow for venules in nL/s.

| | Control | ApoHb | Hp | ApoHb-Hp |
|---|---|---|---|---|
| 25% Percentile | 1.14 | 1.23 | 0.99 | 0.66 |
| Median | 2.52 | 3.35 | 2.12 | 2.23 |
| 75% Percentile | 10.16 | 11.56 | 11.48 | 10.66 |

Taken together, these data suggest that apoHb-Hp may offer an improvement over Hp alone in microvascular function of small, mid-size and large arteriole. These data are potentially the first to demonstrate such an effect in the microcirculation and indicate the potential for a concomitant heme and Hb mediated pathobiology on the vasculature. To better understand this, the systemic and microcirculatory hemodynamic response to pre-treatment with our novel apoHb-Hp construct followed by heme-albumin exposure was studied.

In Vivo Pretreatment with ApoHb-Hp Followed by Heme-Albumin Exposure

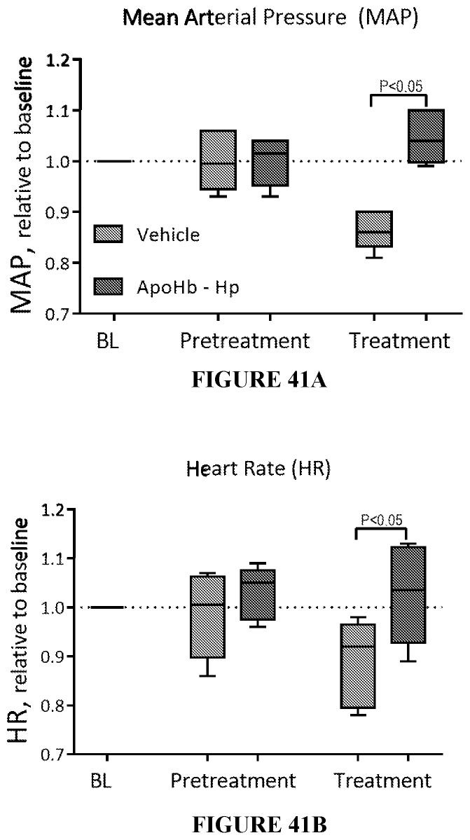

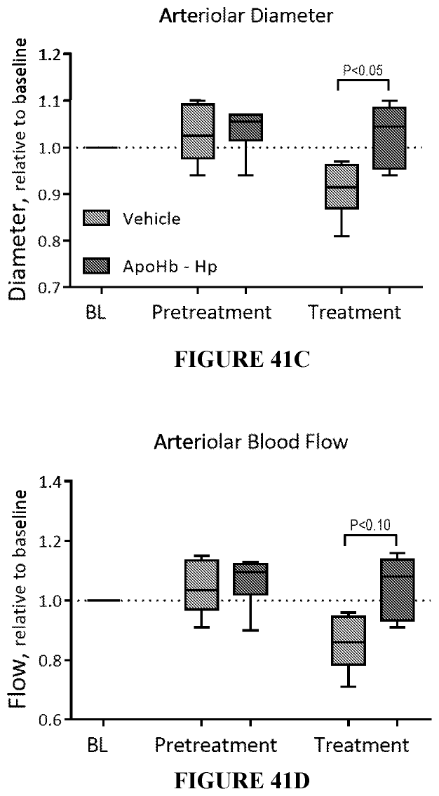

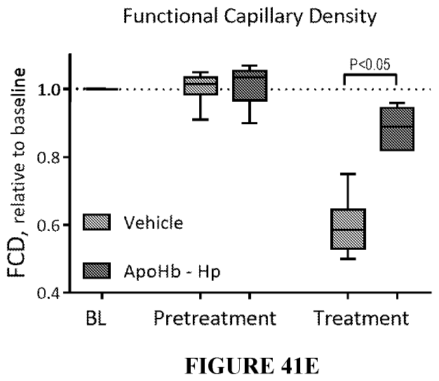

This study was completed in twenty-four (N=24) instrumented animals. Eight animals were randomly assigned to each experimental group. The first experimental group was a control (untreated animals), and the second group was infused with apoHb-Hp. All groups were then administered a single dose of heme-albumin. The impact of apoHb-Hp on mean arterial blood pressure (MAP) and heart rate (HR) following heme-albumin challenge are presented in FIGS. 41A and 41B. Heme-albumin infusion decreased both MAP and HR decreased by 10% in control animals. Pretreatment with apoHb-Hp prevented this decrease in MAP and HR, suggesting modest cardiovascular depression in response to heme. Within the time frame of dosing and measurement (20 minutes), it is possible that heme metabolism by hemoxygenase-1 generated enough carbon monoxide to explain this effect on both the macrovascular circulation and the myocardium. The changes in arteriolar hemodynamics (diameter and flow) are shown in FIGS. 41C and 41D. Similar to MAP, control animals experienced a 20% and 10% decrease in flow and diameter, respectively, relative to baseline in response to heme-albumin. Pretreatment with apoHb-Hp prevented this decline in diameter and flow. The FCD is shown in FIG. 41E and demonstrates a 40% decrease in RBC transit through capillaries in response to heme-albumin exposure for control animals relative to baseline. Pretreatment with apoHb-Hp also prevented this decrease in FCD in response to heme-albumin exposure. This further demonstrates a pathophysiologic response to exposure to heme-albumin at the microcirculatory level and suggests that apoHb-Hp has the ability to attenuate this response.

Discussion

Acellular Hb has multiple pathophysiologic effects when released into the intravascular space during hemolysis. Hb tetramers (α₂β₂) dissociate into αβ dimers quickly in the circulation which can easily oxidize into methemoglobin (metHb) and release free heme. Multiple pathophysiological consequences are associated with heme and Hb. To prevent the adverse effects from Hb and its degradation products, Hp binds dimeric Hb (i.e. αβ dimers), thus preventing Hb from mediating oxidative reactions and from releasing free heme. When Hp is depleted (during extensive acute or low-level chronic hemolysis), heme released by Hb is transferred and bound by Hpx. In addition, albumin serves as a secondary heme binding protein, but does not fully prevent heme-mediated toxicity due to its lower affinity for heme than highly lipophilic LDL, HDL and VLDL. Hb binding to Hp is a biologically irreversible process, creating a stable and high-molecular-weight molecule that is cleared by macrophages and monocytes upon binding to the scavenger receptor, CD163. ApoHb acts as a heme scavenging protein, binding heme with a higher affinity than albumin. Moreover, the apoHb-heme protein can bind to Hp to be cleared through CD163+ macrophages and monocytes. To ensure apoHb-heme can be delivered to monocytes/macrophages apoHb was bound to Hp. This heme capture mechanism provides a specific pathway for heme clearance, in lieu of the described route of heme-Hpx uptake, which occurs through LRP-1 (low density lipoprotein receptor, CD91), a ubiquitous receptor that exists on the surface of numerous cell types and exhibits a multitude of functions.

This example demonstrates in vitro and in vivo holoHb exchange for apoHb when bound to Hp and highlights the unique property of heme intercalation into apoHb-Hp, following holoHb and heme-albumin exposure in guinea pigs. This combination of effects offers a potentially advantageous combination when concomitantly or independently addressing intravascular hemolysis and heme stress. Furthermore, microcirculatory studies in hamsters with apoHb, Hp, and the apoHb-Hp complex reduced adverse macrovascular and microvascular responses to acellular Hb exposure. More specifically, administration of Hp and apoHb-Hp complex prevented hypertension and vasoconstriction, as well as preserved microvascular diameter, blood flow, and functional capillary density across a range of arteriolar sizes. The apoHb-Hp complex appears to demonstrate a complementary mechanism to address Hb vasoactive response by attenuating heme and acellular Hb vascular interactions in the circulation. Unless bound to Hp, Hb dissociates into αβ dimers and extravasates through the blood vessel wall and reacts with or scavenges NO. Furthermore, both heme within Hb and heme bound to albumin participate in oxygen radical reactions that covalently modify proteins, lipids, carbohydrates, and nucleotides, leading to tissue damage. Administration of the apoHb-Hp complex may provide a unique therapeutic strategy to simultaneously mitigate heme stress and plasma Hb exposure across a range of disease states that involve heme-protein circulatory exposures. This approach provides a distinctive physiologic method to target the well-established mechanism of Hb-Hp clearance and intra-cellular Hb/heme detoxification by targeting the monocyte/macrophage CD163 surface receptor.

In the above microcirculatory function studies, infusion of acellular Hb induced arteriolar vasoconstriction and increased vascular resistance in small, mid and large arterioles. As Hb dissociates into αβ dimers it extravasates through the fenestrated capillaries and scavenges NO from the tissue. NO stimulates the production cGMP, decreasing the intracellular Ca²⁺ concentration, resulting in muscle relaxation; without the accumulation of cGMP the smooth muscles around the arterioles constrict. Hb mediated NO consumption leads to vasoconstriction, prevents perfusion of capillary beds, and reduces the number of functional capillaries, preventing metabolite washout. The vasoconstriction in the microcirculation results in an upstream response observed as an increase in MAP. The increase in MAP leads to a decrease in heart rate through the baroreceptor reflex. Finally, arteriole constriction hinders flow and velocity in all sized arterioles suggesting a reduction in tissue oxygen perfusion. The severity of these physiological responses can be observed after a simple exogenously administered acellular Hb intravascular dose. During active intravascular hemolysis, the body's natural levels of scavenger proteins (Hp and Hpx) cannot keep up with the demand to stabilize the Hb dimer or scavenge free heme and its associated iron.

In the above studies, a single injection of Hp, at a concentration of 1.5 g/dL, prior to the 0.1 mL Hb dose resulted a greater reduction in vasoconstriction compared to the control and the apoHb infused animals. This was an expected result, since Hp stabilizes the Hb αβ dimer and prevents the release of heme. Arterioles experienced less vasoconstriction, and flow as well as velocity were preserved to a greater degree. The downstream effect is that MAP and HR are maintained closer to baseline. Hp dosing also prevented the loss of FCD in response to heme and Hb, supporting the concept of intravascular compartmentalization which prevents extravasation of Hb through fenestrated capillaries, less NO scavenging, and preservation of microvascular pressures. For animals pretreated with 3 g/dL of apoHb, hemodynamic side-effects were observed in animals after Hb exposure. Most of the adverse hemodynamic responses were still present, including constriction of the arterioles, a decrease in velocity and flow, elevated systemic blood pressure, decreased heart rate and a reduction in functional capillaries. However, apoHb-Hp pre-dosing followed by Hb exposure lead to the most optimal response in terms of preserving systemic and microhemodynamic parameters close to the values found at baseline. ApoHb-Hp prevented the rise in MAP and decrease in HR. Further apoHb-Hp optimized blood vessel diameter, velocity and flow at baseline levels and the number of functional capillaries were maintained at basal levels. These results demonstrate a proof-of-concept that the apoHb-Hp complex prevents Hb-mediated circulatory dysfunction. In addition, apoHb-Hp was effective at maintaining basal circulatory function when dosed prior to heme-albumin exposure.

Conclusion

The present data suggests that the apoHb-Hp complex can effectively exchange apoHb for holoHb in vitro and in vivo. Further, apoHb situated in the apoHb-Hp complex is an effective heme binding protein complex based on in vitro and in vivo heme-albumin transfer studies and in vivo microcirculatory experiments. These unique properties of the apoHb-Hp complex prevent adverse systemic and microvascular responses to Hb and heme-albumin exposure and introduces a novel therapeutic approach to facilitate simultaneous removal of extracellular Hb and heme.

### Example 6. Apohemoglobin-Haptoglobin Complexes Attenuate the Hypertensive Response to Low Molecular Weight Polymerized Hemoglobin

Polymerized hemoglobin (PolyHb) is a promising hemoglobin (Hb)-based oxygen (O₂) carrier (HBOC) currently undergoing development as a red blood cell (RBC) substitute. Unfortunately, commercially developed products are comprised of low molecular weight (MW) PolyHb molecules, which extravasate, scavenge nitric oxide (NO), and resulted in vasoconstriction and hypertension. The naturally occurring Hb scavenging species, haptoglobin (Hp), combined with the purified heme scavenging species, apohemoglobin (apoHb), is a potential candidate to alleviate the pressor effect of PolyHb. In this example, the protective activity of administration of the apoHb-Hp complex was evaluated to mitigate the vasoactive response induced by the transfusion of low MW PolyHb. Hp binding to PolyHb was characterized in vitro. The effectiveness of apoHb-Hp administration on reducing the vasoconstriction and pressor effects of PolyHb was assessed by measuring systemic and microcirculatory hemodynamics. Transfusion of a low MW PolyHb to vehicle control pretreated animals increased mean arterial pressure (MAP), and decreased arteriolar dimeter, and functional capillary density (FCD). Transfusion of a low MW PolyHb to apoHb-Hp pretreated animals prevented changes in MAP, heart rate, arteriole diameter and blood flow, and FCD relative to before transfusion. These results indicate that the increased size of PolyHb after binding to the apoHb-Hp complex may help compartmentalize PolyHb in the vascular space and thus reduce extravasation, NO scavenging, and toxicity responsible for vasoconstriction and systemic hypertension.

Introduction

During the recent outbreak of severe acute respiratory syndrome coronavirus 2 (SARS-CoV-2), closure of blood donation centers and large exclusions in blood donor pools has resulted in severe blood shortages. A 2017 computer simulation of an influenza outbreak in the United States predicted that over 541,000 units of blood might be lost at the end of a 48-week influenza pandemic with a 65% reduction in blood donation rates. However, current red blood cell (RBC) donation trends, show that the donation rate decay due to SARS-CoV-2 is more severe than the previous influenza models. This deficit in the blood supply emphasizes the need for a RBC substitute when RBC units are not available. Unfortunately, there are currently no RBC substitutes approved for clinical application.

The presence of low molecular weight (MW) hemoglobin (Hb) species in previous generations of Hb-based oxygen (02) carriers (HBOCs) restricted their application as RBC substitutes. Among the myriad of strategies that can produce HBOCs, using glutaraldehyde crosslinking to form polymerized Hb (PolyHb) remains popular due to its low cost and excellent scalability. However, the commercially developed PolyHbs, HBOC-201 (Biopure Corp, Cambridge, Mass., USA) and PolyHeme (Northfield Laboratories Inc., Northfield, Ill., USA), primarily contained fractions of material at or below 250 kDa. Low MW PolyHb and unpolymerized Hb extravasate into the interstitial space where they scavenge nitric oxide (NO), resulting in vasoconstriction and systemic hypertension. Moreover, free heme release from HBCOs can also lead to systemic toxicity. Previously, it was determined that increasing the MW of PolyHb by increasing the molar ratio of glutaraldehyde to Hb attenuated hypertension and renal injury. However, low MW polymers comprise up to 40% of these PolyHb solutions. Unfortunately, removal of these low MW polymers would significantly reduce the yield of PolyHb. Alternatively, recent strategies to mitigate hypertension have used adenosine and nitroglycerine to attenuate the hypertensive response of HBOC-201. While effective at controlling systemic hypertension, administration of adenosine and nitroglycerine must be carefully controlled to prevent hypotension.

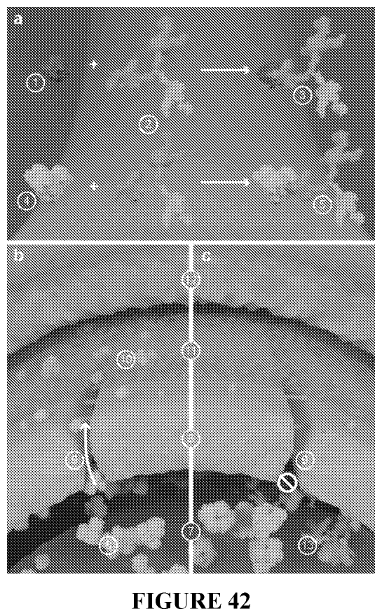

Instead, it is proposed that employing naturally occurring mechanisms of Hb detoxification is a potential strategy to mitigate systematic hypertension resulting from the presence of low MW PolyHb in the circulation. The naturally occurring Hb scavenging protein, haptoglobin (Hp), has a pivotal role in detoxifying stroma free Hb in the blood. A previous study demonstrated that Hp administration normalizes vascular NO signaling after hemolysis. Recently, it was determined that Hp preferentially binds to low MW PolyHb (MW<256 kDa). By binding to Hp, the MW of low MW PolyhHb is effectively increased. This increase in molecular size may reduce tissue extravasation and subsequent NO scavenging by PolyHb. An illustration of this potential detoxification mechanism is shown in FIG. 42.

The second component of stroma-free Hb detoxification is hemopexin (Hpx). Hpx scavenges heme that is released from stroma free Hb after Hb auto-oxidation. By isolating heme within the Hpx complex, the heme is unable to catalyze oxidative reactions with blood and tissue components, thus preventing lipid, protein, and nucleic acid oxidation. A potential low-cost alternative to Hpx is heme-free apohemoglobin (apoHb). This molecule is able to scavenge free heme due to the highly hydrophobic nature of its vacant heme-binding pocket. Combining apoHb with Hp results in a protein complex (apoHb-Hp) that may be able to scavenge both Hb and heme in the plasma.

Because of its ability to compartmentalize stroma-free Hb and scavenge free heme, it was hypothesized that administration of the apoHb-Hp complex could reduce hypertension that results from the transfusion of low MW PolyHb. Hp binding to low MW PolyHb was confirmed in vitro with stop-flow fluorescence spectrometry and size exclusion chromatography. Mean arterial blood pressure (MAP) was used as an indicator of reduced hypertension. Additionally, intravital microscopy was used to examine how administration of apoHb-Hp influences functional capillary density (FCD), vascular tone, and blood flow after PolyHb administration.

Methods

PolyHb Synthesis and Biophysical Properties

The Hb used in this exampler was purified from human RBCs via tangential flow filtration (TFF), as described previously. Hb was polymerized with glutaraldehyde under complete deoxygenation in the tense (T) quaternary state at a 25:1 molar ratio of glutaraldehyde to Hb. After polymerization, PolyHb was clarified, purified, and buffer exchanged into a modified Ringer's lactate solution using TFF. Modified polyester sulfone hollow fiber filters with a molecular weight cutoff (MWCO) of 100 kDa were used to remove unpolymerized Hb from solution. The PolyHb solution was concentrated to 12 g/dL. The cyanomethemoglobin method was used to measure the Hb concentration and the metHb level of Hb/PolyHb solutions. The size distribution of PolyHb, by particle volume, was measured using dynamic light scattering (DLS) (Brookhaven Instrument Inc. BS-200M, Holtsville, N.Y.). The rheology of PolyHb solutions was measured using a DV3T-CP cone and plate rheometer (Brookfield AMETEK, Middleboro, Mass.) with cone spindle CPA-40Z. The O₂-Hb/PolyHb equilibrium binding curves were measured using a Hemox Analyzer (TCS Scientific Corp., New Hope, Pa.). The Hb/PolyHb kinetics of O₂ offloading (k_{off,O}_{₂}) and NO dioxygenation kinetics (k_{ox,NO}) were measured with an Applied Photophysics SF-17 microvolume stopped-flow spectrophotometer (Applied Photophysics Ltd., Surrey, United Kingdom) using protocols previously described by Rameez and Palmer. The molecular weight (MW) distribution was estimated using an Acclaim SEC-1000 column (Thermo Scientific, Waltham, Mass.) on a Thermo Scientific Dionex Ultimate UHPLC system using previously described methods. All measurements were taken from the same sample used in the animal study.

ApoHb Purification and Biophysical Properties

The apoHb used in this study was produced using acidified-ethanol coupled TFF, as described previously. The heme-binding site activity of the resulting apoHb was quantified with a dicyanohemin assay. The total amount of protein in solution was estimated based on the molar extinction coefficient of apoHb.

Hp Purification and Biophysical Properties

The Hp used for this study was purified from human Cohn Fraction IV derived from pooled human plasma. The resulting Hp contained both Hp2-1 and Hp2-2 phenotypes. The total amount of protein in solution was estimated with a Bradford assay. The Hb binding capacity of Hp was assessed by monitoring Hb binding to Hp at 413 nm with HPLC-SEC and was used to quantify the concentration of Hp. The reaction of Hp with either unmodified human Hb or PolyHb synthesized in this study was monitored using an SX-20 stopped stopped-flow spectrophotometer using previously described methods (Applied Photophysics, Leatherhead, UK). Excess Hp (2:1, Hp:PolyhHb on a Hb tetramer binding basis) was allowed to react with PolyHb to form the resulting Hp-PolyHb complex. The resulting changes in size distribution after Hp binding was estimated with HPLC-SEC using previously described methods. All measurements were taken from the same sample used in the animal study.

Animal Model

In vivo evaluation of apoHb-Hp detoxification of PolyHb was performed on 55-65 g male Golden Syrian Hamsters (Charles River Laboratories, Boston, Mass.) fitted with a dorsal skinfold window chamber as previously described. Animals were considered suitable for experiments if systemic parameters were as follows: heart rate (HR)>340 beats/min, mean arterial blood pressure (MAP)>80 mm Hg, systemic Hct>45%, and arterial O₂ partial pressure (pAO₂)>50 mm Hg. Additionally, animals with signs of low perfusion, inflammation, edema, or bleeding in their microvasculature were excluded from the study. Prior to treatment, baseline systemic parameters and microvascular hemodynamics were assessed. Animals were first treated with either (1) 0.1 mL 0.9 wt % saline, (2) 0.1 mL apoHb (24.5 mg/L), or (3) 0.1 mL apoHb (24.5 mg/L) with 0.05 mL Hp (46.5 mg/mL). 20 minutes following the initial treatment, the systemic parameters and microvascular hemodynamics were assessed. Afterward, the animals underwent a 20 percent isovolemic exchange transfusion with a 10 g/dL PolyHb solution. After another 20 minutes, the systemic parameters and microvascular hemodynamics were measured. Mean arterial pressure (MAP) and heart rate (HR) were monitored continuously (MP150, Biopac System Inc., Santa Barbara, Calif.). The sample size of 5 animals per group was calculated based on an expected 10% difference in mean arterial pressure between groups, an α=0.05, and 1-β=0.1, and equal enrollment for all groups. Additionally, microhemodynamic measurements contain data from multiple vessels within the field (5-7 arterioles and venules selected at baseline based on visual clarity), improving the power of these measurements. Each experiment is a repeated measures study, so all experimental timepoints are replicated between all animals and groups. Furthermore, each experimental group contains animals from different litters, improving the replication of these studies. Animals were randomly assigned to their respective group before baseline measurements were taken. Each group contains animals from multiple litters of hamsters to improve randomization. Investigators were not blinded to group allocation during data collection or analysis. Blinding is not possible with these solutions as they have distinct colors. Measurements taken are highly quantitative, so blinding should have little impact.

Intravital Microscopy

The unanesthetized animal was placed in a restraining tube with a longitudinal slit from which the window chamber protruded, then fixed to the microscopic stage for transillumination with the intravital microscope (BX51WI, Olympus, New Hyde Park, N.Y.). Animals were given 20 minutes to adjust to the tube environment and images were obtained using a CCD camera (4815, COHU, San Diego, Calif.). Measurements were carried out using a 40× (LUMPFL-WIR, numerical aperture 0.8, Olympus) water immersion objective.

Microvascular Hemodynamics

Arteriolar and venular blood flow velocities were measured using the photodiode cross-correlation method (Photo-Diode/Velocity, Vista Electronics, San Diego, Calif.). The measured centerline velocity (V) was corrected according to blood vessel size to obtain the mean RBC velocity. A video image-shearing method was used to measure blood vessel diameter (D). Blood flow (Q) was calculated from the measured values as

$$Q = π \times {V ( \frac{D}{2} )}^{2} .$$

Functional Capillary Density (FCD)

Functional capillaries, defined as capillary segments that have RBC transit of at least one RBC in a 60 second period in 10 successive microscopic fields, were assessed in a region of 0.46 mm². FCD (mm⁻¹) is calculated as the total length of RBC perfused capillaries divided by the area (0.46 mm²).

Statistical Analysis

Results are presented as mean±standard deviation. One-way ANOVA was used to analyze data within the same group. All box plots are presented with the median on the center line, the box limits are set to the upper (75%) and lower (25%) quartile. All outliers are shown each plot. Post-hoc analysis was completed with the Dunn multiple comparison test. Data between groups were analyzed with a two-way ANOVA with Bonferroni tests. When possible, in vivo data was compared against baseline in the same animal or same vessel as a ratio relative to the baseline. All statistical calculations and data analyses were performed with R (v. 3.6.2). For all tests, P<0.05 was considered statistically significant. All data is available upon reasonable request.

Results

PolyHb Characterization

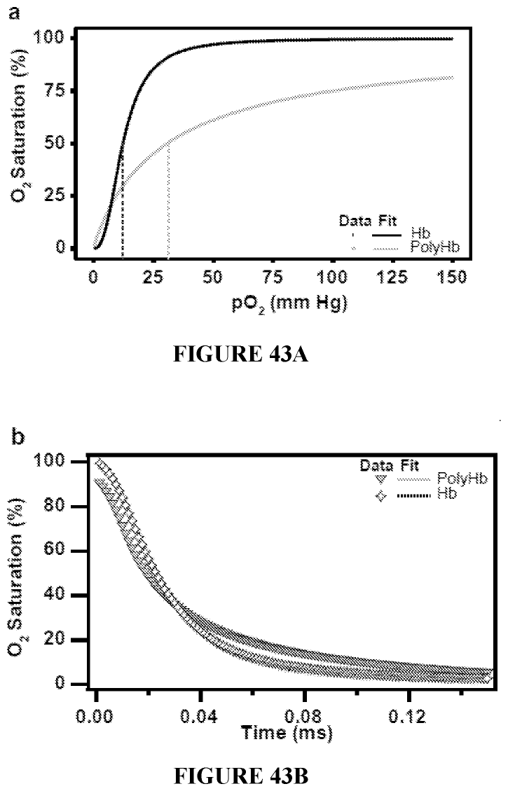

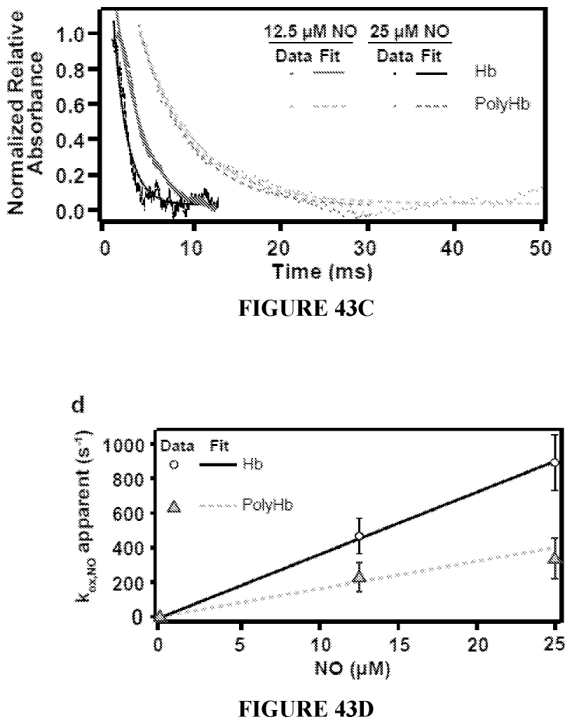

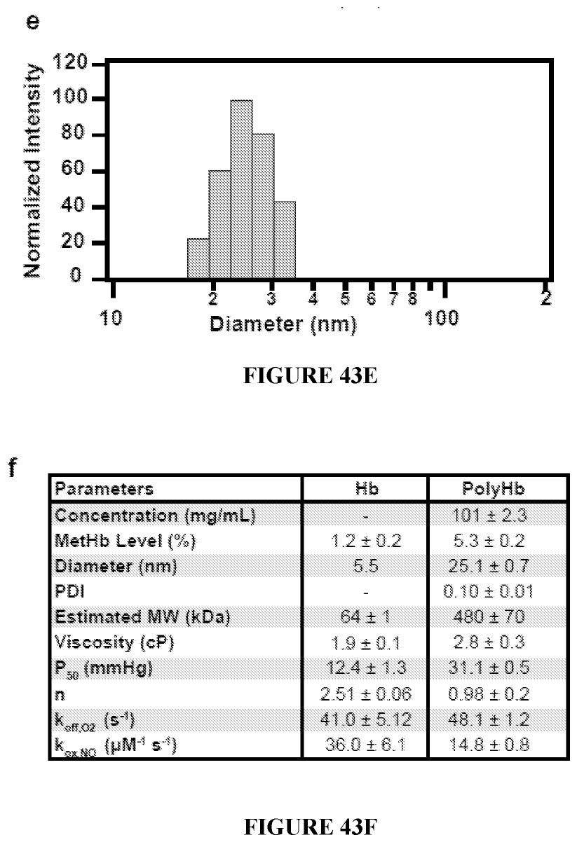

A summary of the biophysical properties of the PolyHb used in this study is shown in FIGS. 43A-43F. Polymerization significantly increased the metHb level (5.3±0.2%) compared to unmodified Hb (1.2±0.2%). The PolyHb used in this study had significantly reduced oxygen affinity (P₅₀=31.1±0.5 mm Hg) and reduced cooperativity (n=0.98±0.21) compared to the oxygen affinity (P₅₀=12.4±1.3 mm Hg) and cooperativity (n=2.51±0.06) of unmodified Hb. After polymerization, the hydrodynamic diameter of PolyHb increased to 25.1 nm. The resulting PolyHb solution had a single peak with low polydispersity (PDI=0.10±0.10). PolyHb had an estimated average MW of 480 kDa. Polymerization significantly increased the viscosity (2.8±0.3 cP) compared to unmodified Hb (1.2±0.2 cP). Polymerization significantly increased the rate of O₂ offloading (k_{off,O}_{₂}=48.1±1.2) compared to unmodified Hb (k_{off,O}_{₂}=41.0±5.12). In contrast, polymerization significantly decreased the rate of NO dioxygenation (k_{ox,NO}=14.8±0.8) compared to unmodified Hb (k_{ox,NO}=36.0±6.1).

Characterization of Hp Binding to PolyHb

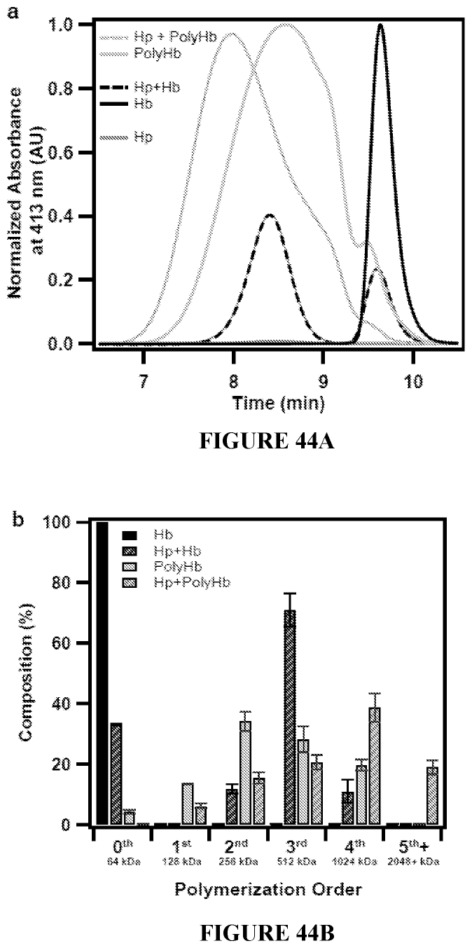

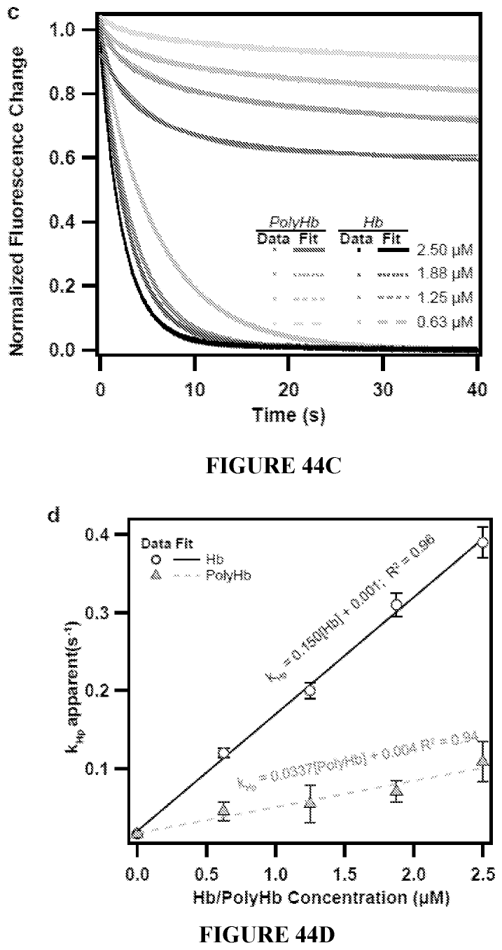

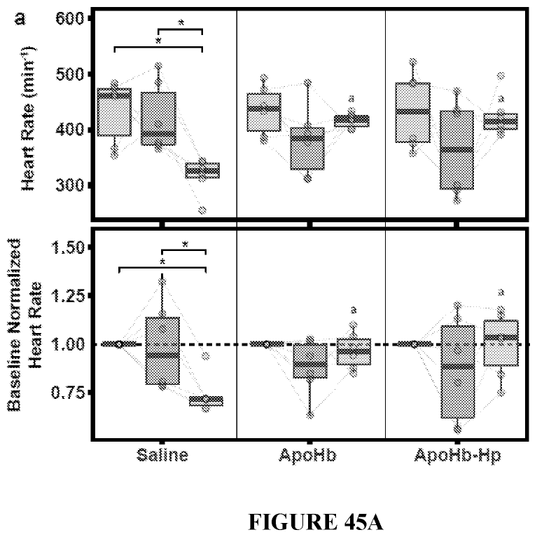

The effect of Hp binding on the size distribution of PolyHb and Hb is shown in FIG. 44A. To verify that Hp completely bound to unmodified hHb, excess Hb was mixed with Hp at a 3:2 molar ratio of Hb to Hp. The central peak for Hb was observed at an elution time of 9.61±0.05 minutes. Hp was slightly larger, with an average elution time of 8.32±0.12 minutes. Compared to Hp and Hb, PolyHb had a relatively broad size distribution. Due to this broad size distribution, a polymer size order decomposition was performed on the resulting peaks to estimate the composition of the Hp polymer species. The results of this analysis are shown in FIG. 44B. After mixing the 3:2 molar ratio of Hb to Hp, 33% of unmodified hHb (size order 64 kDa) remains unbound. This indicates that all of the Hp in solution was able to bind Hb. The resulting Hp-Hb complex was ˜512 kDa in size, which indicates that a single bound Hp increases the size by at least 500 kDa. Analysis of low MW PolyHb fractions (<512 kDa) indicates that only 10% of PolyHb does not bind to Hp. However, the 64 kDa 0^{th} order PolyHb species is completely eliminated by Hp binding. The large MW (1 and 2 MDa) polymer size orders increase by 30%. Representative time courses of the kinetics of Hp binding to Hb and PolyHb are shown in FIG. 44C The dependence of the pseudo-first-order rates on Hb/PolyHb concentration is shown in FIG. 44D. Overall, the kinetics of Hp binding to Hb was much faster (0.150±0.001 μM⁻¹s⁻¹) compared to PolyHb (0.0337±0.001 μM⁻¹s⁻¹).

Systemic Hemodynamics

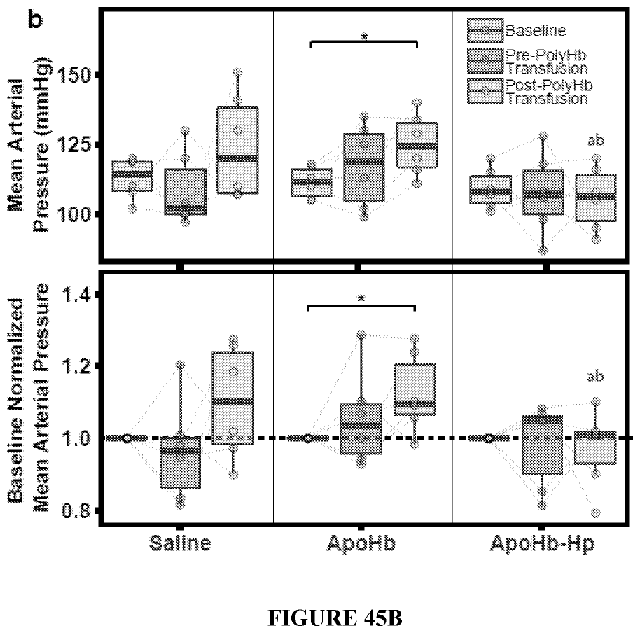

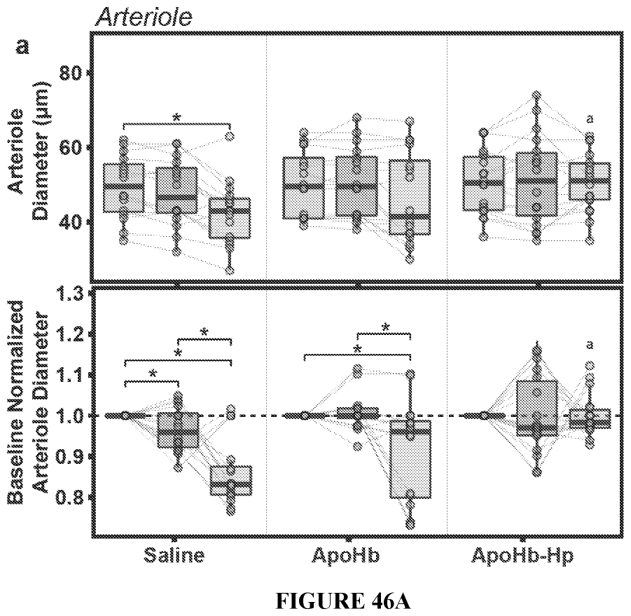

MAP and HR measured throughout the experimental study are shown in FIGS. 45A and 45B. There was no significant difference in baseline conditions for all animals. Prior to transfusion of PolyHb, the administration of saline, apoHb, and apoHb-Hp had no significant effect on MAP or HR compared to each other and the baseline condition. After exchange transfusion of PolyHb in animals that underwent treatment with saline, there was a significant decrease in HR compared to baseline and pretransfusion conditions. For animals that were administered apoHb, there was a significant increase in MAP. For animals that underwent treatment with apoHb-Hp, there was no significant change in MAP or HR compared to baseline and pretransfusion conditions. In animals administered with the apoHb-Hp solution, the post-PolyHb transfusion HR was significantly higher when compared to animals administered with the saline solution. MAP in animals administered either saline or apoHb solutions after PolyHb transfusion was significantly higher compared to animals administered with apoHb-Hp solutions.

Microhemodynamics

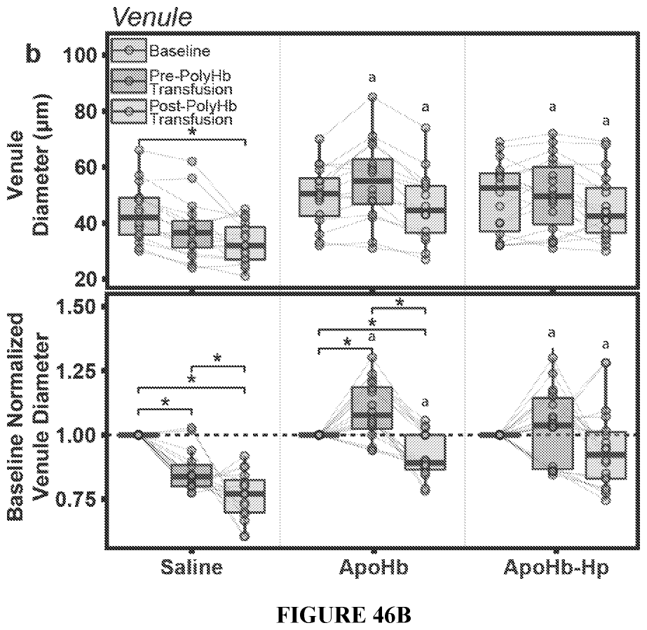

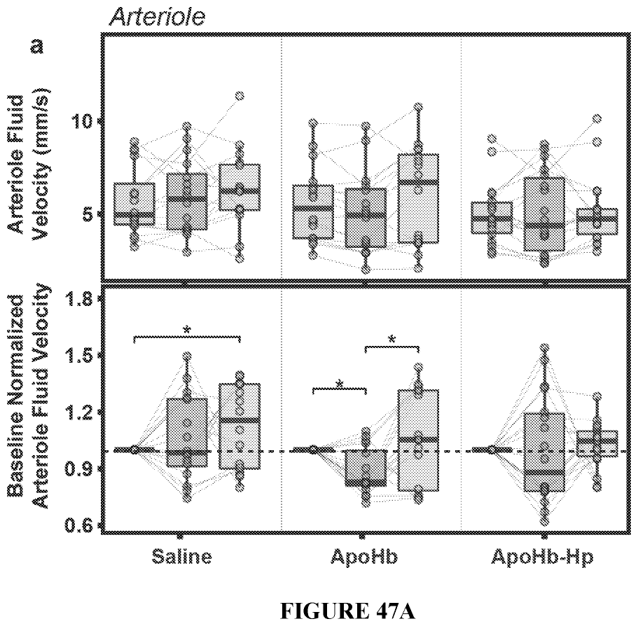

Changes in arteriole and venule diameter as measured with intravital microscopy are shown in FIGS. 46A and 46B. There was a significant decrease in the diameter of arterioles and venules compared to baseline after vehicle administration and PolyHb transfusion in the saline treatment group. For animals in the apoHb treatment group, there was a significant decrease in relative arteriole and venule diameter compared to the baseline after transfusion of PolyHb. For animals in the apoHb-Hp treatment group, there were no significant changes observed in arteriole and venule diameter throughout treatment. The vessel diameters of venules and arterioles in the apoHb-Hp group were significantly larger compared to the saline treatment group.

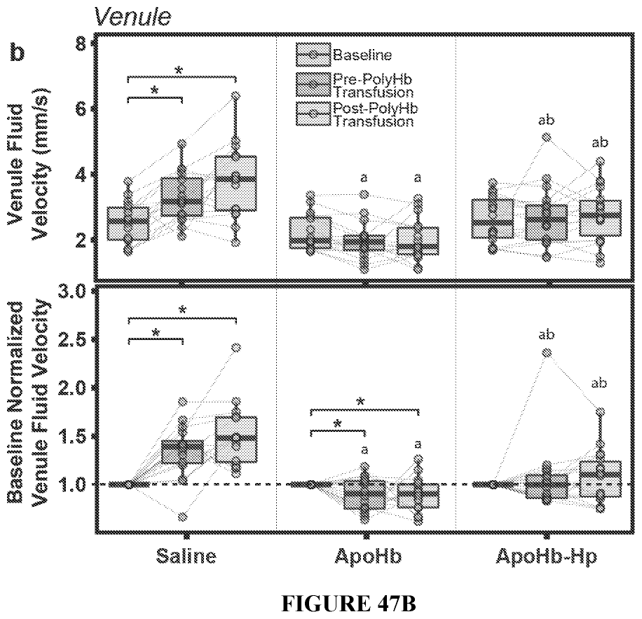

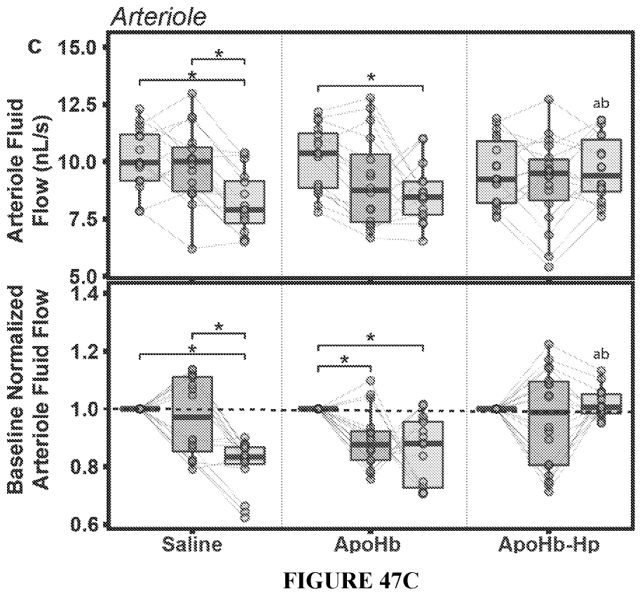

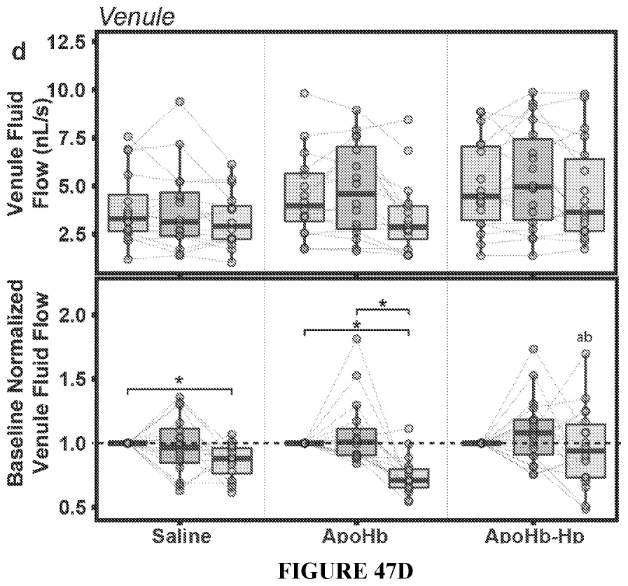

Changes in the blood velocity and volumetric flow rate measured via intravital microscopy are shown in FIGS. 47A-47D. The relative arteriole and venule fluid velocity in the saline group significantly increased after PolyHb transfusion. In contrast, the relative venule fluid velocity significantly decreased after apoHb administration. The venule fluid velocities in the apoHb treatment group were significantly lower than the venule fluid velocities in the saline treatment group. After treatment with apoHb, we observed a significant decrease in the arteriole volumetric flow rate. In the saline and apoHb treatment groups, the volumetric flow rate in the arterioles and venules decreased after PolyHb transfusion.

In the apoHb-Hp treatment group, there were no significant changes in arteriole and venule fluid velocities and volumetric flow rates throughout the study. The venule fluid velocity in the apoHb-Hp treatment group was significantly lower than the saline group and significantly higher than the apoHb treatment group. In the apoHb-Hp group, the arteriole and venule volumetric flow rates after PolyHb administration were significantly higher compared to the saline and apoHb treatment groups.

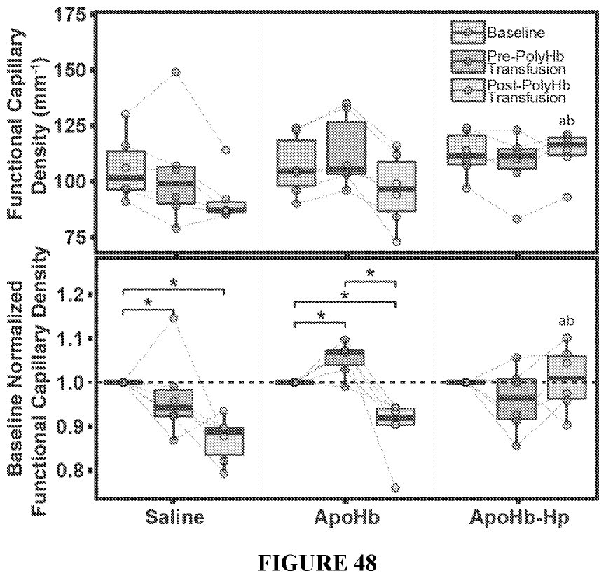

Changes in FCD throughout the intravital microscopy study are displayed in FIG. 48. After treatment with saline, the FCD significantly decreased relative to the baseline. In contrast, treatment with apoHb resulted in a significant increase in FCD. After PolyHb transfusion, FCD significantly decreased in the saline and apoHb treatment groups. There were no significant changes in FCD in the apoHb-Hp treatment group compared to baseline. After PolyHb transfusion, the FCD in the apoHb-Hp treatment group was significantly higher than the FCD in the saline and apoHb treatment groups. There were no significant differences at baseline between treatment groups for any microhemodynamic parameters.

Discussion

The principal finding of this example is that administration of a Hb and heme scavenging material (apoHb-Hp) maintained hemodynamics after a 20% isovolemic exchange transfusion with a low MW PolyHb. In animals administered with apoHb-Hp, there were negligible changes in MAP, HR, and microhemodynamics compared to baseline conditions. When compared to the systemic and microcirculatory changes observed in the apoHb and saline groups, the relatively small changes in the apoHb-Hp group indicate that Hp-based species may serve as appropriate materials to counteract the vasoactive effects of low MW HBOCs that are capable of binding to Hp.

The biophysical properties of the Hp produced in this study was comparable with values measured in the literature. The rate constant for Hp binding to Hb was similar to values reported in the literature for Hb (0.129 μM⁻¹s⁻¹). The rate constant for Hp binding to 25:1 PolyHb was much higher than the values reported in the literature for a PolyHb (0.003 μM⁻¹s⁻¹) and Oxyglobin (0.011 μM⁻¹s⁻¹). However, it was comparable to previously produced PolyHb. Even though the rate of Hp binding to PolyHb was significantly lower than the rate of Hp binding to Hb, systemic and microcirculatory hemodynamics were maintained throughout the studies. This indicates that Hb does not significantly inhibit Hp binding to PolyHb. By observing the change in fluorescence intensity, we were able to calculate the percentage of PolyHb that is capable of binding to Hp. For Hb, we calculated complete binding site saturation when excess Hb was in solution. In contrast, only a fraction of the binding sites was saturated after excess PolyHb was added to the Hp solution. Despite only observing fractional binding site saturation, dramatic increases in the MW and diameter of PolyHb after the Hp was added were still observed. This indicates that Hp is likely capable of binding higher order (MW>64 kDa) PolyHb molecules. Thus, Hp likely has a role in compartmentalizing 0, 1^{st}, and 2^{nd} order PolyHbs in the circulation.

The O₂ affinity of the low MW PolyHb used in this example (31.1±0.5 mm Hg) was similar to the O₂ affinity of Hb in human RBCs (P₅₀=29.3). While the P₅₀ was similar to Hb in RBCs, the lack of cooperativity (n=0.98±0.2) likely results in changes to O₂ offloading in the arterioles. The O₂ affinity was also comparable to previously produced PolyHbs (P₅₀=30.7±1.2 mm Hg) and PolyHeme (P⁵⁰˜29 mm Hg). Despite the fact that the average MW and diameter of the PolyHb produced for this example (AVG MW: 480 kDa), is significantly greater than previously produced commercial products [PolyHeme (64-400 kDa, AVG: 150 kDa) and HBOC-201 (69-500 kDa, AVG: 250 kDa)³], the PolyHbs produced in this example are comprised of approximately 50% low MW species (MW<500 kDa), which are known to be vasoactive than the higher MW species.

The relative changes in MAP and vessel tone compared to baseline in the saline treatment group were, on average, comparable to the changes observed after a top-load dose of HBOC-201. This is expected, given that the low MW PolyHb molecules used in this example had similar size distributions and O₂ affinity compared to HBOC-201. The relatively similar properties of the PolyHb produced make it a promising surrogate for previous commercial products.

Administration of apoHb alone had relatively little effect on maintaining hemodynamics after transfusion of PolyHb. In many cases, apoHb administration resulted in similar changes relative to baseline compared to the saline treatment group. This is expected given that the rate of heme release from PolyHbs is relatively low compared to stroma free Hb. More importantly, since apoHb exists as an αβ dimer (32 kDa), it was expected to be rapidly cleared through the kidneys within 5 minutes to an hour after administration. Moreover, heme binding to apoHb yields Hb which can cause similar effects as low MW PolyHb. However, the circulatory half-life would be increased if the apoHb were to bind plasma Hp. In addition, heme-binding to apoHb bound to Hp would neutralize the effects of free heme. Interestingly, the HR significantly decreased after PolyHb transfusion in the saline treatment group. This effect did not occur in the groups administered with the apoHb or apoHb-Hp solutions.

In contrast to both apoHb and saline, the apoHb-Hp complex was successful at maintaining baseline hemodynamics after transfusion of PolyHb. This indicates that Hp likely can mitigate the pressor effect of PolyHb by compartmentalizing low MW PolyHb within the vascular lumen. This mechanism is likely similar to the previously reported mechanism of cell-free Hb localization which preserved NO signaling. By increasing the MW of PolyHb via exchange of apoHb in the apoHb-Hp complexes with PolyHb, and thus eliminating the presence of small PolyHb species, translocation of PolyHb may be effectively stopped.

Previous studies have attempted to counteract the pressor effect of HBOCs via coadministration of adenosine or nitroglycerin. However, these materials require careful control of the dose to avoid hypotension. In contrast, independent administration of the apoHb-Hp solution did not significantly decrease MAP or alter HR compared baseline conditions. Unlike methods that target NO or endothelin, apoHb-Hp scavenging directly targets low MW stroma free Hb species. By targeting the Hb species directly, there is no need to balance a pressor response.

In conclusion, the results of this example indicate that the apoHb-Hp complex is a promising biomaterial that may make HBOCs safer for clinical application via mitigation of the pressor effect.

### Example 7. Enhanced Photodynamic Therapy Using the Apohemoglobin-Haptoglobin Complex as a Carrier of Aluminum Phthalocyanine

Photodynamic therapy (PDT) has been shown to effectively treat cancer by producing cytotoxic reactive oxygen species (ROS) via excitation of photosensitizers (PS). However, most PS lack tumor cell specificity, possess poor aqueous solubility, and cause systemic photosensitivity. Removing heme from hemoglobin (Hb) yields an apoprotein called apohemoglobin (apoHb) with a vacant heme-binding pocket that can efficiently bind to hydrophobic molecules such as PS. In this example, the PS aluminum phthalocyanine (Al-PC) was bound to the apoHb-haptoglobin (apoHb-Hp) protein complex, forming an apoHb-Al—PC-Hp (APH) complex. The reaction of Al-PC with apoHb prevented Al-PC aggregation in aqueous solution, retaining the characteristic spectral properties of Al-PC. The stability of apoHb-Al-PC was enhanced via binding with Hp to form the APH complex which allowed for repeated Al-PC additions to maximize Al-PC encapsulation. The final APH product had 65% of the active heme-binding sites of apoHb bound to Al-PC and hydrodynamic diameter of 18 nm, that could potentially reduce extravasation of the molecule through the blood vessel wall and prevent kidney accumulation of Al-PC. Furthermore, more than 80% of APH's absorbance spectra was retained when incubated for over a day in plasma at 37° C. Heme displacement assays confirmed Al-PC was bound within the heme-binding pocket of apoHb and binding specificity was demonstrated by ineffective Al-PC binding to human serum albumin, Hp, or Hb. In vitro studies confirmed enhanced singlet oxygen generation of APH over Al-PC in aqueous solution and demonstrated effective PDT on human and murine cancer cells. Taken together, this example provides a method to produce APH for enhanced PDT via improved PS solubility and potential targeted therapy via uptake by CD163+ macrophages and monocytes in the tumor (i.e. tumor associated macrophages). Moreover, this scalable method for site-specific encapsulation of Al-PC into apoHb and apoHb-Hp may be used for other hydrophobic therapeutic agents.

Introduction

Photodynamic therapy (PDT) is a treatment modality that generates reactive oxygen species (ROS) to induce localized cell death by either apoptosis or necrosis. PDT generates ROS by exciting photosensitizer (PS) molecules, in which the irradiated PS stimulates an electron to an unstable excited state. The energy from this electron can be transferred to an organic molecule (type I) or directly to molecular oxygen (type II). The former creates radicals which react with oxygen to form ROS, while the later directly forms singlet oxygen (¹O₂), a specific form of ROS. Even though both mechanisms lead to cell death, the type of damage varies. It has been shown that type I leads to cell death via necrosis, while type II leads to apoptosis. In addition to inducing tumor cell death, PDT has been shown to disrupt the tumor vasculature, and induces an anti-tumor immune response (potentially capable of preventing metastasis). PDT is a minimally invasive technique that has greatly expanded its potential biomedical applications with the development of fiber-based light delivery systems. Moreover, not only has PDT already shown great potential against cancer, but the combination of PDT with other cancer treatment modalities such as surgery or radiotherapy has shown synergistic effects with no cross-resistance. Further, PDT can be used in non-cancerous diseases such as age-related macular degeneration and as an antimicrobial agent.

For successful PDT treatment, potent PS molecules are required. First generation PS's exhibited low ROS production and prolonged photosensitivity, limiting their clinical use. Thus, researchers began developing new PS's for PDT. Phthalocyanines (PCs) are a low cost, second generation PS with high rate of ROS production and improved tissue penetration. PC's are stable and easily synthesized with low toxicity. Additionally, metalized-PCs can induce ROS generation via an alternative pathway by catalyzing the Fenton and Haber-Weiss reactions; as well as, significantly reducing levels of the antioxidant glutathione. One promising PS of this class is aluminum-PC (Al-PC) which has already shown promise as a PDT agent in previous studies and is clinically approved for use in Russia for PDT of stomach, lung, skin, lip, esophagus, oral cavity, tongue, and breast cancers as well as age-related macular degeneration. However, the Russian Al-PC comes in a mixture of sulphonated Al-PC which, although it improves aqueous solubility, has been shown to decrease photodynamic activity.

Even though new potent PS molecules have been developed, treatment has mainly relied on the unspecific tendency of particles to accumulate in tumor cells (attributed to the leaky and tortuous blood vessels of tumors). Furthermore, current applications of PDT are limited by the PS's low aqueous solubility and tendency to aggregate. Thus, even though there has been progress in the field, there is a need for more efficient delivery methods. Some examples of improved delivery include binding to albumin or low-density lipoprotein conjugates, targeting moieties (steroids, peptides, antibodies), liposomal encapsulation and nanocarriers. However, these mechanisms still lack tumor targeting, can lose PS via transfer to serum particles, or have complicated and expensive development. These issues lead to systemic and prolonged photosensitivity in patients, restricting the application of PDT. Moreover, new proposed delivery systems have been able to transport large quantities of PS molecules, but the PS molecules aggregated within the transport vehicle as indicated by the low absorbance of the species.

A promising candidate for transport and delivery of PS molecules is apohemoglobin (apoHb). Prosthetic heme groups are tightly bound inside the hydrophobic heme-binding pocket of hemoglobin (Hb) that can be removed to yield apoHb. The vacant heme-binding pocket of apoHb facilitates binding of hydrophobic molecules to the apoprotein, thus creating an aqueous drug delivery vehicle for hydrophobic molecules. For example, in Hb, apoHb binds to heme, enhancing heme's aqueous solubility and preventing its aggregation so that oxygen can be bound to the heme groups and transported via the circulatory system. Moreover, the use of apoHb for drug delivery has the benefit that the globin structure of apoHb resembles that of Hb and should exhibit little to no immune response. In addition to potentially serving as a drug carrier, the apoprotein clears via the same pathway as cell-free Hb in which CD163+ macrophages and monocytes uptake the protein after haptoglobin (Hp) binding. Due to this specific uptake mechanism, Hb and apoHb-based drug delivery systems have already been used to target CD163+ macrophages and monocytes. Based on these characteristics, apoHb is a promising yet simple targeted drug delivery vehicle for small hydrophobic molecules such as PS molecules.

In this example, the solubility and delivery challenges associated with PS molecules were addressed by using apoHb bound to Hp as a carrier of Al-PC. ApoHb was complexed with the plasma protein Hp to improve the apoprotein's stability in vivo and increase binding of Al-PC to the protein. Hp is the primary scavenger of cell-free Hb and delivering it to CD163+ macrophages and monocytes for recycling into CO, iron and biliverdin. Binding of apoHb-Al-PC to Hp also leads to the formation of a large apoHb-Al—PC-Hp (APH) complex which can prevent extravascular extravasation of apoHb-Al-PC. Furthermore, ex vivo Hp binding to apoHb-Al-PC does not require depletion of endogenous plasma Hp for targeting to CD163+ macrophages and monocytes. Targeting to the CD163 receptor is a promising approach for cancer therapy, since CD163+ macrophages (M2 phenotype) are classified as tumor promoting and are found in high concentration in various tumors.

Targeting tumor associated macrophages (TAMs) is highly relevant due to their role in tumor promotion, metastasis, and immunotherapy. Moreover, TAMs are one of the most abundant cell types in tumors, accounting for approximately 50% of the tumor mass and consisting primarily of the M2 phenotype. Further, TAM targeting has been shown to serve as a drug depot for slow drug release into the tumor microenvironment. Finally, through selective targeting of M2 TAM, the APH complex could help identify the primary and metastatic tumors, potentially serving as theranostic molecule. Thus, the apoHb-Hp complex could serve as a targeted delivery system of therapeutic and imaging agents such as Al-PC for cancer theranostics.

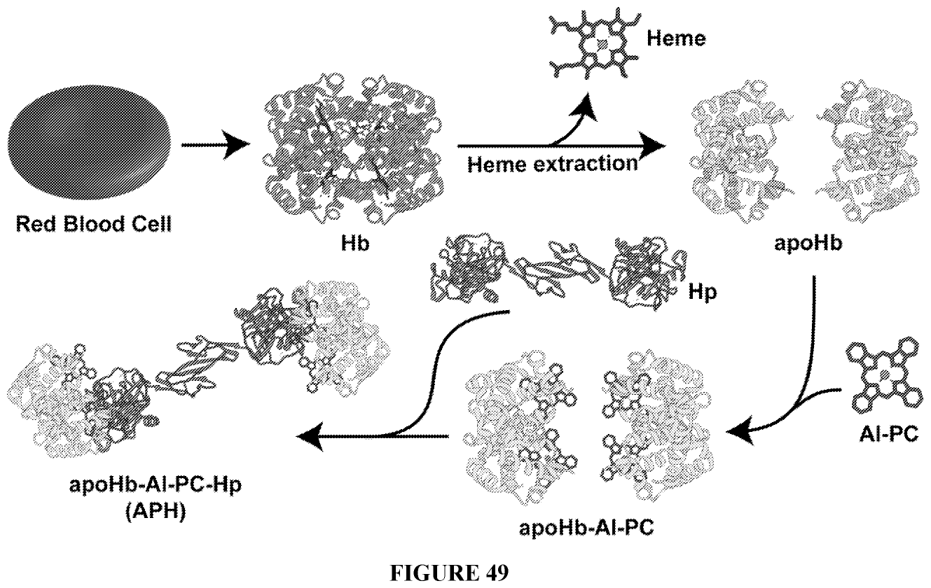

A general diagram of the process used to synthesize APH is shown in FIG. 49.

Materials and Methods

Materials. Sodium phosphate dibasic, sodium phosphate monobasic, aluminum phthalocyanine chloride, and hemin chloride were purchased from Sigma Aldrich (St. Louis, Mo.). Potassium cyanide, hydrochloric acid, sodium chloride, potassium chloride, 0.22 μm nylon syringe filters, and dialysis tubing (pore size: 6-8 kDa) were purchased from Fisher Scientific (Pittsburgh, Pa.). 0.2 μm Millex-GP PES syringe filters were purchased from Merck Millipore (Billerica, Mass.). Ethanol was purchased from Decon Labs (King of Prussia, Pa.). A KrosFlo® Research II tangential flow filtration (TFF) system and hollow fiber (HF) filter modules were obtained from Spectrum Laboratories (Rancho Dominguez, Calif.). Expired units of human red blood cells (RBCs) and thawed human plasma were generously donated by the Transfusion Service in the Wexner Medical Center at The Ohio State University (Columbus, Ohio). Human fraction IV paste was purchased from Seraplex, Inc (Pasadena, Calif.).

Fluorescence and Absorbance Spectroscopy. Ultraviolet-visible spectrometry was performed in quartz cuvettes using a HP 8452A diode array spectrophotometer (Hewlett Packard, CA) and fluorescence spectrometry was measured using a PTI fluorometer (Horiba Scientific, NJ).

Hb Preparation. Human Hb was purified via tangential flow filtration (TFF) as described previously. Hb concentration was determined spectrophotometrically via the Winterbourn equations.

ApoHb Production. Apohemoglobin was produced via TFF as described previously.

Hp Purification. Hp was purified from human Cohn fraction IV. The final protein solution was composed of a mixture of Hp2-1 and Hp2-2 Hp polymers with an average MW of 400-500 kDa.

Hb/ApoHb Binding Capacity of Hp. The binding capacity of Hp to Hb and apoHb was determined using size exclusion high performance liquid chromatography (HPLC-SEC). The change in the area under the curve of free Hb or apoHb chromatograms when Hp was added to the sample was used to quantify the amount of Hb or apoHb bound to Hp.

Total Protein Assay. The total protein concentration of apoHb in solution was measured using the molar extinction coefficient of apoHb at 280 nm (12.7 mM⁻¹cm⁻¹).

ApoHb Activity Assay. The activity of the vacant hydrophobic heme-binding pocket of apoHb was determined via the dicyanohemin (DCNh) incorporation assay as previously developed. The extinction coefficients of DCNh and rHbCN used were 85 mM⁻¹ cm⁻¹ and 114 mM⁻¹ cm⁻¹ at 420 nm, respectively.

Preparation of Al-PC solutions. Stock Al-PC samples were freshly made by dissolving 1 mg of Al-PC in 2 mL of 100% EtOH. The stock solution was then diluted in EtOH prior to addition into apoHb or apoHb-Hp samples in phosphate buffered saline (PBS, 0.1 M, pH 7.4). All samples with Al-PC were kept at 4° C. and wrapped in aluminum foil. The concentration of Al-PC solutions was determined via the extinction coefficient of 294 mM⁻¹ cm⁻¹ at 672 nm in EtOH.

Optimization of EtOH Addition to ApoHb. The stock Al-PC solution was diluted 100× into 100% EtOH. The diluted stock Al-PC was then added in increasing volumes to a constant volume of apoHb in PBS at a concentration of approximately 23 μM. Volume ratios of diluted stock Al-PC in EtOH to apoHb in PBS ranged from 1:40 to 1:4.44.

Stability of ApoHb-Al-PC. The stability of apoHb-Al-PC was assessed by adding 1 mL of Al-PC solution at ˜8 μM to 10 mL of apoHb in PBS at a concentration of ˜23 μM. The sample was then dialyzed against PBS using 6-8 kDa cellulose dialysis membranes to remove any residual EtOH. Dialysis was performed over three days with daily exchange of the PBS buffer. The UV-visible absorbance spectra of samples were measured before and after dialysis.

Optimization of Al-PC Loading to ApoHb Varying dilutions of the stock Al-PC solution were prepared in EtOH (2.78, 7.19, 9.86, 13.2, 16.6, 18.6, 20.1 μM). 200 μL of these solutions were individually added to 2 mL of apoHb in PBS at a concentration of approximately −23 μM. The absorbance spectra of the resulting mixtures were then measured via UV-visible spectrophotometry.

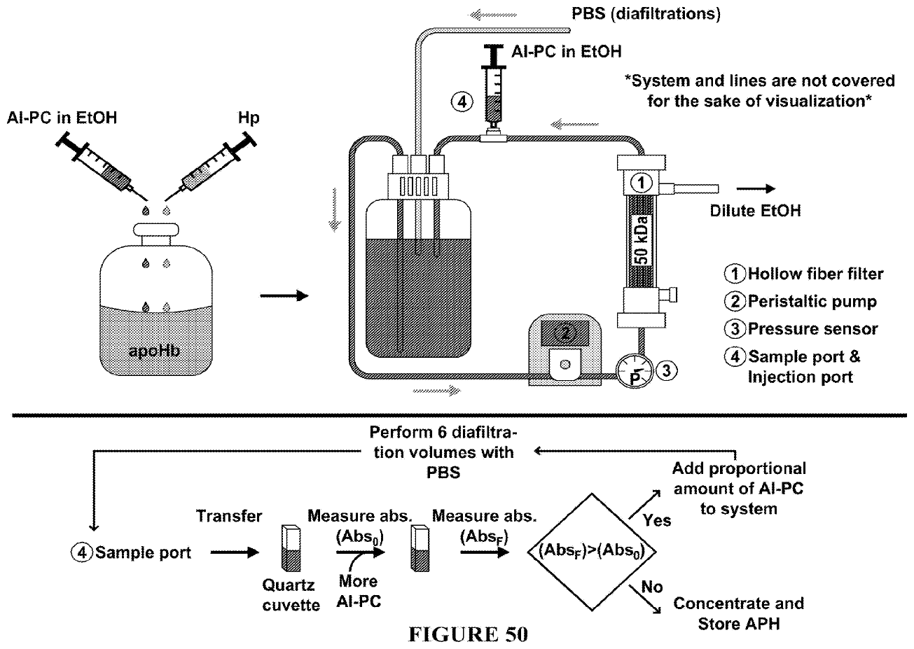

Production of APH Complex. APH was produced via a repeated addition protocol. Starting with 200 mL of apoHb at a concentration of ˜23 μM, 20 mL of Al-PC in EtOH at ˜18 μM was added dropwise into the apoHb solution under constant stirring. Hp was then added at equimolar concentration to bind to apoHb-Al-PC. The mixture was left to react for 1 hour at 4° C. EtOH was removed from the solution via TFF by diafiltration with PBS over a 50 kDa modified polyethersulfone (mPES) hydrophilic TFF filter with inlet pressure maintained at about 6.5 psig. The sample container and process tubing were wrapped with aluminum foil to prevent Al-PC exposure to light. After 6 diafiltration volumes, 2 mL of the product was sampled, and its absorbance spectra was measured. Then, 200 μL of the 18 μM Al-PC stock solution was added to the 2 ml sample from the system to test for further Al-PC binding to apoHb-Hp. If the absorbance peak at 680 nm increased, an additional 20 mL of the 18 μM Al-PC was added to the system via an injection port. This process was repeated until the absorbance at 680 nm did not increase with further Al-PC addition. The sample was then concentrated to approximately 2 AU/cm at 680 nm (˜1.3 mg/mL of APH) and stored at −80° C. with containers wrapped in aluminum foil. A general setup of the process along with a flowchart for the repeated Al-PC additions is shown in FIG. 50.

APH Stability at 37° C. The stability of the apoHb-Al—PC-Hp complex (APH) was assessed in PBS and thawed human plasma at 37° C. APH was diluted four times into sealed cuvettes that were incubated at 37° C. and covered in aluminum foil for a total of 48 h. The absorbance spectra of samples were periodically measured to determine the loss of Al-PC from APH over time. The fraction retained was determined by the ratio of the absorbance at 680 nm compared to the initial absorbance at 680 nm. The effects of incubation time, plasma exposure and the interaction of incubation time and plasma exposure were determined via a mixed-model approach using JMP Pro 13.

Specificity of Al-PC Binding to ApoHb. Binding of Al-PC to human serum albumin (HSA), Hb, reconstituted Hb (rHb), and Hp was tested to assess if binding to Al-PC was specific to the vacant heme-binding pocket of apoHb. Binding studies were performed via the same protocol described in Optimization of EtOH Addition to ApoHb. The concentrations of Hp, HSA, Hb and rHb in solution were 1.0 mg/mL, 0.35 mg/ml (and 3.5 mg/ml), 0.35 mg/mL, and 0.33 mg/mL respectively.

Heme-Albumin Mediated Displacement of Al-PC from APH. A fixed APH concentration (˜0.4 mg/mL) was mixed with increasing concentrations (0, 1.5 and 7.5 μM) of heme-albumin. Samples were kept isolated from light and incubated at both 4° C. and 37° C. Absorbance spectra of the mixtures were periodically measured to determine the displacement of Al-PC from apoHb in the APH complex via a decrease in the 680 nm peak, and transfer of heme from heme-albumin to apoHb in the APH complex via an increase in the 405 nm peak. Pseudo-first order rate constants were determined, which were then adjusted for the concentration of heme-albumin to determine the second order rate constant of Al-PC displacement by heme.

Dynamic Light Scattering. The hydrodynamic diameter of samples was determined using a BI-200SM goniometer (Brookhaven Instruments, Holtsville, N.Y.) at an angle of 90° and wavelength of 637 nm. Samples ware diluted to ˜1 mg/mL concentration in PBS (0.1 M, pH 7.4). The hydrodynamic diameter was calculated from experimental data by using the non-linear least squares (NNLS) algorithm in the instrument software.

HPLC Size Exclusion Chromatography. Samples were separated on an analytical Acclaim SEC-1000 (4.6×300 mm) column (Thermo Fisher Scientific, Waltham, Mass.) attached to a Dionex UltiMate 3000 system (Thermo Fisher Scientific, Waltham, Mass.). The mobile phase consisted of 50 mM potassium phosphate, pH 7.4. The flow rate and UV-visible spectral detection was controlled on Chromeleon 7 software with detection set to λ=280 nm and λ=680 nm to detect protein and Al-PC elution respectively at a flow rate 0.35 mL/min.

Singlet Oxygen Detection. Singlet oxygen was detected using the probe 1,3-diphenylisobenzofuran (DPBF), which reacts irreversibly with singlet oxygen. The DPBF optical density at λ=414 nm decreases directly proportional to the fraction of DPBF reacting with singlet oxygen. Briefly, 10 μL of 4 mM DPBF in ethanol were added to 500 μL aliquots of the test samples. Then, each mixture was irradiated with a laser (λ=670 nm; energy density of 0.75 J/cm²). Absorption of the samples was measured at 414 nm before and after irradiation, and the change in optical densities before and after irradiation was used to quantify singlet oxygen production.

Cell Culture. Cancerous (4T1 [murine] and MDA-MB-231 [human]) and noncancerous (NOR-10 [murine] and MCF-10A [human]) cell lines were purchased from American Type Culture Collection (ATCC, Manassas, Va.). All cells were cultured in DMEM, and supplemented with 10% (v:v) fetal bovine serum and 1% (v:v) antibiotic solution (100 IU/mL penicillin and 100 mg/mL streptomycin). All cells were maintained at 37° C. in 5% CO₂ and in a humidified atmosphere.

Cell Uptake. ApoHb-Al—PC-Hp uptake by cells was monitored over time by measuring cell fluorescence during the incubation period. Briefly, 4T1, MDA-MB-231, NOR-10, and MCF-10A cells were cultured for 24 hours at a density of 2×10³ cells/well. Later, the culture medium was replaced with 200 μL of apoHb-Al—PC-Hp in culture medium at a concentration of 1 μM equivalent concentration of Al-PC and incubated at 37° C. over time. At each time point, the culture medium with apoHb-Al—PC-Hp was removed and stored, the cells were washed twice with PBS, and fresh culture medium was loaded into the well. Next, the fluorescence was read at an excitation of λ=350 nm and emission of λ=680 nm, and the fresh culture media was switched back for culture medium containing apoHb-Al—PC-Hp.

PDT. For PDT, 4T1, MDA-MB-231, NOR-10, and MCF-10A cells were cultured for 24 hours at a density of 5×10³ on coverslips. Next, cells were exposed to Al-PC, apoHb-Al-PC-Hp, and apoHb-Hp in culture medium at different concentrations. Later, the cells were irradiated with a laser (λ=670 nm) at energy densities between 0.0 J/cm2 and 4 J/cm2 for 15 minutes in the dark.

Cell Viability. Cell viability was assessed using the MTT assay, which is converted by the mitochondria of viable cells to an insoluble purple precipitate. Briefly, after receiving their treatment, cells were washed with PBS twice and then incubated with 0.5 mg/mL of MTT in culture medium for 2 hours. Then, the MTT solution was washed away, and the purple precipitate was extracted from the cells with 150 μL of dimethyl sulfoxide (DMSO). The absorption was then measured at λ=595 nm using a spectrophotometer (Spectramax; Molecular Devices).

Detection of Fragmented DNA. DNA fragmentation was measured using propidium iodide (PI), which binds to DNA and allows for cell DNA content to be measured via by flow cytometry. Briefly, 4T1, MDA-MB-231, NOR-10, and MCF-10A cells were cultured at a density of 5×10⁵ cells/well for 24 hours. Then, the culture medium was removed, cells were washed twice with PBS, and replaced with medium containing apoHb-Al—PC-Hp at a equivalent concentration of 0.165 μM of Al-PC for 30 min and exposed to laser irradiation (λ=670 nm) applied at 0, 0.5, or 1 J/cm². After, cells were washed twice with PBS, and the cells were cultured for 24 hours in fresh culture media. The next day, all cells were harvested, centrifuged, and resuspended in PBS. The cell suspension was incubated with PI at 20 μg/mL for 20 minutes in the dark and samples were measured using a FACS scan flow cytometer (BD Biosciences, San Jose, Calif.).

Cell Death Analysis. Cell death by apoptosis or necrosis was analyzed after apoHb-Al—PC-Hp treatment using acridine orange/ethidium bromide double staining. The 4T1, MDA-MB-231, NOR-10, and MCF-10A cells were cultured at a density of 1×10⁴ cells/well for 24 hours. Then, the culture medium was removed, cells were washed twice with PBS, and replaced with medium containing apoHb-Al—PC-Hp at an equivalent concentration of 0.165 μM of Al-PC for 30 min and exposed to laser irradiation (λ=670 nm) applied at 0, 0.5, or 1 J/cm². After, cells were washed twice with PBS, and the cells were cultured for 24 hours in fresh culture media. The next day, all cells were harvested, centrifuged, and resuspended in PBS. The cell suspension was incubated with 50 μg/mL acridine orange and 50 μg/mL ethidium bromide in the dark. Lastly, the cell suspension was evaluated via fluorescence microscopy to measure the percentage of cells experiencing apoptosis or necrosis.

Results and Discussion

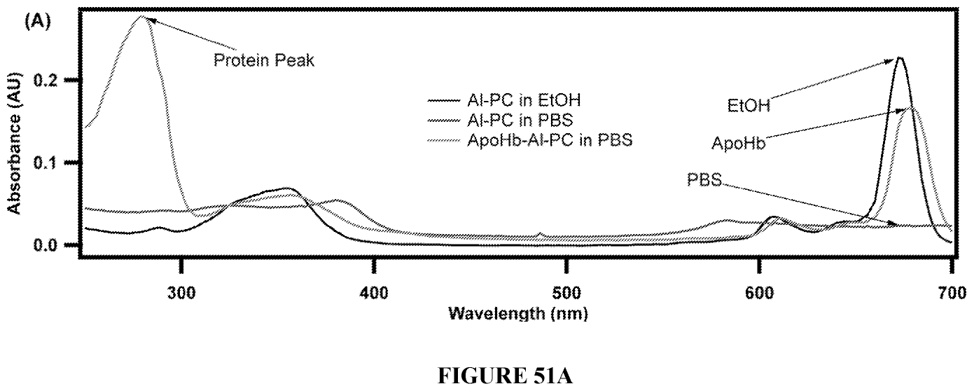

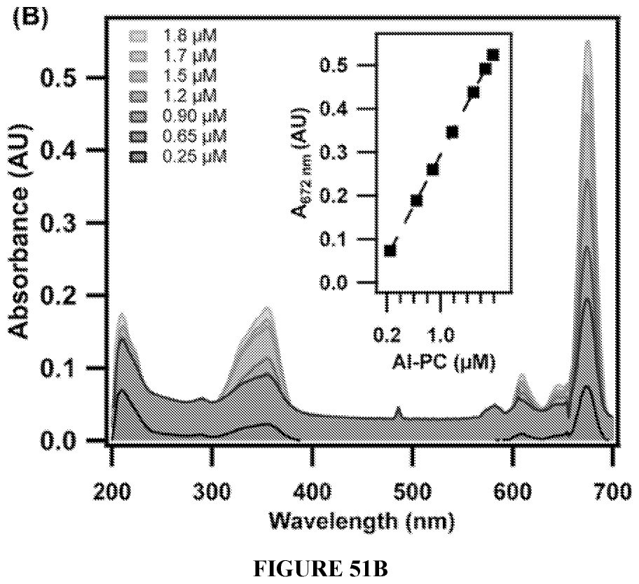

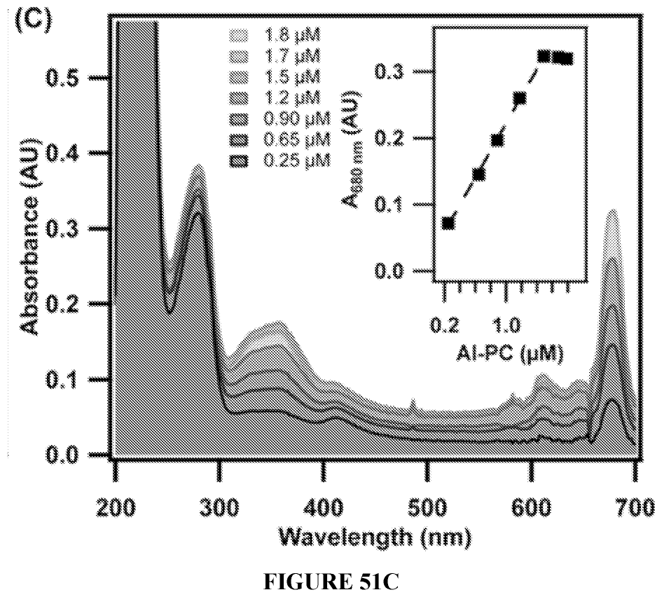

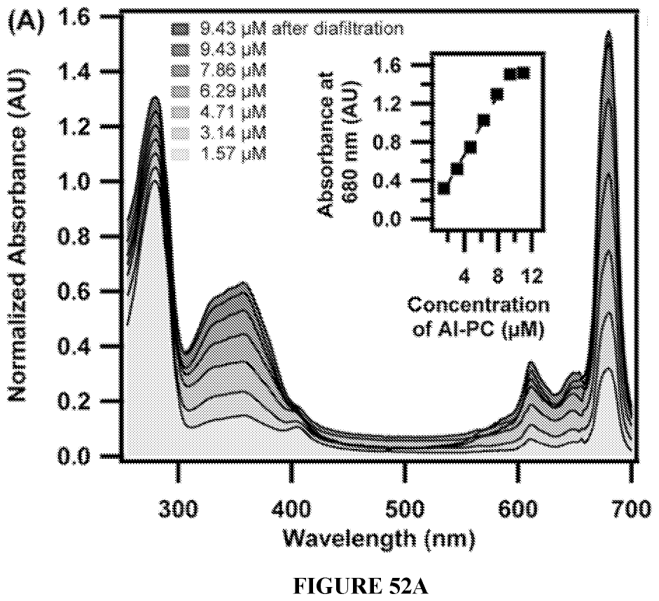

Analysis of Al-PC binding to apoHb. Al-PC in EtOH was prepared as described above and added to PBS or apoHb in PBS. The results of this analysis are shown in FIG. 51A.

As shown in FIG. 51A Al-PC in EtOH exhibited a sharp peak at 672 nm. However, Al-PC lost this characteristic absorbance peak when mixed into PBS. It has been reported that this loss of absorbance corresponds to a dimerized or aggregated state of Al-PC. Loss of this peak hinders the ability of Al-PC to be used in PDT as optimal photoexcitation relies on strong red wavelength absorbance. The longer wavelengths (red) allow for deeper tissue penetration of light and avoids absorbance by the skin pigment, melanin, which absorbs around 500 nm. Although Al-PC had no absorbance peak at 672 nm in PBS, when apoHb was present in the PBS solution, the characteristic Al-PC absorbance peak remained with a slight redshift to 680 nm. This indicated that Al-PC bound to apoHb and prevented Al-PC aggregation by binding to the PS in its monomeric state and forming an apoHb-Al-PC complex. This observation encouraged development of an apoHb-based PC drug delivery vehicle, since previous protein-based drug delivery vehicles have failed to retain the characteristic sharp absorbance peak of PC in aqueous solution.

In this study, EtOH was used to dissolve Al-PC and maintain it in its monomeric form. However, EtOH can denature and precipitate proteins, thus, the maximum volume ratio of EtOH:apoHb solution was determined which maximized binding of Al-PC to apoHb but prevented protein precipitation. The results are shown in FIG. 59. Based on this analysis, an upper limit of 1:10 volume ratio (EtOH:apoHb solution) was determined, where further addition of EtOH lead to protein precipitation. As the system described here may be used for other therapeutic agents, the organic solvent employed to solubilize the hydrophobic drug molecule may be switched and will require re-optimization. With the optimal volume of EtOH identified that avoids apoHb precipitation, the maximum Al-PC concentration in EtOH was assessed by reacting increasing concentrations of Al-PC in EtOH with apoHb in PBS. The resulting mixtures of apoHb and Al-PC were then analyzed via UV-visible spectrometry and compared with the absorbance of Al-PC in pure EtOH. The results from this experiment are shown in FIGS. 51B and 51C.

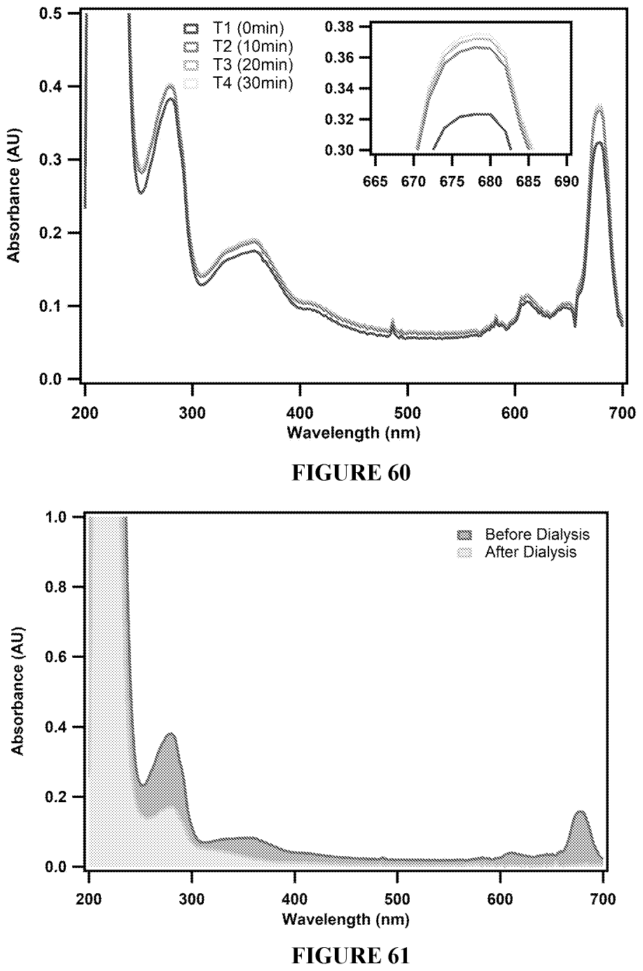

Based on FIG. 51B, there was a linear increase in the absorbance at 672 nm for Al-PC added to EtOH indicating that the absorbance of Al-PC in EtOH followed Beer Lambert's Law. The same linear trend was observed for Al-PC added to apoHb up to an A1-PC concentration of 1.5 μM, indicating that Al-PC bound to apoHb and maintained its monomeric structure in an aqueous environment. However, in FIG. 51C, a sharp inflection point was noted, indicating that 1.5 μM was the highest Al-PC concentration that could effectively be added to the apoHb (when restricted by the 1:10 volume ratio previously determined). Yet, the concentration of Al-PC in aqueous solution was an order of magnitude lower than the concentration of available heme-binding pockets (˜18 μM) in solution. Thus, Al—PC may have partially aggregated while in EtOH (prior to dilution into apoHb in PBS) or the high Al-PC concentration added to PBS favored aggregate formation over binding to apoHb. It is important to note that when binding other therapeutic agents to apoHb, the same protocol may be followed to determine the maximum concentration of the drug in the organic solvent to be reacted with apoHb in aqueous solution. It was also determined that the reaction between Al-PC and apoHb, although almost complete within a few seconds, had slight absorbance increases at 680 nm for up to 30 minutes at room temperature (FIG. 60).

After Al-PC bound to apoHb, fresh apoHb-Al-PC was dialyzed against PBS at 4° C. over the course of three days with buffer exchanges at 24 hour intervals. This process was expected to gradually remove EtOH from the product. Unfortunately, the product after dialysis had visible precipitates. Therefore, the resulting solution was sterile filtered through a 0.2 μm syringe filter and the UV-visible spectra was measured (FIG. 61). Based on the decrease in absorbance at 280 nm (characteristic of proteins) of the product post dialysis, there was an overall loss in protein due to aggregation (FIG. 61). Moreover, the disappearance of absorbance at 680 nm indicated that the Al-PC either precipitated with the apoHb (i.e. as an apoHb-Al-PC complex) or unbound from apoHb and aggregated in solution. This instability was attributed to the Al-PC in the sample and not an intrinsic instability of apoHb given that the apoHb used for this study has been shown to be stable at 4° C. for months. Based on these results, it was determined that apoHb-Al-PC was not stable in aqueous solution, hindering its potential biomedical applications.

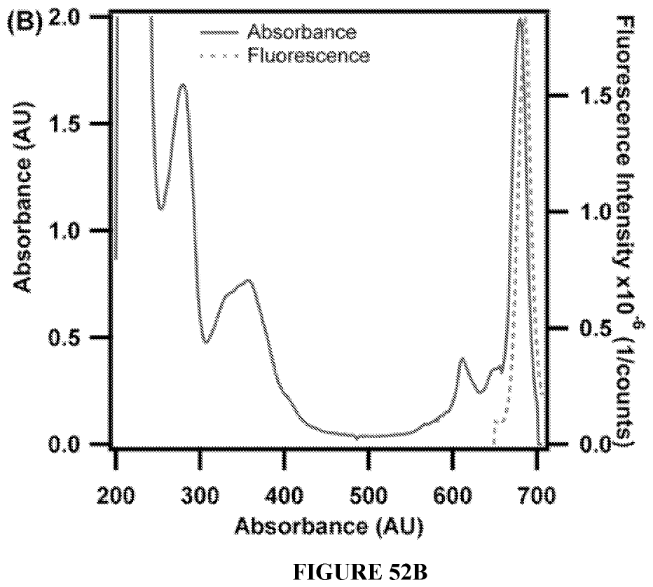

Synthesis of apoHb-Al-PC-Hp. Given apoHb-Al-PC's instability in aqueous solution, Hp was added at an equimolar ratio to apoHb-Al-PC to stabilize it. Hp is known to bind to apoHb, stabilizing the apoprotein, and to prevent heme release from Hb. Thus, it was hypothesized that formation of the apoHb-Al-PC-Hp complex (APH) would yield a stable protein species. Preliminary results demonstrated that APH was more stable than apoHb-Al-PC, retaining Al-PC absorbance after employing the same dialysis protocol that was used with apoHb-Al-PC. Based on these findings, an Al-PC binding protocol was developed to synthesize APH at large scales as described previously (FIG. 50). The normalized absorbance spectra of the sample after each Al-PC addition as well as the final sample after being subject to diafiltration is shown in FIG. 52A.

Even though, Al-PC concentration and EtOH volume addition were maximized, the results from FIG. 52A showed that further Al-PC binding was possible to the APH complex after ethanol removal in the diafiltration process. This observation was not surprising since, as noted earlier, the molar ratio of active heme-binding sites to Al-PC added in each addition was ˜10:1. As shown by the inflection point on the absorbance at 680 nm graph, a total of six Al-PC additions were necessary to maximize the binding of Al-PC to APH. The final APH product was concentrated to ˜27 μM (based on the apoHb concentration), reaching an absorbance of ˜2.0 AU/cm at 680 nm (˜1.3 mg/mL of total APH with ˜13 μM of Al-PC). The resulting APH product absorbance and fluorescence spectra are shown in FIG. 52B.

From the absorbance and fluorescence spectra of APH (FIG. 52B), the complex retained the absorbance and fluorescence spectra of native Al-PC in EtOH. Retention of these spectral properties should enable photoexcitation of Al-PC. Unfortunately, aggregation of Al-PC in aqueous solution leads to loss of both the absorption and fluorescence peaks of Al-PC at ˜700 nm that are present when Al-PC is in EtOH. However, as mentioned previously, PDT relies on the high absorption band above 500 nm to prevent interference from melanin that is present in skin tissue. Most studies indicate that the optimal range of absorption is within the 620-800 nm region, making APH well within the range for optimal PDT.

Assuming that all the added Al-PC bound to apoHb, the total amount of Al-PC added corresponded to an occupation of ˜65% of the active heme-binding sites of apoHb. Interestingly, when considering the total protein content of apoHb and not just the active heme-binding sites, ˜50% of apoHb bound to Al-PC. This may indicate that Al-PC preferentially bound to either the α or β chains of apoHb and did not depend on interactions the proximal histidine in apoHb. Such asymmetric binding could be explained by the higher heme affinity of α chains which may indicate they were the only chains capable of Al-PC incorporation. Moreover, it has been shown that the α chain has a lower helical content than the 0 chain when bound to Hp. A lower helical content (less structure) may allow a chains to more easily bind to heme-like (macrocycles) molecules such as Al-PC. Furthermore, there was no appreciable loss of protein as the 280 nm peak did not decrease during processing (there was a slight increase upon Al-PC addition).

The current repeated Al-PC binding protocol (FIG. 50) may be optimized in future studies to run continuously in a TFF system. Briefly, this could be accomplished by reacting apoHb with Hp followed by a slow infusion of Al-PC into the TFF system while performing continuous diafiltration with PBS buffer. With a larger flowrate of PBS into the system versus Al-PC, the reaction vessel containing the apoHb and Hp proteins could be maintained at a low EtOH concentration. Thus, this protocol would simplify the overall process to form APH. Moreover, it could be adapted for binding of other therapeutic agents to apoHb and apoHb-Hp.

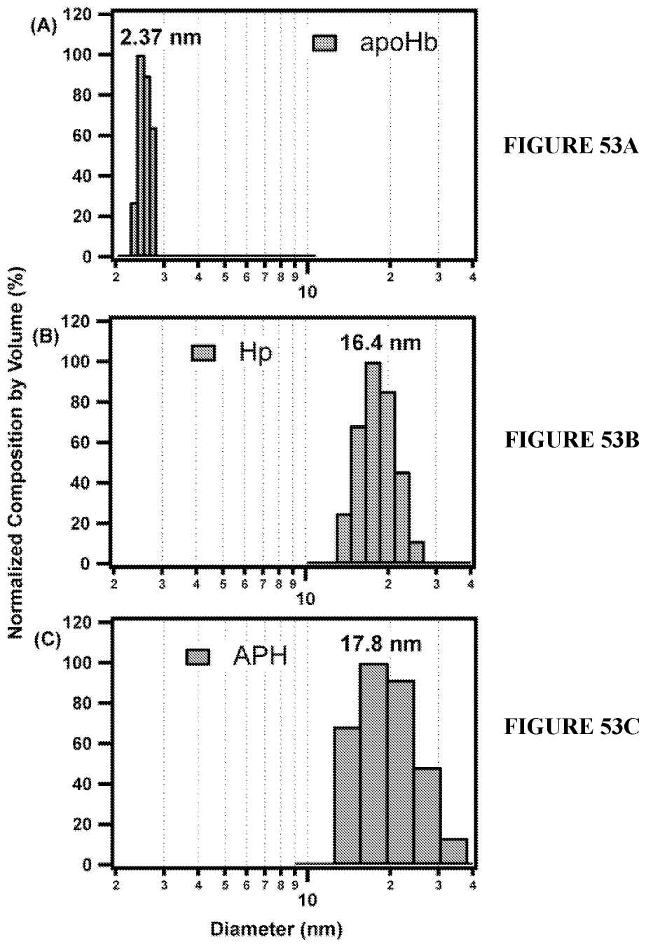

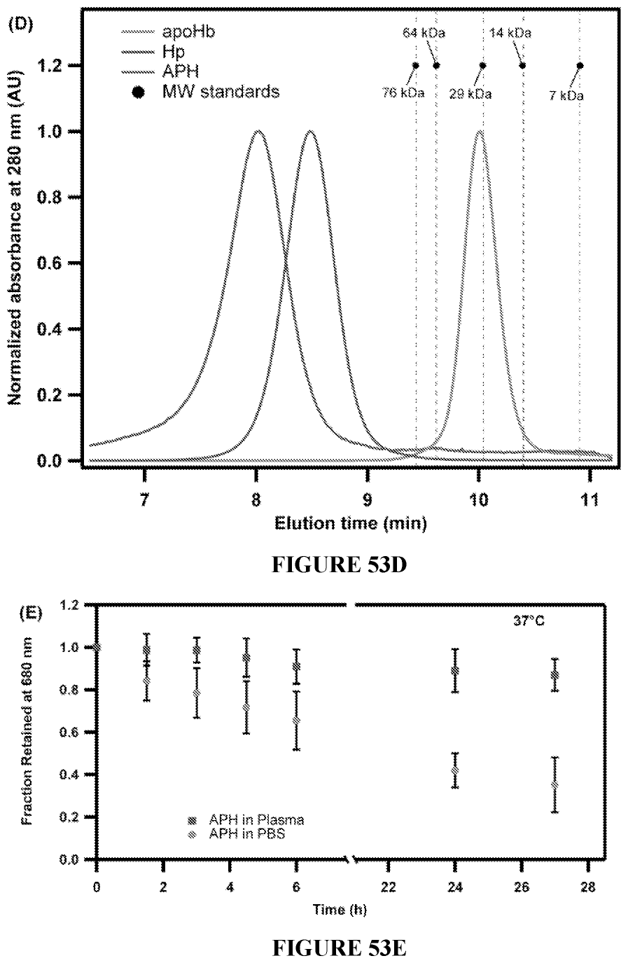

Size and stability of APH. The hydrodynamic size, apparent MW and stability of APH were characterized. These results are shown in FIG. 53.

Hemoglobin (Hb) tetramers (α₂β₂) have a diameter of ˜5.5 nm. Upon heme removal from Hb, the apoprotein forms αβ dimers, yielding an expected diameter of ˜2.7 nm. Thus, the DLS measurement of 2.4 nm was similar to the expected size of apoHb. From the DLS measurement, Hp had a hydrodynamic diameter of ˜16 nm with a wide distribution ranging from 12 nm to 26 nm. The wide distribution was expected, since the mixture of Hp2-2 and Hp2-1 polymers used in this study can be composed of different numbers of subunits ranging from 200-900 kDa (αβ Hp dimers). Moreover, after binding apoHb-Al-PC to Hp, a detectable increase of ˜2 nm in diameter was observed which corresponded to apoHb-Al-PC's attachment to Hp. Similar to DLS analysis, the HPLC-SEC chromatograms showed an increase in the apparent MW (i.e. lower elution time) of APH (˜700 kDa) compared to Hp (˜400 kDa). This increase in MW indicated that more than one apoHb was attaching to each Hp binding site which was expected due to the polymeric nature of the Hp species used. Moreover, the final MW of APH was more than an order of magnitude larger than apoHb (MW ˜32 kDa). Thus, in addition to stabilizing the apoHb-Al-PC complex, Hp binding increased the size of the final product (i.e. APH). The larger size of APH can potentially minimize the extravasation of apoHb-Al-PC through blood vessel walls or kidneys which are common pathological traits of cell-free Hb which has a similar size compared to apoHb.

To test the stability of Al-PC in APH, the complex was incubated in PBS and human plasma at 37° C. The decrease in absorbance at 680 nm over time was measured to model the loss of Al-PC activity from the APH complex. The results of this experiment are shown in FIG. 53E. APH was more stable in plasma than in PBS, retaining more than 80% of the initial Al-PC absorbance in plasma versus ˜40% in PBS over a one-day period. Moreover, statistical analysis showed that, in addition to significant effects of incubation time and plasma exposure, there was a significant (α<0.05) effect of the interaction of incubation time and plasma exposure on spectral retention. This confirmed that the effect of incubation time differed between the PBS and plasma conditions. Loss of Al-PC activity likely occurs through photodegradation or removal of Al-PC from APH. Thus, it was not expected that plasma could affect Al-PC stability. Yet, this high plasma stability enhances the potential in vivo stability, and in turn efficiency, of APH for PDT.

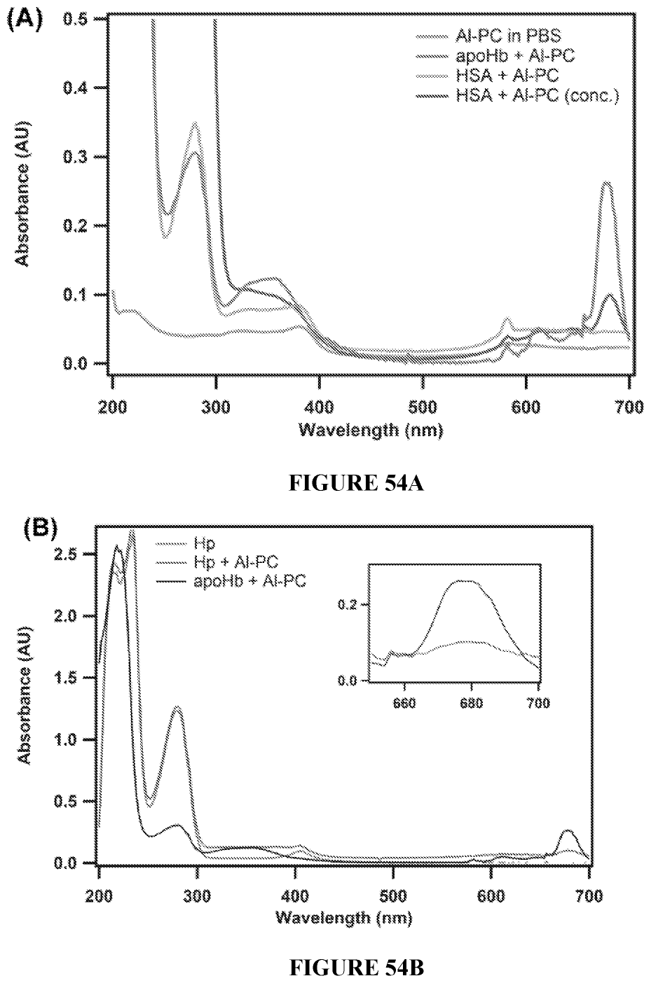

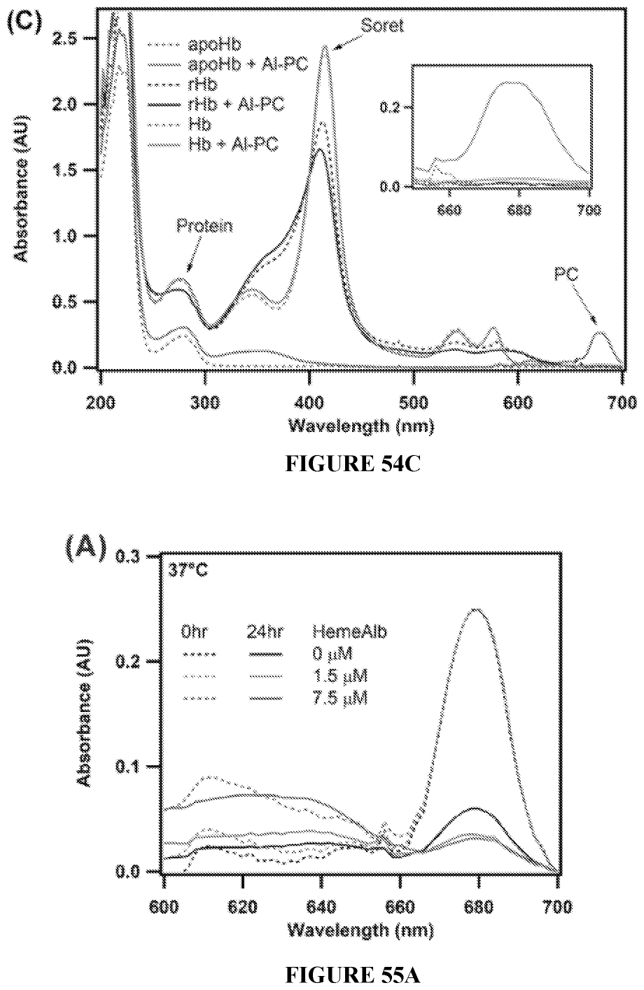

Specificity and Retention of Al-PC Binding. Given that heme-binding activity did not correlate to Al-PC binding, the binding reaction of Al-PC was performed with Hp, human serum albumin (HSA), Hb, and reconstituted Hb (rHb; prepared by reinstating heme in apoHb) to assess if Al-PC would bind to proteins without the vacant heme-binding pocket of apoHb. This analysis is shown in FIG. 54.

Addition of Al-PC to Hp resulted in a slight increase in the absorbance at 680 nm. This binding event can be attributed to the residual apoHb present in the Hp sample as Hp has been shown previously to not have heme-binding pockets. As shown FIG. 54B, the Hp sample had residual Hb (small absorbance peak at 404 nm). This residual Hb could have lost its' heme during the processing of human plasma with acidic ethanol required for production of Cohn Fraction IV. Interestingly HSA had almost no binding to Al-PC at a similar mass concentration to apoHb. HSA is known to bind to a variety of ligands (including heme) and has been used previously for transport of PS molecules. Yet, HSA has only one heme-binding site per 66.5 kDa while apoHb has two heme-binding sites per 32 kDa αβ dimer. Thus, HSA has ˜4× fewer heme-binding sites than apoHb at the same mass. This lower binding capacity of HSA could explain the lack of Al-PC binding. Yet, even when the concentration of HSA was increased by an order of magnitude (i.e. 2.5× more heme-binding sites than the apoHb experiment), there was only a slight increase in the absorbance at 680 nm, demonstrating that apoHb was more capable of binding Al-PC than HSA. This was a promising observation as lack of Al-PC binding to HSA may indicate that a very small amounts of Al-PC would transfer to other serum proteins from apoHb-Al-PC. This was verified by the plasma stability study in FIG. 53E. Moreover, there was no appreciable binding of Al-PC to either Hb or rHb, indicating that Al-PC was binding to the vacant heme-binding pocket of apoHb and not to other hydrophobic regions on the Hb globin chains.

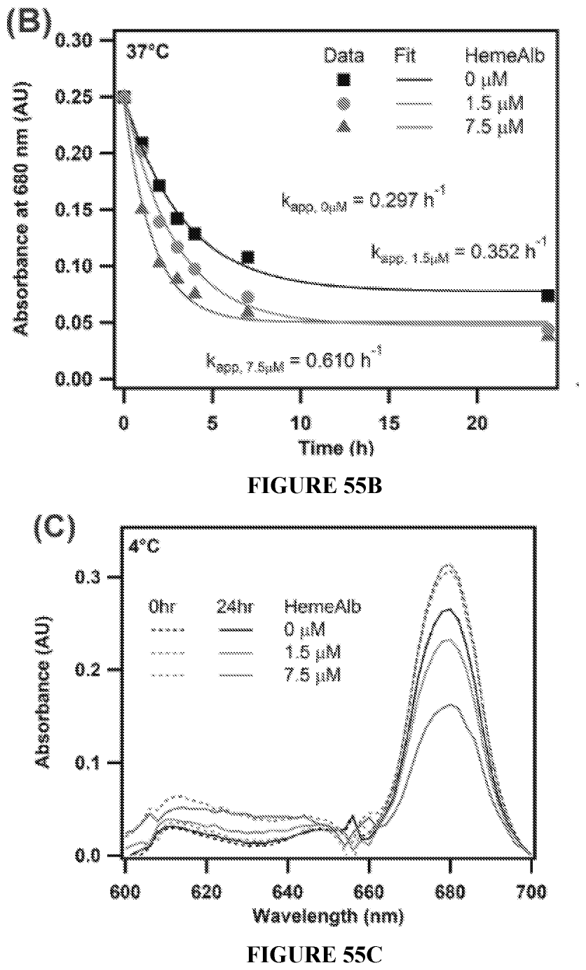

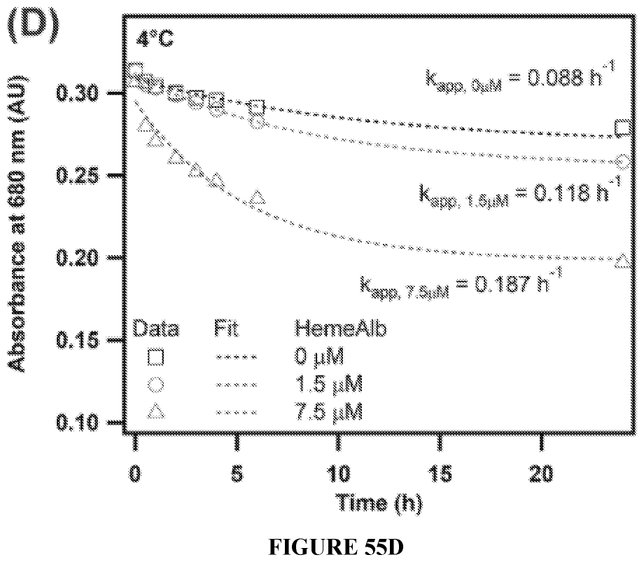

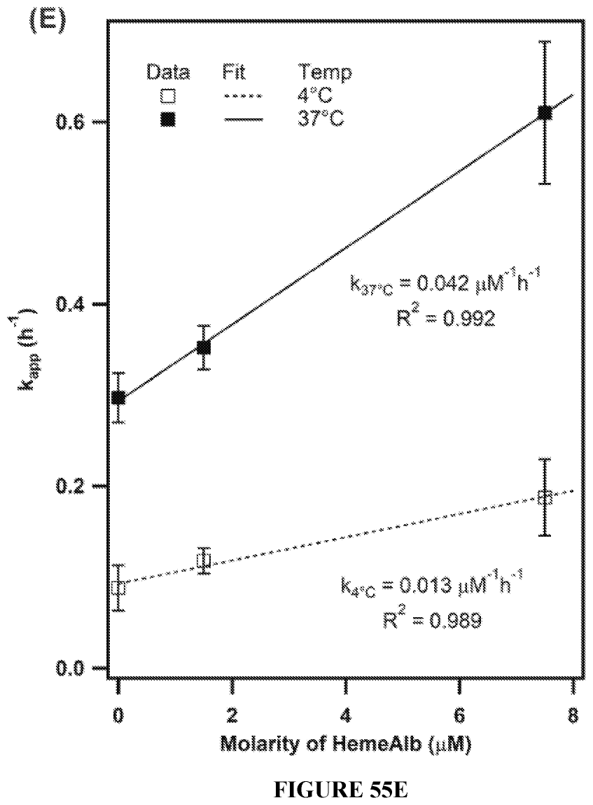

To further assess the binding site of Al-PC in apoHb, the APH complex was mixed with heme-albumin at concentrations of 1.5 and 7.5 μM at 37° C. and 4° C. Heme-albumin acted as a carrier for heme that could potentially displace of Al-PC from the heme-binding pocket of apoHb. The change in absorbance of APH at 680 nm was monitored to assess Al-PC loss from APH and the results are shown in FIG. 55.

Based on the results of FIGS. 55A and 55B, there was a noticeably greater loss of Al-PC absorbance at 37° C. compared to 4° C. at all heme-albumin concentrations tested. Assessment of the pseudo-first order kinetic rate constants for the loss of absorbance at 680 nm confirmed this observation as the rate constants were three times higher at 37° C. than at 4° C. (FIGS. 55B and 55D). Moreover, when second order rate constants were determined, it was noticed that there was an effect of heme-albumin concentration on the replacement of Al-PC by heme leading to faster loss of absorbance at 680 nm (FIG. 55E). As shown previously in FIG. 53E, there was loss of Al-PC in PBS without heme-albumin. This is shown by the non-zero rate constants when the concentration of heme-albumin was zero. Notably, the second order rate constant for heme-albumin mediated displacement of Al-PC from the APH complex was ˜4× higher at 37° C. than at 4° C. demonstrating the lower stability of the APH complex under in vivo conditions. Heme transfer from heme-albumin to APH also demonstrated the selective binding of Al-PC in the vacant heme-binding pocket of apoHb as heme displaced Al-PC in the apoHb contained within the APH complex. Therefore, Al-PC binding to apoHb had a lower affinity than heme, as heme bound to albumin could displace bound Al-PC from APH.

Assessment of the Al-PC binding site in apoHb via heme-displacement is an important assay for characterizing apoHb complexes. For example, improper analysis of the binding sites has led to a previous study concluding that there were five binding sites per apoHb tetramer (α₂β₂). In the aforementioned study, apoHb was bound to a Zn-substituted porphyrin moiety. Unfortunately, the previous study employed an incorrect extinction coefficient of apoHb (value ˜1.25× larger) for the apoHb protein, and did not quantify its heme-binding capacity or performed heme-displacement assays. Based on this error, the authors underestimated the quantity of protein in their samples leading to an overestimation of the heme-binding capacity. Furthermore, although Zn-substituted Hb could diminish heme-oxygenase-1 (HO-1) antioxidant activity in tumor cells, the porphyrin provides similar photodynamic characteristics to that of native Hb (absorbance peak between 400-500 nm), and therefore was not an optimal photosensitizer for PDT (the PS should have an absorbance peak at wavelengths above 500 nm to prevent the skin pigment melanin from absorbing light).

Other studies have aimed to improve PDT by binding PS molecules to apomyoglobin. Unfortunately, these studies did not analyze the binding activity of the apoprotein prior to its use or confirm the heme-binding pocket specificity via heme displacement measurements. Moreover, although apomyoglobin-PS led to enhanced photodynamic properties by preventing PS aggregation, the use of apomyoglobin-PS would likely lead to a significant loss of PS through the vascular wall and kidneys given the small MW of myoglobin. Thus, systemic administration of myoglobin-like compounds without addressing its inherently small MW could lead to systemic photosensitivity and toxicity.

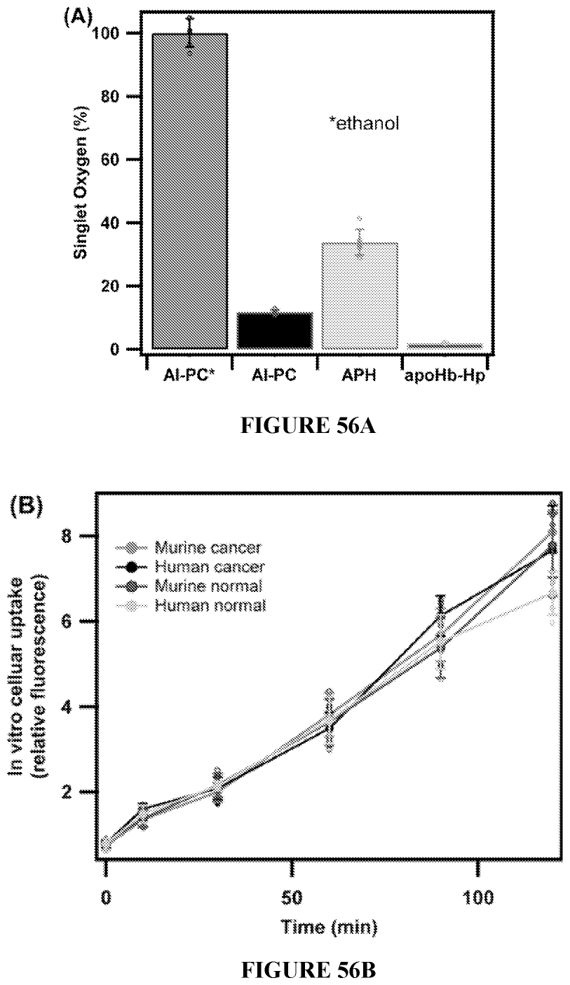

In vitro analysis of PDT potential of APH. To analyze the PDT potential of APH, singlet oxygen generation from Al-PC, APH and apoHb-Hp in aqueous solution was assessed and compared to Al-PC in EtOH. Moreover, to determine if APH could be up taken up by normal and human cancer cells, the amount of intracellular Al-PC was tracked after incubating APH with human and murine cancer cell lines. The results from these analyses are shown in FIG. 56.

As shown in FIG. 56A, APH was able to significantly retain more of singlet oxygen generating potential compared to pure Al-PC in aqueous solution. These results corroborated the idea that Al-PC binding to apoHb-Hp prevented Al-PC aggregation and suppression of PDT activity. Furthermore, as expected, irradiation of apoHb-Hp itself did not lead to any singlet oxygen generation. Moreover, as shown in the cellular uptake studies (FIG. 56B), Al-PC delivered via APH was continuously taken up by the various cancer cell lines over the two-hour period. These results did not show a difference in uptake between normal and cancerous cell lines. However, the results from FIG. 56 demonstrated that, although APH is a large complex, it can deliver Al-PC to cells and has enhanced singlet oxygen production over pure Al-PC in aqueous solution for intracellular PDT effects. Moreover, targeted drug delivery mediated through apoHb-Hp would not be expected to have enhanced uptake by tumor cells. The mechanism relies on uptake through CD163+ macrophages within the tumor microenvironment (i.e. TAM). This targeted cellular uptake has been extensively investigated in Hb clearance studies and Hb-based drug delivery systems.

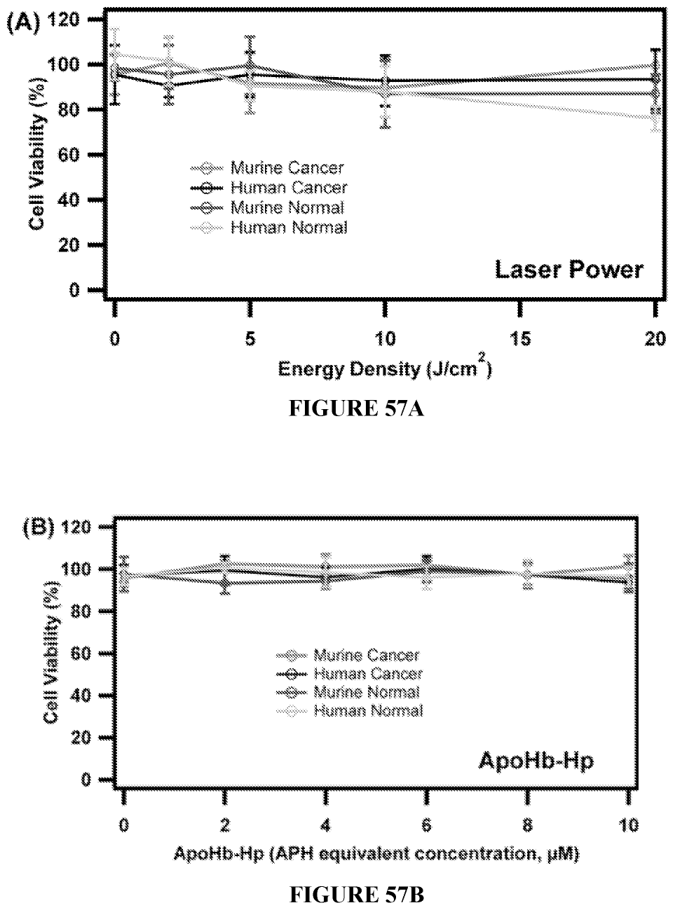

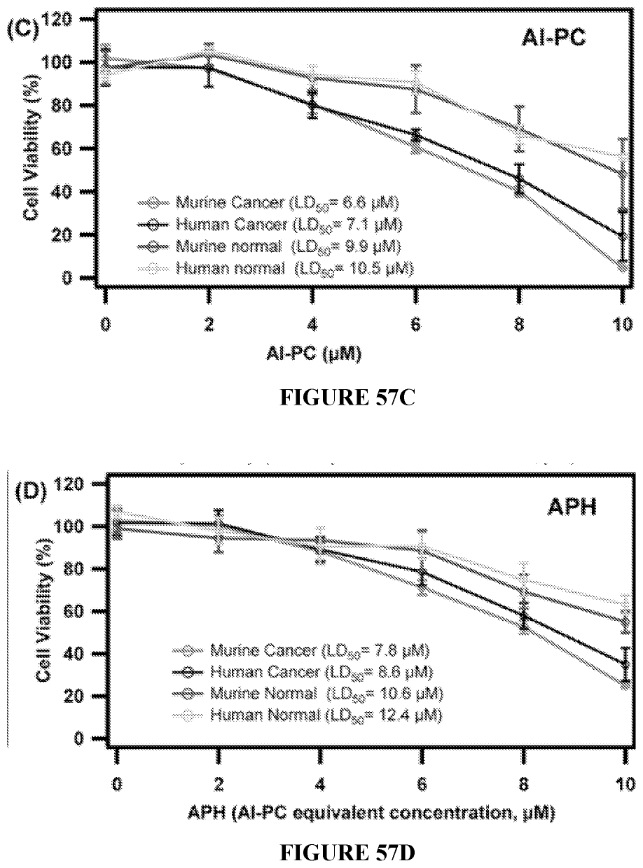

With the promising singlet oxygen generating potential of APH and cellular uptake of Al-PC via delivery from APH confirmed, the cytotoxicity of APH, Al-PC, and apoHb-Hp without laser irritation (dark toxicity) as well as the effect of laser irradiation alone were first analyzed. The results from these experiments are shown in FIG. 57.

As shown in FIGS. 57A and 57B, laser irradiation alone or apoHb-Hp incubation did not lead to cytotoxicity. This was expected as the cells should not have been light sensitive themselves nor should the apoHb-Hp complex lead to cell death. On the other hand, incubation of cells in the absence of irradiation with either Al-PC or APH (FIGS. 57C and 57D) lead to a dose-dependent cytotoxicity in the dark. The estimated lethal dose for 50% cell-death (LD₅₀) of Al-PC was 7 μM in cancerous cells and 10 μM in normal cells. APH lowered the toxicity, increasing the LD₅₀ to 8 μM in cancerous cells and 11 μM in normal cells. Moreover, APH was practically non-cytotoxic at concentrations lower than 2 μM of Al-PC equivalent, which provides a window of concentrations that could be used for PDT-mediated cell-death. Further, it was noticeable that both Al-PC and APH were more toxic to cancerous cell-lines compared to normal cells, indicating that non-irradiated treatment with Al-PC based molecules could preferentially kill cancerous cells.

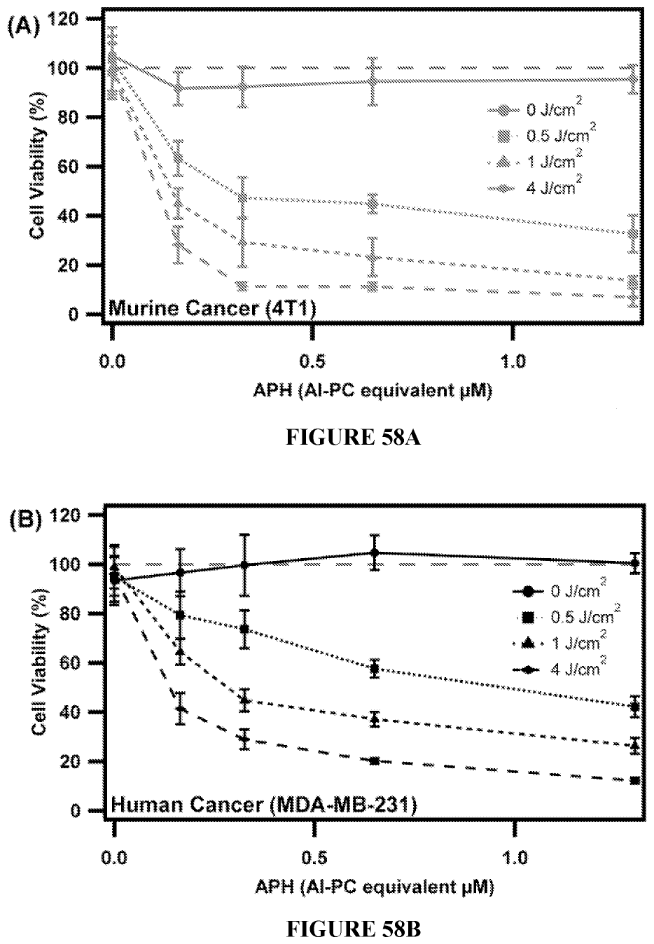

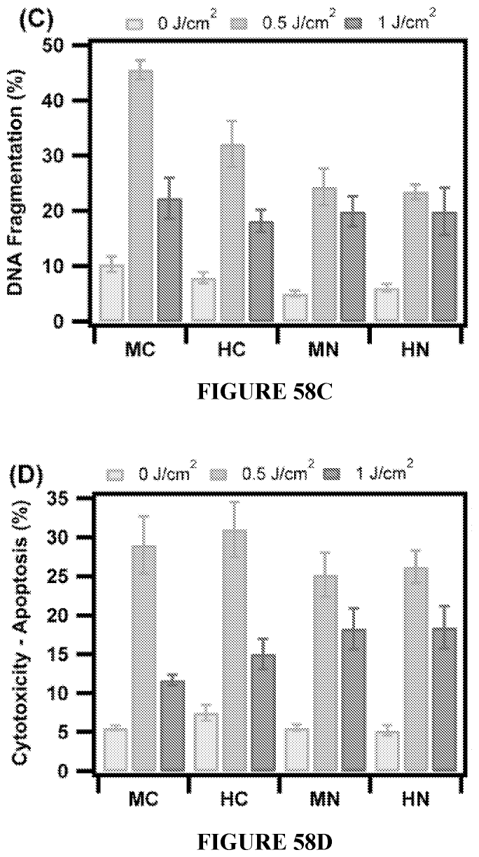

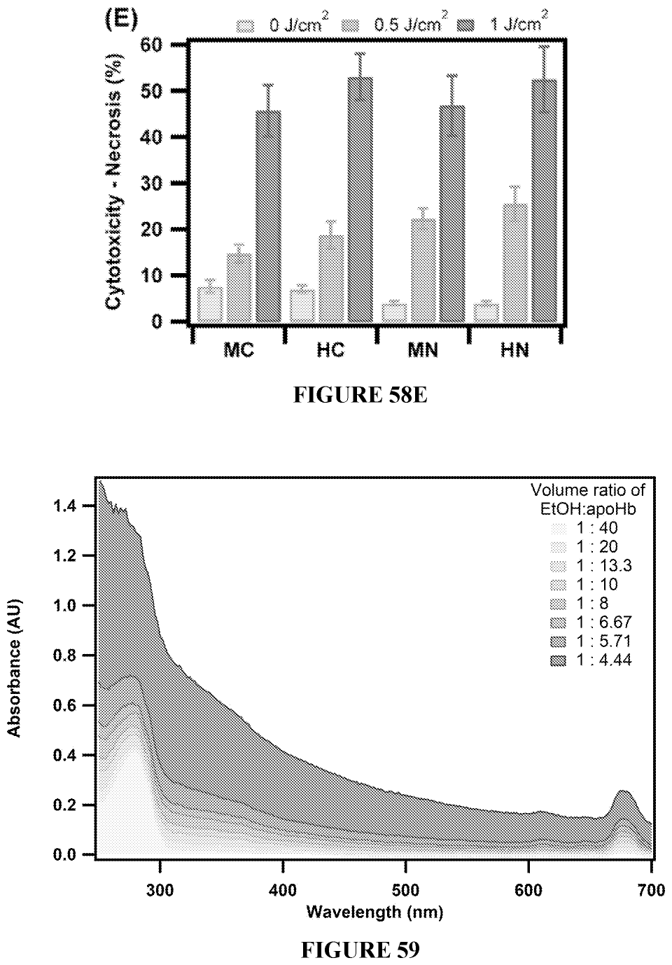

Based on the results from FIG. 57, PDT treatment of the cell-lines was performed with concentrations lower than 2 μM to restrict cell-death that was not induced by PDT. The results from PDT treatment on the murine and human cancer cells at various concentrations of APH and laser intensity is shown in FIGS. 58A and 58B. To further characterize the PDT cytotoxicity mediated by APH, DNA fragmentation levels, and markers of apoptosis or necrosis were assessed on cells incubated with 0.165 μM of APH at varying laser intensities and the results are presented in FIGS. 58C-58E.

The results from FIGS. 58A and 58B confirmed that APH was non-cytotoxic at concentrations lower than 2 μM with no irradiation. However, when APH treated cells were irradiated, cytotoxicity was noticeable even at the lowest concentration tested of 0.165 μM. This observation indicated that APH had a large therapeutic window, since PDT was effective even at concentrations one order of magnitude lower than the levels required for cytotoxicity in the absence or lase irradiation. Moreover, as the radiation intensity increased this led to more cell death as more radicals and singlet oxygen were produced. From the cell death characterization assays, two major observations could be made. First, at low energy densities, there was more apoptotic cell death as demonstrated by the increase in apoptosis markers and increased percentage of DNA fragmentation. On the other hand, at low energy densities, necrotic cell death was favored. This was expected as previous PDT studies have demonstrated that exposure of highly potent PS molecules to high energy densities favor necrotic cell death. Moreover, it was noticeable that at the 0.5 J/cm² laser density, the cancerous cell lines were more susceptible to apoptotic cell death. Based on these results, APH was confirmed as a potential PDT agent. Future studies will aim to analyze its effectiveness and toxicity in vivo.

Conclusion

The potential of PDT for cancer therapy has been known for more than 25 years with recent studies showing its success against various cancers with some therapies now in clinical trials. However, PS solubility and targeting are the main roadblocks limiting clinical application of PDT. In addition to the known benefits of PDT, recent developments of fiber-based interstitial, intravesical and endoscopic light delivery systems have expanded possible applications of PDT. Moreover, new and more potent PSs, such as Al-PCs, already exist, but efficient delivery and targeting mechanisms are needed to minimize systemic photosensitivity, prevent aggregation and increase aqueous solubility. Thus, there is a need to develop systems to deliver PSs to cancer cells, while maintaining photoactivity of the PS. In this example, it was demonstrated how to use apohemoglobin (apoHb) as an efficient carrier of Al-PC by forming the apoHb-Al—PC-Hp (APH) complex. The process developed here is scalable and easily translatable to other PSs or hydrophobic molecules. Further, binding of apoHb-Al-PC to Hp enhances the stability of the complex and provides a potential targeting and fast clearance mechanism via CD163+ macrophage uptake, which are tumor promoting M2 tumor associated macrophages highly expressed in various cancer types (such as rectal, pancreatic, lymphoma, oral squamous cell carcinoma, ovarian, hepatocellular carcinoma, prostate, lung, mesothelioma, brain, and thyroid). Targeted drug delivery could lower the overall systemic toxicity of PS molecules, and the fast clearance could prevent prolonged phototoxicity. Moreover, the large MW and size of APH should prevent extravasation into the tissue space and clearance via the kidneys. Finally, in vitro analysis confirmed APH could generate singlet oxygen and induce light-induced cytotoxicity with minimal dark toxicity. Taken together, this example details a photodynamic agent for PDT. Moreover, the encapsulation protocol described herein may be made continuous and other therapeutic agents may be encapsulated into the apoHb-Hp complex.

### Example 8. Preparation of Apohemoglobin-Haptoglobin-(Therapeutic/Diagnostic) Complexes

The apoHb-Hp-(therapeutic/diagnostic) complex can be made by reacting the apoHb-(therapeutic/diagnostic) conjugate with Hp, or reacting the Hp-(therapeutic/diagnostic) conjugate with apoHb, or conjugating the therapeutic/diagnostic molecule to the apoHb-Hp complex.

Materials and Methods

Apohemoglobin-Haptoglobin Complex Preparation. The apoHb-Hp complex was made by reacting apoHb with Hp. The high binding affinity drives the reaction for complex formation. A Hp solution with a hemoglobin binding capacity (HbBC) of 49.7 mg/mL was mixed with an apoHb solution with 30.3 mg/mL of active apoHb at a 1:2 volume ratio (10 μl of Hp and 20 μl of apoHb in 1 mL of 50 mM phosphate buffer, pH 7.4). The resultant mixture (apoHb-Hp complex+excess apoHb) was separated on a size exclusion chromatography (SEC) column for analysis. Large molecular weight Hp (Hp2-2 and Hp2-1) was mixed with apoHb with a molecular weight of about 31 kDa (dimeric apoHb) and separated on an analytical Acclaim SEC-1000 (4.6×300 mm) column (Thermo Fisher Scientific, Waltham, Mass.) attached to a Dionex UltiMate 3000 system (Thermo Fisher Scientific, Waltham, Mass.). The mobile phase consisted of 50 mM potassium phosphate, pH 7.4. The flow rate and UV-visible spectral detection was controlled using Chromeleon 7 software with detection set to λ=280 nm to detect protein elution at a flow rate 0.35 mL/min. The percent change in the area under the curve between a pure apoHb solution and a mixture of apoHb-Hp with excess apoHb was used to determine the percentage of apoHb that was bound to Hp. This percentage was compared to the mass of pure apoHb loaded to determine the Hp binding capacity of apoHb. This value was compared to the HbBC of the Hp sample.

Apohemoglobin-Haptoglobin-(Therapeutic/Diagnostic) Complex Preparation. To form an apoHb-Hp-(therapeutic/diagnostic) complex, either pure apoHb or apoHb-Hp was mixed with a therapeutic/diagnostic containing solutions. The therapeutic/diagnostic molecule in this case binds to the vacant heme binding pocket of apoHb.

Diagnostic/therapeutic solutions tested here consisted of either Mn-porphyrin IX chloride (Mn-IX) or aluminum phthalocyanine (Al-PC) chloride. The Mn-IX solution was made by dissolving 2.5 mg of Mn-IX in 0.5 mL of 0.1 M NaOH. For Al-PC, 1 mg of Al-PC was dissolved in 1 mL of pure EtOH, then 50 μL of the saturated solution was diluted into 1 mL of pure EtOH (the second dilution ensured that the Al-PC is monomeric in solution). The apoHb-Hp complexes were formed by mixing 20 μL of apoHb (30.3 mg/mL of active apoHb) and 10 μL of Hp (HbBC of 49.7 mg/mL) in PB (50 mM, pH 7.4) for the Mn-IX trials, and 10 μL of apoHb (30.3 mg/mL of active apoHb) and 10 μL of Hp (HbBC of 49.7 mg/mL) in PB (50 mM, pH 7.4) for the Al-PC trials. From the stock diagnostic/therapeutic solutions, either 2 μl of the stock Mn-IX or 100 μl of the Al-PC solutions was added to the protein samples. The resultant mixtures had their absorbance spectras measured via UV-visible spectroscopy and were separated via size exclusion chromatography (SEC) using an Acclaim SEC-1000 (4.6×300 mm) column (Thermo Fisher Scientific, Waltham, Mass.) attached to a Dionex UltiMate 3000 system (Thermo Fisher Scientific, Waltham, Mass.). The mobile phase consisted of 50 mM potassium phosphate, pH 7.4. The flow rate and UV-visible spectral detection was controlled using Chromeleon 7 software with detection set to λ=280 nm (protein), λ=405 nm (residual heme of apoHb and Hp), λ=486 nm (Soret peak of Mn-IX-apoHb), and λ=680 nm (peak of Al-PC-apoHb) at a flow rate 0.35 mL/min.

Results

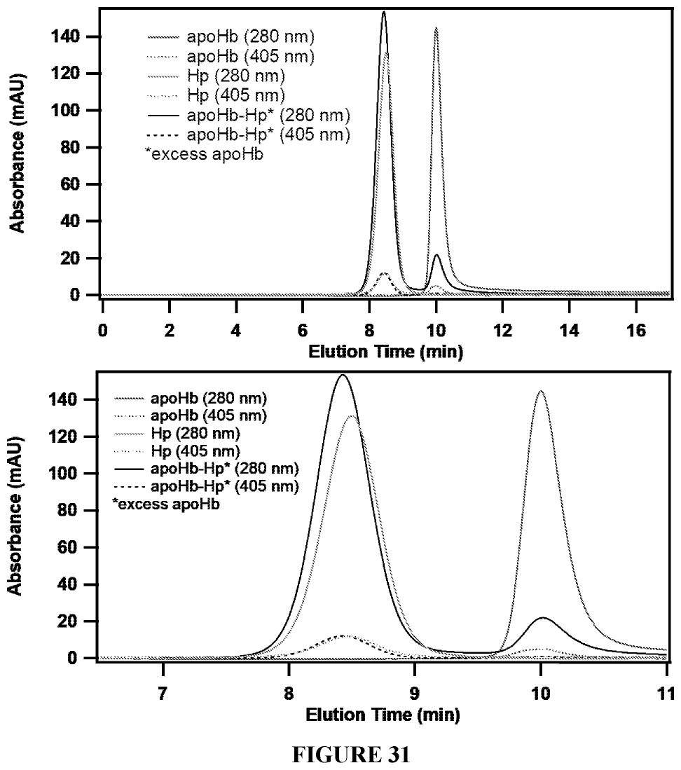

ApoHb-Hp complex formation is shown in FIG. 31. As seen in FIG. 31, upon addition of Hp, the amount of free apoHb which elutes at about 10 min was reduced compared to a pure apoHb solution. Similar to previously shown data, the HbBC displayed less than 2% difference compared to Hp binding capacity of active apoHb (based on the change in the area under the curve of the pure apoHb solution compared to the apoHb-Hp complex with excess apoHb). Both apoHb and Hp had minimal Soret absorbance (400-500 nm region) compared to the protein peak absorbance (280 nm), but it was possible to note the elution of these proteins based on the absorbance at 405 nm. Furthermore, the apoHb-Hp complex eluted at a slightly lower time due to the increase in the MW of the complex compared to pure apoHb or Hp.

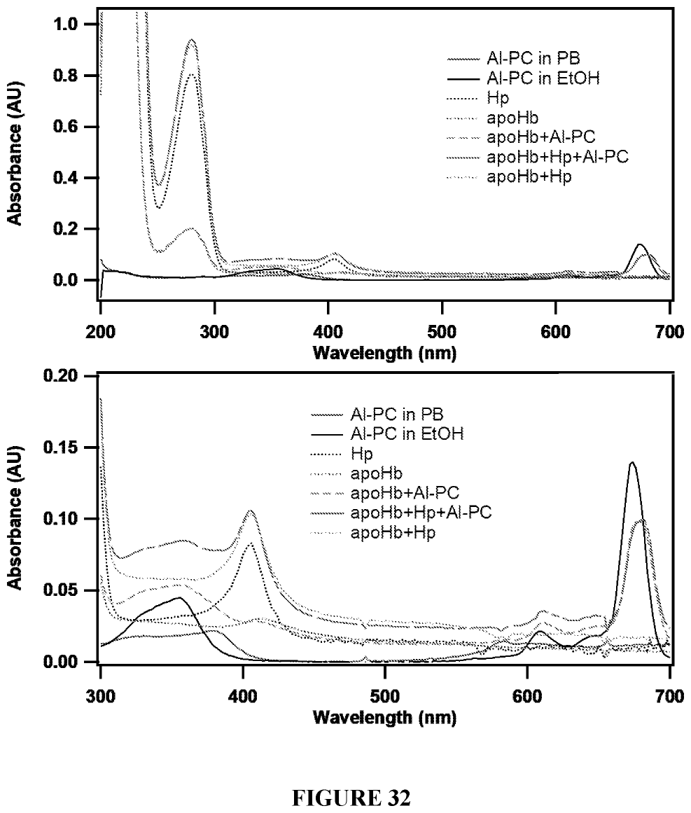

Prior to HPLC-SEC, the absorbance spectra of each of the samples tested was measured using UV-visible spectroscopy. The results using the Al-PC solution are shown in FIG. 32. Based on the absorbance, Al-PC formed aggregates in solution, leading to practically no absorbance in the phosphate buffer (PB) solution. Yet, when dissolved in pure ethanol (EtOH), a sharp peak at 678 nm appears. This peak provides the important photodynamic characteristics for use of Al-PC in photodynamic therapy. When Al-PC is bound to either apoHb or apoHb-Hp, the peak at 678 nm corresponding to free Al-PC shifts to 680 nm.

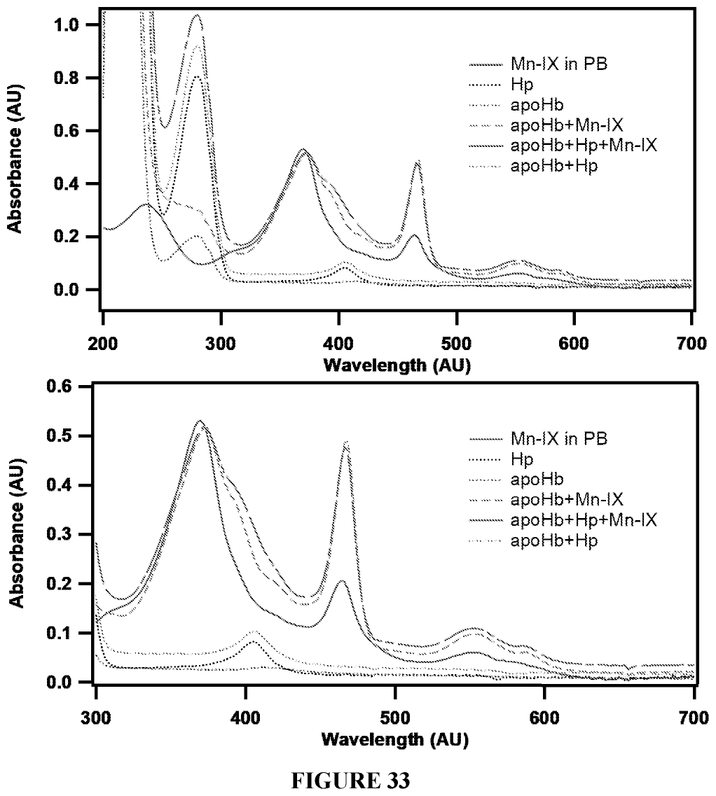

The same absorbance spectral analysis was done with the samples made from the Mn-IX solution. These results are shown in FIG. 33. Similar to Al-PC, without apoHb or apoHb-Hp, there was a lower absorbance of the test molecule. Addition of Mn-IX to either apoHb or apoHb-Hp solutions lead to an increase in absorbance of a sharp peak at 468 nm.

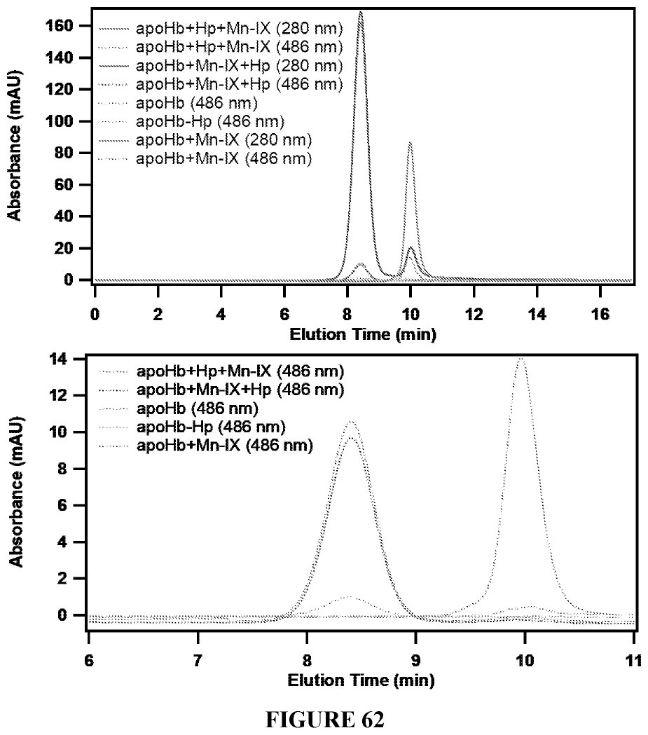

Using the Mn-IX solution, the HPLC-SEC of the complexes formed are shown in FIG. 62. The results showed that, as expected, initially, practically no absorbance was observed at 486 nm for the pure apoHb or apoHb-Hp complex. Addition of Mn-IX to apoHb led to the formation of apoHb-Mn-IX complexes, which eluted at 10 min with the apoHb protein. Furthermore, when complexed with Hp, the apoHb-Mn-IX-Hp complexes had an elution peak at 8.6 min when detecting at 468 nm, indicating that the apoHb-Mn-IX complex bound to Hp. The Mn-IX solution could also be added after formation of the apoHb-Hp complex. The elution of these species (apoHb-Hp-Mn-IX) was practically identical to addition of Mn-IX to apoHb prior to Hp binding (apoHb-Mn—IX-Hp). The smaller peak at 10 min corresponded to free apoHb which was detected at 280 nm was due to an excess of apoHb used to create the apoHb-Hp/apoHb-Mn—IX-Hp complex. Interestingly, most of the apoHb-Mn-IX bound to Hp with practically no Mn-IX eluting in the form of free apoHb-Mn-IX. This indicated that Hp had a higher affinity for apoHb-Mn-IX than to pure apoHb. This observation agreed with the previously shown higher affinity of Hp for Hb compared to apoHb. Thus, the higher affinity of Hp to apoHb-Mn-IX may be due to the higher structural similarity of apoHb-Mn-IX to Hb than pure apoHb as apoHb-Mn-IX has Mn-IX bound in the heme-binding pocket.

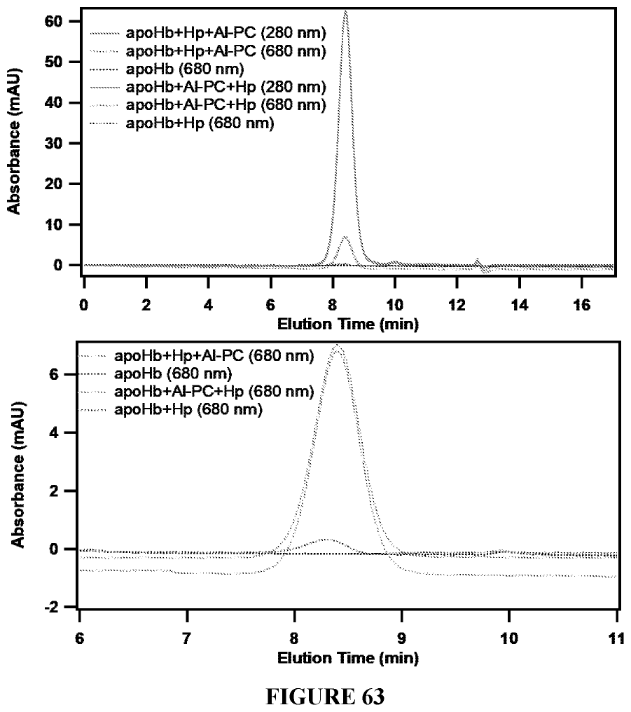

Using the Al-PC solution, the HPLC-SEC of the complexes formed are shown in FIG. 63. Similar to Mn-IX, pure apoHb or pure Hp had practically no absorbance at the wavelength for detection of the desired species (680 nm in this experiment). Yet, when A1-PC was added to the apoHb-Hp solutions, the complex eluted with an absorbance at 680 nm indicating that monomeric Al-PC was bound to the complex. This was observed when A1-PC was added to apoHb either prior to Hp binding (apoHb-PC-Hp) or after Hp binding (apoHb-Hp-PC). No difference between the spectras of apoHb-PC-Hp or apoHb-Hp-PC was noted. The small peak at around 13 min and the offset of the absorbance at 680 nm of apoHb-PC-Hp was attributed to the low absorbance of the species in these experiments.

### Example 9. Apohemoglobin-Haptoglobin Complex Alleviates Iron Toxicity in Mice with β-Thalassemia Via Scavenging of Cell-Free Hemoglobin and Heme

β-thalassemia is a genetic hemoglobin (Hb) disorder that affects millions of people world-wide. It is characterized by defective erythropoiesis and anemia, with patients suffering from low levels of abnormal red blood cells (RBCs). The continuous oxygen deficit leads to life-long blood transfusion regimens, which results in iron accumulation toxicity. Moreover, the abnormal RBCs are prone to hemolytic events that release cell-free Hb, heme, and iron, causing oxidative organ and tissue damage. In this study, β-thalassemic mice were treated with the apohemoglobin-haptoglobin (apoHb-Hp) complex for six weeks to simultaneously scavenge cell-free Hb and free heme. Animal weight and RBC parameters were measured throughout the study. Moreover, total iron levels, transferrin concentration and transferrin saturation were measured at the third and sixth week of treatment. At the end of the experiment, spleen and liver weights were measured and markers of liver function were assessed. Furthermore, the total iron content of the liver and spleen was quantified. ApoHb-Hp treatment lowered hepatosplenomegaly, and lowered markers of liver damage. Moreover, apoHb-Hp treatment lead to improved RBC levels, reduced cell fraction of reticulocytes, and prevented an increase in red-blood cell distribution width. Remarkably, apoHb-Hp treatment reduced circulating iron levels, transferrin saturation, increased overall transferrin levels, and lowered iron accumulation within the liver and spleen. These results indicate that scavenging of cell-free Hb and free heme with apoHb-Hp treatment in beta-thalassemia reduced hepatosplenomegaly, normalized RBC levels, and lowered iron accumulation. Based on these outcomes, a mechanism for iron removal via scavenging of cell-free Hb and heme was proposed. Taken together, this study demonstrated that apoHb-Hp can reduce iron toxicity and normalize RBC levels in β-thalassemic animals via scavenging of cell-free Hb and heme. Thus, apoHb-Hp may be a viable treatment strategy to normalize RBCs and iron levels in β-thalassemic patients.

Methods

Apohemoglobin Preparation. The apoHb used in this study was prepared via tangential flow filtration based on the acidic-ethanol heme-extraction procedure as previously described in the literature The heme-binding capacity of apoHb preparations was approximately 80%, with less than 1% residual heme present.

Haptoglobin Preparation. Human Hp was purified from human Cohn fraction IV (FIV) purchased from Seraplex (Pasadena, Calif.) via tangential flow filtration as previously described in the literature. The final protein solution was composed of a mixture of Hp2-1 and Hp2-2 Hp polymers, with an average MW of 400-500 kDa and >95% purity.

Animal Model and Treatment. Thalassemic mice consisted of C57BL/6 heterozygous for the Hbb β-globin gene deletion (Hbb^{td3th/}BrjK) (beta-thalassemia, Jackson Laboratory). Animals were treated q.o.d. for six weeks with the apoHb-Hp complex (Hp 22.5 mg/mL, apoHb 7.5 mg/mL, 50 μL, n=8), or vehicle (PBS, equal volume as study group, n=8) via tail vein injection. Animal body weight was monitored at alternate treatment days. Animals were sacrificed after the final dose for analysis.

Hematological Parameters. Blood samples were obtained at baseline and every two weeks by retro-orbital puncture under isoflurane (2% for maintenance, Drägerwerk AG, Lübeck, Germany). Complete blood counts (CBC) were measured on an Hemavet blood analyzer (Drew Scientific, Oxford, Conn.) and confirmed via flow cytometry on selected samples.

Serum Iron Content and Tf Saturation. Serum iron and unsaturated iron-binding capacity (UIBC) were measured in non-hemolyzed mouse serum using an iron and total iron-binding capacity (TIBC) assay according to the manufacturer's instructions (LabCorp, Burlington, N.C.). TIBC and transferrin saturation were calculated from the measured serum iron and UIBC.

Determination of Tissue Iron Content. After the experimental end point was reached, half of the mice (n=4/group) were euthanized and transcardially perfused via the aorta with PBS, and tissue non-heme iron content was determined with a colorimetric method using BPS (4,7-diphenyl-1,10-phenantroline disulfonic acid) as the chromogen. Briefly, 0.2 g of tissues were incubated overnight in a mixture of trichloroacetic (10%) and hydrochloric (4 N) acids, and 100 μl of supernatant reduced with thioglycolic acid (Sigma-Aldrich) and acetic acid-acetate buffer (pH 4.5). The ferrous iron content was determined spectrophotometrically (535 nm) with the addition of BPS and after 1 hr incubation at 37° C. The results are expressed as μg iron/g dry tissue weight.

Histology and Iron Staining. After the experimental end point was reached, the other half of the mice (n=4/group) were transcardially perfused via the aorta with PBS followed by perfusion of a fixative solution (4% paraformaldehyde in PBS). The liver and spleen were harvested and were continued to be fixed in the same fixative solution (4 hrs at 4° C.). Tissues were washed in PBS, and cryoprotected by immersion in sucrose overnight. Tissues were cut and the free-floating sections were stored in cryoprotective solution at −20° C. until processed. Iron was detected using Perl's staining for non-heme ferric iron (Fe (III)) followed by 3,3 diamino-benzidine (DAB, Sigma-Aldrich) in methanol. Iron staining was developed by incubation of tissues with DAB and hydrogen peroxide, and then transferred onto gelatin-coated slides, rinsed in PBS, counterstained with hematoxylin, dehydrated and mounted. Quantification of iron positive cells was performed with an Olympus BX51WI microscope equipped with a high-resolution digital CCD ORCA-285 camera (Hamamatsu, Hamamatsu City, Japan). Images for Perl's stained areas and Hoechst stained areas were prepared using Wasabi Imaging Software (Hamamatsu). The ratio of pixels stained for Perl's Prussian Blue in each region compared to the total cellular area of the image was calculated. Ten images were analyzed, by sections, and the results were pooled to determine the mean and SD. To indicate the colocalization of Perl's Prussian Blue and Hoechst in cells, and positive cells were counted.

Statistical Analysis. Results are presented as Tukey box plots. Some data are presented as absolute values and relative to baseline. A ratio of 1.0 signifies no change from baseline, whereas lower or higher ratios are indicative of changes proportionally lower or higher than baseline. Data analysis between groups and time points were analyzed via two-way analysis of variance (ANOVA), with Tukey post-hoc test when appropriate. Before experiments were initiated, sample sizes were calculated based on α=0.05, and power=0.9 to detect differences between primary end points (serum iron and transferrin saturation). All statistics were performed in GraphPad Prism 7 (GraphPad, San Diego, Calif.). Results were considered statistically significant if P<0.05.

Results

This study was completed in sixteen Hbb^{th3/+} mice. Eight animals were randomly assigned to each experimental group. The first experimental group was untreated, receiving only PBS (vehicle group). The second group was treated with the apoHb-Hp complex (apoHb-Hp group). All animals were confirmed positive for β-thalassemia via genotyping performed by the vendor and tolerated the experiments without signs of pain or discomfort.

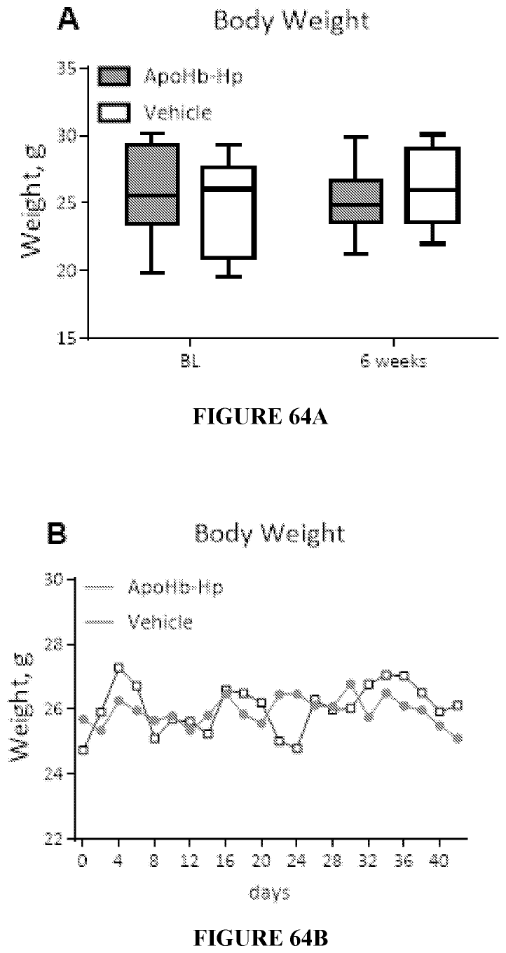

Six-weeks of apoHb-Hp treatment does not show signs of toxicity. The toxicity associated with continuous apoHb-Hp treatment was assessed by measuring the body weight of animals during the experiment. FIG. 64A shows the body weight of mice at the beginning and end of the study, while FIG. 64B shows the changes in weight of mice every four days during the study. As shown in FIG. 64, the treated and vehicle control mice showed similar weight fluctuations over the length of the study with no significant differences. Moreover, there was no significant change in body weight over the study period for both groups. Based on this analysis, the continuous apoHb-Hp treatment group did not show any signs of toxicity.

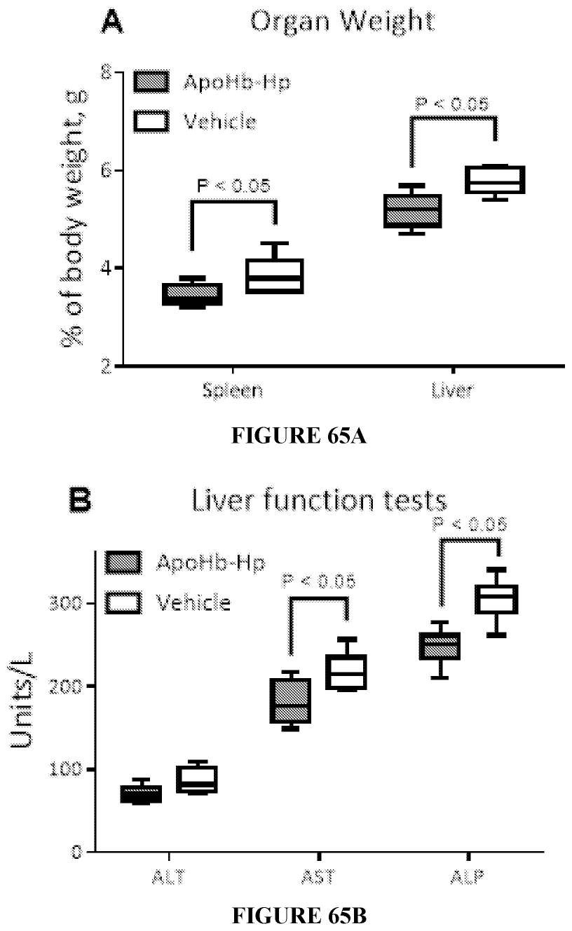

ApoHb-Hp treatment reduces splenomegaly and hepatomegaly induced by β-thalassemia. The enlargement of the spleen (splenomegaly) and liver (hepatomegaly) are common pathological traits of β-thalassemia. FIG. 65A compares the weight of the spleen and the liver at the end of the experiment for the apoHb-Hp complex treated group and vehicle control group. The spleen and liver weight for the apoHb-Hp complex treated group was significantly lower compared to the vehicle control group, suggesting that treatment with the apoHb-Hp complex prevents splenomegaly and hepatomegaly associated with β-thalassemia. FIG. 65B shows key markers of liver function [alanine amino transferase (ALT), aspartate amino transferase (AST) and alkaline phosphatase (ALP)]. AST and ALP levels were significantly decreased in the apoHb-Hp complex treated group compared to the vehicle control group. ApoHb-Hp treatment also decreased ALT values, but the difference was not significant. Based on these results, apoHb-Hp treatment lowered the levels of these liver enzymes, indicating that apoHb-Hp treatment could reduce liver damage elicited in β-thalassemia.

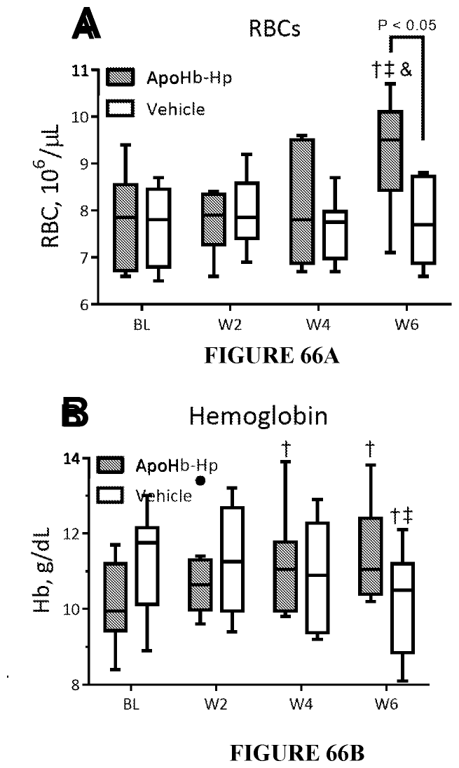

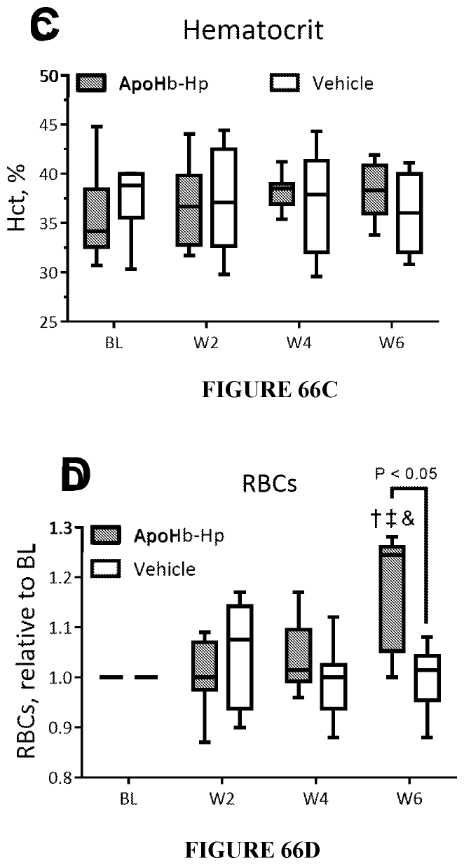

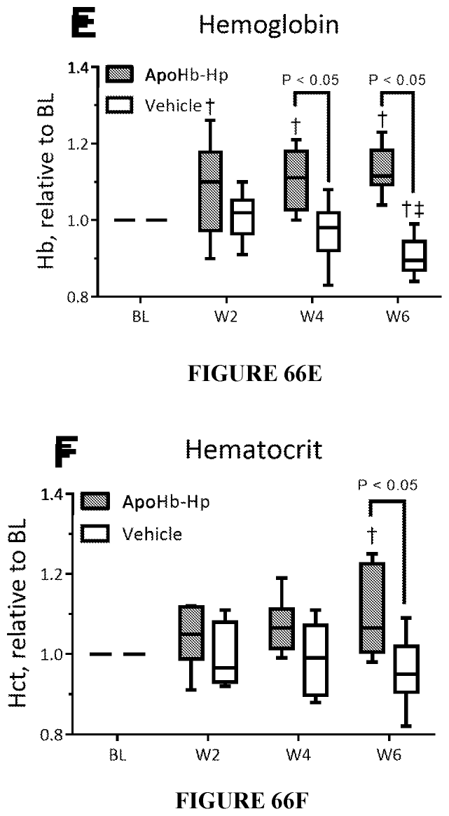

ApoHb-Hp treatment recovers RBC levels in β-thalassemic mice. FIGS. 66A, 66B, and 66C show the RBC count, total Hb concentration (tHb), and hematocrit (Hct), respectively. FIGS. 66D, 66E, and 66F show these parameters (RBC count, tHb, and Hct) normalized to baseline levels. The absolute RBC count and tHb were increased compared to baseline after six weeks of treatment with apoHb-Hp. Moreover, at the end of treatment, the apoHb-Hp group had higher absolute RBC count compared to the control group, and the control group had decreased tHb levels compared to baseline. Finally, there were no significant differences in the absolute Hct of treated and untreated groups.

When normalized to baseline, the effects of apoHb-Hp treatment were more pronounced. After six weeks of treatment, the relative RBC count, tHb level and Hct of the apoHb-Hp treated group increased compared to baseline and were significantly higher than the control group. Moreover, the relative tHb level of the apoHb-Hp treated group was significantly higher than baseline and vehicle controls at 4 and 6 weeks of treatment. Finally, the relative tHb levels of the control group decreased over the experimental study, becoming significantly lower than baseline at six weeks. These results indicate that apoHb-Hp treatment improved RBC levels, reducing the severity of anemia in β-thalassemic mice. Although the increase in tHb levels also indicated an improvement over the control, the increase could be an artifact of higher cell-free Hb retention when bound to the apoHb-Hp complex. Moreover, the relative decrease in tHb of the control group is indicative of worsening of anemia in β-thalassemic animals, which was not observed in the apoHb-Hp treated animals.

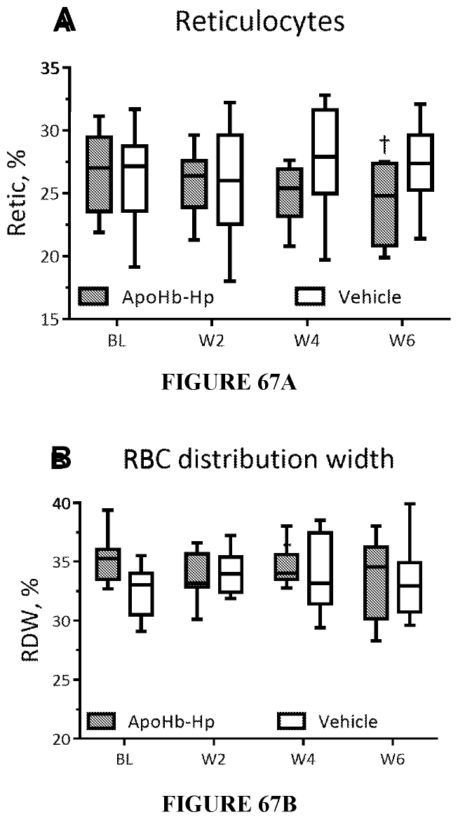

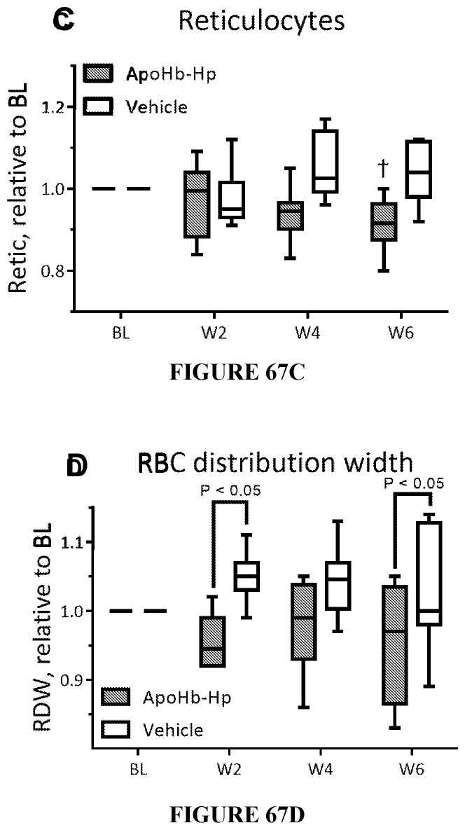

FIGS. 67A, and 67B show the percentage of reticulocytes (Retic) and percentage RBC distribution width (RDW). FIGS. 67C and 67D shows the Retic and RDW normalized to baseline. Both the absolute and relative change in Retic showed a gradual increase for the control and a gradual decrease for the apoHb-Hp treated group over time. At six weeks, the absolute and relative Retic of the apoHb-Hp treated group was significantly lower than baseline. In hemolytic anemias, the Retic levels are increased to compensate for the high rate of hemolysis. Thus, the increase in total hemoglobin, and the increase in RBCs, and the decrease in reticulocytes indicated that apoHb-Hp treatment reduced hemolysis and extended the half-life of RBCs. The absolute RDW did not show any significant changes or differences during the six-week treatment. Yet, the relative RDW demonstrated that the untreated group had a significantly higher RDW compared to the apoHb-Hp treated animals at the second and sixth week of treatment. The higher RDW is indicative of the presence of various shaped and deformed RBCs which is consistent with thalassemia. Thus, apoHb-Hp treatment allowed for a normalization of RBC shape in the circulation.

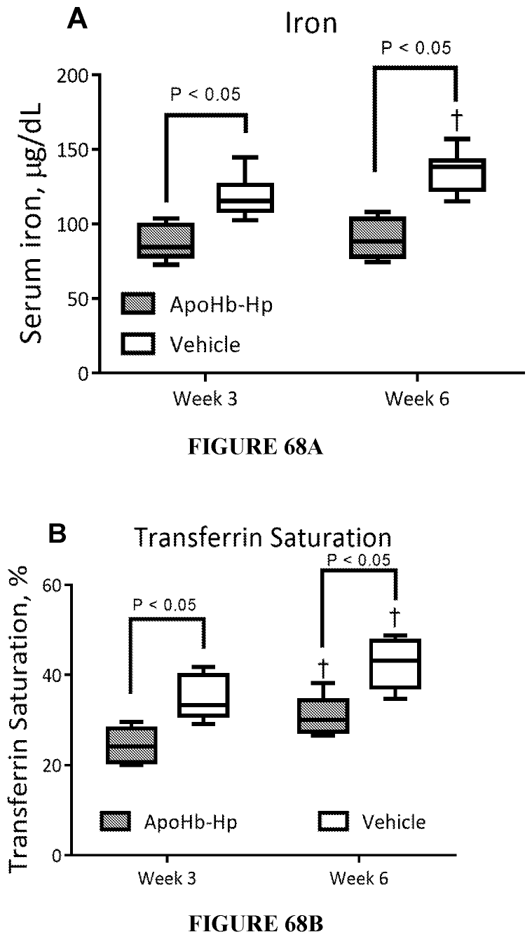

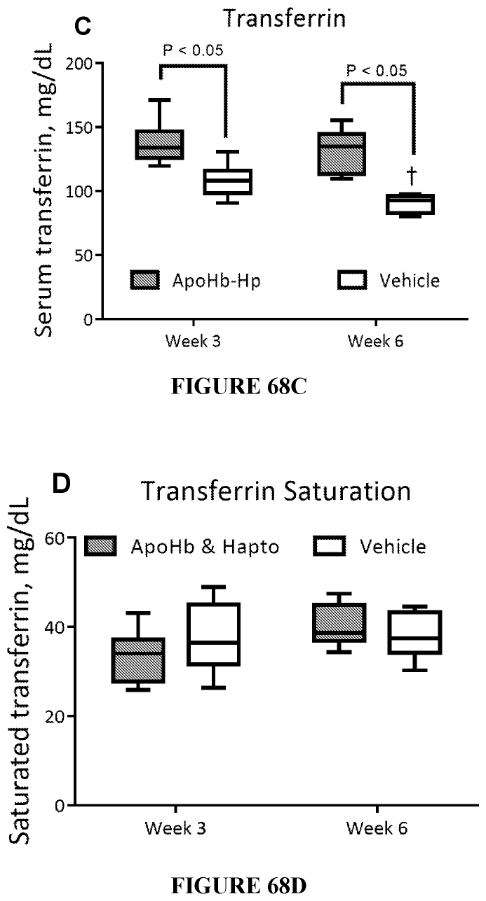

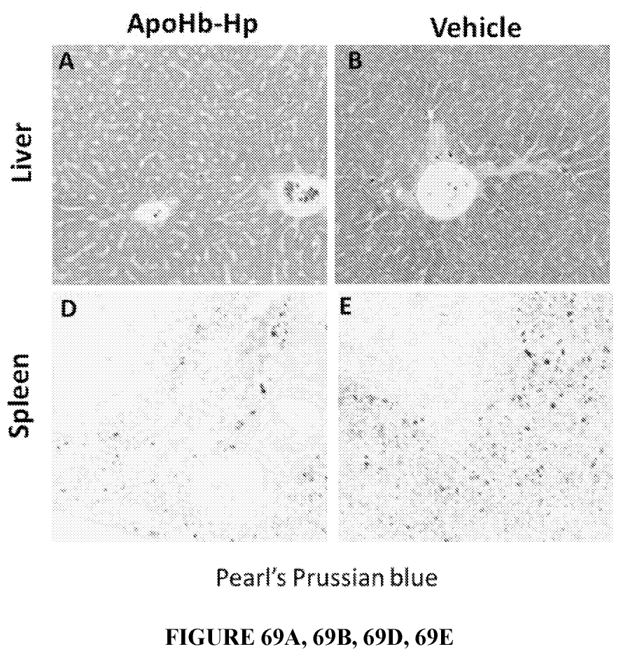

ApoHb-Hp treatment lowers total iron levels and increases serum iron binding capacity of transferrin. FIG. 68A shows the serum iron concentration, while FIG. 68B shows the transferrin (Tf) saturation, and FIG. 68C shows the serum Tf concentration. The serum iron concentration and Tf saturation in the apoHb-Hp treated group was significantly lower than the control after three weeks and six weeks of treatment. Moreover, Tf levels were significantly higher in the apoHb-Hp treated group. The results also showed that serum iron significantly increased comparing the third and sixth week of the control group, but not for the apoHb-Hp group. Furthermore, Tf levels significantly decreased comparing the third and sixth week of treatment for the control group. Tf saturation increased for both the apoHb-Hp and control treated groups comparing the third and sixth week. These results indicated that apoHb-Hp treatment lowered the free iron load in circulation and increased the total iron binding capacity of the serum, reducing the characteristic hemochromatosis associated with thalassemia.

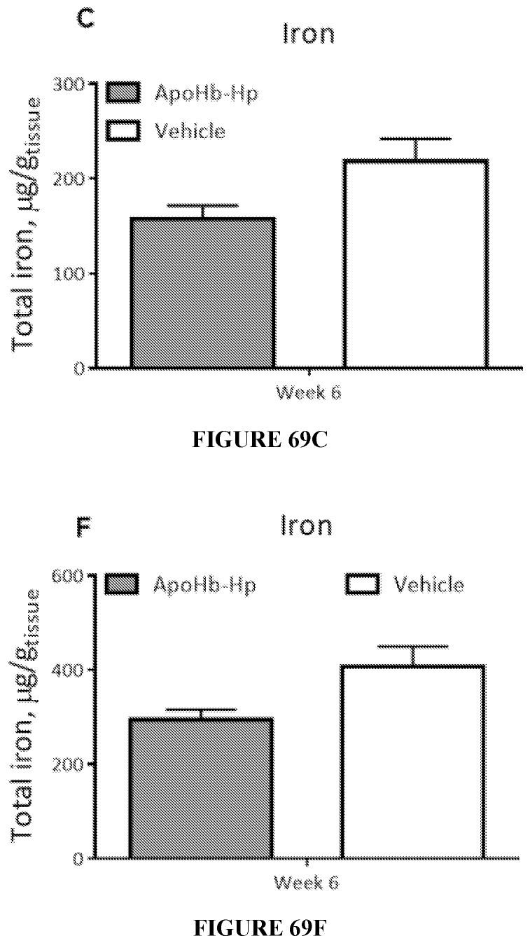

ApoHb-Hp treatment reduces iron accumulation in the liver and spleen. FIG. 69 shows iron staining in the liver and spleen after six weeks of treatment. There was only a slight decrease in iron staining in images of both the liver (FIG. 69AB) and spleen (FIG. 69DE) for the apoHb-Hp treated animals. When total iron was quantified (FIGS. 69C and 69F), this decrease became more apparent, but the difference was not statistically significant. This data corroborates the results shown previously that apoHb-Hp treatment reduces iron accumulation in β-thalassemic mice.

Discussion

ApoHb-Hp treatment reduced spleen size compared to untreated mice (vehicle control) who most likely suffered from splenomegaly (i.e. enlarged spleen) as it is a common characteristic in β-thalassemic patients. The enlargement of the spleen is generally a result of the increased accumulation of RBCs and iron. This occurs since one of the spleen's primary functions is to remove aged, damaged, or abnormal RBCs from the circulation. The spleen accomplishes this function via its splenic cords in which young, flexible RBCs pass through the epithelial cells of the splenic cord, while senescent RBCs are trapped and phagocytosed by tissue resident macrophages. When presented with an abnormally large number of senescent RBCs, the spleen can become clogged, preventing all RBC passage, and thus RBCs accumulate, contributing to splenomegaly. Moreover, the accumulation of senescent RBCs in the spleen increases macrophage levels which can further block cellular passage, augment splenomegaly, and contribute to increased rates of splenic hemolysis. Notably, it has also been shown that vaso-occlusion induced by hemolysis can increase capture of RBCs within the spleen. Thus, by reducing the hemolysis induced side-effects of β-thalassemia, apoHb-Hp treatment may reduce RBC capture in the spleen, leading to the observed decrease in the size of the organ.

Reduction of splenomegaly is vital for thalassemic patients as it lowers the rate of RBC turnover, by reducing the rate of RBC destruction. The bone marrow in β-thalassemic patients cannot produce enough RBCs to maintain demand, thus causing other organs, such as the spleen and liver, to create RBCs via extramedullary hematopoiesis. Thus, in addition to facilitating the accumulation of damaged RBCs in the spleen, the organ is also stressed due to the need to produce RBCs. These events can lead to a hyperactive spleen (hypersplenism). In hypersplenism, the spleen destroys RBCs at an even faster rate, exacerbating iron toxicity to the point where chelation therapy cannot mitigate it. Treatment at severe stages of hypersplenism primarily consists of splenectomy, which can make patients highly susceptible to infection and sepsis. As shown in the RBC data, apoHb-Hp treatment lead to increased RBC count, lower reticulocyte counts, and lower RBC distribution width. The increase in RBC count and lower RBC distribution width indicates that the RBCs had an increased lifespan in the circulation. Moreover, the decrease in reticulocyte count suggested a suppression of bone marrow erythropoiesis and extramedullary hemoatopoiesis. In addition to directly improving RBC levels, it is interesting to note that apoHb-Hp treatment reduced iron levels within the spleen, liver, and circulation (serum iron and transferrin saturation). These results further corroborate that the overall rate of hemolysis within β-thalassemic mice was reduced given that CD163 expression (receptor responsible for uptake of the Hb-Hp complex) primarily occurs in the liver and spleen, which are the regions of highest RBC/iron accumulation. Thus, these results indicated that apoHb-Hp could reduce the destruction rate of RBCs in β-thalassemic mice, lowering the demand for RBC production and, therefore, aiding in further reduction of splenomegaly, while also normalizing RBC levels.

Lowering the rate of erythropoiesis in β-thalassemia is important, since a common effect of the severe anemia in β-thalassemic patients is inhibition of the hormonal regulator of iron metabolism, hepcidin. Hepcidin binds to ferroportin and induces its intracellular degradation. This mechanism protects tissues from iron overload as it prevents iron efflux from iron-releasing cells such as macrophages and duodenal enterocytes (responsible for absorption of iron in the intestine). In normal physiological states, high circulatory levels of iron and inflammation stimulate hepcidin expression to prevent iron overload in tissues by restricting iron to macrophages and duodenal enterocytes. However, during thalassemia, the continuous expression of erythropoietin blocks hepcidin expression, leading to high ferroportin levels and subsequent iron overload due to high levels of iron absorption. Since apoHb-Hp treatment normalized RBC levels and reduced the erythropoietic drive, the treatment likely normalized hepcidin function, leading to regularization of iron levels in the liver and spleen, and the observed reduction in transferrin iron saturation.

In addition to splenomegaly, β-thalassemic patients commonly suffer from hepatomegaly (enlarged liver). Under normal conditions, the liver stores a majority of iron, but the excess iron pool in β-thalassemic patients is associated with cellular toxicity and leads to enlargement of the liver. Furthermore, similar to splenomegaly, extramedullary hematopoiesis can also occur in the liver, further increasing liver size. Moreover, the chronic hemolytic environment associated with β-thalassemia can lead to excess heme uptake within the liver, resulting in liver damage and congestion due to inflammation and oxidative stress. This causes chronic liver injury and fibrosis in β-thalassemic patients. One of the consequences from extensive liver injury and fibrosis is portal hypertension, which leads to further splenomegaly and increases the risk of hypersplenism. Markers of dysregulated liver function include AST which, when elevated, is indicative of hemolysis or defective erythropoiesis, which is consistent with thalassemia. In this study, apoHb-Hp treatment reduced liver size and reduced the levels of liver damage markers (AST and ALP) indicating that treatment was capable of reducing hemolysis-mediated damage to the organ.

As mentioned previously, the primary cause of splenomegaly in β-thalassemic patients is the accumulation of large quantities of abnormal RBCs in the spleen, which can congest the splenic cords. In β-thalassemia, these abnormalities in RBCs are not only derived from the genetic deficiency in β-globin production, but the hemolytic species (i.e. Hb, heme, and iron) can also lower the lifespan of RBCs. This occurs due to exposure to high levels of ROS, resulting oxidative stress of the RBC membrane, leading to premature macrophage capture. Moreover, liver disease can also lead to abnormal RBCs as the liver controls lipid metabolism. Thus, the damaged liver and excess hemolytic species (i.e. Hb, heme, and iron) can lead to abnormal RBCs, further increasing congestive splenomegaly and anemia.

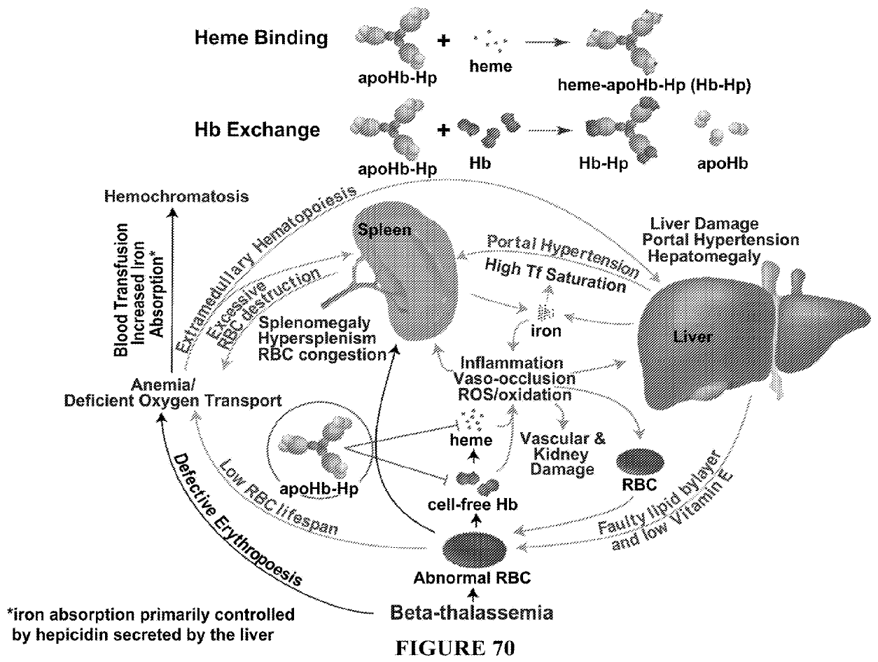

Based on the results shown in this study and the previously described mechanism of β-thalassemia toxicity, a mechanism of action for apoHb-Hp treatment was proposed and is illustrated in FIG. 70.

Consistent with the data presented here and the proposed mechanism of action for apoHb-Hp, previous studies have demonstrated that Hp and Hpx double knock-out mice suffer from splenomegaly and severe liver inflammation and fibrosis. In these prior studies, splenomegaly was primarily caused by the accumulation of RBCs, which was attributed to free heme, leading to vascular alterations that caused adhesion of RBCs to the endothelium. Additionally, Hpx treatment has been shown to reduce liver damage and endothelial function in β-thalassemia and sickle-cell disease. However, Hpx treatment alone did not improve RBC levels in β-thalassemic mice, unlike the apoHb-hp treatment implemented in our study. Moreover, unlike the results shown in our study, one month of Hpx treatment alone increased iron levels in the liver in β-thalassemic mice. While this increase in liver weight would be favorable, as it suggests that the excess-heme was directed to cells specialized in the detoxification of heme, the study demonstrated that Hpx treatment alone could not prevent the morbidities of β-thalassemia.

Conclusion

This study postulated the use of a specific protein complex, apoHb-Hp, to alleviate the toxicities observed in β-thalassemic mice over a six-week treatment period. Treatment with apoHb-Hp did not show any obvious signs of toxicity as indicated by the steady body weight of the animals throughout the study. Furthermore, our data conclusively show a positive outcome in reducing the toxicity that is normally observed in the liver and spleen in patients with β-thalassemia. Moreover, RBC levels were shown to improve compared to untreated mice, which was attributed to normalization of the rate of RBC destruction. Taken together, these data show that the apoHb-Hp complex promotes a positive outcome for animals suffering from β-thalassemia.

The compositions, systems, and methods of the appended claims are not limited in scope by the specific compositions, systems, and methods described herein, which are intended as illustrations of a few aspects of the claims. Any compositions, systems, and methods that are functionally equivalent are intended to fall within the scope of the claims. Various modifications of the compositions, systems, and methods in addition to those shown and described herein are intended to fall within the scope of the appended claims. Further, while only certain representative compositions, systems, and method steps disclosed herein are specifically described, other combinations of the compositions, systems, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A pharmaceutical composition comprising an apohemoglobin-haptoglobin complex.

2. The composition of claim 1, wherein the apohemoglobin-haptoglobin complex comprises apohemoglobin and haptoglobin at a weight ratio of from 1:1 to 1:3.

3. The composition of any of claims 1-2, wherein the haptoglobin has an average molecular weight of from 80 kDa to 1,000 kDa, such as from 100 kDa to 1,000 kDa.

4. The composition of any of claims 1-3, wherein the apohemoglobin is characterized by a residual Soret peak having a maximum absorption at 411-417 nm, such as 412 nm.

5. The composition of any of claims 1-4, wherein the composition further comprises an additional protein chosen from transferrin, hemopexin, haptoglobin, apohemoglobin, or a combination thereof.

6. A method of treating hemolysis in a subject comprising administering the composition of any of claims 1-5.

7. The method of claim 6, wherein the hemolysis is associated with sickle cell anemia, malaria, a red blood cell transfusion, thalassemia, an autoimmune disorder, bone marrow failure, an infection, a surgical procedure, a burn, an acute lung injury, sepsis, organ perfusion, the administration of a pharmaceutical agent such as drug-induced hemolytic anemia, the administration of radiation therapy, or a combination thereof.

8. A method of treating a disease characterized by elevated levels of iron, elevated levels of heme, elevated levels of hemoglobin, or a combination thereof, in a subject, the method comprising administering the composition of any of claims 1-5.

9. A method of stabilizing a composition comprising red blood cells, the method comprising adding the composition of any of claims 1-5 to the composition comprising red blood cells.

10. A method of improving safety of a hemoglobin-based substitute comprising adding the composition of any of claims 1-5 to the hemoglobin-based substitute or co-infusing the composition of any of claims 1-5 with the hemoglobin-based substitute.

11. A method of treating a wound in a subject, the method comprising contacting the wound or a region proximate thereto with the composition of any of claims 1-5.

12. The method of claim 11, wherein contacting the wound or a region proximate thereto comprises topically applying the composition of any of claims 1-5 to the wound or a region proximate thereto.

13. The method of claim 11, wherein contacting the wound or a region proximate thereto comprises injecting the composition of any of claims 1-5 into the wound or a region proximate thereto.

14. A method for treating a microbial infection in a subject, the method comprising administering a therapeutically effective amount of the composition of any of claims 1-5 to the subject.

15. The method of claim 14, wherein the composition is administered locally to a site of the microbial infection.

16. The method of any of claims 14-15, wherein the microbial infection comprises an antibiotic-resistant bacterial strain.

17. The method of any of claims 14-16, wherein the composition of any of claims 1-5 is administered in amount effective to decrease favorability of bacterial growth at a site of the microbial infection.

18. A method of reducing hemolysis in an organ or tissue transplanted or to be transplanted, the method comprising perfusing the organ or tissue with the composition of any of claims 1-5.

19. The method of claim 18, wherein the organ or tissue is perfused ex vivo with red blood cells or a hemoglobin-based blood substitute.

20. A pharmaceutical composition comprising an apohemoglobin-haptoglobin complex and an active agent coordinated thereto.

21. The composition of claim 20, wherein the active agent comprises a diagnostic agent.

22. The composition of claim 21, wherein the diagnostic agent comprises an imaging agent.

23. The composition of claim 22, wherein the imaging agent comprises an MRI contrast agent.

24. The composition of any of claims 21-23, wherein the active agent comprises a porphyrin-based imaging agent.

25. The composition of any of claims 21-23, wherein the active agent comprises a phthalocyanine-based imaging agent.

26. The composition of claim 20, wherein the active agent comprises a therapeutic agent.

27. The composition of claim 26, wherein the active agent comprises a porphyrin-based photodynamic therapy agent.

28. The composition of claim 26, wherein the active agent comprises a phthalocyanine-based photodynamic therapy agent.

29. The composition of claim 26, wherein the therapeutic agent comprises an agent to treat or prevent a disease or disorder associated with the overexpression of CD163.

30. The composition of claim 26, wherein the therapeutic agent comprises an agent to treat or prevent a disease which involves macrophages or monocytes

31. The composition of claim 26, wherein the therapeutic agent comprises an agent to treat or prevent hemolytic anemia or other conditions characterized by or associated with hemolysis.

32. The composition of any of claims 26-31, wherein the therapeutic agent comprises an anti-cancer agent, an anti-inflammatory agent, an agent that treats or prevents an infection, or a combination thereof.

33. The composition of any of claims 20-32, wherein the apohemoglobin-haptoglobin complex comprises apohemoglobin and haptoglobin at a weight ratio of from 1:1 to 1:3.

34. The composition of any of claims 20-33, wherein the haptoglobin has an average molecular weight of from 80 kDa to 1,000 kDa, such as from 100 kDa to 1,000 kDa.

35. The composition of any of claims 20-34, wherein the apohemoglobin is characterized by a residual Soret peak having a maximum absorption at 411-417 nm, such as 412 nm.

36. The composition of any of claims 20-35, wherein the composition further comprises an additional protein chosen from transferrin, hemopexin, haptoglobin, apohemoglobin, or a combination thereof.

37. The composition of any of claims 20-36, wherein the active agent is non-covalently associated with the apohemoglobin-haptoglobin complex.

38. The composition of any of claims 20-36, wherein the active agent is covalently associated with the apohemoglobin-haptoglobin complex.

39. The composition of claim 38, wherein the active agent is covalently bound to the apohemoglobin.

40. The composition of claim 38, wherein the active agent is covalently bound to the haptoglobin.

41. The composition of any of claims 38-40, wherein active agent is covalently bound via a cleavable linker, such as a hydrolysable linker.

42. The composition of any of claims 20-36, wherein the active agent is covalently bound to an apohemoglobin binding molecule associated with the apohemoglobin in the apohemoglobin-haptoglobin complex.

43. The composition of claim 42, wherein the apohemoglobin binding molecule comprises heme.

44. The composition of claims 42-43, wherein the active agent is covalently bound via a cleavable linker, such as a hydrolysable linker.

45. The composition of claim 44, wherein the cleavable linker is pH sensitive.

46. The composition of claim 45, wherein the cleavable linker is cleaved at endosomal pH.

47. A method of treating a disease in a subject in need thereof comprising administering to the subject a therapeutically effective amount of a pharmaceutical composition defined by any of claims 26-46.

48. The method of claim 47, wherein the disease comprises a disease characterized by the overexpression of CD163.

49. The method of any of claims 47-48, wherein the disease comprises cancer, liver cirrhosis, type 2 diabetes, macrophage activation syndrome, Gaucher's disease, sepsis, HIV infection, and rheumatoid arthritis.

50. The method of claim 49, wherein the disease comprises breast cancer.

51. The method of claim 49, wherein the disease comprises Hodgkin Lymphoma.

52. The method of any of claims 47-51, wherein the disease involves macrophages or monocytes.

53. The method of claim 52, wherein the disease comprises heart disease, HIV infection, cancer, fibrotic diseases (e.g., cystic fibrosis), asthma, inflammatory bowel disease, rheumatoid arthritis, or a disease in which macrophages or monocytes function as hosts for intracellular pathogens such as malaria, tuberculosis, leishmaniasis, chikungunya, adenovirus, Legionnaires' disease, coronavirus (e.g., SARS-CoV-2), or infections caused by bacteria in the genus Brucella.

54. The method of any of claims 47-53, wherein the disease comprises hemolytic anemia and other condition characterized by or associated with hemolysis.

55. The method of claim 54, wherein the disease comprises hemolysis associated with sickle cell anemia, malaria, a red blood cell transfusion, thalassemia, an autoimmune disorder, bone marrow failure, an infection, a surgical procedure, a burn, an acute lung injury, sepsis, organ perfusion, the administration of a pharmaceutical agent such as drug-induced hemolytic anemia, the administration of radiation therapy, or a combination thereof.

56. The method of any of claims 47-55, wherein the composition defined by any of claims 26-46 is administered as an immunotherapy targeting CD163+ macrophages and monocytes.

57. The method of any of claims 47-56, wherein the active agent comprises a TLR agonist, such as a TLR7 agonist or a TLR9 agonist, that is carried by the complex for receptor mediated uptake and immune activation within the endosome.

58. The method of claim 57, wherein the active agent comprises a TLR7 agonist.

59. The method of claim 58, wherein the TLR7 agonist comprises an imidazoquinoline such as imiquimod.

60. The method of any of claims 47-56, wherein the active agent comprises a cytokine.

61. The method of any of claims 47-60, wherein the apohemoglobin-haptoglobin complex and an active agent coordinated thereto is administered in combination with an immunotherapy agent, such as an immune checkpoint inhibitor.

62. The method of claim 61, wherein the immune checkpoint inhibitor is an anti-PD1 or anti-PDL1 monoclonal antibody.

63. The method of claim 61, wherein the immune check-point inhibitor is an anti-CTLA4 monoclonal antibody.

64. The method of any of claims 47-63, wherein the active agent comprises a HO-1 enzyme agonist or a HO-1 enzyme antagonist.

65. The method of claim 64, wherein the active agent comprises a protoporphyrin IX complex, such as zinc protoporphyrin IX or tin protoporphyrin IX, that is a HO-1 antagonist.

66. The method of any of claims 64-65, wherein the active agent comprises a HO-1 enzyme antagonist, and wherein the disease comprises cancer.

67. The method of claim 64, wherein the active agent comprises a HO-1 enzyme agonist, and wherein the disease comprises an inflammatory condition.

68. The method of any of claims 64-67, wherein the apohemoglobin-haptoglobin complex and an active agent coordinated thereto is administered in combination with a reactive oxygen species inducer, such as cisplatin, doxorubicin and 5-fluorouracil.

69. The method of claim 64, wherein the active agent comprises a HO-1 enzyme agonist, and wherein the disease comprises cellular iron accumulation and ferroptosis.

70. The method of claim 69, wherein the apohemoglobin-haptoglobin complex and the HO-1 enzyme agonist coordinated thereto are administered to treat cancer.

71. The method of claim 69, wherein the apohemoglobin-haptoglobin complex and the HO-1 enzyme agonist coordinated thereto are administered in combination with a ferropototic agent, such as Bay117085 or withaferin A.
